SEQUENCE LISTING (25 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 232 aa, Mus musculus
MSTESMTRDVELAEEALPQKMGGFQNSRRCLCLSLFSFLLVAGATTLFCLLNFGVIGPQRDEKFPNGLPLISSMAQTLSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 3 - protein, 8 aa, synthetic construct
GFIFSNHW

SEQ ID NO: 4 - protein, 10 aa, synthetic construct
IRSKSINSAT

SEQ ID NO: 5 - protein, 11 aa, synthetic construct
SRNYYGSTYDY

SEQ ID NO: 6 - protein, 5 aa, synthetic construct
NHWMN

SEQ ID NO: 7 - protein, 19 aa, synthetic construct
EIRSKSINSATHYAESVKG

SEQ ID NO: 8 - protein, 9 aa, synthetic construct
NYYGSTYDY

SEQ ID NO: 9 - protein, 450 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 6 aa, synthetic construct
QFVGSS

SEQ ID NO: 11 - skipped

SEQ ID NO: 12 - protein, 9 aa, synthetic construct
QQSHSWPFT

SEQ ID NO: 13 - protein, 11 aa, synthetic construct
RASQFVGSSIH

SEQ ID NO: 14 - protein, 7 aa, synthetic construct
YASESMS

SEQ ID NO: 15 - protein, 9 aa, synthetic construct
QQSHSWPFT

SEQ ID NO: 16 - protein, 214 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 16 aa, synthetic construct
GWSCIILFLVATATGV

SEQ ID NO: 18 - protein, 18 aa, synthetic construct
GWSCIILFLVATATGVHS

SEQ ID NO: 19 - protein, 233 aa, synthetic construct
MGWSCIILFLVATATGVHSDILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 469 aa, synthetic construct
MGWSCIILFLVATATGVHSEVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 52 bp, synthetic construct
  source 1..52: mol_type = other DNA
ggctggtcctgcatcatcctgtttctggtggccaccgccaccggcgtgcact

SEQ ID NO: 22 - DNA, 54 bp, synthetic construct
  source 1..54: mol_type = other DNA
ggctggtcctgcatcatcctgtttctggtggccaccgccaccggcgtgcactcc

SEQ ID NO: 23 - DNA, 718 bp, synthetic construct
  source 1..718: mol_type = other DNA
gctagcgaattccaccatgggctggtcctgcatcatcctgtttctggtggccaccgccaccggcgtgcactccgacatcctgctgacccagtcccccgccatcctgtccgtgtctcccggcgagcgggtgtccttctcctgccgggcctcccagttcgtgggctcctccatccactggtatcagcagcggaccaacggctcccctcggctgctgatcaagtacgcctccgagtccatgtccggcatcccttcccggttctccggctccggctctggcaccgacttcaccctgtccatcaacaccgtggagtccgaggatattgccgactactactgccagcagtcccactcctggcctttcaccttcggctccggcaccaacctggaggtgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 24 - DNA, 1450 bp, synthetic construct
  source 1..1450: mol_type = other DNA
gctagcgatatcgaattcttttcttttgttttgtttccaccatgggctggtcctgcatcatcctgtttctggtggccaccgccaccggcgtgcactccgaggtgaagctggaggagtctggcggcggactggtgcagcctggcggctccatgaagctgtcctgcgtggcctccggcttcatcttctccaaccactggatgaactgggtgcgccagtcccctgagaagggcctggagtgggtggccgagatccggtccaagtccatcaactccgccacccactacgccgagtccgtgaagggccggttcaccatctcccgggacgactccaagtccgccgtgtacctgcagatgaccgacctgcggaccgaggacaccggagtgtactactgctcccggaactactacggctccacctacgactactggggccagggcaccacactgaccgtgagctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccgcaacaccaaggtggacaagaaagtcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagtaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 25 - protein, 5 aa, synthetic construct
HHHHH

(19) United States
(12) Patent Application Publication
(10) Pub. No.: US 2026/0042831 A1
(43) Pub. Date: Feb. 12, 2026

(54) METHODS OF TREATING SARCOIDOSIS WITH A TNF-A ANTIBODY AND ASSOCIATED COMPOSITIONS AND METHODS

(71) Applicant: XENTRIA, INC., Chicago, IL (US)

(72) Inventors: Kirsten ANDERSON, Seattle, WA (US); Thomas MATTHEWS, Chicago, IL (US); Tom SHEA, Park Ridge, IL (US); Noopur SINGH, Chicago, IL (US)

(21) Appl. No.: 19/133,859

(22) PCT Filed: Dec. 8, 2023
(86) PCT No.: PCT/US2023/083191

§ 371 Date: May 29, 2025

Related U.S. Application Data
(60) Provisional application No. 63/431,607, filed on Dec. 9, 2022.
(60) Provisional application No. 63/487,060, filed on Feb. 27, 2023.

(51) Int. Cl. C07K 16/24; A61K 39/00; A61P 37/06

(52) U.S. Cl. CPC C07K 16/241; A61P 37/06; A61K 2039/505; A61K 2039/545; C07K 2317/33; C07K 2317/41; C07K 2317/732; C07K 2317/92

(57) ABSTRACT

Provided are compositions comprising an anti-TNFα antibody sialylated with one or more N-acetylneuraminic acid molecules, and associated methods. In some embodiments, provided are methods for treating or preventing sarcoidosis and/or granuloma size or formation in patients in need thereof.

### CROSS-REFERENCE TO RELATED APPLICATION(S)

This application claims the benefit of U.S. Provisional Patent Application No. 63/431,607, filed on Dec. 9, 2022, and of U.S. Provisional Patent Application No. 63/487,060, filed on Feb. 27, 2023. The contents of each of these provisional applications are incorporated by reference in their entirety.

### SEQUENCE LISTING INCORPORATED BY REFERENCE

This application contains an ST.26 compliant Sequence Listing, which is submitted concurrently in xml format via Patent Center and is hereby incorporated by reference in its entirety. The .xml copy, created on Dec. 7, 2023, is named 140505-8004WO00_SL.xml and is 0.026 MB (25.2 KB) (25,856 bytes) in size.

### BACKGROUND

Sarcoidosis is a chronic, multisystem inflammatory disorder of unknown etiology that is characterized by the presence of non-caseating epithelioid granulomas, accompanied by infiltration of mononuclear cells and destruction of microarchitecture. This rare disease affects the skin, eye, heart, and central nervous system, and >90% of cases involve the lungs. Subjects with lung manifestations may progress to loss of lung function, and immune-modulating therapy may be beneficial in these cases. Current treatment options are suboptimal due to adverse side effects, limited efficacy, or inability to access the most effective treatments.

Macrophage-derived tumor necrosis factor α (TNFα) participates in the induction and maintenance of granulomas, and high levels of TNFα (and high levels of TNFα released from alveolar macrophages) seem to correlate with disease progression. In chronic sarcoidosis, prevention of granuloma formation and limitation of tissue injury and fibrosis are the goals of therapy. In some cases, these may be achieved through targeted immunosuppression of pro-inflammatory cytokines such as TNFα. Infliximab is a monoclonal antibody (mAb) targeting TNFα that is being used off-label to treat sarcoidosis. In a randomized controlled study of subjects with chronic refractory pulmonary sarcoidosis, low- and high-dose intravenous (IV) infusion of infliximab produced a 2.5% increase in predicted forced vital capacity at 24 weeks. Notably, other TNF inhibitors have been shown to be ineffective for the treatment of sarcoidosis or may paradoxically cause sarcoidosis-like drug reactions.

First-line agents include corticosteroids (tapered over time), followed by second- and third-line immunomodulatory agents, which are carefully selected to mitigate side effects, particularly related to systemic steroid exposure. Although corticosteroids are effective in resolving the granulomatous inflammation in sarcoidosis, up to 20% of subjects treated with systemic steroid therapy continue to exhibit a persistent granulomatous inflammatory process, with progression to irreversible tissue remodeling and fibrosis.

In subjects with advanced or severe refractory sarcoidosis who do not respond to conventional immunosuppressive treatment, TNFα inhibitors are recommended. However, there has been limited pharmacokinetic analysis of TNFα inhibitor use in sarcoidosis. As such, there remains a need to conduct dose range finding to determine a safe and efficacious dose regimen for the sarcoidosis population.

### SUMMARY

In some aspects, the present technology comprises methods of treating or reducing sarcoidosis in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every four weeks for a first period of time.

In some aspects, the present technology comprises methods of treating or reducing sarcoidosis in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every two weeks for a first period of time.

In some aspects, the present technology comprises methods of reducing granuloma size or granuloma formation in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every four weeks for a first period of time.

In some aspects, the present technology comprises methods of reducing granuloma size or granuloma formation in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every two weeks for a first period of time.

In some aspects, the present technology comprises methods of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules; the first dose comprising about 2 mg/kg of the composition administered once every four weeks for a first period of time; and the second dose comprising (i) about 2 mg/kg of the composition administered once every two weeks, or (iii) about 4 mg/kg of the composition administered once every 4 weeks, for a second period of time.

In some aspects, the present technology comprises methods of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules; the first dose comprising about 2 mg/kg of the composition administered once every four weeks for a first period of time; and the second dose comprising (i) about 2 mg/kg of the composition administered once every two weeks, or (iii) about 4 mg/kg of the composition administered once every 4 weeks, for a second period of time.

In some aspects, the present technology comprises compositions comprising an anti-TNFα antibody sialylated with one or more N-acetylneuraminic acid molecules for use in a method of treating or reducing sarcoidosis in a subject in need thereof relative to a control, wherein the composition is administered as a first dose of about 0.5 mg/kg to about 6.0 mg/kg about once every four weeks for a first period of time.

In some aspects, the present technology comprises compositions comprising an anti-TNFα antibody sialylated with one or more N-acetylneuraminic acid molecules for use in a method of treating or reducing sarcoidosis in a subject in need thereof relative to a control, wherein the composition is administered as a first dose of about 0.5 mg/kg to about 6.0 mg/kg about once every two weeks for a first period of time.

In some aspects, the present technology comprises compositions comprising an anti-TNFα antibody sialylated with one or more N-acetylneuraminic acid molecules for use in a method of reducing granuloma size or granuloma formation in a subject in need thereof relative to a control, wherein the composition is administered as a first dose of about 0.5 mg/kg to about 6.0 mg/kg about once every four weeks for a first period of time.

In some aspects, the present technology comprises compositions comprising an anti-TNFα antibody sialylated with one or more N-acetylneuraminic acid molecules for use in a method of reducing granuloma size or granuloma formation in a subject in need thereof relative to a control, wherein the composition is administered as a first dose of about 0.5 mg/kg to about 6.0 mg/kg about once every two weeks for a first period of time.

In some embodiments, the first period of time is about 6 weeks to about 24 weeks.

In some embodiments, the first dose is about 2 mg/kg of the composition.

In some embodiments, the first dose is about 4 mg/kg of the composition.

In some embodiments, the methods further comprise administering to the subject a second dose of the composition for second period of time.

In some embodiments, the second period of time is about 6 weeks to about 16 weeks.

In some embodiments, wherein the second dose comprises about 1 mg/kg to about 5 mg/kg.

In some embodiments, the second dose is about two times greater than the first dose.

In some embodiments, the administration frequency of the second dose comprises the same administration frequency of the first dose.

In some embodiments, the first dose and the second dose are each administered about once every four weeks.

In some embodiments, the second dose is about the same dose as the first dose.

In some embodiments, the first dose and the second dose are about 2 mg/kg.

In some embodiments, the administration frequency of the second dose is about two times greater than the administration frequency of the first dose.

In some embodiments, the first dose is administered about once every four weeks and the second dose is administered about once every two weeks.

In some embodiments, the anti-TNFα antibody comprises an asparagine-linked glycosylation site within a constant Fc region on at least one of the heavy chains.

In some embodiments, the anti-TNFα antibody is sialylated at Asn 297.

In some embodiments, the anti-TNFα antibody comprises no N-Glycolylneuraminic acid (Neu5Gc).

In some embodiments, the anti-TNFα antibody is an immunoglobulin G (IgG) antibody.

In some embodiments, the IgG antibody is an IgG1 antibody.

In some embodiments, the IgG1 antibody comprises a kappa isotype.

In some embodiments, the anti-TNFα antibody is a chimeric antibody.

In some embodiments, the anti-TNFα antibody comprises a C-terminal lysine on at least one of the heavy chains.

In some embodiments, the C-terminal lysine is not clipped.

In some embodiments, the composition is administered by an infusion.

In some embodiments, the infusion is an intravenous (IV) infusion.

In some embodiments, the infusion is administered over a period of at least about 2 hours.

In some embodiments, the subject receives a cumulative dose of about 6 mg/kg, 8 mg/kg, or 16 mg/kg of the composition during the first period of time.

In some embodiments, the subject receives a cumulative dose of about 10 mg/kg to about 16 mg/kg of the composition during the second period of time.

In some embodiments, the subject receives a cumulative dose of about 28 mg/kg to about 60 mg/kg of the composition.

In some embodiments, the subject is administered at least 3, 4, 6, or 7 doses of the composition.

In some embodiments, the at least 3, 4, 6, or 7 doses are administered over about a 4-week to about a 28-week period of time.

In some embodiments, administration of the composition reduces a level of one or more transcripts selected from the group consisting of angiotensin-converting enzyme 2 (ACE2), interleukin 6 (IL6), interleukin 2 receptor (IL2R), tumor necrosis factor alpha (TNFα), C-Reactive Protein (CRP), interleukin 1B (IL1B), interleukin 12 (IL12), interleukin 18 (IL18), interferon gamma (IFNG), interleukin 8 (IL8), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JunB), mitogen-activated protein kinase (MKP1), TNF alpha induced protein 1 (TNFAIP1), TNF alpha induced protein 2 (TNFAIP2), TNF alpha induced protein 3 (TNFAIP3), syndecan 4 (SDC4), superoxide dismutase 2 (SOD2), cytochrome c oxidase subunit II (COX2), interleukin 32 (IL32), intercellular adhesion molecule 1 (ICAM-1), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), and vascular adhesion molecule 1 (VCAM-1), relative to a control.

In some embodiments, administration of the composition reduces a level of one or more proteins selected from the group consisting of angiotensin-converting enzyme 2 (ACE2), interleukin 6 (IL-6), interleukin 2 receptor (IL-2R), tumor necrosis factor alpha (TNF-α), C-Reactive Protein (CRP), interleukin 1B (IL-1B), interferon gamma (IFN-γ), interleukin 8 (IL-8), interleukin 12 (IL-12), interleukin 18 (IL-18), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JUNB), mitogen-activated protein kinase phosphatase 1 (MKP-1), TNF alpha-induced protein 1 (TNFAIP1), TNF alpha-induced protein 2 (TNFAIP2), TNF alpha-induced protein 3 (TNFAIP3), syndecan 4 (SDC4), superoxide dismutase 2 (SOD2), cytochrome c oxidase subunit II (COX2), interleukin 32 (IL-32), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), intercellular adhesion molecule 1 (ICAM-1), and vascular adhesion molecule 1 (VCAM-1) relative to a control.

In some embodiments, administration of the composition increases one or more of a Forced Vital Capacity (FVC) measurement, a Forced Expiratory Volume in 1 second (FEV1) measurement, a Leicester Cough Questionnaire (LCQ) score, a Short Form 36 Health Survey (SF-36) score, a King's Sarcoidosis Questionnaire (KSQ) score, a Steroid-toxicity scale (STS) score, or a 6-minute walk test (6MWT) distance relative to a control.

In some embodiments, administration of the composition reduces one or more of a Patient Global Assessment (PGA) score, a Fatigue Assessment Scale (FAS) score, Modified Medical Research Council (mMRC) Dyspnea Scale score, a Saint George's Respiratory Questionnaire (SGRQ) score, or a Borg's CR10 dyspnea score relative to a control.

In some embodiments, administration of the composition reduces a need for an anti-inflammatory medication relative to a control.

In some embodiments, the subject has received or is receiving an anti-inflammatory medication.

In some embodiments, the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.

In some embodiments, the corticosteroid is selected from the group consisting of prednisone, prednisolone, dexamethasone, hydrocortisone, methylprednisolone, betamethasone, cortisone, fludrocortisone, and triamcinolone.

In some embodiments, administration of the composition reduces a granuloma size or a granuloma formation in the subject.

In some embodiments, the granuloma is a skin granuloma, a lung granuloma, a lymph node granuloma, an ocular granuloma, a liver granuloma, a spleen granuloma, a cardiac granuloma, or a rheumatoid granuloma.

In some embodiments, administration of the composition reduces a calcitriol level in the subject relative to a control.

In some embodiments, administration of the composition reduces a neopterin level in the subject relative to a control.

In some embodiments, administration of the composition reduces a lysozyme level in the subject relative to a control.

In some embodiments, administration of the composition reduces a soluble IL-2R (sIL-2R) level in the subject relative to a control.

In some embodiments, the subject has one or more of acute sarcoidosis, chronic sarcoidosis, progressive sarcoidosis, pulmonary sarcoidosis, remitting sarcoidosis, refractory sarcoidosis, advanced sarcoidosis, ocular sarcoidosis, cutaneous sarcoidosis, or neurosarcoidosis.

### BRIEF DESCRIPTION OF THE DRAWINGS

FIG. 1 illustrates TNFα-mediated cytotoxicity of anti-TNFα antibody XTMAB-16 relative to infliximab in A375 human melanoma cells. DP: Drug Product; TNFα: tumor necrosis factor alpha.

FIG. 2 illustrates transmembrane tumor necrosis factor alpha (mTNFα) binding on the surface of transformed SP2/0 cells. Results are shown for XTMAB-16 (squares) and infliximab (circles).

FIG. 3 is a sensorgram illustrating the bind kinetics of XTMAB-16 to TNFα at different concentrations.

FIG. 4 illustrates the arthritis score over time in animals treated with 20 mg/kg XTMAB-16 or 40 mg/kg XTMAB-16, relative to vehicle-treated controls. Data shown are means±standard error.

FIGS. 5A and 5B provide sensorgrams illustrating the binding kinetics of XTMAB-16 to FcγRI for batch A (FIG. 5A) and batch B (FIG. 5B). DS: Drug Substance; RS: Reference Standard.

FIGS. 6A and 6B provide sensorgrams illustrating the binding kinetics of XTMAB-16 to FcγRIIIa for batch A (FIG. 6A) and batch B (FIG. 6B). DS: Drug Substance; RS: Reference Standard.

FIGS. 7A and 7B provide sensorgrams illustrating the binding kinetics of XTMAB-16 to FcγRIIb for batch A (FIG. 7A) and batch B (FIG. 7B). DS: Drug Substance; RS: Reference Standard.

FIGS. 8A and 8B provide sensorgrams illustrating the binding kinetics of XTMAB-16 to FcRn for batch A (FIG. 8A) and batch B (FIG. 8B). DS: Drug Substance; RS: Reference Standard.

FIG. 9 illustrates XTMAB-16 serum concentration versus time under 10 mg/kg, 20 mg/kg, or 40 mg/kg XTMAB-16 treatment.

FIG. 10 illustrates XTMAB-16 dose proportionality in terms of mean maximum observed concentration (Cₘₐₓ).

FIG. 11 illustrates XTMAB-16 dose proportionality in terms of mean area under the curve (AUC_{(0-∞)}).

FIG. 12 illustrates the concentrations of XTMAB-16 on day 15 after dosing on days 1, 8, and 15 in ICR mice (N=1 to 2 per time point). ICR: Institute of Cancer Research.

FIG. 13 illustrates pharmacokinetic results for XTMAB-16 serum concentration versus time in non-sarcoidosis subjects. Results are shown for linear (FIG. 13A) and logarithmic (FIG. 13B) scales. All results are presented as mean±SD. LLOQ: lower limit of quantitation (400 ng/mL).

FIG. 14 illustrates pharmacokinetic results for XTMAB-16 serum concentration versus time in non-sarcoidosis subjects by treatment and ADA incidence. Results are shown for linear (FIG. 14A) and logarithmic (FIG. 14B) scales. All results are presented as mean±SD. LLOQ: lower limit of quantitation (400 ng/mL).

FIG. 15 illustrates a schematic of generating an in vitro granuloma model of sarcoidosis. PBMC: peripheral blood mononuclear cells.

FIG. 16 illustrates representative photomicrographs from an in vitro model of sarcoidosis of granuloma-like cell aggregates. PPD: purified protein derivative; UNC: uncoated beads.

FIGS. 17A-17C illustrate a group analysis of granuloma formation in High Responder subjects (FIG. 17A), Low Responder subjects (FIG. 17B) subjects with sarcoidosis (FIG. 17C). N=5 per group; *p<0.001, compared to the uncoated bead (UNC) treatment group; †p<0.01, ∀p<0.001, and § p<0.05 relative to the PPD-treated group. Area Fraction=cumulative granuloma area as a percentage of the total area of the image. Bars are mean (μg/mL)±SD. PPD: purified protein derivative; PRED: prednisone; UNC: uncoated beads.

FIGS. 18A-18C illustrate supernatant release of TNFα (FIG. 18A), IL-113 (FIG. 18B), and IFNγ (FIG. 18C) in High Responder subjects with sarcoidosis. N=5 per group; *p<0.001, compared to the uncoated bead (UNC) treatment group; †p<0.01, ∀p<0.001 (p<0.05 in bottom), and § p<0.05 relative to the PPD-treated group. Bars are mean (μg/mL)±SD. IFN: interferon; IL: interleukin; PPD: purified protein derivative; PRED: prednisone; SD: standard deviation; TNF: tumor necrosis factor; UNC: uncoated beads.

FIGS. 19A and 19B illustrate XTMAB-16 concentration versus area fraction relative to PPD (FIG. 19A) and XTMAB-16 concentration versus IL-1β levels (FIG. 19B) in an in vitro granuloma model. CI: confidence interval; 1050: half maximal inhibitor concentration; IL: interleukin; PPD: purified protein derivative

FIGS. 20A and 20B illustrate XTMAB-16 concentration versus time by dose for both a linear scale (FIG. 20A) and a semi-logarithmic scale (FIG. 20B). BLQ: below the limit of quantitation; SD: standard deviation; Tₘₐₓ: time to maximum concentration.

FIGS. 21A-21F. illustrate model-predicted lung XTMAB-16 concentration in virtual subjects. FIGS. 21A and 21B illustrate population predictions. FIGS. 21C and 21D illustrate individual predictions. FIG. 21E illustrates the conditional weighted residual versus population prediction. FIG. 21F illustrates the conditional weighted residual analysis versus time after dose. IC50: concentration at half-maximal MIPAR area fraction defined as the cumulative granuloma area as a percentage of the total area of the image; Q2W: every 2 weeks; Q4W: every 4 weeks.

FIG. 22 illustrates a prediction-correlated visual predictive check for XTMAB-16 concentrations over time. CL: clearance; VPC: visual predictive check.

FIG. 23 illustrates a model-predicted lung XTMAB-16 concentration in a 70 kg individual at various dosing regimens. Q2W: every 2 weeks; Q4W: every 4 weeks.

FIG. 24 illustrates a high-level overview of assessing XTMAB-16 in human subjects by using both a multiple ascending dose (MAD) study and a proof of concept (POC) study.

FIG. 25 illustrates a MAD study schematic for assessing XTMAB-16 in human subjects. kg: kilogram; mg: milligram; Q2W: every 2 weeks; Q4W: every four weeks.

FIG. 26 illustrates a POC study schematic for assessing XTMAB-16 in human subjects. Open circles represent a Q2W dosing schedule. Black closed circles represent a Q4W dosing schedule. kg: kilogram; mg: milligram; Q2W: every 2 weeks; Q4W: every four weeks; TBD: to be determined.

FIG. 27 illustrates a schematic for dose escalation in the MAD study of FIG. 25. kg: kilogram; mg: milligram; Q2W: every 2 weeks; Q4W: every four weeks.

### DETAILED DESCRIPTION

While the present technology is capable of being embodied in various forms, the description below of several embodiments is made with the understanding that the present technology is to be considered as an exemplification of the present technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art. Furthermore, the term “about,” as used herein when referring to a measurable value such as an amount or concentration and the like, is meant to encompass variations of 20%, 10%, 5%, 1%, 0.5%, or even 0.1% of the specified amount.

Also, the ranges of the present technology are intended as a continuous range, including every value between the minimum and maximum values recited, as well as any ranges that may be formed by such values. The present technology comprises any and all (and ranges of any such ratios) that may be formed by dividing a numeric value of the present technology into any other numeric value of the present technology. Accordingly, the skilled person will appreciate that many such ratios, ranges, and ranges of ratios may be unambiguously derived from the numerical values presented herein and in all instances, such ratios, ranges, and ranges of ratios represent various embodiments of the present technology.

The terms “bind” and “binding” refer to all types of physical and chemical binding, reactions, complexing, attraction, chelating and the like.

“Amino acids” are molecules containing an amine group, a carboxylic acid group, and a side-chain that is specific to each amino acid. The key elements of an amino acid are carbon, hydrogen, oxygen, and nitrogen and have the generic formula H₂N—CHR—COOH, wherein R represents a side chain group. The various α-amino acids differ in the side-chain moiety that is attached to the α-carbon. The “amino acids” of the present technology include the known naturally occurring protein amino acids, which are referred to by both their common three letter abbreviation and single letter abbreviation. See generally Synthetic Peptides: A User's Guide, G. A. Grant, editor, W.H. Freeman & Co., New York (1992), the teachings of which are incorporated herein by reference, including the text and table set forth at pages 11 through 24. As set forth above, the term “amino acid” also includes stereoisomers and modifications of naturally occurring protein amino acids, non-protein amino acids, post-translationally modified amino acids, enzymatically synthesized amino acids, derivatized amino acids, constructs or structures designed to mimic amino acids, and the like. Modified and unusual amino acids are described generally in Synthetic Peptides: A User's Guide, supra; Hruby et al., Biochem. J. 268:249-262 (1990); and Toniolo, Int. J. Peptide Protein Res. 35:287-300 (1990); the teachings of all of which are incorporated herein by reference.

“Antibody” refers to any form of antibody that exhibits the desired biological activity, such as inhibiting binding of a ligand to its receptor, or by inhibiting ligand-induced signaling of a receptor. Thus, “antibody” is used in the broadest sense and specifically covers, but is not limited to, monoclonal antibodies (including full length monoclonal antibodies), polyclonal antibodies, and multispecific antibodies (e.g., bispecific antibodies).

“Antibody fragment” and “antibody binding fragment” mean antigen-binding fragments and analogues of an antibody, typically including at least a portion of the antigen binding or variable regions (e.g., one or more CDRs) of the parental antibody. An antibody fragment retains at least some of the binding specificity of the parental antibody. Typically, an antibody fragment retains at least 10% of the parental binding activity when that activity is expressed on a molar basis. Preferably, an antibody fragment retains at least 20%, 50%, 70%, 80%, 90%, 95% or 100% or more of the parental antibody's binding affinity for the target. Examples of antibody fragments include, but are not limited to, Fab, Fab′, F(ab′)2, and Fv fragments; diabodies; linear antibodies; single-chain antibody molecules, e.g., sc-Fv, unibodies (technology from Genmab); nanobodies (technology from Domantis); domain antibodies (technology from Ablynx); and multispecific antibodies formed from antibody fragments. Engineered antibody variants are reviewed in Holliger and Hudson (2005) Nat. Biotechnol. 23:1126-1136.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

An antibody fragment, as used herein, may comprise a sufficient portion of the constant region to permit dimerization (or multimerization) of heavy chains that have reduced disulfide linkage capability, for example where at least one of the hinge cysteines normally involved in inter-heavy chain disulfide linkage is altered as described herein. In another embodiment, an antibody fragment, for example one that comprises the Fc region, retains at least one of the biological functions normally associated with the Fc region when present in an intact antibody, such as FcRn binding, antibody half-life modulation, Antibody Dependent Cell-Mediated Cytotoxicity (ADCC) function, and/or complement binding (for example, where the antibody has a glycosylation profile necessary for ADCC function or complement binding).

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}² domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}² domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody” (or “scFv antibody”) refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448.

A “domain antibody fragment” is an immunologically functional immunoglobulin fragment containing only the variable region of a heavy chain or the variable region of a light chain. In some instances, two or more V_{H} regions are covalently joined with a peptide linker to create a bivalent domain antibody fragment. The two V_{H} regions of a bivalent domain antibody fragment may target the same or different antigens.

The term “chimeric” antibody refers to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (See, for example, U.S. Pat. No. 4,816,567 and Morrison et al., 1984, Proc. Nat. Acad. Sci. USA 81:6851-6855).

“Humanized” forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present technology may be made by the hybridoma method first described by Kohler et al., 1975, Nature 256:495, or may be made by recombinant DNA methods (see, for example, U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example. The monoclonal antibodies herein specifically include “chimeric” antibodies.

As used herein, a sequence “variant” refers to a sequence that differs from the sequence of the present technology at one or more amino acid residues but which retains the biological activity of the resulting molecule.

As used herein, “% identity” between two sequences refers to a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm. For example, the percent identity between two amino acid sequences can be determined using the algorithm of E. Meyers and W. Miller (Comput. App. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

“Specifically” binds, when referring to a ligand/receptor, antibody/antigen, or other binding pair, indicates a binding reaction which is determinative of the presence of the protein, e.g., TNFα, in a heterogeneous population of proteins and/or other biologics. Thus, under designated conditions, a specified ligand/antigen binds to a particular receptor/antibody and does not bind in a significant amount to other proteins present in the sample.

As used herein, a “pharmaceutically acceptable carrier” of a composition refers to a carrier or diluent that does not cause significant irritation to an organism, does not abrogate the biological activity and properties of the administered active ingredient, and/or does not interact in a deleterious manner with the other components of the composition in which it is contained. The term “carrier” encompasses any excipient, binder, diluent, filler, salt, buffer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety). Some examples of physiologically acceptable carriers include antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.). An “excipient” of a composition refers to an inert substance added to a composition to further facilitate administration of a compound. Examples, without limitation, of excipients include calcium carbonate, calcium phosphate, various sugars and types of starch, cellulose derivatives, gelatin, vegetable oils, and polyethylene glycols.

As used herein, a “control” when referring to an antibody may refer to a composition lacking the anti-TNFα antibodies of the present technology or a composition having an antibody comprising different features from the anti-TNFα antibodies of the present technology. For example, the control may comprise an anti-TNFα antibody having a different amino acid sequence or a different post-translational modification status from the anti-TNFα antibodies of the present technology.

As used herein, a “control” when referring to a subject may refer to the subject at baseline (i.e., prior to administration of a composition of the present technology or another therapeutic intervention), a subject that does not have sarcoidosis and/or a granuloma, an untreated subject, a subject administered a placebo, or a subject administered a composition that is different from the composition that is administered to the treated subject.

### Anti-TNFα Antibodies

### Amino Acid Sequences

The present technology provides compositions comprising an anti-TNFα antibody or an antigen binding fragment thereof. The anti-TNFα antibodies of the present technology specifically bind at least a portion of a TNFα protein or peptide. The TNFα protein or peptide may be produced by macrophages, monocytes, neutrophils, T cells, NK-cells, or fibroblasts, and may be membrane-bound or soluble. The anti-TNFα antibodies may bind a human or a nonhuman target. In some embodiments, the anti-TNFα antibodies of the present technology bind a protein listed in Table 1. In some embodiments, the anti-TNFα antibodies bind a protein comprising an amino acid sequence that is about 75%, 80%, 85%, 90%, 95%, 99%, or 100% Identical to SEQ. ID NO: 1 or SEQ ID NO: 2. In some embodiments, the anti-TNFα antibodies bind a protein comprising an amino acid sequence that is at least 75%, 80%, 85%, 90%, 95%, 99%, or 100% Identical to SEQ. ID NO: 1 or SEQ ID NO: 2. In some embodiments, the anti-TNFα antibodies bind a protein comprising an amino acid sequence that is at least about 75%, 80%, 85%, 90%, 95%, 99%, or 100% Identical to SEQ. ID NO: 1 or SEQ ID NO: 2.

TABLE 1
TNFα Proteins

| | | SEQ ID | |
|---|---|---|---|
| Protein | Amino Acid Sequence | NO | Source |
| Human | MSTESMIRDVELAEEALPKKTGGPQGS | 1 | NCBI |
| TNFα | RRCLFLSLFSFLIVAGATTLFCLLHFGVI | | GenBank: |
| | GPQREEFPRDLSLISPLAQAVRSSSRTP | | CAA78745.1 |
| | SDKPVAHVVANPQAEGQLQWLNRRAN | | |
| | ALLANGVELRDNQLVVPSEGLYLIYSQV | | |
| | LFKGQGCPSTHVLLTHTISRIAVSYQTKV | | |
| | NLLSAIKSPCQRETPEGAEAKPWYEPIY | | |
| | LGGVFQLEKGDRLSAEINRPDYLDFAES | | |
| | GQVYFGIIAL | | |
| Murine | MSTESMTRDVELAEEALPQKMGGFQN | 2 | NCBI |
| TNFα | SRRCLCLSLFSFLLVAGATTLFCLLNFG | | GenBank: |
| | VIGPQRDEKFPNGLPLISSMAQTLSSSQ | | AAB65593.1 |
| | NSSDKPVAHVVANHQVEEQLEWLSQR | | |
| | ANALLANGMDLKDNQLVVPADGLYLVY | | |
| | SQVLFKGQGCPDYVLLTHTVSRFAISYQ | | |
| | EKVNLLSAVKSPCPKDTPEGAELKPWY | | |
| | EPIYLGGVFQLEKGDQLSAEVNLPKYLD | | |
| | FAESGQVYFGVIAL | | |

The anti-TNFα antibodies may comprise a heavy chain comprising one or more amino acid sequences that is about 75%, 80%, 85%, 90%, 95%, 99%, or 100% identical to a sequence listed in Table 2.

The anti-TNFα antibodies of the present technology comprise a heavy chain having at least one Complementarity Determining Region (CDR) sequence. The at least one CDR sequence confers binding to a specific antigen (e.g., a TNFα peptide).

In some embodiments, the at least one CDR sequence comprises an amino acid sequence that is about 75%, 80%, 85%, 90%, 95%, 99%, or 100% identical to a CDR sequence listed in Table 2 (e.g., SEQ ID NO: 3, 4, 5, 6, 7, or 8). In some embodiments, the anti-TNFα antibodies comprise a heavy chain having a sequence that comprises CDR sequences about 75%, 80%, 85%, 90%, 95%, 99%, or 100% identical to SEQ ID NO: 3, 4, 5, 6, 7, and 8. The CDR sequences may be distinct from other CDR sequences (e.g., do not overlap another CDR sequence) or at least a portion of a CDR sequence may overlap with a portion of another CDR sequence (e.g., two CDR sequences share at least a portion of the amino acid sequence).

In some embodiments, the anti-TNFα antibodies comprise a heavy chain comprising a CDR having an amino acid sequence that is about 75%, 80%, 85%, 90%, 95%, 99%, or 100% Identical to SEQ. ID NO: 3.

In some embodiments, the anti-TNFα antibodies comprise a CDR having an amino acid sequence that is at least 75%, 80%, 85%, 90%, 95%, 99%, or 100% Identical to SEQ. ID NO: 3.

In some embodiments, the anti-TNFα antibodies comprise a CDR having an amino acid sequence that is at least about 75%, 80%, 85%, 90%, 95%, 99%, or 100% Identical to SEQ. ID NO: 3.

In some embodiments, the anti-TNFα antibodies comprise a CDR having an amino acid sequence that is about 75%, 80%, 85%, 90%, 95%, 99%, or 100% Identical to SEQ. ID NO: 4.

In some embodiments, the anti-TNFα antibodies comprise a CDR having an amino acid sequence that is at least 75%, 80%, 85%, 90%, 95%, 99%, or 100% Identical to SEQ. ID NO: 4.

In some embodiments, the anti-TNFα antibodies comprise a CDR having an amino acid sequence that is at least about 75%, 80%, 85%, 90%, 95%, 99%, or 100% Identical to SEQ. ID NO: 4.

In some embodiments, the anti-TNFα antibodies comprise a CDR having an amino acid sequence that is about 75%, 80%, 85%, 90%, 95%, 99%, or 100% Identical to SEQ. ID NO: 5.

In some embodiments, the anti-TNFα antibodies comprise a CDR having an amino acid sequence that is at least 75%, 80%, 85%, 90%, 95%, 99%, or 100% Identical to SEQ. ID NO: 5.

In some embodiments, the anti-TNFα antibodies comprise a CDR having an amino acid sequence that is at least about 75%, 80%, 85%, 90%, 95%, 99%, or 100% Identical to SEQ. ID NO: 5.

In some embodiments, the anti-TNFα antibodies comprise a CDR having an amino acid sequence that is about 75%, 80%, 85%, 90%, 95%, 99%, or 100% Identical to SEQ. ID NO: 6.

In some embodiments, the anti-TNFα antibodies comprise a CDR having an amino acid sequence that is at least 75%, 80%, 85%, 90%, 95%, 99%, or 100% Identical to SEQ. ID NO: 6.

In some embodiments, the anti-TNFα antibodies comprise a CDR having an amino acid sequence that is at least about 75%, 80%, 85%, 90%, 95%, 99%, or 100% Identical to SEQ. ID NO: 6.

In some embodiments, the anti-TNFα antibodies comprise a CDR having an amino acid sequence that is about 75%, 80%, 85%, 90%, 95%, 99%, or 100% Identical to SEQ. ID NO: 7.

In some embodiments, the anti-TNFα antibodies comprise a CDR having an amino acid sequence that is at least 75%, 80%, 85%, 90%, 95%, 99%, or 100% Identical to SEQ. ID NO: 7.

In some embodiments, the anti-TNFα antibodies comprise a CDR having an amino acid sequence that is at least about 75%, 80%, 85%, 90%, 95%, 99%, or 100% Identical to SEQ. ID NO: 7.

In some embodiments, the anti-TNFα antibodies comprise a CDR having an amino acid sequence that is about 75%, 80%, 85%, 90%, 95%, 99%, or 100% Identical to SEQ. ID NO: 8.

In some embodiments, the anti-TNFα antibodies comprise a CDR having an amino acid sequence that is at least 75%, 80%, 85%, 90%, 95%, 99%, or 100% Identical to SEQ. ID NO: 8.

In some embodiments, the anti-TNFα antibodies comprise a CDR having an amino acid sequence that is at least about 75%, 80%, 85%, 90%, 95%, 99%, or 100% Identical to SEQ. ID NO: 8.

In some embodiments, the anti-TNFα antibodies comprise a variable heavy (V_{H}) domain having an amino acid sequence that is about 75%, 80%, 85%, 90%, 95%, 99%, or 100% Identical to SEQ. ID NO: 9.

In some embodiments, the anti-TNFα antibodies comprise a V_{H} domain having an amino acid sequence that is at least 75%, 80%, 85%, 90%, 95%, 99%, or 100% Identical to SEQ. ID NO: 9.

In some embodiments, the anti-TNFα antibodies comprise a V_{H} having an amino acid sequence that is at least about 75%, 80%, 85%, 90%, 95%, 99%, or 100% Identical to SEQ. ID NO: 9.

TABLE 2
Heavy Chain Anti-TNFα antibody Sequences

| | | SEQ |
|---|---|---|
| Antibody | | ID |
| Region | Amino Acid Sequence | NO |
| CDR1A | GFIFSNHW | 3 |
| CDR2A | IRSKSINSAT | 4 |
| CDR3A | SRNYYGSTYDY | 5 |
| CDR1B | NHWMN | 6 |
| CDR2B | EIRSKSINSATHYAESVKG | 7 |
| CDR3B | NYYGSTYDY | 8 |
| Variable | EVKLEESGGGLVQPGGSMKLSCVASGFIF | 9 |
| Heavy | SNHWMNWVRQSPEKGLEWVAEIRSKSIN | |
| (V_{H}) | SATHYAESVKGRFTISRDDSKSAVYLQMT | |
| Chain | DLRTEDTGVYYCSRNYYGSTYDYWGQGT | |
| | TLTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSCDKTHTCPPC | |
| | PAPELLGGPSVFLFPPKPKDTLMISRTPEV | |
| | TCVVVDVSHEDPEVKFNWYVDGVEVHNA | |
| | KTKPREEQYNSTYRVVSVLTVLHQDWLNG | |
| | KEYKCKVSNKALPAPIEKTISKAKGQPREP | |
| | QVYTLPPSRDELTKNQVSLTCLVKGFYPS | |
| | DIAVEWESNGQPENNYKTTPPVLDSDGSF | |
| | FLYSKLTVDKSRWQQGNVFSCSVMHEAL | |
| | HNHYTQKSLSLSPGK | |

The anti-TNFα antibodies may comprise a light chain comprising one or more amino acid sequences that is about 75%, 80%, 85%, 90%, 95%, 99%, or 100% identical to a sequence listed in Table 3.

The anti-TNFα antibodies of the present technology comprise a light chain having at least one CDR sequence. The at least one CDR sequence confers binding to a specific antigen (e.g., a TNFα peptide).

In some embodiments, the at least one CDR sequence comprises an amino acid sequence that is about 75%, 80%, 85%, 90%, 95%, 99%, or 100% identical to a CDR sequence listed in Table 2 (e.g., SEQ ID NO: 10, 12, 13, 14, 15, or YA). In some embodiments, the anti-TNFα antibodies comprise a light chain having a sequence that comprises CDR sequences about 75%, 80%, 85%, 90%, 95%, 99%, or 100% identical to SEQ ID NO: 10, 12, 13, 14, 15, and YA. The CDR sequences may be distinct (e.g., do not overlap another CDR sequence) or may overlap (e.g., share at least a portion of an amino acid sequence).

In some embodiments, the anti-TNFα antibodies comprise a light chain comprising a CDR having an amino acid sequence that is about 75%, 80%, 85%, 90%, 95%, 99%, or 100% Identical to SEQ. ID NO: 10.

In some embodiments, the anti-TNFα antibodies comprise a CDR having an amino acid sequence that is at least 75%, 80%, 85%, 90%, 95%, 99%, or 100% Identical to SEQ. ID NO: 10.

In some embodiments, the anti-TNFα antibodies comprise a CDR having an amino acid sequence that is at least about 75%, 80%, 85%, 90%, 95%, 99%, or 100% Identical to SEQ. ID NO: 10.

In some embodiments, the anti-TNFα antibodies comprise a CDR having an amino acid sequence that is about 75%, 80%, 85%, 90%, 95%, 99%, or 100% Identical to YA.

In some embodiments, the anti-TNFα antibodies comprise a CDR having an amino acid sequence that is at least 75%, 80%, 85%, 90%, 95%, 99%, or 100% Identical to SEQ. ID NO: YA.

In some embodiments, the anti-TNFα antibodies comprise a CDR having an amino acid sequence that is at least about 75%, 80%, 85%, 90%, 95%, 99%, or 100% Identical to SEQ. ID NO: YA.

In some embodiments, the anti-TNFα antibodies comprise a CDR having an amino acid sequence that is about 75%, 80%, 85%, 90%, 95%, 99%, or 100% Identical to SEQ. ID NO: 12.

In some embodiments, the anti-TNFα antibodies comprise a CDR having an amino acid sequence that is at least 75%, 80%, 85%, 90%, 95%, 99%, or 100% Identical to SEQ. ID NO: 12.

In some embodiments, the anti-TNFα antibodies comprise a CDR having an amino acid sequence that is at least about 75%, 80%, 85%, 90%, 95%, 99%, or 100% Identical to SEQ. ID NO: 12.

In some embodiments, the anti-TNFα antibodies comprise a CDR having an amino acid sequence that is about 75%, 80%, 85%, 90%, 95%, 99%, or 100% Identical to SEQ. ID NO: 13.

In some embodiments, the anti-TNFα antibodies comprise a CDR having an amino acid sequence that is at least 75%, 80%, 85%, 90%, 95%, 99%, or 100% Identical to SEQ. ID NO: 13.

In some embodiments, the anti-TNFα antibodies comprise a CDR having an amino acid sequence that is at least about 75%, 80%, 85%, 90%, 95%, 99%, or 100% Identical to SEQ. ID NO: 13.

In some embodiments, the anti-TNFα antibodies comprise a CDR having an amino acid sequence that is about 75%, 80%, 85%, 90%, 95%, 99%, or 100% Identical to SEQ. ID NO: 14.

In some embodiments, the anti-TNFα antibodies comprise a CDR having an amino acid sequence that is at least 75%, 80%, 85%, 90%, 95%, 99%, or 100% Identical to SEQ. ID NO: 14.

In some embodiments, the anti-TNFα antibodies comprise a CDR having an amino acid sequence that is at least about 75%, 80%, 85%, 90%, 95%, 99%, or 100% Identical to SEQ. ID NO: 14.

In some embodiments, the anti-TNFα antibodies comprise a CDR having an amino acid sequence that is about 75%, 80%, 85%, 90%, 95%, 99%, or 100% Identical to SEQ. ID NO: 15.

In some embodiments, the anti-TNFα antibodies comprise a CDR having an amino acid sequence that is at least 75%, 80%, 85%, 90%, 95%, 99%, or 100% Identical to SEQ. ID NO: 15.

In some embodiments, the anti-TNFα antibodies comprise a CDR having an amino acid sequence that is at least about 75%, 80%, 85%, 90%, 95%, 99%, or 100% Identical to SEQ. ID NO: 15.

In some embodiments, the anti-TNFα antibodies comprise a variable light (V_{L}) domain having an amino acid sequence that is about 75%, 80%, 85%, 90%, 95%, 99%, or 100% Identical to SEQ. ID NO: 16.

In some embodiments, the anti-TNFα antibodies comprise a V_{L} domain having an amino acid sequence that is at least 75%, 80%, 85%, 90%, 95%, 99%, or 100% Identical to SEQ. ID NO: 16.

In some embodiments, the anti-TNFα antibodies comprise a V_{L} having an amino acid sequence that is at least about 75%, 80%, 85%, 90%, 95%, 99%, or 100% Identical to SEQ. ID NO: 16.

TABLE 3
Light Chain Anti-TNFα antibody Sequences

| | | SEQ |
|---|---|---|
| Antibody | | ID |
| Region | Amino Acid Sequence | NO |
| CDR1A | QFVGSS | 10 |
| CDR2A | YA | |
| CDR3A | QQSHSWPFT | 12 |
| CDR1B | RASQFVGSSIH | 13 |
| CDR2B | YASESMS | 14 |
| CDR3B | QQSHSWPFT | 15 |
| Variable | DILLTQSPAILSVSPGERVSFSCRASQFVG | 16 |
| Light | SSIHWYQQRTNGSPRLLIKYASESMSGIPS | |
| (V_{L}) | RFSGSGSGTDFTLSINTVESEDIADYYCQ | |
| Chain | QSHSWPFTFGSGTNLEVKRTVAAPSVFIF | |
| | PPSDEQLKSGTASVVCLLNNFYPREAKVQ | |
| | WKVDNALQSGNSQESVTEQDSKDSTYSL | |
| | SSTLTLSKADYEKHKVYACEVTHQGLSSP | |
| | VTKSFNRGEC | |

### Anti-TNFα Antibody Functions

Avidity of the anti-TNFα antibodies for the TNFα protein or peptide is determined by amino acid sequences on the heavy and/or light chain of each antibody. These amino acid sequences may comprise one or more CDRs. Binding of the anti-TNFα antibodies to the TNFα protein or peptide may reduce an activity of TNFα. For example, the anti-TNFα antibodies may reduce or prevent an outcome of TNFα signaling, including, but not limited to, upregulation of nuclear factor kappa-light-chain-enhancer of activated B cells (NF-κB) signaling, mitogen-activated protein kinase (MAPK) signaling, pro-apoptotic signaling, cytokine and/or chemokine production (e.g., Interleukin-1 (IL-1), Interleukin-6 (IL-6), Interleukin-8 (IL-8), Interleukin-12 (IL-12), Interleukin-18 (IL-18), Tumor Necrosis Factor Beta (TNF-β), chemokine (C—X—C motif) ligands 1, 2, 5, 9, 10, and chemokine (C—C motif) ligands 2, 5, and 20)), upregulation of cell adhesion molecules (e.g., Intercellular Adhesion Molecule 1 (ICAM-1), Vascular Cell Adhesion Molecule 1 (VCAM-1), E-Selectin, and P-Selectin), and reactive oxygen species (ROS) production (e.g., superoxide anions, hydrogen peroxide, and hydroxy radicals).

In some embodiments, binding of the anti-TNFα antibodies to the TNFα protein or peptide may reduce a concentration of TNFα in the plasma or other tissues of the subject. For example, the anti-TNFα antibodies may reduce membrane TNFα and/or cytoplasmic TNFα of resident, migrating, infiltrating, and/or intravascular cells (e.g., mononuclear, epithelial, endothelial, glomerular, myocyte, neural, or myoepithelial cells) in the bladder, cervix, esophagus, large intestine, liver (Kupffer cells), lymph node, lung, pancreas, placenta (Hofbauer cells), parathyroid, salivary gland, skin, small intestine, spleen, stomach, thyroid, thymus, tonsil, and uterus.

### Complement Dependent Cytotoxicity and Antibody Dependent Cell-Mediated Cytotoxicity:

The anti-TNFα antibodies may mediate Complement Dependent Cytotoxicity (CDC) and/or Antibody Dependent Cell-Mediated Cytotoxicity (ADCC) of mTNF-expressing cells. CDC or ADCC may be mediated through binding of the anti-TNFα antibodies to one or more of C1q, Fc receptors, FcγRI, FcγRIIb, or FcRn. CDC may determine the ability of the anti-TNFα antibodies to induce cytotoxic effects on cells expressing TNFα in the presence of complement factors. ADCC may be mediated by NK cells as part of the humoral immune response. ADCC of the anti-TNFα antibodies may promote lysis of cells expressing TNFα proteins or peptides thereof in the presence of effector cells of the immune system such as NK cells, myocytes, macrophages, or eosinophil cells.

C1q Binding: C1q, the first subcomponent of the C1 complex of the classical pathway of complement activation, is thought to bind to sites on the CH2 domain of IgG. C1q comprises the C1qA (Accession: NP057075.1), C1qB (Isoform 1 Accession: NP_000482.3; Isoform 2 Accession: NP_001365085.1), and C1qC (Isoform 1 Accession: NP_758957.2; Isoform 2 Accession: NP_001334549.1) peptide chains. Together with 2 serine proteases, C1r (Isoform 1 Accession: NP_001724.4; Isoform 2 Accession: NP_001341275.1) and C1s (Isoform 1 Accession: NP_001725.1; Isoform 2 Accession: NP_001333779.1), C1q forms the complex C1, the first component of the complement. Activation of the complement cascade system generates 03 and 05 convertase enzymes. These enzymes generate the pro-inflammatory C3a and C5a anaphylatoxins and catalyze formation of the pore-like membrane attack complex (C5-9) that may insert into cell membranes causing damage by lytic or sublytic mechanisms.

In some embodiments, the Fc portion of the anti-TNFα antibodies or the antigen binding fragment thereof comprising the Fc portion may bind C1q or at least a portion of C1q (e.g., C1qA, C1qB, C1qC, or peptides thereof). In some embodiments, binding occurs at the CH2 domain of the anti-TNFα antibodies. Binding to C1q or at least the portion of C1q may block C1q function and/or signaling. Nonlimiting examples of C1q signaling include activation of the complement cascade system.

Fc Receptor Binding: CDC and/or ADCC may occur through binding of the anti-TNFα antibodies to Fc receptors (e.g., Fcγ RI, RII, and RIII), also known as CD64 (GenBank: AA152384.1), CD32 (GenBank: AA148274.1), and 0016 (NCBI: NP_001373379.1), respectively. In some embodiments, the Fc portion of the anti-TNFα antibodies or the antigen binding fragment thereof comprising the Fc portion may bind a Fc receptor or at least a portion of an Fc receptor (e.g., Fcγ RI, RII, and RIII, or peptides thereof). Binding to Fc receptors or at least the portion of the Fc receptor may trigger ADCC and cytotoxic cell activation and may lead to destruction of the cells expressing TNFα.

FcγRI Binding: FcγRI (also known as CD64 (GenBank: AAI52384.1)) is a 72 kDa glycoprotein, expressed on mononuclear phagocytes, and may binds IgG via the Fc binding domain. FcγRI exhibits high affinity for IgG, Ka=108-109 M−1 and may bind monomeric IgG. FcγRI that is released to biological fluids may function as an immunoglobulin binding factor. FcγRI expression may be induced by interferon (IFN)-gamma and may play a role in ADCC and clearance of immune complexes. FcγRI binding may also be implicated in the pathogenesis of immune complexes and auto-antibody mediated diseases. In some embodiments, the Fc portion of the anti-TNFα antibodies or the antigen binding fragment thereof comprising the Fc portion may bind FcγRI or at least a portion of FcγRI. Binding to FcγRI may trigger ADCC and cytotoxic cell activation and may lead to destruction of the cells expressing TNFα.

FcRn Binding: FcRn (Accession: AAF72596.1) is composed of 2 subunits (a subunit and 132 microglobulin). The a subunit is structurally related to major histocompatibility complex (MHC) class I proteins and may also associate with 32 microglobulin. Binding of IgG to FcRn, which is pH dependent, protects the antibody from degradation via an endocytosis salvage pathway, thereby prolonging the half-life of the IgG in the serum.

In some embodiments, the anti-TNFα antibodies or the antigen binding fragment thereof may bind FcRn at least a portion of FcRn (e.g., a subunit, 132 microglobulin, or a peptide thereof). Binding to FcRn may trigger protect the anti-TNFα antibodies from degradation and/or prolong the anti-TNFα antibodies half-life.

FcγRIIb Binding: Binding of anti-TNFα antibodies to FcγRIIb (isoform 1 Accession: NP_001381406.1) may modulate immune responses such as ADCC. FcγRIIb shows weak affinity for monomeric IgG, Kₐ≤107 M−1, and may only interact effectively with multimeric immune complexes. In some embodiments, the anti-TNFα antibodies or the antigen binding fragment thereof may bind FcγRIIb or at least a portion of FcγRIIb. Binding to FcγRIIb may trigger ADCC and cytotoxic cell activation and may lead to destruction of the cells expressing TNFα.

### Post-Translational Modifications

The anti-TNFα antibodies of the present technology may comprise post-translational modifications (e.g., glycosylation, N-terminal glutaminyl cyclization, C-terminal lysine clipping, sialylation, methylation, acetylation, ubiquitination, sumoylation, phosphorylation, proteolytic cleavage) that may influence features of the antibody (e.g., immunogenicity, stability, avidity, dissociation rates). For example, the anti-TNFα antibodies may be sialylated with one of more N-acetylneuraminic acid (Neu5Ac) molecules and/or may lack N-glycolylneuraminic acid (Neu5Gc). Neu5Ac is the predominant sialic acid found in human cells, whereas the main sialic acid form of other TNF inhibitors is Neu5Gc, which is only found in non-human cells and may be immunogenic.

In some embodiments, the anti-TNFα antibodies are sialylated at Asn 297. In some embodiments, Asn 297 is sialylated with Neu5Ac.

In some embodiments, the anti-TNFα antibodies of the present technology are glycosylated. In some embodiments, the anti-TNFα antibodies are glycosylated at an asparagine residue (e.g., Asn 300) on at least one of the heavy chains. In some embodiments, the anti-TNFα antibodies are glycosylated at an asparagine residue (e.g., Asn 300) on both of the heavy chains.

In some embodiments, the anti-TNFα antibodies of the present technology are fucosylated.

In some embodiments, the anti-TNFα antibodies of the present technology comprise one or more disulfide bonds (e.g., intra-chain or inter-chain). In some embodiments, the one or more disulfide bonds enhance the stability and/or structural integrity of the antibody relative to an antibody lacking disulfide bonds. In some embodiments, the anti-TNFα antibodies comprise 16 disulfide bonds (12 intra-chain and 4 inter-chain).

In some embodiments, the anti-TNFα antibodies of the present technology comprise a C-terminal lysine on at least one of the heavy chains. In some embodiments, the anti-TNFα antibodies of the present technology comprise a C-terminal lysine on both of the heavy chains. In some embodiments, the C-terminal lysine is not clipped. In some embodiments, the charge of the anti-TNFα antibodies may be modified by C-terminal lysine clipping on one or both of the heavy chains.

In some embodiments, the anti-TNFα antibodies may be generated using a biological system which influences the types of post translational modifications made to the anti-TNFα antibodies. For example, the anti-TNFα antibodies may be generated using a Chinese hamster ovary (CHO) cell line rather than a cell line that is used in the production of other TNFα inhibitors (e.g., SP2 cell line). Nonlimiting examples of cell lines which may be used to generate the anti-TNFα antibodies include CHO cells, NS0 cells, hybridoma cells, SP2/0 cells, human embryonic kidney (HEK) cells, PER.C6 cells, yeast cells, and myeloma cells.

### Pharmacodynamics and Pharmacokinetics

The anti-TNFα antibodies of the present technology may comprise features that modify pharmacodynamic parameters (e.g., binding, efficacy, and or potency) and/or pharmacokinetic parameters (e.g., absorption, distribution, metabolism, or elimination) relative to a control (e.g., an anti-TNFα antibody comprising a different sequence and/or a different post translational modification status from the anti-TNFα antibodies of the present technology). Features such as the amino acid sequences or the post translational modifications of the anti-TNFα antibodies may contribute to the pharmacodynamic or pharmacokinetic differences relative to the control.

In some embodiments, the anti-TNFα antibodies of the present technology have an increased affinity for a TNFα protein or peptide thereof, relative to a control. In some embodiments, the anti-TNFα antibodies of the present technology comprise an increased affinity for C1q, Fc receptors, FcγRI, FcγRIIb, or FcRn proteins or peptides thereof.

The affinity may be assessed as a dissociation constant (K_{D}) value. In some embodiments, the anti-TNFα antibodies of the present technology comprise a reduced K_{D} from a TNFα protein or peptide thereof, relative to a control. In some embodiments, the anti-TNFα antibodies of the present technology comprise a reduced K_{D} from C1q, Fc receptors, FcγRI, FcγRIIb, or FcRn proteins or peptides thereof.

In some embodiments, the reduction in K_{D} is a reduction by about 5%, 10%, 20%, 30%, 40%, 50%, 60%, 70%, 80%, 90%, or 99% relative to the control.

In some embodiments, the reduction in K_{D} is a reduction by at least 5%, 10%, 20%, 30%, 40%, 50%, 60%, 70%, 80%, 90%, or 99% relative to the control.

In some embodiments, the reduction in K_{D} is a reduction by at least about 5%, 10%, 20%, 30%, 40%, 50%, 60%, 70%, 80%, 90%, or 99% relative to the control.

In some embodiments, the anti-TNFα antibodies of the present technology comprise an increased half-life relative to a control. The control may comprise an anti-TNFα antibody or other TNFα inhibitor that lacks a post-translational modification possessed by the anti-TNFα antibodies of the present technology.

In some embodiments, the increase in half-life is an increase by about 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the increase in half-life is an increase by at least 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the increase in half-life is an increase by at least about 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the anti-TNFα antibodies of the present technology comprise a reduced clearance (CL) relative to a control. The control may comprise an anti-TNFα antibody or other TNFα inhibitor that lacks a post-translational modification possessed by the anti-TNFα antibodies of the present technology. “Clearance” may refer to the elimination, absorption, and/or metabolism of the anti-TNFα antibodies in the subject's plasma. Clearance may be assessed as volume of plasma cleared of the anti-TNFα antibodies over time (e.g., mL/min, L/hr, or L/day) and/or may be normalized to body weight of the subject (e.g., mL/min/kg). Reduced clearance may also be represented by an increase in half-life or volume of distribution (V_{d}). In some embodiments, measuring clearance comprises measuring a terminal elimination rate constant (λ_{z}) or an inter-compartmental clearance (Q).

In some embodiments, the reduction in clearance comprises a measurement about 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the reduction in clearance comprises a measurement at least 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the reduction in clearance comprises a measurement at least about 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the reduction in clearance comprises a measurement during administration of the anti-TNFα antibodies.

In some embodiments, the reduction in clearance comprises a measurement at the completion of administration of the anti-TNFα antibodies.

In some embodiments, the reduction in clearance is a reduction by about 5%, 10%, 20%, 30%, 40%, 50%, 60%, 70%, 80%, 90%, or 100% relative to the control.

In some embodiments, the reduction in clearance is a reduction by at least 5%, 10%, 20%, 30%, 40%, 50%, 60%, 70%, 80%, 90% or 100% relative to the control.

In some embodiments, the reduction in clearance is a reduction by at least about 5%, 10%, 20%, 30%, 40%, 50%, 60%, 70%, 80%, 90%, or 100% relative to the control.

In some embodiments, the anti-TNFα antibodies of the present technology comprise an increased tissue, plasma, and/or serum concentration relative to a control. The control may be an anti-TNFα antibody or other TNFα inhibitor that lacks a post-translational modification possessed by the anti-TNFα antibodies of the present technology. “Concentration” may comprise a measurement reflecting one or more of the absolute amounts of the anti-TNFα antibodies, the absorption of the anti-TNFα antibodies, the metabolism of anti-TNFα antibodies, or the elimination of anti-TNFα antibodies.

The increased tissue, plasma, and/or serum concentration may be an increase in concentration of the anti-TNFα antibodies at a given time point relative to a control administered at the same dose and measured at the same time point. The concentration may be measured at an intermediate time point or a final time point and may be measured as a mean residence time (MRT), an average concentration (C_{avg}), a trough concentration (C_{trough}), or a concentration at the end of administration (e.g., infusion) time (C_{T}).

In some embodiments, the increased concentration is reflected by an increase in peak plasma concentration (Cₘₐₓ). An increase in Cₘₐₓ may suggest increased absorption, reduced metabolism, or slower elimination of the anti-TNFα antibodies, relative to a control. In some embodiments, Cₘₐₓ comprises a dose normalized Cₘₐₓ (DNCₘₐₓ).

In some embodiments, the increased concentration is reflected by a reduction in time to reach Cₘₐₓ (Tₘₐₓ). A reduced Tₘₐₓ may suggest increased absorption, reduced metabolism, or slower elimination of the anti-TNFα antibodies, relative to a control.

In some embodiments, the increased concentration is reflected by an increase in area under the curve (AUC) An increase in AUC may signify increased exposure to the anti-TNFα antibodies and/or may suggest increased absorption, reduced metabolism, or slower elimination of the anti-TNFα antibodies, relative to a control. The AUC measurement may comprise an Area Under the Curve for Concentration of Drug in Non-Compartmental Analysis (DNAUC).

In some embodiments, the increase in tissue, plasma, and/or serum concentration comprises a measurement about 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the increase in tissue, plasma, and/or serum concentration comprises a measurement at least 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the increase in tissue, plasma, and/or serum concentration comprises a measurement at least about 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the increase in tissue, plasma, and/or serum concentration comprises a measurement during administration of the anti-TNFα antibodies.

In some embodiments, the increase in tissue, plasma, and/or serum concentration comprises a measurement at the completion of administration of the anti-TNFα antibodies.

In some embodiments, the increase in tissue, plasma, and/or serum concentration is an increase by about 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the increase in tissue, plasma, and/or serum concentration is an increase by at least 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the increase in tissue, plasma, and/or serum concentration is an increase by at least about 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the anti-TNFα antibodies of the present technology comprise an increased distribution relative to a control. The control may be an anti-TNFα antibody or other TNFα inhibitor that lacks a post-translational modification possessed by the anti-TNFα antibodies of the present technology.

The increased distribution may be an increase in distribution of the anti-TNFα antibodies at a given time point relative to a control administered at the same dose and measured at the same time point. The distribution may be measured at an intermediate time point or a final time point.

In some embodiments, the measurement of distribution comprises measuring a volume of distribution at the terminal phase (V_{d} or V_{dB}), a central volume of distribution (V), a peripheral volume of distribution (V2), an apparent volume of distribution (V_{z}), or a measurement of distribution comprises measuring a volume of distribution at steady state (Vₛₛ). A high or increased V_{d}, V_{dB}, V_{z}, and/or Vₛₛ may suggest large distribution beyond the tissue, plasma, and/or serum compartment, relative to the control.

In some embodiments, the increase in distribution comprises a measurement about 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the increase in distribution comprises a measurement at least 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the increase in distribution comprises a measurement at least about 10 minutes, 20 minutes, 30 minutes, 40 minutes, 50 minutes, 1 hour, 1.5 hours, 2 hours, 3 hours, 4 hours, 5 hours, 6 hours, 12 hours, 1 day, 2 days, 3 days, 4 days, 5 day, 6 days, or 1 week after administration of the anti-TNFα antibodies.

In some embodiments, the increase in distribution comprises a measurement during administration of the anti-TNFα antibodies.

In some embodiments, the increase in distribution comprises a measurement at the completion of administration of the anti-TNFα antibodies.

In some embodiments, the increase in distribution is an increase by about 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the increase in distribution is an increase by at least 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

In some embodiments, the increase in distribution is an increase by at least about 5%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, or 1000% relative to the control.

### Signal Peptides

The anti-TNFα antibodies of the present technology may comprise one or more signal peptides. The signal peptides may be present in precursor and/or mature forms of the anti-TNFα antibodies and may direct peptides of the anti-TNFα antibodies to specific cellular locations. Nonlimiting examples of signal peptides include signal peptides that direct peptides to organelles (e.g., endoplasmic reticulum), signal peptides that promote peptide secretion, signal peptides that regulate protein sorting to subcellular components, signal peptides that promote peptide translocation across membranes, and signal peptides that promote peptide insertion into membranes. In some embodiments, the signal peptide is cleaved.

In some embodiments, the signal peptides comprise SEQ ID NO: 17 or SEQ ID NO: 18, listed in Table 4.

In some embodiments, the anti-TNFα antibodies comprise a signal peptide having an amino acid sequence that is about 75%, 80%, 85%, 90%, 95%, 99%, or 100% Identical to SEQ. ID NO: 17.

In some embodiments, the anti-TNFα antibodies comprise a signal peptide having an amino acid sequence that is at least 75%, 80%, 85%, 90%, 95%, 99%, or 100% Identical to SEQ. ID NO: 17.

In some embodiments, the anti-TNFα antibodies comprise a signal peptide having an amino acid sequence that is at least about 75%, 80%, 85%, 90%, 95%, 99%, or 100% Identical to SEQ. ID NO: 17.

In some embodiments, the anti-TNFα antibodies comprise a signal peptide having an amino acid sequence that is about 75%, 80%, 85%, 90%, 95%, 99%, or 100% Identical to SEQ. ID NO: 18.

In some embodiments, the anti-TNFα antibodies comprise a signal peptide having an amino acid sequence that is at least 75%, 80%, 85%, 90%, 95%, 99%, or 100% Identical to SEQ. ID NO: 18.

In some embodiments, the anti-TNFα antibodies comprise a signal peptide having an amino acid sequence that is at least about 75%, 80%, 85%, 90%, 95%, 99%, or 100% Identical to SEQ. ID NO: 18.

In some embodiments, the anti-TNFα antibodies comprise a signal peptide on a peptide chain having an amino acid sequence that is about 75%, 80%, 85%, 90%, 95%, 99%, or 100% Identical to SEQ. ID NO: 19.

In some embodiments, the anti-TNFα antibodies comprise a signal peptide on a peptide chain having an amino acid sequence that is at least 75%, 80%, 85%, 90%, 95%, 99%, or 100% Identical to SEQ. ID NO: 19.

In some embodiments, the anti-TNFα antibodies comprise a signal peptide on a peptide chain having an amino acid sequence that is at least about 75%, 80%, 85%, 90%, 95%, 99%, or 100% Identical to SEQ. ID NO: 19.

In some embodiments, the anti-TNFα antibodies comprise a signal peptide on a peptide chain having an amino acid sequence that is about 75%, 80%, 85%, 90%, 95%, 99%, or 100% Identical to SEQ. ID NO: 20.

In some embodiments, the anti-TNFα antibodies comprise a signal peptide on a peptide chain having an amino acid sequence that is at least 75%, 80%, 85%, 90%, 95%, 99%, or 100% Identical to SEQ. ID NO: 20.

In some embodiments, the anti-TNFα antibodies comprise a signal peptide on a peptide chain having an amino acid sequence that is at least about 75%, 80%, 85%, 90%, 95%, 99%, or 100% Identical to SEQ. ID NO: 20.

TABLE 4
Signal Peptides

| Signal Peptide | | SEQ |
|---|---|---|
| (Sequence Name) | Amino Acid Sequence | ID NO |
| Signal peptide U16 | GWSCIILFLVATATGV | 17 |
| Kappa | | |
| Signal Peptide U16 | GWSCIILFLVATATGVHS | 18 |
| Gamma | | |
| U16 Kappa | MGWSCIILFLVATATGVHSDILLTQSPAILSVSPGER | 19 |
| | VSFSCRASQFVGSSIHWYQQRTNGSPRLLIKYASE | |
| | SMSGIPSRFSGSGSGTDFTLSINTVESEDIADYYCQ | |
| | QSHSWPFTFGSGTNLEVKRTVAAPSVFIFPPSDEQ | |
| | LKSGTASVVCLLNNFYPREAKVQWKVDNALQSGN | |
| | SQESVTEQDSKDSTYSLSSTLTLSKADYEKHKVYA | |
| | CEVTHQGLSSPVTKSFNRGEC | |
| U16 Gamma | MGWSCIILFLVATATGVHSEVKLEESGGGLVQPGG | 20 |
| | SMKLSCVASGFIFSNHWMNWVRQSPEKGLEWVAE | |
| | IRSKSINSATHYAESVKGRFTISRDDSKSAVYLQMT | |
| | DLRTEDTGVYYCSRNYYGSTYDYWGQGTTLTVSS | |
| | ASTKGPSVFPLAPSSKSTSGGTAALGCLVKDYFPE | |
| | PVTVSWNSGALTSGVHTFPAVLQSSGLYSLSSVVT | |
| | VPSSSLGTQTYICNVNHKPSNTKVDKKVEPKSCDK | |
| | THTCPPCPAPELLGGPSVFLFPPKPKDTLMISRTPE | |
| | VTCVVVDVSHEDPEVKFNWYVDGVEVHNAKTKPR | |
| | EEQYNSTYRVVSVLTVLHQDWLNGKEYKCKVSNK | |
| | ALPAPIEKTISKAKGQPREPQVYTLPPSRDELTKNQ | |
| | VSLTCLVKGFYPSDIAVEWESNGQPENNYKTTPPV | |
| | LDSDGSFFLYSKLTVDKSRWQQGNVFSCSVMHEA | |
| | LHNHYTQKSLSLSPGK | |

### Nucleic Acid Sequences

In some embodiments, the anti-TNFα antibodies comprise a signal peptide encoded by a nucleic acid sequence that is about 75%, 80%, 85%, 90%, 95%, 99%, or 100% Identical to SEQ. ID NO: 21.

In some embodiments, the anti-TNFα antibodies comprise a signal peptide encoded by a nucleic acid sequence that is at least 75%, 80%, 85%, 90%, 95%, 99%, or 100% Identical to SEQ. ID NO: 21.

In some embodiments, the anti-TNFα antibodies comprise a signal peptide encoded by a nucleic acid sequence that is at least about 75%, 80%, 85%, 90%, 95%, 99%, or 100% Identical to SEQ. ID NO: 21.

In some embodiments, the anti-TNFα antibodies comprise a signal peptide encoded by a nucleic acid sequence that is about 75%, 80%, 85%, 90%, 95%, 99%, or 100% Identical to SEQ. ID NO: 22.

In some embodiments, the anti-TNFα antibodies comprise a signal peptide encoded by a nucleic acid sequence that is at least 75%, 80%, 85%, 90%, 95%, 99%, or 100% Identical to SEQ. ID NO: 22.

In some embodiments, the anti-TNFα antibodies comprise a signal peptide encoded by a nucleic acid sequence that is at least about 75%, 80%, 85%, 90%, 95%, 99%, or 100% Identical to SEQ. ID NO: 22.

In some embodiments, the anti-TNFα antibodies comprise a signal peptide on a peptide chain encoded by a nucleic acid sequence that is about 75%, 80%, 85%, 90%, 95%, 99%, or 100% Identical to SEQ. ID NO: 23.

In some embodiments, the anti-TNFα antibodies comprise a signal peptide on a peptide chain encoded by a nucleic acid sequence that is at least 75%, 80%, 85%, 90%, 95%, 99%, or 100% Identical to SEQ. ID NO: 23.

In some embodiments, the anti-TNFα antibodies comprise a signal peptide on a peptide chain encoded by a nucleic acid sequence that is at least about 75%, 80%, 85%, 90%, 95%, 99%, or 100% Identical to SEQ. ID NO: 23.

In some embodiments, the anti-TNFα antibodies comprise a signal peptide on a peptide chain encoded by a nucleic acid sequence that is about 75%, 80%, 85%, 90%, 95%, 99%, or 100% Identical to SEQ. ID NO: 24.

In some embodiments, the anti-TNFα antibodies comprise a signal peptide on a peptide chain encoded by a nucleic acid sequence that is at least 75%, 80%, 85%, 90%, 95%, 99%, or 100% Identical to SEQ. ID NO: 24.

In some embodiments, the anti-TNFα antibodies comprise a signal peptide on a peptide chain encoded by a nucleic acid sequence that is at least about 75%, 80%, 85%, 90%, 95%, 99%, or 100% Identical to SEQ. ID NO: 24.

TABLE 5
Nucleic Acid Sequences

| Nucleic Acid | | SEQ ID |
|---|---|---|
| Sequence | Nucleic Acid Sequence | NO |
| Signal peptide U16 | GGCTGGTCCTGCATCATCCTGTTTCTGGTG | 21 |
| Kappa Nucleic Acid | GCCACCGCCACCGGCGTGCACT | |
| Sequence | | |
| Signal Peptide U16 | GGCTGGTCCTGCATCATCCTGTTTCTGGTG | 22 |
| Gamma Nucleic | GCCACCGCCACCGGCGTGCACTCC | |
| Acid Sequence | | |
| U16 Kappa Nucleic | GCTAGCGAATTCCACCATGGGCTGGTCCTG | 23 |
| Acid Sequence | CATCATCCTGTTTCTGGTGGCCACCGCCAC | |
| | CGGCGTGCACTCCGACATCCTGCTGACCCA | |
| | GTCCCCCGCCATCCTGTCCGTGTCTCCCGG | |
| | CGAGCGGGTGTCCTTCTCCTGCCGGGCCT | |
| | CCCAGTTCGTGGGCTCCTCCATCCACTGGT | |
| | ATCAGCAGCGGACCAACGGCTCCCCTCGG | |
| | CTGCTGATCAAGTACGCCTCCGAGTCCATG | |
| | TCCGGCATCCCTTCCCGGTTCTCCGGCTCC | |
| | GGCTCTGGCACCGACTTCACCCTGTCCATC | |
| | AACACCGTGGAGTCCGAGGATATTGCCGAC | |
| | TACTACTGCCAGCAGTCCCACTCCTGGCCT | |
| | TTCACCTTCGGCTCCGGCACCAACCTGGAG | |
| | GTGAAGCGTACGGTGGCTGCACCATCTGTC | |
| | TTCATCTTCCCGCCATCTGATGAGCAGTTG | |
| | AAATCTGGAACTGCCTCTGTTGTGTGCCTG | |
| | CTGAATAACTTCTATQCCAGAGAGGCCAAA | |
| | GTACAGTGGAAGGTGGATAACGCCCTCCAA | |
| | TCGGGTAACTCCCAGGAGAGTGTCACAGAG | |
| | CAGGACAGCAAGGACAGCACCTACAGCCT | |
| | CAGCAGCACCCTGACGCTGAGCAAAGCAG | |
| | ACTACGAGAAACACAAAGTCTACGCCTGCG | |
| | AAGTCACCCATCAGGGCCTGAGCTCGCCC | |
| | GTCACAAAGAGCTTCAACAGGGGAGAGTGT | |
| | TAA | |
| U16 Gamma | GCTAGCGATATCGAATTCTTTTCTTTTGTTTT | 24 |
| Nucleic Acid | GTTTCCACCATGGGCTGGTCCTGCATCATC | |
| Sequence | CTGTTTCTGGTGGCCACCGCCACCGGCGT | |
| | GCACTCCGAGGTGAAGCTGGAGGAGTCTG | |
| | GCGGCGGACTGGTGCAGCCTGGCGGCTCC | |
| | ATGAAGCTGTCCTGCGTGGCCTCCGGCTTC | |
| | ATCTTCTCCAACCACTGGATGAACTGGGTG | |
| | CGCCAGTCCCCTGAGAAGGGCCTGGAGTG | |
| | GGTGGCCGAGATCCGGTCCAAGTCCATCAA | |
| | CTCCGCCACCCACTACGCCGAGTCCGTGAA | |
| | GGGCCGGTTCACCATCTCCCGGGACGACT | |
| | CCAAGTCCGCCGTGTACCTGCAGATGACCG | |
| | ACCTGCGGACCGAGGACACCGGAGTGTAC | |
| | TACTGCTCCCGGAACTACTACGGCTCCACC | |
| | TACGACTACTGGGGCCAGGGCACCACACT | |
| | GACCGTGAGCTCCGCCTCCACCAAGGGCC | |
| | CATCGGTCTTCCCCCTGGCACCCTCCTCCA | |
| | AGAGCACCTCTGGGGGCACAGCGGCCCTG | |
| | GGCTGCCTGGTCAAGGACTACTTCCCCGAA | |
| | CCGGTGACGGTGTCGTGGAACTCAGGCGC | |
| | CCTGACCAGCGGCGTGCACACCTTCCCGG | |
| | CTGTCCTACAGTCCTCAGGACTCTACTCCC | |
| | TCAGCAGCGTGGTGACCGTGCCCTCCAGC | |
| | AGCTTGGGCACCCAGACCTACATCTGCAAC | |
| | GTGAATCACAAGCCCGCAACACCAAGGTGG | |
| | ACAAGAAAGTCGAGCCCAAATCTTGTGACA | |
| | AAACTCACACATGCCCACCGTGCCCAGCAC | |
| | CTGAACTCCTGGGGGGACCGTCAGTCTTCC | |
| | TCTTCCCCCCAAAACCCAAGGACACCCTCA | |
| | TGATCTCCCGGACCCCTGAGGTCACATGCG | |
| | TGGTGGTGGACGTGAGCCACGAAGACCCT | |
| | GAGGTCAAGTTCAACTGGTACGTGGACGGC | |
| | GTGGAGGTGCATAATGCCAAGACAAAGCCG | |
| | CGGGAGGAGCAGTACAACAGCACGTACCG | |
| | TGTGGTCAGCGTCCTCACCGTCCTGCACCA | |
| | GGACTGGCTGAATGGCAAGGAGTACAAGT | |
| | GCAAGGTCTCCAACAAAGCCCTCCCAGCCC | |
| | CCATCGAGAAAACCATCTCCAAAGCCAAAG | |
| | GGCAGCCCCGAGAACCACAGGTGTACACC | |
| | CTGCCCCCATCCCGGGATGAGCTGACCAA | |
| | GAACCAGGTCAGCCTGACCTGCCTGGTCAA | |
| | AGGCTTCTATCCCAGCGACATCGCCGTGGA | |
| | GTGGGAGAGTAATGGGCAGCCGGAGAACA | |
| | ACTACAAGACCACGCCTCCCGTGCTGGACT | |
| | CCGACGGCTCCTTCTTCCTCTACAGCAAGC | |
| | TCACCGTGGACAAGAGCAGGTGGCAGCAG | |
| | GGGAACGTCTTCTCATGCTCCGTGATGCAT | |
| | GAGGCTCTGCACAACCACTACACGCAGAAG | |
| | AGCCTCTCCCTGTCTCCGGGTAAATAA | |

### Other Anti-TNFα Antibody Features

The anti-TNFα antibodies of the present technology may comprise an isotype that influences functional properties and/or structural features of the antibody. In some embodiments the anti-TNFα antibodies comprise an isotype selected from the group consisting of IgG, IgM, IgA, IgD, and IgE. The light chain may comprise isotype K or isotype A.

In some embodiments, the anti-TNFα antibodies of the present technology are chimeric antibodies. Nonlimiting examples of chimeric antibodies include human-murine antibodies, human-rat antibodies, human-hamster antibodies, human-rabbit antibodies, human-bovine antibodies, and human-goat antibodies. In some embodiments, the chimeric antibodies comprise a humanized antibody.

### Compositions and Formulations

One or more anti-TNFα antibodies of the present technology may be present in a composition (e.g., a pharmaceutical composition). In some embodiments, the composition comprises a pharmaceutically acceptable carrier and/or excipient. The pharmaceutically acceptable carrier and/or excipient may comprise aqueous or non-aqueous carriers that may facilitate dissolution of the anti-TNFα antibodies. Nonlimiting examples of pharmaceutically acceptable carriers and/or excipients include sucrose, polysorbate 80, monobasic sodium phosphate, monohydrate, dibasic sodium phosphate, dihydrate. The composition may comprise a filler or diluent (e.g., lactose, mannitol, and microcrystalline cellulose), a disintegrant (e.g., croscarmellose sodium, crospovidone, starches), a buffer (e.g., phosphate buffer or acetate buffers), or a solvent (e.g., water, alcohol, glycerin).

In some embodiments, the composition does not comprise a preservative.

In some embodiments, the composition is sterilized (e.g., sterilization by filtration).

In some embodiments, the compositions comprise one or more anti-TNFα antibodies of the present technology in an amount of about 0.1% w/v or w/w of the composition, at least about 1% w/v or w/w of the composition; about 10% w/v or w/w of the composition; about 20% w/v or w/w of the composition; about 30% w/v or w/w of the composition; about 40% w/v or w/w of the composition; about 50% w/v or w/w of the composition; about 60% w/v or w/w of the composition; about 70% w/v or w/w of the composition; about 80% w/v or w/w of the composition; about 90% w/v or w/w of the composition; about 95% w/v or w/w of the composition; or about 99% w/v or w/w of the composition.

In some embodiments, the compositions comprise one or more anti-TNFα antibodies of the present technology in an amount of at least 0.1% w/v or w/w of the composition, at least at least 1% w/v or w/w of the composition; at least 10% w/v or w/w of the composition; at least 20% w/v or w/w of the composition; at least 30% w/v or w/w of the composition; at least 40% w/v or w/w of the composition; at least 50% w/v or w/w of the composition; at least 60% w/v or w/w of the composition; at least 70% w/v or w/w of the composition; at least 80% w/v or w/w of the composition; at least 90% w/v or w/w of the composition; at least 95% w/v or w/w of the composition; or at least 99% w/v or w/w of the composition.

In some embodiments, the compositions comprise one or more anti-TNFα antibodies of the present technology in an amount of at least about 0.1% w/v or w/w of the composition, at least about 1% w/v or w/w of the composition; at least about 10% w/v or w/w of the composition; at least about 20% w/v or w/w of the composition; at least about 30% w/v or w/w of the composition; at least about 40% w/v or w/w of the composition; at least about 50% w/v or w/w of the composition; at least about 60% w/v or w/w of the composition; at least about 70% w/v or w/w of the composition; at least about 80% w/v or w/w of the composition; at least about 90% w/v or w/w of the composition; at least about 95% w/v or w/w of the composition; or at least about 99% w/v or w/w of the composition.

In some embodiments, the composition comprises a lyophilized drug product (lyophilized powder or lyophilized cake of the anti-TNFα antibody). In some embodiments, the lyophilized drug product is reconstituted (e.g., using water or saline) prior to administration.

In some embodiments, the reconstituted composition provides a solution having a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments, the reconstituted composition provides a solution having a pH of at least 5.0, at least 5.1, at least 5.2, at least 5.3, at least 5.4, at least 5.5, at least 5.6, at least 5.7, at least 5.8, at least 5.9, at least 6.0, at least 6.1, at least 6.2, at least 6.3, at least 6.4, at least 6.5, at least 6.7, at least 6.8, at least 6.9, at least 7.0, at least 7.1, at least 7.2, at least 7.3, at least 7.4, at least 7.5, at least 7.6, at least 7.7, at least 7.8, at least 7.9, or at least 8.0.

In some embodiments, the reconstituted composition provides a solution having a pH of at least about 5.0, at least about 5.1, at least about 5.2, at least about 5.3, at least about 5.4, at least about 5.5, at least about 5.6, at least about 5.7, at least about 5.8, at least about 5.9, at least about 6.0, at least about 6.1, at least about 6.2, at least about 6.3, at least about 6.4, at least about 6.5, at least about 6.7, at least about 6.8, at least about 6.9, at least about 7.0, at least about 7.1, at least about 7.2, at least about 7.3, at least about 7.4, at least about 7.5, at least about 7.6, at least about 7.7, at least about 7.8, at least about 7.9, or at least about 8.0.

In some embodiments, the reconstituted composition provides a solution having a concentration of an anti-TNFα antibody that is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL about 13 mg/mL, about 14 mg/mL, about 15 mg/mL, about 20 mg/mL, about 25 mg/ML, about 30 mg/mL, or about 40 mg/mL, about 50 mg/mL, about 60 mg/mL, about 70 mg/mL, about 80 mg/mL, about 90 mg/mL, or about 100 mg/mL.

In some embodiments, the reconstituted composition provides a solution having a concentration of an anti-TNFα antibody that is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, at least 10 mg/mL, at least 11 mg/mL, at least 12 mg/mL at least 13 mg/mL, at least 14 mg/mL, at least 15 mg/mL, at least 20 mg/mL, at least 25 mg/ML, at least 30 mg/mL, or at least 40 mg/mL, at least 50 mg/mL, at least 60 mg/mL, at least 70 mg/mL, at least 80 mg/mL, at least 90 mg/mL, or at least 100 mg/mL.

In some embodiments, the reconstituted composition provides a solution having a concentration of an anti-TNFα antibody that is at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, at least about 10 mg/mL, at least about 11 mg/mL, at least about 12 mg/mL at least about 13 mg/mL, at least about 14 mg/mL, at least about 15 mg/mL, at least about 20 mg/mL, at least about 25 mg/ML, at least about 30 mg/mL, or at least about 40 mg/mL, at least about 50 mg/mL, at least about 60 mg/mL, at least about 70 mg/mL, at least about 80 mg/mL, at least about 90 mg/mL, or at least about 100 mg/mL.

In some embodiments, the composition or the solution is diluted for administration. Nonlimiting examples of diluents include sodium chloride, water (e.g., sterile water for injection (SWFI)), dextrose solutions, bacteriostatic solutions, or Ringer's solutions (e.g., solutions comprising electrolytes, lactated Ringer's solution).

The compositions may be formulated for intraperitoneal, intravenous, parenteral, subcutaneous, intramuscular, intracerebroventricular, or oral administration. In some embodiments, the compositions formulated for parenteral administration comprise the composition in the form of aqueous or non-aqueous isotonic sterile injection solutions or suspensions. The term “parenteral”, as used herein, includes subcutaneous, intravenous, intraperitoneal, intramuscular, and intralesional, or infusion techniques

### Associated Methods

The present technology provides methods comprising administering one or more compositions of the present technology to a subject in need thereof. In some embodiments, the present technology comprises methods of treating or reducing sarcoidosis in a subject in need thereof relative to a control. In some embodiments, the present technology comprises methods of reducing granuloma size or granuloma formation in a subject in need thereof relative to a control. The control may refer to the subject at baseline, the subject at an intermediate timepoint, a second subject not receiving treatment, a second subject at an intermediate time point, or a second subject at baseline.

In some embodiments, the subject has one or more of acute sarcoidosis, chronic sarcoidosis, progressive sarcoidosis, pulmonary sarcoidosis (e.g., with or without extrapulmonary manifestations), remitting sarcoidosis, refractory sarcoidosis, advanced sarcoidosis, ocular sarcoidosis, cutaneous sarcoidosis, or neurosacoidosis. In some embodiments, the sarcoidosis comprises high responder or low responder sarcoidosis, wherein the designation of high or low responder sarcoidosis may indicate the response to a therapeutic intervention or a response to purified protein derivative (PPD)-coated beads in terms of in vitro granuloma formation.

In some embodiments, the subject has one or more granulomas. In some embodiments, the one or more granulomas comprises a skin granuloma, a lung granuloma, a lymph node granuloma, an ocular granuloma, a liver granuloma, a spleen granuloma, a cardiac granuloma, or a rheumatoid granuloma.

In some embodiments, the subject has a Modified Medical Research Conference (mMRC) Dyspnea Scale of ≥1.

In some embodiments, the subject has a Scadding CXR grade determined by based on chest X-ray methods. In some embodiments, the subject has a Scadding CXR grade selected from the group consisting of Stage 0, Stage II, Stage III, or Stage IV.

In some embodiments, the subject is at least 18 years of age. In some embodiments, the subject is between about 18 and about 80 years of age. In some embodiments, the subject weighs at least 45 kg. In some embodiments, the subject weights between about 45 and about 160 kg.

In some embodiment, the subject has sarcoidosis and/or one or more granulomas and one or more additional inflammatory conditions. In some embodiments, the inflammatory condition is an autoimmune disorder. Nonlimiting examples of autoimmune disorders include Rheumatoid Arthritis (RA), Systemic Lupus Erythematosus (SLE), Multiple Sclerosis (MS), Type 1 Diabetes (T1D), psoriasis, celiac disease, Hashimoto's thyroiditis, Graves' disease, Crohn's disease, and Myasthenia Gravis (MG).

In some embodiments, the subject comprises anti-drug antibodies (ADA). The ADAs may be produced by the subject's immune system in response to a therapeutic (e.g., a therapeutic comprising a TNFα inhibitor). The ADAs may recognize and/or bind the therapeutic, potentially reducing the efficacy of the therapeutic (e.g., neutralizes the therapeutic and/or triggers immune reactions against the therapeutic).

In some embodiments, the subject comprises neutralizing antibodies (nAB), which may inhibit the biological activity of a therapeutic (e.g., a therapeutic comprising a TNFα inhibitor). When nAb are present, they may prevent the therapeutic from binding to its target or interfere with its function, reducing its efficacy.

In some embodiments, the subject has received an anti-inflammatory medication. In some embodiments, the subject is receiving an anti-inflammatory medication. In some embodiments, the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.

In some embodiments, the corticosteroid is selected from the group consisting of prednisone, prednisolone, dexamethasone, hydrocortisone, methylprednisolone, betamethasone, cortisone, fludrocortisone, and triamcinolone. In some embodiments, the subject has received or is receiving about 7.5 to about 25 mg/day of the corticosteroid.

In some embodiments, the subject has received or is receiving a therapeutic selected from the group consisting of a short-acting beta-agonist (SABA) (e.g., albuterol, salbutamol), a Short-acting muscarinic antagonist (SAMA) (e.g., ipratropium bromide), a Long-acting beta-agonist (LABA) (e.g., formoterol or salmeterol), an Ultra-long-acting beta-agonist (Ultra-LABA) (e.g., indacaterol, vilanterol, or olodaterol), and a Long-acting muscarinic antagonist (LAMA) (e.g., tiotropium, umeclidinium, aclidinium, or glycopyrronium).

### Doses/Phases

In some embodiments, the composition is administered to the subject as a first dose. The first dose may comprise administering the composition to the subject in an amount of about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, or about 6.0 mg/kg.

In some embodiments, the composition is administered to the subject as a first dose in an amount of at least 0.5 mg/kg, at least 1.0 mg/kg, at least 1.5 mg/kg, at least 2.0 mg/kg, at least 2.5 mg/kg, at least 3.0 mg/kg, at least 3.5 mg/kg, at least 4.0 mg/kg, at least 4.5 mg/kg, at least 5.0 mg/kg, at least 5.5 mg/kg, or at least 6.0 mg/kg.

In some embodiments, the composition is administered to the subject as a first dose in an amount of at least about 0.5 mg/kg, at least about 1.0 mg/kg, at least about 1.5 mg/kg, at least about 2.0 mg/kg, at least about 2.5 mg/kg, at least about 3.0 mg/kg, at least about 3.5 mg/kg, at least about 4.0 mg/kg, at least about 4.5 mg/kg, at least about 5.0 mg/kg, at least about 5.5 mg/kg, or at least about 6.0 mg/kg.

In some embodiments, the first dose of composition is administered about once a week, about once every two weeks, about once every three weeks, about once every four weeks, about once every 5 weeks, or about once every 6 weeks for a first period of time.

In some embodiments, the first dose of composition is administered at least once a week, at least once every two weeks, at least at least once every three weeks, at least once every four weeks, at least once every 5 weeks, or at least once every 6 weeks for a first period of time.

In some embodiments, the first dose of composition is administered at least about once a week, at least about once every two weeks, at least about once every three weeks, at least about once every four weeks, at least about once every 5 weeks, or at least about once every 6 weeks for a first period of time.

In some embodiments, the first period of time is about 6 weeks, about 8 weeks, about 10 weeks, about 12 weeks, about 14 weeks, about 15 weeks, about 16 weeks, about 18 weeks, about 20 weeks, about 22 weeks, or about 24 weeks.

In some embodiments, the first period of time is at least 6 weeks, at least 8 weeks, at least 10 weeks, at least 12 weeks, at least 14 weeks, at least 15 weeks, at least 16 weeks, at least 18 weeks, at least 20 weeks, at least 22 weeks, or at least 24 weeks.

In some embodiments, the first period of time is at least about 6 weeks, at least about 8 weeks, at least about 10 weeks, at least about 12 weeks, at least about 14 weeks, at least about 15 weeks, at least about 16 weeks, at least about 18 weeks, at least about 20 weeks, at least about 22 weeks, or at least about 24 weeks.

In some embodiments, the subject is administered a cumulative dose of about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg of the composition during the first period of time.

In some embodiments, the subject is administered a cumulative dose of at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, at least 15 mg/kg, at least 16 mg/kg, at least 17 mg/kg, at least 18 mg/kg, at least 19 mg/kg, or at least 20 mg/kg of the composition during the first period of time.

In some embodiments, the subject is administered a cumulative dose of at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, or at least about 20 mg/kg of the composition during the first period of time.

In some embodiments, the composition is administered to the subject as a second dose. In some embodiments, the second dose comprises administering the composition to the subject in an amount of about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, or about 6.0 mg/kg.

In some embodiments, the composition is administered to the subject as a second dose in an amount of at least 0.5 mg/kg, at least 1.0 mg/kg, at least 1.5 mg/kg, at least 2.0 mg/kg, at least 2.5 mg/kg, at least 3.0 mg/kg, at least 3.5 mg/kg, at least 4.0 mg/kg, at least 4.5 mg/kg, at least 5.0 mg/kg, at least 5.5 mg/kg, or at least 6.0 mg/kg.

In some embodiments, the composition is administered to the subject as a second dose in an amount of at least about 0.5 mg/kg, at least about 1.0 mg/kg, at least about 1.5 mg/kg, at least about 2.0 mg/kg, at least about 2.5 mg/kg, at least about 3.0 mg/kg, at least about 3.5 mg/kg, at least about 4.0 mg/kg, at least about 4.5 mg/kg, at least about 5.0 mg/kg, at least about 5.5 mg/kg, or at least about 6.0 mg/kg.

In some embodiments, the second dose of composition is administered about once a week, about once every two weeks, about once every three weeks, about once every four weeks, about once every 5 weeks, or about once every 6 weeks for a second period of time.

In some embodiments, the second dose of composition is administered at least once a week, at least once every two weeks, at least at least once every three weeks, at least once every four weeks, at least once every 5 weeks, or at least once every 6 weeks for a second period of time.

In some embodiments, the second dose of composition is administered at least about once a week, at least about once every two weeks, at least about once every three weeks, at least about once every four weeks, at least about once every 5 weeks, or at least about once every 6 weeks for a second period of time.

In some embodiments, the second period of time is about 6 weeks, about 8 weeks, about 10 weeks, about 12 weeks, about 14 weeks, about 15 weeks, about 16 weeks, about 18 weeks, about 20 weeks, about 22 weeks, or about 24 weeks.

In some embodiments, the second period of time is at least 6 weeks, at least 8 weeks, at least 10 weeks, at least 12 weeks, at least 14 weeks, at least 15 weeks, at least 16 weeks, at least 18 weeks, at least 20 weeks, at least 22 weeks, or at least 24 weeks.

In some embodiments, the second period of time is at least about 6 weeks, at least about 8 weeks, at least about 10 weeks, at least about 12 weeks, at least about 14 weeks, at least about 15 weeks, at least about 16 weeks, at least about 18 weeks, at least about 20 weeks, at least about 22 weeks, or at least about 24 weeks.

In some embodiments, the second dose is about two times greater than the first dose.

In some embodiments, the second dose is at least two times greater than the first dose.

In some embodiments, the second dose is at least about two times greater than the first dose.

In some embodiments, the second dose is about the same at the first dose.

In some embodiments, the administration frequency of the second dose comprises the same administration frequency of the first dose.

In some embodiments, the administration frequency of the second dose consists of the same administration frequency of the first dose.

In some embodiments, the administration frequency of the second dose is about two times greater than the administration frequency of the first dose

In some embodiments, the first dose and the second dose are each administered about once every four weeks.

In some embodiments, the first dose is administered about once every four weeks and the second dose is administered about once every two weeks.

In some embodiments, the subject is administered a cumulative dose of about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg of the composition during the second period of time.

In some embodiments, the subject is administered a cumulative dose of at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, at least 15 mg/kg, at least 16 mg/kg, at least 17 mg/kg, at least 18 mg/kg, at least 19 mg/kg, or at least 20 mg/kg of the composition during the second period of time.

In some embodiments, the subject is administered a cumulative dose of at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, or at least about 20 mg/kg of the composition during the second period of time.

In some embodiments, the composition is administered to the subject as a third dose. In some embodiments, the third dose comprises administering the composition to the subject in an amount of about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, or about 6.0 mg/kg.

In some embodiments, the composition is administered to the subject as a third dose in an amount of at least 0.5 mg/kg, at least 1.0 mg/kg, at least 1.5 mg/kg, at least 2.0 mg/kg, at least 2.5 mg/kg, at least 3.0 mg/kg, at least 3.5 mg/kg, at least 4.0 mg/kg, at least 4.5 mg/kg, at least 5.0 mg/kg, at least 5.5 mg/kg, or at least 6.0 mg/kg.

In some embodiments, the composition is administered to the subject as a third dose in an amount of at least about 0.5 mg/kg, at least about 1.0 mg/kg, at least about 1.5 mg/kg, at least about 2.0 mg/kg, at least about 2.5 mg/kg, at least about 3.0 mg/kg, at least about 3.5 mg/kg, at least about 4.0 mg/kg, at least about 4.5 mg/kg, at least about 5.0 mg/kg, at least about 5.5 mg/kg, or at least about 6.0 mg/kg.

In some embodiments, the third dose of composition is administered about once a week, about once every two weeks, about once every three weeks, about once every four weeks, about once every 5 weeks, or about once every 6 weeks for a third period of time.

In some embodiments, the third dose of composition is administered at least once a week, at least once every two weeks, at least at least once every three weeks, at least once every four weeks, at least once every 5 weeks, or at least once every 6 weeks for a third period of time.

In some embodiments, the third dose of composition is administered at least about once a week, at least about once every two weeks, at least about once every three weeks, at least about once every four weeks, at least about once every 5 weeks, or at least about once every 6 weeks for a third period of time.

In some embodiments, the third period of time is about 6 weeks, about 8 weeks, about 10 weeks, about 12 weeks, about 14 weeks, about 15 weeks, about 16 weeks, about 18 weeks, about 20 weeks, about 22 weeks, or about 24 weeks.

In some embodiments, the third period of time is at least 6 weeks, at least 8 weeks, at least 10 weeks, at least 12 weeks, at least 14 weeks, at least 15 weeks, at least 16 weeks, at least 18 weeks, at least 20 weeks, at least 22 weeks, or at least 24 weeks.

In some embodiments, the third period of time is at least about 6 weeks, at least about 8 weeks, at least about 10 weeks, at least about 12 weeks, at least about 14 weeks, at least about 15 weeks, at least about 16 weeks, at least about 18 weeks, at least about 20 weeks, at least about 22 weeks, or at least about 24 weeks.

In some embodiments, the subject is administered a cumulative dose of about 20 mg/kg, about 22 mg/kg, about 24 mg/kg, about 25 mg/kg, about 26 mg/kg, about 28 mg/kg, about 30 mg/kg, about 35 mg/kg, about 40 mg/kg, about 44 mg/kg, about 46 mg/kg, about 48 mg/kg, about 50 mg/kg, about 52 mg/kg, about 54 mg/kg, about 56 mg/kg, about 58 mg/kg, about 60 mg/kg, about 65 mg/kg, about 70 mg/kg, or about 75 mg/kg of the composition, across all periods of time.

In some embodiments, the subject is administered a cumulative dose of at least 20 mg/kg, at least 22 mg/kg, at least 24 mg/kg, at least 25 mg/kg, at least 26 mg/kg, at least 28 mg/kg, at least 30 mg/kg, at least 35 mg/kg, at least 40 mg/kg, at least 44 mg/kg, at least 46 mg/kg, at least 48 mg/kg, at least 50 mg/kg, at least 52 mg/kg, at least 54 mg/kg, at least 56 mg/kg, at least 58 mg/kg, at least 60 mg/kg, at least 65 mg/kg, at least 70 mg/kg, or at least 75 mg/kg of the composition, across all periods of time.

In some embodiments, the subject is administered a cumulative dose of at least about 20 mg/kg, at least about 22 mg/kg, at least about 24 mg/kg, at least about 25 mg/kg, at least about 26 mg/kg, at least about 28 mg/kg, at least about 30 mg/kg, at least about 35 mg/kg, at least about 40 mg/kg, at least about 44 mg/kg, at least about 46 mg/kg, at least about 48 mg/kg, at least about 50 mg/kg, at least about 52 mg/kg, at least about 54 mg/kg, at least about 56 mg/kg, at least about 58 mg/kg, at least about 60 mg/kg, at least about 65 mg/kg, at least about 70 mg/kg, or at least about 75 mg/kg of the composition, across all periods of time.

In some embodiments, the compositions are administered using an infusion (e.g., intravenous infusion). In some embodiments, the infusion is administered over a period of about 30 minutes, about 1 hour, about 2 hours, about 3 hours, about 4 hours, or about 5 hours.

In some embodiments, the infusion is administered over a period of at least 30 minutes, at least 1 hour, at least 2 hours, at least 3 hours, at least 4 hours, or at least 5 hours.

In some embodiments, the infusion is administered over a period of at least about 30 minutes, at least about 1 hour, at least about 2 hours, at least about 3 hours, at least about 4 hours, or at least about 5 hours.

### Outcomes of Administration and Uses of the Compositions

In some embodiments, the subject administered a composition of the present technology experiences a reduction in a level of one or more transcripts selected from the group consisting of angiotensin-converting enzyme 2 (ACE2), interleukin 6 (IL6), interleukin 2 receptor (IL2R), tumor necrosis factor alpha (TNFα), C-Reactive Protein (CRP), interleukin 1B (IL1B), interleukin 12 (IL12), interleukin 18 (IL18), interferon gamma (IFNG), interleukin 8 (IL8), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JunB), mitogen-activated protein kinase (MKP1), TNF alpha induced protein 1 (TNFAIP1), TNF alpha induced protein 2 (TNFAIP2), TNF alpha induced protein 3 (TNFAIP3), syndecan 4 (SDC4), superoxide dismutase 2 (SOD2), cytochrome c oxidase subunit II (COX2), interleukin 32 (IL32), intercellular adhesion molecule 1 (ICAM-1), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), and vascular adhesion molecule 1 (VCAM-1), relative to a control

In some embodiments, the subject administered a composition of the present technology experiences a reduction in a level of one or more proteins selected from the group consisting of angiotensin-converting enzyme 2 (ACE2), interleukin 6 (IL-6), interleukin 2 receptor (IL-2R), tumor necrosis factor alpha (TNF-α), C-Reactive Protein (CRP), interleukin 1B (IL-1B), interferon gamma (IFN-γ), interleukin 8 (IL-8), interleukin 12 (IL-12), interleukin 18 (IL-18), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JUNB), mitogen-activated protein kinase phosphatase 1 (MKP-1), TNF alpha-induced protein 1 (TNFAIP1), TNF alpha-induced protein 2 (TNFAIP2), TNF alpha-induced protein 3 (TNFAIP3), syndecan 4 (SDC4), superoxide dismutase 2 (SOD2), cytochrome c oxidase subunit II (COX2), interleukin 32 (IL-32), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), intercellular adhesion molecule 1 (ICAM-1), and vascular adhesion molecule 1 (VCAM-1) relative to a control.

In some embodiments, the subject administered a composition of the present technology experiences an increase in one or more of a Forced Vital Capacity (FVC) measurement, a Forced Expiratory Volume in 1 second (FEV1) measurement, a Leicester Cough Questionnaire (LCQ) score, a Short Form 36 Health Survey (SF-36) score, a King's Sarcoidosis Questionnaire (KSQ) score (e.g., a KSQ General Health Status Module or a KSQ lung score), a Steroid-toxicity scale (STS) score, or a 6-minute walk test (6MWT) distance relative to a control.

In some embodiments, the subject administered a composition of the present technology experiences a reduction in one or more of a Patient Global Assessment (PGA) score, a Fatigue Assessment Scale (FAS) score, Modified Medical Research Council (mMRC) Dyspnea Scale score, a Saint George's Respiratory Questionnaire (SGRQ) score, or a Borg's CR10 dyspnea score relative to a control.

In some embodiments, the subject administered a composition of the present technology experiences a reduction in stage on the Scadding CXR relative to a control.

In some embodiments, the subject administered a composition of the present technology experiences an improved quality of life relative to a control. In some embodiments, the quality of life is assessed by one or more of a KSQ score, a LCQ score, a PGA score, a FAS score, a mMRC Dyspnea Scale score, a SF-36 score, or a steroid toxicity questionnaire (STQ) score.

In some embodiments, the subject administered a composition of the precent technology experiences an improvement in one or more limitations selected from the group consisting of a limitation in physical activities because of health problems, a limitation in social activities because of physical or emotional problems, limitations in usual role activities because of physical health problems, and a limitation s in usual role activities because of emotional problems, relative to a control.

In some embodiments, the subject administered a composition of the precent technology experiences a reduction in bodily pain relative to a control.

In some embodiments, the subject administered a composition of the precent technology experiences an improvement in mental health relative to a control.

In some embodiments, the subject administered a composition of the precent technology experiences an increase in vitality (e.g., increase in energy and/or reduction in fatigue) relative to a control.

In some embodiments, the subject administered a composition of the present technology experiences an improvement in general health perception relative to a control.

In some embodiments, the subject administered a composition of the present technology experiences an increase in lifespan relative to a control.

In some embodiments, the subject administered a composition of the present technology experiences a reduced need (e.g., frequency of, dosage of, or duration of administration of) for an anti-inflammatory medication relative to a control. In some embodiments, the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab. In some embodiments, the corticosteroid is selected from the group consisting of prednisone, prednisolone, dexamethasone, hydrocortisone, methylprednisolone, betamethasone, cortisone, fludrocortisone, and triamcinolone.

In some embodiments, the subject administered a composition of the present technology experiences a reduction in a granuloma formation or a granuloma size relative to a control. In some embodiments, the granuloma is a skin granuloma, a lung granuloma, a lymph node granuloma, an ocular granuloma, a liver granuloma, a spleen granuloma, a cardiac granuloma, or a rheumatoid granuloma.

In some embodiments, the subject administered a composition of the present technology experiences a reduction in a calcitriol level, a neopterin level, a soluble IL-2R (sIL-2R) level, or a lysozyme level, in the subject relative to a control.

In some embodiments, the subject administered a composition of the present technology experiences a reduction in immune cell anergy relative to a control.

In some embodiments, the subjected administered a dose of one or more compositions of the present technology exhibits one or more of:
(a) a reduction in an ACE2 transcript level by about 10%, 20%, 30%, 40%, 50%, 60%, 70%, 80%, 90%, or 100% relative to a control;
(b) a reduction in an IL6 transcript level by about 10%, 20%, 30%, 40%, 50%, 60%, 70%, 80%, 90%, or 100% relative to a control;
(c) a reduction in an IL2R transcript level by about 10%, 20%, 30%, 40%, 50%, 60%, 70%, 80%, 90%, or 100% relative to a control;
(d) a reduction in a TNFα transcript level by about 10%, 20%, 30%, 40%, 50%, 60%, 70%, 80%, 90%, or 100% relative to a control;
(e) a reduction in a CRP transcript level by about 10%, 20%, 30%, 40%, 50%, 60%, 70%, 80%, 90%, or 100% relative to a control;
(f) a reduction in an IL1B transcript level by about 10%, 20%, 30%, 40%, 50%, 60%, 70%, 80%, 90%, or 100% relative to a control;
(g) a reduction in an IL12 transcript level by about 10%, 20%, 30%, 40%, 50%, 60%, 70%, 80%, 90%, or 100% relative to a control;
(h) a reduction in an IL18 transcript level by about 10%, 20%, 30%, 40%, 50%, 60%, 70%, 80%, 90%, or 100% relative to a control;
(i) a reduction in an IFNG transcript level by about 10%, 20%, 30%, 40%, 50%, 60%, 70%, 80%, 90%, or 100% relative to a control;
(j) a reduction in an IL8 transcript level by about 10%, 20%, 30%, 40%, 50%, 60%, 70%, 80%, 90%, or 100% relative to a control;
(k) a reduction in a MCP-1 transcript level by about 10%, 20%, 30%, 40%, 50%, 60%, 70%, 80%, 90%, or 100% relative to a control;
(l) a reduction in a CXCL1 transcript level by about 10%, 20%, 30%, 40%, 50%, 60%, 70%, 80%, 90%, or 100% relative to a control;
(m) a reduction in a CXCL2 transcript level by about 10%, 20%, 30%, 40%, 50%, 60%, 70%, 80%, 90%, or 100% relative to a control;
(o) a reduction in a CXCL3 transcript level by about 10%, 20%, 30%, 40%, 50%, 60%, 70%, 80%, 90%, or 100% relative to a control;
(p) a reduction in an EGR1 transcript level by about 10%, 20%, 30%, 40%, 50%, 60%, 70%, 80%, 90%, or 100% relative to a control;
(q) a reduction in a JunB transcript level by about 10%, 20%, 30%, 40%, 50%, 60%, 70%, 80%, 90%, or 100% relative to a control;
(r) a reduction in a MKP1 transcript level by about 10%, 20%, 30%, 40%, 50%, 60%, 70%, 80%, 90%, or 100% relative to a control;
(s) a reduction in a TNFAIP1 transcript level by about 10%, 20%, 30%, 40%, 50%, 60%, 70%, 80%, 90%, or 100% relative to a control;
(t) a reduction in a TNFAIP2 transcript level by about 10%, 20%, 30%, 40%, 50%, 60%, 70%, 80%, 90%, or 100% relative to a control;
(u) a reduction in a TNFAIP3 transcript level by about 10%, 20%, 30%, 40%, 50%, 60%, 70%, 80%, 90%, or 100% relative to a control;
(v) a reduction in a SDC4 transcript level by about 10%, 20%, 30%, 40%, 50%, 60%, 70%, 80%, 90%, or 100% relative to a control;
(w) a reduction in a SOD2 transcript level by about 10%, 20%, 30%, 40%, 50%, 60%, 70%, 80%, 90%, or 100% relative to a control;
(x) a reduction in a COX2 transcript level by about 10%, 20%, 30%, 40%, 50%, 60%, 70%, 80%, 90%, or 100% relative to a control;
(y) a reduction in a IL32 transcript level by about 10%, 20%, 30%, 40%, 50%, 60%, 70%, 80%, 90%, or 100% relative to a control;
(z) a reduction in an ICAM-1 transcript level by about 10%, 20%, 30%, 40%, 50%, 60%, 70%, 80%, 90%, or 100% relative to a control;
(aa) a reduction in a KL-6 transcript level by about 10%, 20%, 30%, 40%, 50%, 60%, 70%, 80%, 90%, or 100% relative to a control;
(bb) a reduction in a CXCL10 transcript level by about 10%, 20%, 30%, 40%, 50%, 60%, 70%, 80%, 90%, or 100% relative to a control;
(cc) a reduction in a CXCL9 transcript level by about 10%, 20%, 30%, 40%, 50%, 60%, 70%, 80%, 90%, or 100% relative to a control;
(dd) a reduction in a CHIT1 transcript level by about 10%, 20%, 30%, 40%, 50%, 60%, 70%, 80%, 90%, or 100% relative to a control;
(ee) a reduction in a SCGB1A1 transcript level by about 10%, 20%, 30%, 40%, 50%, 60%, 70%, 80%, 90%, or 100% relative to a control;
(ff) a reduction in a SAA1 transcript level by about 10%, 20%, 30%, 40%, 50%, 60%, 70%, 80%, 90%, or 100% relative to a control;
(gg) a reduction in a VCAM-1 transcript level by about 10%, 20%, 30%, 40%, 50%, 60%, 70%, 80%, 90%, or 100% relative to a control;
(hh) a reduction in an ACE2 protein level by about 10%, 20%, 30%, 40%, 50%, 60%, 70%, 80%, 90%, or 100% relative to a control;
(ii) a reduction in an IL-6 protein level by about 10%, 20%, 30%, 40%, 50%, 60%, 70%, 80%, 90%, or 100% relative to a control;
(jj) a reduction in an IL-2R protein level by about 10%, 20%, 30%, 40%, 50%, 60%, 70%, 80%, 90%, or 100% relative to a control;
(kk) a reduction in a TNF-α protein level by about 10%, 20%, 30%, 40%, 50%, 60%, 70%, 80%, 90%, or 100% relative to a control;
(ll) a reduction in a CRP protein level by about 10%, 20%, 30%, 40%, 50%, 60%, 70%, 80%, 90%, or 100% relative to a control;
(mm) a reduction in an IL-1B protein level by about 10%, 20%, 30%, 40%, 50%, 60%, 70%, 80%, 90%, or 100% relative to a control;
(nn) a reduction in a MCP-1 protein level by about 10%, 20%, 30%, 40%, 50%, 60%, 70%, 80%, 90%, or 100% relative to a control;
(oo) a reduction in a CXCL1 protein level by about 10%, 20%, 30%, 40%, 50%, 60%, 70%, 80%, 90%, or 100% relative to a control;
(pp) a reduction in a CXCL2 protein level by about 10%, 20%, 30%, 40%, 50%, 60%, 70%, 80%, 90%, or 100% relative to a control;
(qq) a reduction in a CXCL3 protein level by about 10%, 20%, 30%, 40%, 50%, 60%, 70%, 80%, 90%, or 100% relative to a control;
(rr) a reduction in an EGR1 protein level by about 10%, 20%, 30%, 40%, 50%, 60%, 70%, 80%, 90%, or 100% relative to a control;
(ss) a reduction in a JUNB protein level by about 10%, 20%, 30%, 40%, 50%, 60%, 70%, 80%, 90%, or 100% relative to a control;
(tt) a reduction in a MKP-1 protein level by about 10%, 20%, 30%, 40%, 50%, 60%, 70%, 80%, 90%, or 100% relative to a control;
(uu) a reduction in a TNFAIP1 protein level by about 10%, 20%, 30%, 40%, 50%, 60%, 70%, 80%, 90%, or 100% relative to a control;
(vv) a reduction in a TNFAIP2 protein level by about 10%, 20%, 30%, 40%, 50%, 60%, 70%, 80%, 90%, or 100% relative to a control;
(ww) a reduction in a TNFAIP3 protein level by about 10%, 20%, 30%, 40%, 50%, 60%, 70%, 80%, 90%, or 100% relative to a control;
(xx) a reduction in a SDC4 protein level by about 10%, 20%, 30%, 40%, 50%, 60%, 70%, 80%, 90%, or 100% relative to a control;
(yy) a reduction in a SOD2 protein level by about 10%, 20%, 30%, 40%, 50%, 60%, 70%, 80%, 90%, or 100% relative to a control;
(zz) a reduction in a COX2 protein level by about 10%, 20%, 30%, 40%, 50%, 60%, 70%, 80%, 90%, or 100% relative to a control;
(aaa) a reduction in an IL-32 protein level by about 10%, 20%, 30%, 40%, 50%, 60%, 70%, 80%, 90%, or 100% relative to a control;
(bbb) a reduction in a KL-6 protein level by about 10%, 20%, 30%, 40%, 50%, 60%, 70%, 80%, 90%, or 100% relative to a control;
(ccc) a reduction in a CXCL10 protein level by about 10%, 20%, 30%, 40%, 50%, 60%, 70%, 80%, 90%, or 100% relative to a control;
(ddd) a reduction in a CXCL9 protein level by about 10%, 20%, 30%, 40%, 50%, 60%, 70%, 80%, 90%, or 100% relative to a control;
(eee) a reduction in a CHIT1 protein level by about 10%, 20%, 30%, 40%, 50%, 60%, 70%, 80%, 90%, or 100% relative to a control;
(fff) a reduction in a SCGB1A1 protein level by about 10%, 20%, 30%, 40%, 50%, 60%, 70%, 80%, 90%, or 100% relative to a control;
(ggg) a reduction in a SAA1 protein level by about 10%, 20%, 30%, 40%, 50%, 60%, 70%, 80%, 90%, or 100% relative to a control;
(hhh) a reduction in an ICAM-1 protein level by about 10%, 20%, 30%, 40%, 50%, 60%, 70%, 80%, 90%, or 100% relative to a control;
(iii) a reduction in a VCAM-1 protein level by about 10%, 20%, 30%, 40%, 50%, 60%, 70%, 80%, 90%, or 100% relative to a control;
(jjj) an increase in an FVC measurement by at least about 10%, 20%, 30%, 40%, 50%, 60%, 70%, 80%, 90%, or 100% relative to a control;
(kkk) an increase in an FEV1 measurement by at least about 10%, 20%, 30%, 40%, 50%, 60%, 70%, 80%, 90%, or 100% relative to a control;
(lll) an increase in a LCQ score by at least about 1, 2, 3, 4, 5, 6, 7, 8, 9, 10, 11, 12, 13, 14, 15, 16, 17, or 18 points relative to a control;
(mmm) an increase in a SF-36 score by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to a control;
(nnn) an increase in a KSQ score by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to a control;
(ooo) an increase in a STS score by at least 1, 2, 3, 4, 5, or 6 points relative to a control;
(ppp) an increase in 6MWT distance by at least by 10 m, 20 m, 30 m, 40 m, 50 m, 60 m, 70 m, 80 m, 90 m, 100 m, 125 m, 150 m, 175 m, 200 m, 250 m, 300 m, 350 m, 400 m, 450 m, or 500 m relative to a control;
(qqq) a reduction in a PGA score by at least about 1, 2, 3, 4, 5, 6, 7, 8, 9, or 10 points relative to a control;
(rrr) a reduction in a FAS score by at least about 1, 2, 3, or 4 points relative to a control;
(sss) a reduction in a mMRC Scale score by at least about 1, 2, 3, or 4 grades relative to a control;
(ttt) a reduction in a SGRQ score by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to a control;
(uuu) a reduction in a Borg's CR10 dyspnea score by at least about 1, 2, 3, 4, 5, 6, 7, 8, 9, or 10 points relative to a control.
(vvv) a reduction in a granuloma formation by about 1%, 2%, 3%, 4%, 5%, 10%, 15%, 20%, 25%, 30%, 40%, 50%, 60%, 70%, 80%, 90%, or 100%, relative to a control;
(www) a reduction in a granuloma size by about 1%, 2%, 3%, 4%, 5%, 10%, 15%, 20%, 25%, 30%, 40%, 50%, 60%, 70%, 80%, 90%, or 100%, relative to a control;
(xxx) a reduction in a calcitriol level by at least about 5%, 10%, 15%, 20%, 25%, 30%, 40%, 50%, 60%, 70%, 80%, 90%, or 100% relative to a control;
(yyy) a reduction in a neopterin level by at least about 5%, 10%, 15%, 20%, 25%, 30%, 40%, 50%, 60%, 70%, 80%, 90%, or 100% relative to a control;
(zzz) a reduction in a lysozyme level by at least about 5%, 10%, 15%, 20%, 25%, 30%, 40%, 50%, 60%, 70%, 80%, 90%, or 100% relative to a control;
(aaaa) a reduction in a sIL-2R level by at least about 5%, 10%, 15%, 20%, 25%, 30%, 40%, 50%, 60%, 70%, 80%, 90%, or 100% relative to a control; and
(bbbb) an increase in a Scadding CXR stage by at least 1, 2, 3, or 4 stages relative to a control.

### Example 1: Assessing Primary Pharmacodynamics of XTMAB-16

### Inhibition of TNFα-Mediated Cytotoxicity

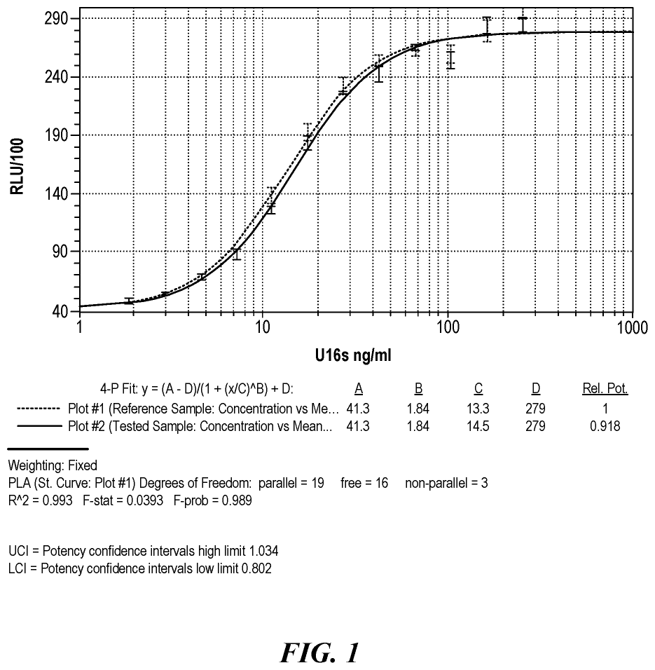

XTMAB-16 is an immunosuppressive and immunomodulatory agent due to its binding to TNFα. The potency of XTMAB-16 was tested by measuring inhibition of TNFα-induced cytotoxicity in a melanoma cell line expressing tumor necrosis factor receptor (TNFR). Cells from the melanoma cell line A375 (ATCC CRL-1619) were incubated with TNFα, actinomycin-D, and concentrations of XTMAB-16 from 1 to 1000 ng/mL for 24 hours. Cell density was then determined by luminescence. The potency of XTMAB-16 was compared with another TNFα blocker (infliximab), as shown in FIG. 1.

The potency of experimental batches comprising XTMAB-16 and Infliximab are shown in Table 6. The potency of XTMAB-16 batches were 90% to 103% relative to Infliximab.

TABLE 6
Relative Potency of XTMAB-16

| | Reference | Relative |
|---|---|---|
| XTMAB-16 Batch | Material | Potency (%) |
| A DS (35.36 mg/mL) | Infliximab | 93 |
| A DS (34.72 mg/mL) | Infliximab | 92 |
| A DS (34.72 mg/mL) | XTMAB-16: | 113 |
| | Batch C | |
| A, DS 1 month at 2-8° C. (34.72 mg/mL) | Infliximab | 97 |
| A, DS 1 month at 2-8° C. (34.72 mg/mL) | XTMAB-16: | 101 |
| | Batch C | |
| A, DS 2 months at 2-8° C. (34.72 mg/mL) | Infliximab | 95 |
| A, DS 2 months at 2-8° C. (34.72 mg/mL) | XTMAB-16: | 96 |
| | Batch C | |
| A, DS 3 months at 2-8° C. (34.72 mg/mL) | Infliximab | 101 |
| B DS (35.34 mg/mL) | Infliximab | 93 |
| C RS (10 mg/mL) | Infliximab | 91 |
| C RS (10 mg/mL) | Infliximab | 90 |
| C RS (10 mg/mL) | Infliximab | 103 |
| D DP (9.5 mg/mL) | Infliximab | 92 |

Abbreviations:
DP = Drug Product;
DS = Drug Substance;
RS = Reference Standard

Transmembrane TNFα (mTNFα) Binding

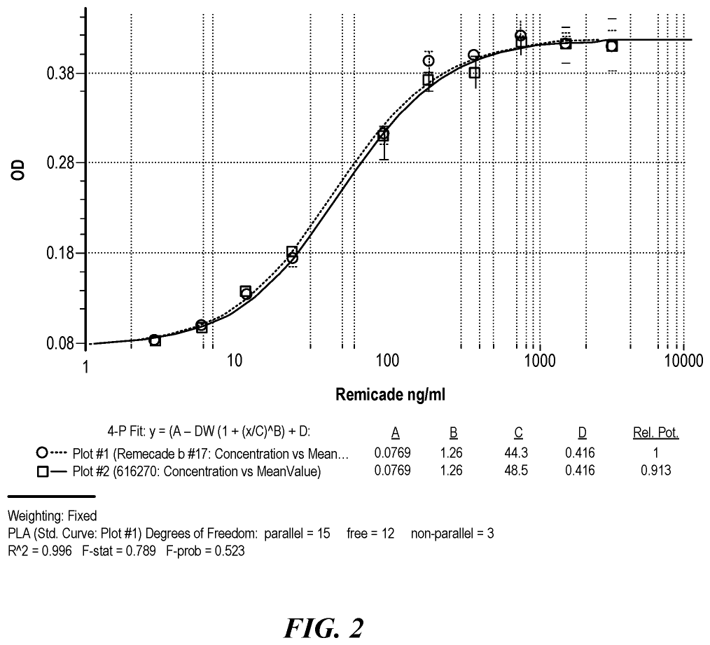

A cell-based enzyme-linked immunosorbent assay (ELISA) was used to test binding of XTMAB-16 to mTNFα. The assay used a cell line expressing mTNFα (T3-33) that was developed from SP2/0 cells (mouse myeloma cells). The T3-33 cells were incubated with 5 to 3000 ng/mL XTMAB16 or Infliximab in 3% bovine serum albumin (BSA). Bound antibody was detected using horseradish peroxidase (HRP) conjugated to mouse anti-human IgG1 and chromogenic 3,3′,5,5′tetramethylbenzidine (TMB). Binding of XTMAB-16 to the TNFα expressed on cell membranes was then detected at 450 nm (FIG. 2). XTMAB-16 bound mTNFα at 91% to 95%, as shown in Table 7.

TABLE 7
mTNFα Binding, Cell-based ELISA

| Sample | Batch No. | Relative Binding to mTNF (%) |
|---|---|---|
| XTMAB-16 | E | 95 |
| | F | 94 |
| | G | 94 |
| | H | 91 |

Abbreviations: ELISA = enzyme-linked immunosorbent assay; mTNFα = transmembrane tumor necrosis factor alpha; No. = number.
Note:
Results from repeat assays are presented as average values.
^{a} Binding relative to Infliximab

### TNFα Binding Kinetics

Biosensors coated with anti-human IgG fragment crystallizable region of an antibody (Fc) was used to capture antibodies and immobilize XTMAB-16, which was incubated with different levels of recombinant TNFα for determination of the binding kinetics between TNFα and the fragment, antigen binding region of an antibody (Fab) domain of XTMAB-16.

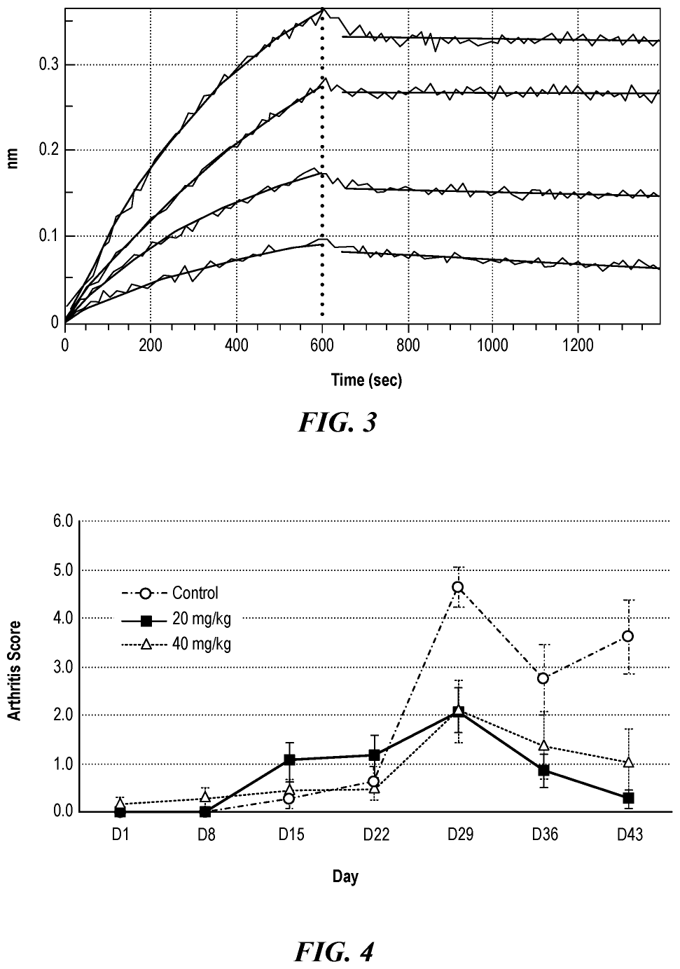

Representative kinetic curves of TNFα binding to XTMAB-16 are presented in FIG. 3. Kinetic parameters for different batches of XTMAB16 are presented in Table 8.

The binding kinetics of TNFα to XTMAB16 were determined, with affinity constant (K_{D}) of 5.68 to 6.64×10⁻¹¹M.

TABLE 8
TNFα Binding Kinetics

| Sample | Batch No. | k_{D} (M) | kₒₙ (1/Ms) | k_{dis} (1/s) |
|---|---|---|---|---|
| XTMAB-16 | E | 6.47 × 10⁻¹¹ | 1.36 × 10⁶ | 6.05 × 10⁻⁵ |
| | F | 6.53 × 10⁻¹¹ | 5.57 × 10⁵ | 8.50 × 10⁻⁵ |
| | G | 5.68 × 10⁻¹¹ | 1.01 × 10⁶ | 8.75 × 10⁻⁵ |
| | H | 6.64 × 10⁻¹¹ | 1.38 × 10⁶ | 1.30 × 10⁻⁴ |
| | Batch No. | k_{D} (M) | kₒₙ (1/Ms) | k_{dis} (1/s) |
| XTMAB-16 | E | 6.47 × 10⁻¹¹ | 1.36 × 10⁶ | 6.05 × 10⁻⁵ |
| | F | 6.53 × 10⁻¹¹ | 5.57 × 10⁵ | 8.50 × 10⁻⁵ |
| | G | 5.68 × 10⁻¹¹ | 1.01 × 10⁶ | 8.75 × 10⁻⁵ |
| | H | 6.64 × 10⁻¹¹ | 1.38 × 10⁶ | 1.30 × 10⁻⁴ |

Abbreviations: k_{D} = affinity constant; k_{dis} = dissociation constant; kₒₙ = association constant; No. = number; TNFα = tumor necrosis factor alpha.

### Cross-Species Binding Studies

Binding of XTMAB16 to human TNFα was confirmed by indirect sandwich ELISA and direct ELISA assays. Binding of XTMAB16 to human TNFα was observed above 1,000 μg/mL with a calculated half-maximal effective concentration (EC₅₀) of 60,675 pg/mL by indirect sandwich ELISA. As determined by the direct ELISA method, binding of XTMAB16 to human TNFα was observed above 10,000 μg/mL with a calculated EC₅₀ of 5.78×10⁵ pg/mL. Binding to human TNFα by human capture antibody was observed above 10,000 μg/mL with a calculated EC₅₀ of 7.37×10⁶ pg/mL. XTMAB-16 did not bind to TNFα from mouse, rat, dog, or rhesus or cynomolgus monkey. These data suggested that none of the traditional nonclinical species were considered relevant for assessing the safety of XTMAB16.

### Tissue Cross-Reactivity Study

The cross-reactivity of XTMAB-16 in cryosections from a full panel of normal human tissues as well as human sarcoidosis tissue was investigated in a good laboratory practice (GLP) study. The tissue panel used as the test system included all of the tissues on the “suggested list of human tissues to be used for immunohistochemical or cytochemical investigations of crossreactivity of monoclonal antibodies” in Annex I of the European Medicines Agency (EMA) document Guideline on Development, Production, Characterisation, and Specifications for Monoclonal Antibodies and Related Products, adopted by the Committee for Medicinal Products for Human Use (CHMP), and all of the tissues recommended in the US Food and Drug Administration (FDA), Center for Biologics Evaluation and Research (CBER) document Points to Consider in the Manufacture and Testing of Monoclonal Antibody Products for Human Use. Human tonsil, human lung and lymph node with sarcoidosis, and human small intestine with Crohn's disease were also included in the study.

Samples from 1 to 3 separate donors from each of the listed tissues were evaluated. Fresh, unfixed tissue samples were placed into molds, filled with Tissue-Tek® OCT Compound, and frozen at −85 to −70° C. until sectioning. Sections were cut at approximately 5 μm and fixed in acetone for 10 minutes at room temperature. Human blood smears were stored at −85° to −70° C. until fixation and staining. XTMAB16 (primary antibody) was incubated overnight with biotinylated donkey anti-human IgG secondary labeled antibody. XTMAB16 and the control article, hIgG1, were tested at concentration of 5 and 20 μg/mL. Immunopathology was assessed by light microscopy.

XTMAB16 produced weak to strong membrane and cytoplasmic staining of rare to occasional positive control T3-33 (TNFα-expressing) cells at both staining concentrations. The antibody did not react specifically to negative control cryosections. The control article did not specifically react to positive or negative control materials, and there was no staining of control slides. The specific reactions of XTMAB16 in all staining runs with the positive control material and the lack of specific reactivity with the negative control material, as well as the lack of reactivity of the control article, indicated that the assay was sensitive, specific, and reproducible.

Results for these studies are shown in Table 9.

TABLE 9
XTMAB-16 Binding to Normal Human Tissue.

| Sample | Tissues Showing Evidence of Staining |
|---|---|
| T3-33 (TNFα-expressing) cells | Positive control cells (+) |
| Sp2/0-Ag14 cells | Negative control cryosections (−) |
| Mononuclear cells in human tonsil, | Ancillary control tissues (+) |
| small intestine with Crohn's | |
| disease, lung with sarcoidosis, and | |
| lymph node with sarcoidosis | |
| Mononuclear cells | Membrane and cytoplasm of resident, migrating, |
| | infiltrating, and/or intravascular mononuclear |
| | cells in the bladder, cervix, esophagus, large |
| | intestine, liver (Kupffer cells), lymph node, |
| | lung, pancreas, placenta (Hofbauer cells), |
| | parathyroid, salivary gland, skin, small intestine, |
| | spleen, stomach, thyroid, thymus, tonsil, and uterus. |
| Epithelial cells | Membrane and cytoplasm of epithelial cells in the |
| | liver (hepatocytes, bile ducts) and uterus |
| | (endometrial glands). |
| | Cytoplasm only of epithelial cells stained |
| | with XTMAB-16 in the esophagus (submucosal |
| | glands), Fallopian tube (mucosa), kidney |
| | (tubules, parietal), lung (bronchioles), pancreas |
| | (acini), pituitary (adenohypophysis, Rathke's), |
| | salivary gland (acini), skin (sweat glands), and |
| | stomach (mucosa). |
| Myoepithelial cells | Membrane and cytoplasm of myoepithelial cells |
| | in the mammary gland. |
| Endothelial cells | Cytoplasm of endothelial cells in the lymph node. |
| Neural elements | Neuropil and the cytoplasm of glial cells and |
| | processes in the spinal cord. |
| Glomerular tuft cells | Cytoplasm of renal glomerular tuft cells |
| Smooth myocytes | Cytoplasm of vascular and/or intrinsic smooth |
| | myocytes in the adrenal, bladder, cerebellum, |
| | cerebral cortex, cervix, kidney, large intestine, |
| | liver, lymph node, lung, striated (skeletal) muscle, |
| | nerve, ovary, pancreas, placenta, prostate, |
| | parathyroid, skin, small intestine, spleen, stomach, |
| | testis, thymus, tonsil, and uterus. |

The mononuclear, glial, and spindle cell staining observed with XTMAB-16 was consistent with TNFα expression on macrophages, T lymphocytes, NK cells, microglia, and fibroblasts. Additionally, the staining observed with XTMAB-16 on endometrial epithelial cells and placenta decidual cells was consistent with the reported expression of TNFα. The membrane and cytoplasmic binding of XTMAB-16 in epithelial cells in the liver and myoepithelial cells in the breast were not described in the literature. Although the subcellular localization of the XTMAB-16 binding to neuropil could not be definitively determined with light microscopy, the binding was considered of limited in vivo significance due to restricted access of mAbs across the blood brain barrier.

The remaining staining with XTMAB-16 was cytoplasmic in nature and according to ICH S6(R1) and other references, mAb binding to cytoplasmic sites generally is considered of little to no toxicologic significance due to the general inability of monoclonal therapeutics to access the cytoplasmic compartment in vivo. Also, of note, the majority of non-mononuclear cell binding in this study with XTMAB-16 was low affinity (i.e., binding observed at only the higher concentration of XTMAB-16 in most tissues).

In tissues obtained from subjects, mononuclear cells in human tonsil, small intestine with Crohn's disease, lung with sarcoidosis, and lymph node with sarcoidosis were selected as ancillary controls due to the reported expression of TNFα in mononuclear cells in these tissues. The purpose of the ancillary control tissues was to demonstrate XTMAB-16-specific binding when the relevant cell types were present. XTMAB-16 weakly to strongly stained the membrane and cytoplasm of rare to occasional mononuclear cells in human tonsil at the higher staining concentration, with weak to strong and rare staining noted at the lower concentration. XTMAB-16 also weakly to strongly stained the membrane and cytoplasm of rare to occasional mononuclear cells in human small intestine with Crohn's disease, lung with sarcoidosis, and lymph node with sarcoidosis at the higher staining concentration, with weak to moderate and rare staining at the lower concentration. There was no staining of the ancillary control tissue in the assay control slides.

This immunohistochemistry study provides support related to the mechanism of XTMAB-16 as a TNFα blocker. TNFα is a pleiotropic cytokine produced by macrophages, monocytes, neutrophils, T cells, NK cells, and fibroblasts. Alveolar macrophage-derived TNFα participates in the induction and maintenance of granulomas in sarcoidosis and high levels of TNFα (and high levels of TNFα released from alveolar macrophages) seem to correlate with disease progression. Similar inflammatory mechanisms are believed to underlie sarcoidosis involvement in other organs, such as the skin. Human tissue cross-reactivity with XTMAB-16 in this study was as expected for both normal and diseased samples. Therefore, these immunohistochemistry results support XTMAB-16 use in subjects with sarcoidosis and is not expected to cause toxicity in tissues not affected by sarcoidosis.

### In Vivo Primary Pharmacology

In vivo pharmacology of XTMAB-16 was assessed in transgenic TC (TgTC) mice (rheumatoid arthritis model) in the 6-week, repeat-dose, toxicity study. TgTC mice (10/sex/dose) were administered biweekly injections of XTMAB-16 at doses of 0, 20, and 40 mg/kg/dose on Days 1, 15, and 29 with a 2-week recovery period.

The inhibitory effect of XTMAB-16 as a TNFα blocker and the potential pharmacological benefit of XTMAB-16 were assessed by comparing the joint arthritis scores, as well as histopathological examinations, between vehicle and test article-treated animals. The study revealed that there was statistically significant improvement in arthritis scores in 20 and 40 mg/kg/dose XTMAB-16-treated animals as compared to the vehicle control-treated animals. This effect was observed at the end of treatment period on Day 29 as well as at the end of recovery period on Day 43 (p<0.01), as shown in FIG. 4. There were no significant differences (t test: p>0.05) in the arthritis scores between 20 and 40 mg/kg/dose, presumably because these dose levels exceeded the maximum pharmacological effect dose.

### Example 2: Evaluating Secondary Pharmacodynamics of XTMAB-16

A mechanism of action for XTMAB-16 may involve neutralization of TNFα by binding to soluble or membrane-bound TNFα. Under certain conditions, TNFα-binding molecules may mediate Complement Dependent Cytotoxicity (CDC) and Antibody Dependent Cell-Mediated Cytotoxicity (ADCC) of mTNF-bearing cells. Therefore, activation of the immune responses ADCC and CDC may serve as secondary mechanisms of action of XTMAB-16 and may also influence drug safety, and binding affinity to C1q and FcγR family and to the neonatal Fc receptor (FcRn) may influence antibody half-life (T_{1/2}).

### C1q Binding

A C1q-ELISA assay was performed using plates coated with C1q, and an XTMAB-16 binding curve ranging from 4 to 2000 μg/mL. However, C1q binding results using this ELISA method were not predictive of the relative activity results in the CDC assay. Since the ELISA for C1q binding is performed with relatively high concentrations, it may be subject to interference. Additional development to determine the appropriate conditions and binding curve range for the assay will be conducted.

### Complement Dependent Cytotoxicity

The CDC of XTMAB-16 was assayed using cells expressing mTNFα incubated with human serum as a source of complement factors. Cells were incubated in the presence of human serum (Quidel) and increasing amounts of XTMAB-16 and incubated for 4 hours. Cell density was determined by luminescence after addition of CellTiterGlo reagent. Luminescence signal is proportional to cell density.

CDC activity is presented in Table 10. XTMAB-16 batches were stable over 2 months at 2-8° C. with 92% to 93% CDC activity. Similar results were achieved previously, demonstrating CDC activity of 4 different XTMAB-16 lots that ranged between 92% and 119% compared with Infliximab.

TABLE 10
Relative CDC activity of XTMAB-16 DS and DP

| XTMAB-16 Batch | Reference Material | Relative Activity (%) |
|---|---|---|
| A DP | Infliximab | 110 |
| A DP | XTMAB-16: B RS | 97 |
| A DS | XTMAB-16: B RS | 92 |
| A, DS 1 month at 2-8° C. | XTMAB-16: B RS | 93 |
| A, DS 2 months at 2-8° C. | XTMAB-16: B RS | 92 |

Abbreviations: CDC = complement-dependent cytotoxicity; Abbreviations: DP = Drug Product; DS = Drug Substance; RS = Reference Standard.

### Antibody Dependent Cell-Mediated Cytotoxicity

An ADCC assay was conducted using isolated peripheral blood mononuclear cells (PBMCs). Target T3-33 cells stably expressing mTNFα (3.75×10⁴ cells/well) were incubated with different concentrations of XTMAB-16 and 375,000 signal is proportional to cell death. ADCC activity for the reference standard (RS), Batch B, was 74% to 79% compared with Infliximab, and the ADCC activity of Batch A was 73% to 113% relative to Batch B (Table 11).

TABLE 11
Relative ADCC activity of XTMAB-16
DS and DP compared with B

| | Relative ADCC | Average Relative |
|---|---|---|
| XTMAB-16 Batch | Activity (%) | ADCC Activity (%) |
| A DP | 89 | 94 |
| A DP | 100 | |
| A DS | 73 | 87 |
| A DS | 98 | |
| A DS | 90 | |
| A DS, 1 month at 2-8° C. | 113 | 102 |
| A DS, 1 month at 2-8° C. | 90 | |
| A DS, 2 months at 2-8° C. | 91 | 89 |
| A DS, 2 months at 2-8° C. | 86 | |

Abbreviations: ADCC = antibody-dependent cytotoxicity; DP = Drug Product; DS = Drug Substance; RS = Reference Standard.

### Fc Receptor Binding

Receptors that recognize the Fc portion of anti-TNFα antibodies may be expressed on leucocytes and were divided into 3 groups designated: Fcγ RI, RII, and RIII (also known as CD64 (GenBank: AA152384.1), CD32 (GenBank: AA148274.1), and CD16 (NCBI: NP_001373379.1), respectively). FcγRI exhibits high affinity for IgG (10-8 to 10-9 M) and may bind monomeric IgG. In contrast, FcγRII and FcγRIII may show weaker affinity for monomeric IgG, association constant (Kₐ≤10-7 M) and may only interact effectively with multimeric immune complexes. Fcγ RI, RII, and RIII may be involved in ADCC.

Evaluation of different aspects of XTMAB-16 Fc binding by the Fc receptors may therefore provide insight into possible conformational changes. In general, for the Fc binding assays, label-free analysis with the Octet QK384 system was used to detect binding kinetics of XTMAB-16, and the resulting signals are converted to affinity parameters by the Octet analysis software (Forte'Bio Data Acquisition 9.0 software and Forte'Bio Data Analysis 9.0 software). Two methods were employed for comparative analysis of Fc binding: ELISA for evaluation of relative binding, and analysis of binding kinetics using the Octet QK384 system and SoftMaxPro software.

### Binding to FcγRI

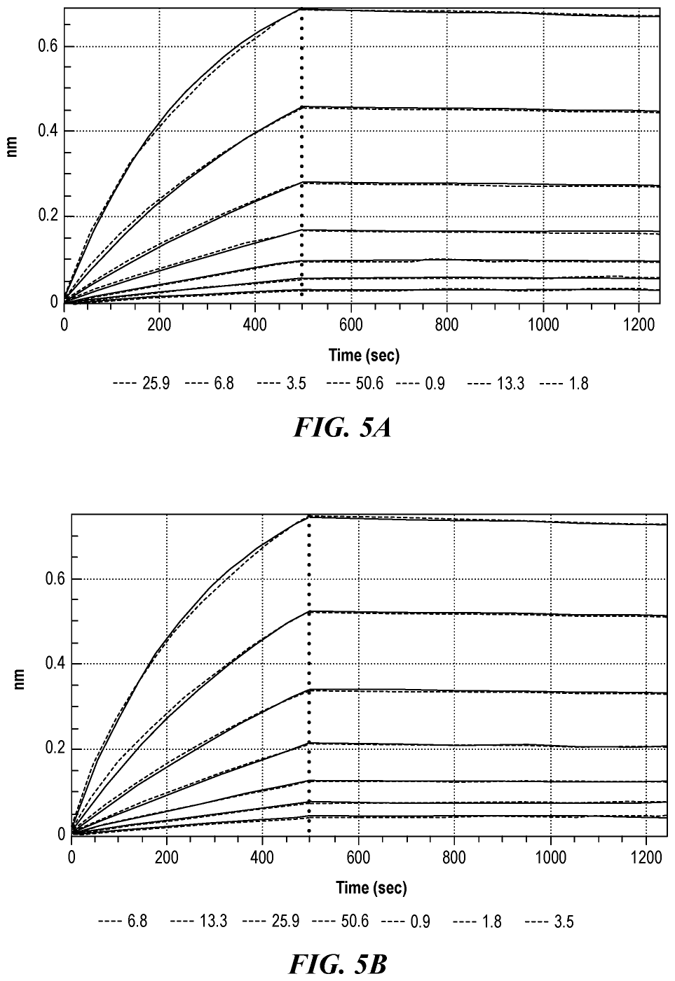

Binding kinetics of XTMAB-16 to FcγRI (also known as CD64 (GenBank: AAI52384.1)) were determined using the Octet QK384 system. Briefly, FcγRI was immobilized to biosensors coupled to nickel (Ni-NTA). XTMAB-16 binding kinetics to FcγRI was analyzed at 7 concentrations of XTMAB-16, and binding was determined with a 1:1 model fit. Sensorgrams showing binding kinetics of XTMAB-16 batches to FcγRI are shown in FIGS. 5A and 5B. Binding affinity of XTMAB-16 to FcγRI is presented in Table 12.

TABLE 12
Binding Affinity of XTMAB-16 to FcγRI

| Batch | K_{D} (nM) |
|---|---|
| XTMAB-16 D DS | 0.68 |
| XTMAB-16 B RS | 0.63 |
| XTMAB-16 DS | 0.68 |
| XTMAB-16 RS | 0.63 |

Abbreviations: DS = Drug Substance; K_{D} = affinity constant; RS = Reference Standard.

The K_{DS} of XTMAB-16 batches for FcγRI were within the range of 5 to 9×10-10 M.

An ELISA of the relative binding of XTMAB-16 to the FcγRI receptor was conducted using His tab-labeled recombinant purified FcγRI bound to ELISA plates through anti-His tab antibodies and incubated with XTMAB-16 or Infliximab at 3 to 3000 ng/mL. Binding was detected with peroxidase-conjugated F(ab′)2 goat anti-human IgG an HRP chromogenic substrate TMB using a microplate reader set to 450 nm.

### Binding to FcγRIIIa

ADCC may be initiated through interactions of the Fc region with FcγRIIIa. FcγRIIIa (CD16) is a 50 to 70 kDa membrane glycoprotein that may be expressed by neutrophils, eosinophils, NK cells, and tissue macrophages. FcγRIII shows weak affinity for monomeric IgG, Ka≤10⁷ M⁻¹, and hence may only interact effectively with multimeric immune complexes.

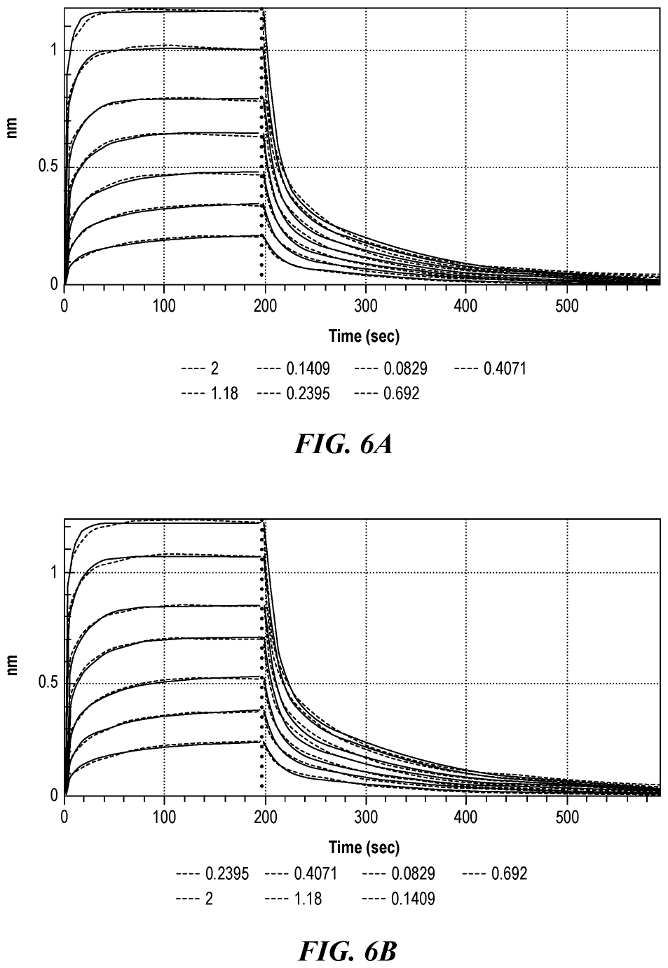

To determine binding kinetics of XTMAB-16 to FcγRIIIa, His-tagged FcγRIIIa was immobilized to biosensors bound to anti penta-His (SEQ ID NO: 25) antibodies. XTMAB-16 binding kinetics to FcγRIIIa was analyzed at 7 XTMAB-16 concentrations, and kinetic analyses was conducted using the Octet QK384 (Forte'Bio) instrument with a 2:1 heterogeneous ligand model fit. Results are shown in FIGS. 6A and 6B. and Table 13.

TABLE 13
Binding Affinity of XTMAB-16 to FcγRIIIa

| Batch | K_{D} (M) |
|---|---|
| XTMAB-1 DS | 6.5 × 10⁻⁷ |
| XTMAB-16 RS | 5.2 × 10⁻⁷ |

Abbreviations: DS = Drug Substance; K_{D} = affinity constant; RS = Reference Standard

The steady-state K_{DS}, determined from the binding curves of the different XTMAB-16 batches to FcγRIIIa, were within the range of 3 to 7×10⁻⁷M.

An ELISA was conducted to evaluate the comparative binding of XTMAB-16 to FcγRIIIa. XTMAB-16 exhibited 79% to 92% binding to FcγRIIIa binding relative to infliximab. This lower binding may be attributed to lower levels of afucosylated glycans in XTMAB-16 batches compared with infliximab. The fucosylated IgG1 may have a lower affinity to FcγRIIIa, which may be attributed to steric hindrance caused by the fucose.

### Binding to FcγRIIb

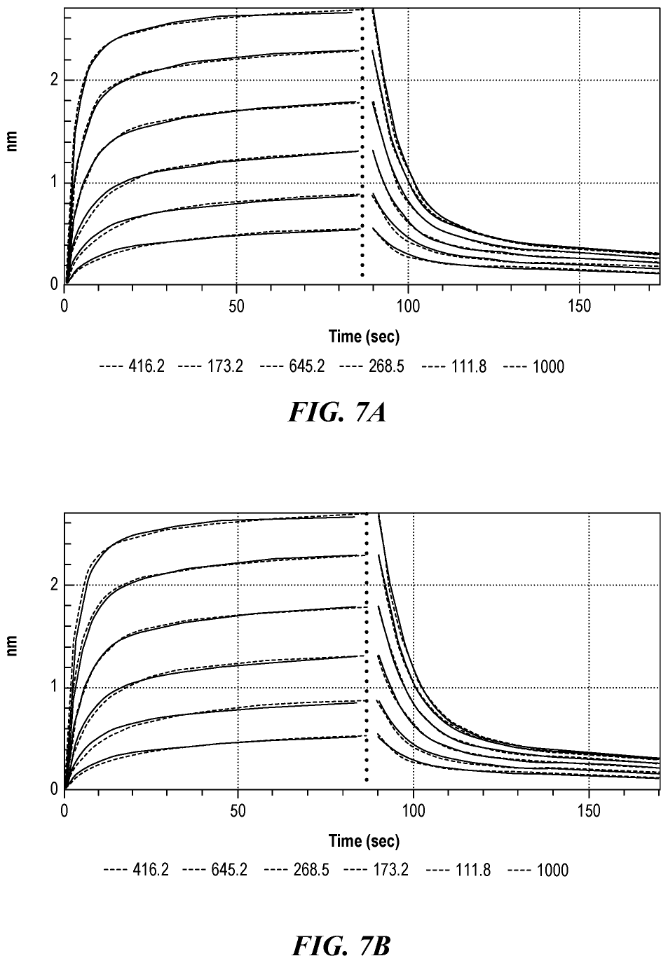

To determine XTMAB-16 binding kinetics to FcγRIIb, FcγRIIb was immobilized to biosensors coupled to nickel (Ni-NTA), and XTMAB-16 binding was analyzed at 6 concentrations. Kinetic analysis was conducted using the Octet QK384 (Forte'Bio) instrument with a 1:2 heterogeneous ligand model fit. Results are summarized in FIGS. 7A, FIG. 7B, and Table 14.

TABLE 14
Binding Affinity of XTMAB 16 to FcγRIIb

| Batch | K_{D} (M) |
|---|---|
| XTMAB-16 DS | 5.6 × 10⁻⁷ |
| XTMAB-16 RS | 6.4 × 10⁻⁷ |

Abbreviations: DS = Drug Substance; K_{D} = affinity constant; RS = Reference Standard.

An ELISA assay conducted to compare XTMAB-16 and infliximab binding showed XTMAB-16 had 97% to 114% binding to FcγRIIb relative to infliximab.

### Binding to FcRn

Binding of IgG to FcRn may protect the antibody from degradation, targeting the antibody to an endocytosis salvage pathway that releases the antibody back into the serum, thereby prolonging the T₁₁₂ of the IgG. Binding of the IgG and FcRn may be pH dependent: at pH 6 FcRn binds IgG with nanomolar affinity, whereas IgG binding to FcRn is not detectable at pH 7.

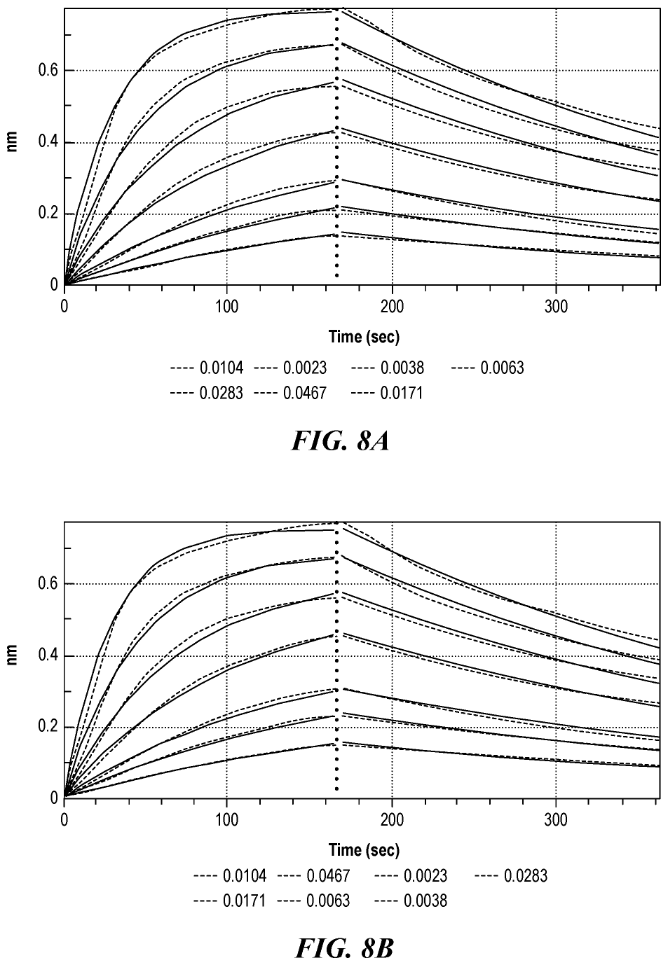

Binding kinetics of XTMAB-16 to FcRN were determined through the Fc domain. His-tag labeled FcRn was immobilized to biosensors coated with anti penta-His (SEQ ID NO: 25) antibodies. XTMAB-16 binding kinetics to FcRn was analyzed at 7 concentrations of XTMAB-16. Kinetic analysis was determined using the Octet QK384 (Forte'Bio) instrument with a 1:1 heterogeneous ligand model fit. Results are shown in FIG. 8A, FIG. 8B, and Table 15.

TABLE 15
Binding Affinity of XTMAB 16 to FcRn

| Batch | K_{D} (nM) |
|---|---|
| XTMAB-16 D DS | 4.7 |
| XTMAB-16 D DS, 1 month at 2-8° C. | 3.4 |
| XTMAB-16 D DS, 2 months at 2-8° C. | 3.5 to 4.5 |
| XTMAB-16 B RS | 3.4 to 4.3 |

Abbreviations: DS = Drug Substance; K_{D} = affinity constant; RS = Reference Standard.

K_{DS} determined for binding affinity to FcRn, of XTMAB-16 batches, were close to the preliminary target range for similarity to infliximab (4.5 to 7 nM).

Two methods were employed for analysis of the FcRn binding of XTMAB-16: ELISA for evaluation of relative binding, and analysis of binding kinetics using the Octet QK384 (Forte'Bio) instrument.

The ELISA assay used purified, biotinylated FcRn that was bound to the assay plate with streptavidin. The XTMAB-16 curve was 4.1 to 1100 μg/mL, bound to the FcRn at pH 6.0. Level of bound XTMAB-16 was determined by HRP-conjugated anti-F(ab′)2 antibodies detected with TMB at 450 nm. The relative binding affinity in 2 independent experiments was 95% to 96%.

The binding kinetics parameters, K_{D}, association constant (Kₒₙ), and dissociation constant (K_{dis}) of XTMAB-16, were determined using the Octet QK384 (Forte'Bio) instrument and a biotinylated recombinant FcRn. Biotinylated FcRn was immobilized to streptavidin-coated biosensors. Kinetics of XTMAB-16 binding to FcRn was performed at pH 6.0.

A 2:1 heterogeneous ligand fit model was found to be suitable for analysis of the results, indicating the existence of 2 separate binding sites on the FcRn molecule with different affinities. Binding affinity of XTMAB-16 Batches E, G, and H to FcRn was similar to that of infliximab, with similar K_{D}, Kₒₙ, and K_{dis}. XTMAB-16 Batch F had higher affinity to FcRn compared to other batches, with a 56-fold higher K_{D}1.

### Example 3: Assessing Safety Pharmacology

Mice were given a single dose of placebo or 10, 20, or 40 mg/kg XTMAB-16 (IV infusion in a volume of 10 mL/kg) on Day 1 and evaluated at predose and 3 and 312 hours (Day 14) postdose. Randomization and experimental groups are outlined in Table 16.

TABLE 16
Randomization and Groups

| | | | | Dose | Dose | Dose | | |
|---|---|---|---|---|---|---|---|---|
| | Animal | | | Level | Conc. | Volume | | |
| Group | Male | Female | Test Article | (mg/kg) | (mg/mL) | (mL/kg) | Animal No./Sex | Dosing |

Toxicity/CNS Safety Pharmacology Study*

| | | | | | | | | |
|---|---|---|---|---|---|---|---|---|
| 1 | 10 | 10 | Placebo | 0 | 0 | 10 | n01~n10/Male | Intravenous |
| 2 | 10 | 10 | XTMAB-16 | 10 | 1 | 10 | n11~n20/Female | injection; |
| 3 | 10 | 10 | XTMAB-16 | 20 | 2 | 10 | | only Day 1 |
| 4 | 10 | 10 | XTMAB-16 | 40 | 4 | 10 | | dose |

Respiratory Safety Pharmacology Study

| | | | | | | | | |
|---|---|---|---|---|---|---|---|---|
| 5 | 5 | 5 | Placebo | 0 | 0 | 10 | n01~n05/Male | Intravenous |
| 6 | 5 | 5 | XTMAB-16 | 10 | 1 | 10 | n06~n10/Female | injection; |
| 7 | 5 | 5 | XTMAB-16 | 20 | 2 | 10 | | only Day 1 |
| 8 | 5 | 5 | XTMAB-16 | 40 | 4 | 10 | | dose |

Abbreviations: CNS = central nervous system.
*The last 10 (5M/5F) randomized animals were selected for CNS evaluations.

In the toxicity/CNS safety pharmacology study, there were no significant differences in abbreviated functional observational battery parameters between the placebo control group and the XTMAB-16 treatment groups at 10, 20, 40 mg/kg and at pre-dose, 2-4, and 312 hours post dose, respectively.

In the respiratory safety pharmacology study, there were no significant differences in respiratory rate, tidal volume, minute volume, enhanced pause between the placebo control group and XTMAB-16 treatment groups at 10, 20, 40 mg/kg and at pre-dose, approximately 3 and 312 hours post dose, respectively.

In summary, there were no adverse effects on the CNS or respiratory system in TgTC mice treated with XTMAB-16 after a single dose ranging from 10 to 40 mg/kg.

Cardiovascular safety of XTMAB-16 has not been evaluated in any nonclinical study because there is no appropriate species (dog, monkey, swine, rabbit, ferret, guinea pig) for which XTMAB-16 is pharmacologically relevant. In lieu of these assessments, cardiovascular safety was carefully evaluated in XTMAB-16-101 and no findings were noted.

In vitro examination of human tissue cross-reactivity was performed using XTMAB-16 in normal and diseased human tissues. The objective of this study was to determine the potential cross-reactivity of XTMAB-16, a chimeric (human/murine) IgG1 mAb directed against TNFα, with cryosections from a full panel of normal human tissues, as well as human sarcoidosis tissue (1-3 donors per tissue, where available, at 2 concentrations [5 and 20 μg/mL]).

TNFα is a pleiotropic cytokine produced by macrophages, monocytes, neutrophils, T cells, NK-cells, and fibroblasts. However, cells of the monocytic lineage—such as macrophages, astroglia, microglia, Langerhans cells, Kupffer cells, and alveolar macrophages—may be the primary synthesizers of TNFα. Both soluble and transmembrane forms of TNFα forms have been identified. TNFα may be involved in cellular proliferation, differentiation, growth, and the immune response.

In normal human tissues, XTMAB-16 stained the membrane and cytoplasm of resident, migrating, infiltrating, and/or intravascular mononuclear cells in the bladder, cervix, esophagus, large intestine, liver (Kupffer cells), lymph node, lung, pancreas, placenta (Hofbauer cells), parathyroid, salivary gland, skin, small intestine, spleen, stomach, thyroid, thymus, tonsil, and uterus, as expected. The mononuclear cell staining was weak to strong or weak to moderate and frequent, occasional to frequent, occasional, rare to occasional, or rare at the higher concentration, with comparable, reduced, or no staining at the lower concentration.

In tissues obtained from subjects, mononuclear cells in human tonsil, small intestine with Crohn's disease, lung with sarcoidosis, and lymph node with sarcoidosis were selected as ancillary controls due to the reported expression of TNFα in mononuclear cells in these tissues. The purpose of the ancillary control tissues was to demonstrate XTMAB-16-specific binding when the relevant cell types were present. XTMAB-16 weakly to strongly stained the membrane and cytoplasm of rare to occasional mononuclear cells in human tonsil at the higher staining concentration, with weak to strong and rare staining noted at the lower concentration.

XTMAB-16 also weakly to strongly stained the membrane and cytoplasm of rare to occasional mononuclear cells in human small intestine with Crohn's disease, lung with sarcoidosis, and lymph node with sarcoidosis at the higher staining concentration, with weak to moderate and rare staining at the lower concentration. There was no staining of the ancillary control tissue in the assay control slides.

The following human tissues were assessed in this cross-reactivity study:
1. Positive Control:
   Cryosections of T3-33 (TNFα-expressing) cells
2. Negative Control:
   Cryosections of Sp2/0-Ag14 cells
3. Ancillary Controls:
   Cryosections of human tonsil (mononuclear cells; 2 donors)
   Cryosections of human small intestine with Crohn's disease (mononuclear cells; 1 donor)
   Cryosections of human lung with sarcoidosis (mononuclear cells; 1 donor)
   Cryosections of human lymph node with sarcoidosis (mononuclear cells; 1 donor)

Normal human tissue types tested are shown in Table 17.

TABLE 17
Normal Human Tissue Types Tested (3 Donors)

| | | |
|---|---|---|
| Adrenal | Kidney (glomerulus, | Skin |
| | tubule) | |
| Bladder (urinary) | Liver | Spinal Cord |
| Blood Cells ^{a} | Lung | Spleen |
| Blood Vessels | Lymph Node | Striated Muscle |
| (endothelium) ^{b} | | (skeletal) |
| Bone Marrow | Ovary | Testis |
| Brain - cerebellum | Pancreas | Thymus |
| Brain - cerebral cortex | Parathyroid | Thyroid |
| Breast (mammary gland) | Peripheral Nerve | Tonsil |
| Eye | Pituitary | Ureter |
| Fallopian Tube (oviduct) | Placenta | Uterus - cervix |
| Gastrointestinal (GI) Tract ^{c} | Prostate | Uterus - endometrium |
| Heart | Salivary Gland | |

^{a} Evaluated from peripheral blood smears.
^{b} Evaluated from all tissues where present.
^{c} Included esophagus, large intestine/colon, small intestine, and stomach (including underlying smooth muscle).

This immunohistochemistry assessment provides support related to the mechanism of XTMAB-16 as a TNFα blocker. As previously noted, TNFα is a pleiotropic cytokine produced by macrophages, monocytes, neutrophils, T cells, NK-cells, and fibroblasts. Alveolar macrophage-derived TNFα participates in the induction and maintenance of granulomas in sarcoidosis and high levels of TNFα (and high levels of TNFα released from alveolar macrophages) seem to correlate with disease progression. Similar inflammatory mechanisms may underlie sarcoidosis involvement in other organs, such as the skin.

Human tissue cross-reactivity with XTMAB-16 in this study was as expected for both normal and diseased samples. Therefore, these immunohistochemistry results support XTMAB-16 use in subjects with sarcoidosis and is not expected to cause toxicity in tissues not affected by sarcoidosis.

### Example 4: Assessing Pharmacokinetics, Absorption, Distribution, Metabolism, and Excretion of XTMAB-16

### Absorption and Pharmacokinetics

In the 3-week non-GLP study in ICR mice, tₘₐₓ for a single IV dose of XTMAB-16 at 60 mg/kg was 5 minutes. XTMAB-16 dosed weekly via IV demonstrated a dose-dependent relationship, with mean plasma concentrations 15 minutes after the third weekly dose determined to be 512.88, 889.42, 1598.13, and 1976.38 μg/mL for the 30, 40, 60, and 100 mg/kg doses, respectively.

From the 6-week, GLP, toxicology and TK study in TgTC mice, the Cmax and AUC0-∞ increased in a dose-dependent fashion after single dosing. After a single IV dose, Cmax was 174.8, 354.2, and 791.0 μg/mL for the 10, 20, and 40 mg/kg doses, respectively. The AUC0-∞ after single-dose administration demonstrated linearity with values of 31755.5, 74776.5, and 144168.4 μg/mL*hr for the 10, 20, and 40 mg/kg groups, respectively. The tmax was 0.08 hours for all doses, and the t½ of XTMAB-16 ranged between 226.9 hours (9.45 days) and 327.2 hours (13.63 days) for the 10 mg/kg and 40 mg/kg doses, respectively.

From the 13-week nonclinical GLP toxicology study in TgTC mice (Study 14042-20005), the repeated TK parameters after 6 of the 7 bi-weekly IV injections of XTMAB-16 at 10, 20 and 40 mg/kg were characterized. After 40 mg/kg IV injection for 6 doses, the Cₘₐₓ was 1104.9 μg/mL, the AUC0-∞ was 624865.9 μg*hr/m L, and the AUC0-τ was 124878.7 μg*hr/mL. The systemic exposure in terms of Cmax and AUC0-∞ increased in a dose proportional manner in the dose range from 10 to 40 mg/kg of XTMAB-16. All serum samples were negative for ADA up to Day 83 in TgTC mice after 6 repeated bi-weekly IV injections of 10, 20 and 40 mg/kg XTMAB-16.

### Single-Dose Pharmacokinetics

Initial single-dose PK was examined in Study 14042-20003 in ICR mice at the 50 mg/kg XTMAB-16 IV dosing level. A total of 4 animals (2 animals/sex) were injected with 50 mg/kg XTMAB-16 on Day 1 and plasma concentrations were assessed at 2, 5, 10, and 15 minutes postdose. Mean plasma concentrations are summarized in Table 18. Based on the time points assessed in this study, the tmax was determined to be 5 minutes postdose.

TABLE 18
Mean XTMAB-16 Plasma Concentrations
After 50 mg/kg IV Dosing in ICR Mice

| Time Postdose | 2 Min | 5 Min | 10 Min | 15 Min |
|---|---|---|---|---|
| Mean Plasma | 1057.92 | 1077.23 | 723.23 | 822.86 |
| Concentration | μg/mL | μg/mL | μg/mL | μg/mL |

Abbreviations: ICR = Institute of Cancer Research; IV = intravenous.

Single-dose PK was examined in TgTC mice. Blood samples were collected predose and at 0.083, 8, 24, 48, 96, 168, 336, 672, 840, and 1008 hours postdose on Day 1 and processed to serum. After a single IV dose of 10, 20, or 40 mg/kg XTMAB-16, the serum concentrations were determined, and the major PK parameters are summarized in Table 19. Data were combined for both genders (2 animals/sex/timepoint, except for baseline, which was 1 animal/sex/timepoint). A single dose of XTMAB-16 resulted in dose-proportional increases in Cₘₐₓ, AUC_{0-τ}, and AUC0-∞ over the dose range of 10 to 40 mg/kg. Tₘₐₓ was consistent across doses at 0.08 hr and CL was consistent across doses at 0.3 mL/h/kg.

TABLE 19

| | Single Dose XTMAB-16 Pharmacokinetic Parameters in TgTC Mice | | | | | | | |
|---|---|---|---|---|---|---|---|---|
| XTMAB-16 | t½ | tmax | Cmax | AUC0-∞ | AUC0-τ | CL | Vz | MRT0-∞ |
| Dose | (hr) | (hr) | (μg/mL) | (hr*μg/mL) | (hr*μg/mL) | (mL/h/kg) | (mL/kg) | (hr) |
| 10 mg/kg | 226.9 | 0.08 | 174.8 | 30901.0• | 31755.5 | 0.3 | 103.1 | 316.8 |
| 20 mg/kg | 315.2 | 0.08 | 354.2 | 65435.9 | 74776.5 | 0.3 | 121.6 | 453.3 |
| 40 mg/kg | 327.2 | 0.08 | 791.0 | 124257.2 | 144168.4 | 0.3 | 131.0 | 485.4 |

Abbreviations: AUC = area under the curve; CL = clearance; LLOQ = lower limit of quantitation; MRT = mean residence time; TgTC = hTNF knock-in transgenic; t½ = apparent terminal half-life; tmax = time to maximum observed concentration; Vz = volume of distribution at the terminal phase.
LLOQ: 31.3 ng/mL

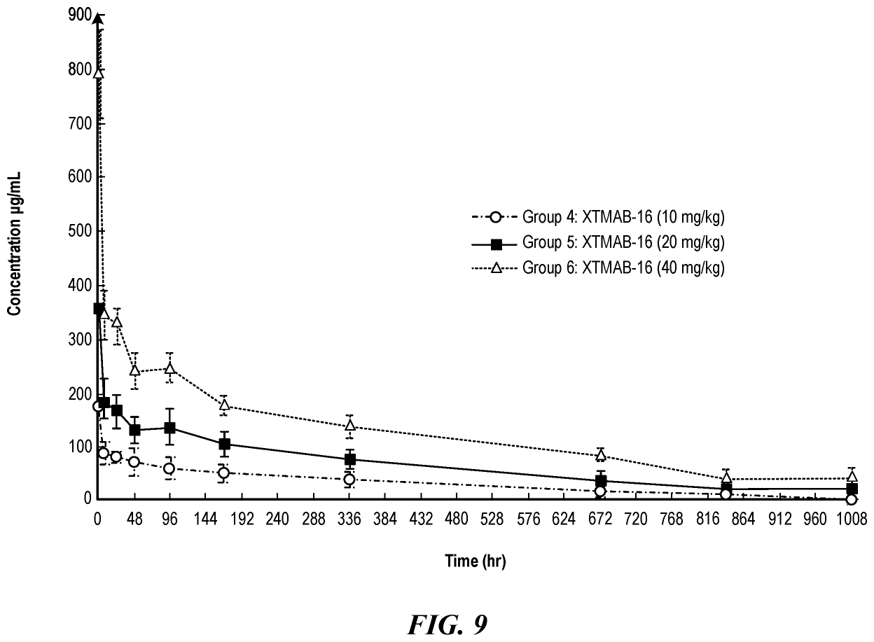

The XTMAB-16 serum concentration versus time profile is shown in FIG. 9.

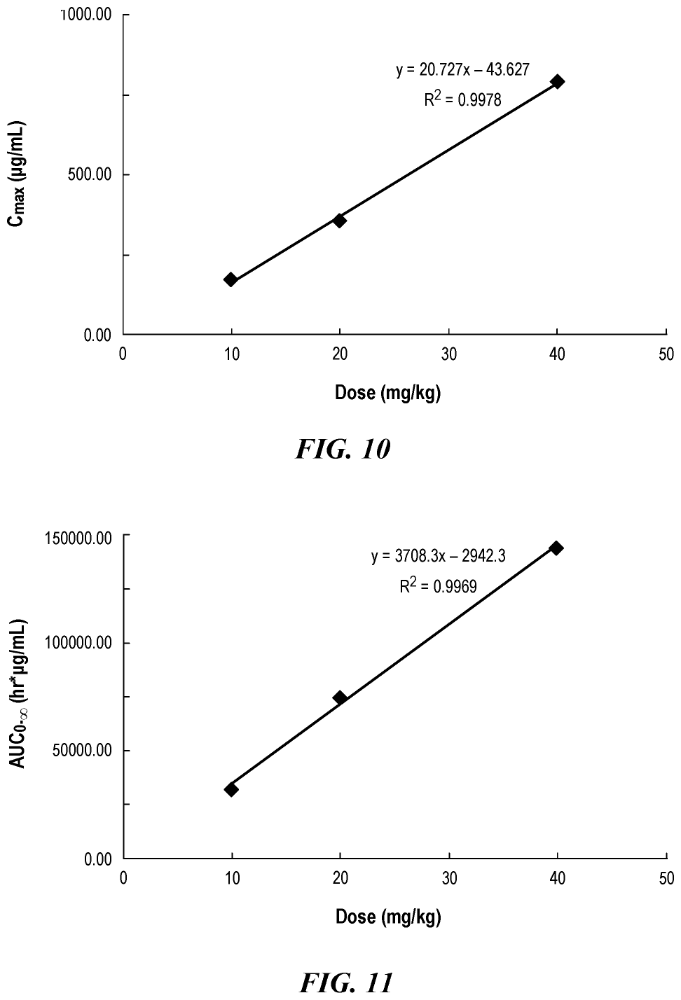

The systemic exposure in terms of Mean Cₘₐₓ and AUC were proportionally increased at doses of 10, 20, and 40 mg/kg of XTMAB-16 as shown in FIG. 10 and FIG. 11.

### Repeat-Dose Pharmacokinetics

In the 3-week ICR mouse exploratory toxicity study, repeat-dose PK was determined for animals dosed with 60 mg/kg XTMAB-16 via intraperitoneal (IP) injection and with 30, 40, 60, and 100 mg/kg XTMAB-16 via IV injection. For animals dosed via IP injection, 4 animals (2 male/2 female) received doses of 60 mg/kg on Days 1, 8, and 15 and samples for PK were collected on Day 15 at predose and 1, 2, and 3 hours postdose (N=1 animal per time point). Plasma concentrations of XTMAB-16 are provided in Table 20. Based on the available time points, the tₘₐₓ for IP injection in this study was determined to be ≥3 hours.

TABLE 20
XTMAB-16 Plasma Concentrations on Day 15 After 60 mg/kg Dosing
on Days 1, 8, and 15 (N = 1 per time point) in ICR Mice

| | | 1 hr | 2 hr | 3 hr |
|---|---|---|---|---|
| Time | Predose | Postdose | Postdose | Postdose |
| Mean Plasma | 332.00 | 696.12 | 587.44 | 952.45 |
| Concentration | μg/mL | μg/mL | μg/mL | μg/mL |

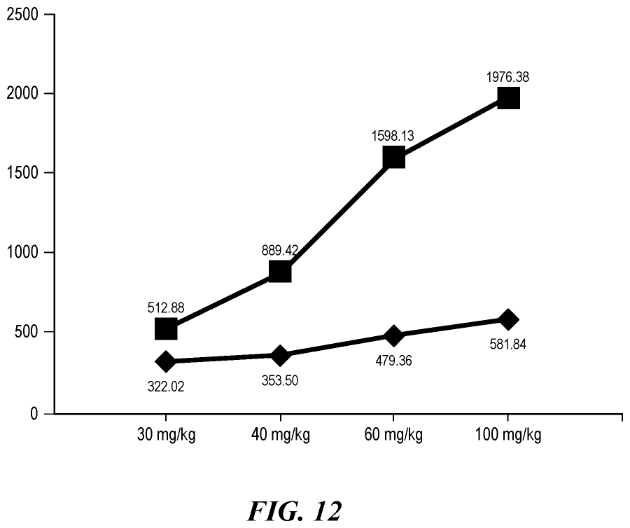

For animals dosed via IV injection, 4 animals (2/sex/dose group) in each dose group received IV injections of 30, 40, 60, and 100 mg/kg of XTMAB-16 on Days 1, 8, and 15 and samples for PK were collected on Day 15 at predose and 15 minutes postdose. The plasma concentrations at 15 minutes postdose on Day 15 (n=1-2 mice/time point) showed a dose-related increase in concentrations with mean values of 512.88 μg/mL at 30 mg/kg/dose, 889.42 μg/mL at 40 mg/kg/dose, 1598.13 μg/mL at 60 mg/kg/dose, and 1976.38 μg/mL at 100 mg/kg/dose. The Day 15 predose (trough) plasma concentrations (n=1 mouse/time point) also exhibited a dose-related increase with values of 322.02 μg/mL at 30 mg/kg/dose, 353.50 μg/mL at 40 mg/kg/dose, 479.36 μg/mL at 60 mg/kg/dose, and 581.84 μg/mL at 100 mg/kg/dose. The predose data suggest that the analyte had not been fully eliminated from systemic circulation in the 1-week period between the second and third doses. The dose-response data are summarized in FIG. 12.

Pharmacokinetic (PK) samples were also collected from the 7 IV-dosed animals that were found dead on Day 8, and one 60 mg/kg male that was found dead on Day 1 (plasma concentration of 276.15 μg/mL) and was replaced. The Day 8 concentrations of XTMAB-16 in these mice were generally comparable at 40 to 60 mg/kg/dose (624.49 μg/mL at 40 mg/kg/dose; 359.9 to 614.40 μg/mL at 50 mg/kg/dose; and 430.99 to 455.01 μg/mL at 60 mg/kg/dose) and were increased at 100 mg/kg/dose (805.53 μg/mL).

Repeat-dose TK was examined in the completed 13-week TgTC mouse toxicology study. A total of 40 animals per group (20 animals/sex/dose group) received doses of 10, 20, or 40 mg/kg of XTMAB-16 biweekly on Days 1, 15, 29, 43, 57, and 71. The repeated TK parameters after 6 of the 7 bi-weekly IV injections of XTMAB-16 at 10, 20 and 40 mg/kg were characterized. After 40 mg/kg IV injection for 6 doses, the Cₘₐₓ was 1104.9 μg/mL, the AUC0-∞ was 624865.9 μg*hr/mL, and the AUC0-τ was 124878.7 μg*hr/mL. The systemic exposure in terms of Cmax and AUC0-∞ increased in a dose proportional manner in the dose range from 10 to 40 mg/kg of XTMAB-16.

### PK-Related Safety Margin

Using mg/kg scaling from the 40 mg/kg TgTC mouse NOAEL in the 13-wk toxicology study, the 2 and 4 mg/kg human dose cohorts provide a 5 and 10-fold safety margin respectively. Projected human safety margins were also calculated by comparing the NOAEL exposure in the toxicology species (TgTC mice dosed at 40 mg/kg) to the projected clinical exposures. Using TK data generated from the 6-week, GLP, toxicology study in TgTC mice (Table 21), a 2-compartmental model with first order absorption and elimination using a mixed ratio error model was developed for XTMAB-16. The model was allometrically scaled to humans using published allometric exponents. The allometrically scaled human model was then used to simulate the planned FIH doses for XTMAB-16 for a 70 kg human.

The projected human doses were then compared to the calculated Cₘₐₓ and AUC_{0-∞} from the 13-week NOAEL to generate exposure-based safety margins. Using a model built from the 6-week toxicology study comparing the AUC_{0-∞} (624865.9 hr·mg/mL) of the 40 mg/kg group dosed with XT-MAB16 in TgTC model toxicology study to the estimated AUC_{0-∞} of the 1 mg/kg dose in humans (1400 hr·mg/mL) the safety margin for the starting dose is approximately 45-fold. Comparing the Cmax (1104.9 mg/mL) of 40 mg/kg dose of XTMAB-16 in TgTC to the estimated Cmax (47.8 mg/mL) of 2 mg/kg dose in humans, the safety margin for the starting dose is approximately 23-fold.

TABLE 21
Human Safety Margins Compared to the Calculated
Cmax and AUC from TgTC Mice

| | 2 mg/kg | 4 mg/kg |
|---|---|---|
| Human Cₘₐₓ (μg/mL) | 47.8 | 95.7 |
| Safety Margin Cₘₐₓ | 23 | 12 |
| Human AUC_{0-∞} (μg/mL · hr) | 14000 | 28000 |
| Safety Margin AUC_{0-□} | 45 | 22 |

Abbreviations: AUC = area under the curve; Cₘₐₓ = maximum observed concentration; TgTC = hTNF knock-in transgenic.

### Excretion

Clearance (CL) of XTMAB-16 after a single dose of 10, 20, or 40 mg/kg IV injection in TgTC mice was calculated to be 0.3 mLs/hr/kg. The CL after 6 doses in the 13-week toxicology study was calculated to be 0.12 at steady state.

### Example 5: Assessing Toxicology of XTMAB-16

In the 3-week, non-GLP, ICR mouse exploratory toxicology study, 25 animals were scheduled to receive weekly doses (Days 1, 8, and 15) of IV XTMAB-16 at 30, 40, 50, 60, or 100 mg/kg. Eight of the 21 animals were found dead within 1 hour after dosing on Day 8 as follows: 0/4 at 30 mg/kg; 1/4 at 40 mg/kg; 3/4 at 50 mg/kg; 2/4 at 60 mg/kg; and 1/4 at 100 mg/kg. Following weekly IV dosing, 40 mg/kg was determined to be the highest dose to be used for subsequent nonclinical studies.

In the 6-week, GLP, TgTC mouse toxicology study (4-week treatment period, 2-week recovery period; 60 animals (10 animals/sex/dose level) received repeat doses of IV XTMAB-16 at 0, 20, or 40 mg/kg on Days 1, 15, and 29. An additional 132 animals received a single dose of IV XTMAB-16 at 10, 20, or 40 mg/kg on Day 1 and were assigned to the TK satellite groups. There were no animal deaths in the toxicology group. In the TK satellite group, 3 animals were found dead: 1 male at 7 days after dosing (10 mg/kg group) and 2 females at Day 15 after dosing (20 mg/kg group). These deaths were not considered to be related to XTMAB-16 as the deaths occurred 1 to 2 weeks post-dosing with no dose-response relationship. The NOAEL of XTMAB-16 is considered 40 mg/kg/dose after 3 biweekly IV injections in TgTC mice.

In the 13-week nonclinical GLP toxicology study in TgTC mice, XTMAB-16 was well tolerated up to 40 mg/kg after 7 bi-weekly IV injections in TgTC animals in the toxicity study with no meaningful test article related clinical and pathological abnormalities observed. There were disease model related abnormalities. Animal deaths were observed at all doses up to and including 40 mg/kg; however, there was no dose or time dependency for these mortalities and no pathological changes that allowed for a cause of death to be determined. Overall, no dosed animals died upon first dose, and all XTMAB-16-dosed animals survived for at least 4 weeks following the first dose, with most surviving for 9 weeks or longer. Given that 4 untreated back-up mice died, the deaths were not considered to be test article related. The no observed adverse effect level (NOAEL) of XTMAB-16 was 40 mg/kg.

### Example 6: Additional Pharmacokinetic Studies of XTMAB-16

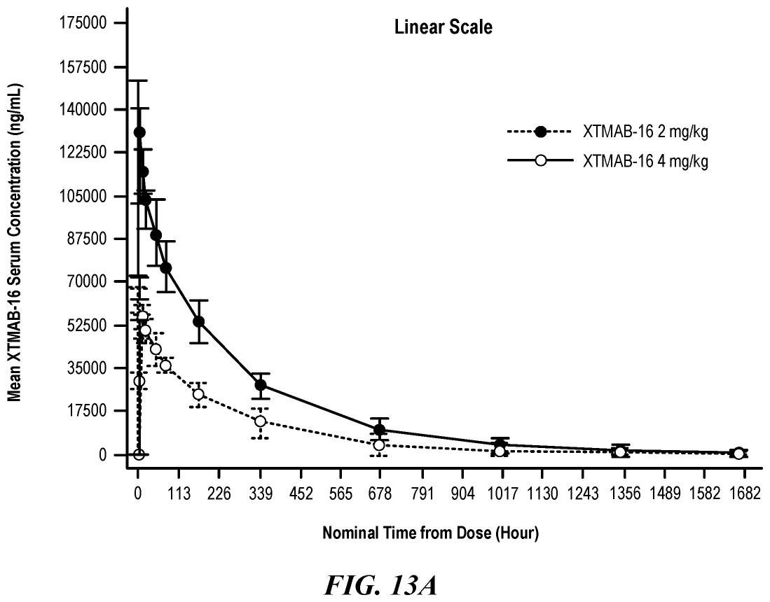

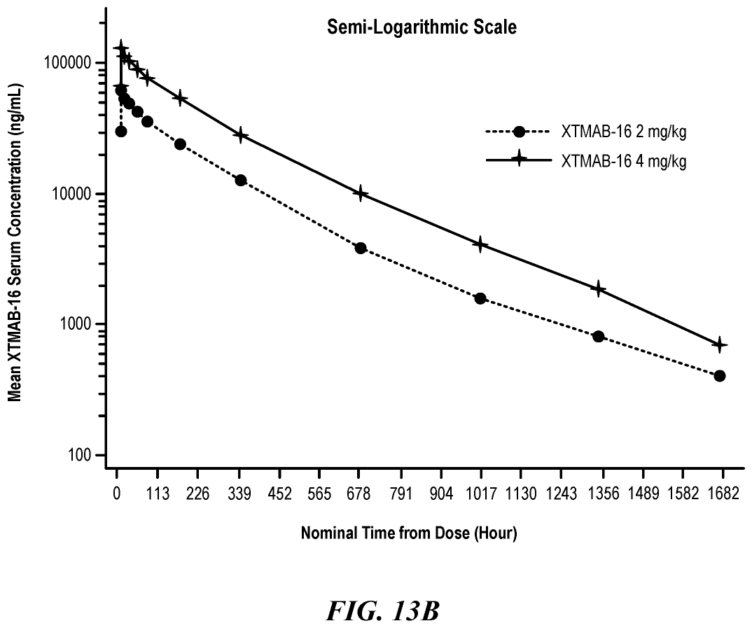

The overall mean XTMAB-16 concentration versus nominal time linear and semi-logarithmic profiles are presented overlaid for both 2 mg/kg and 4 mg/kg groups were assessed in non-sarcoidosis subjects (FIGS. 13A and 13B). These PK profiles of 2 mg/kg and 4 mg/kg groups showed a rapid rise in the plasma concentration during infusion with a tₘₐₓ between 2 to 3 hours and eliminated slowly in a biphasic disposition manner with an overall geometric mean half-life of 7.8 hours and 9.6 hours in the 2 mg/kg and the 4 mg/kg groups, respectively.

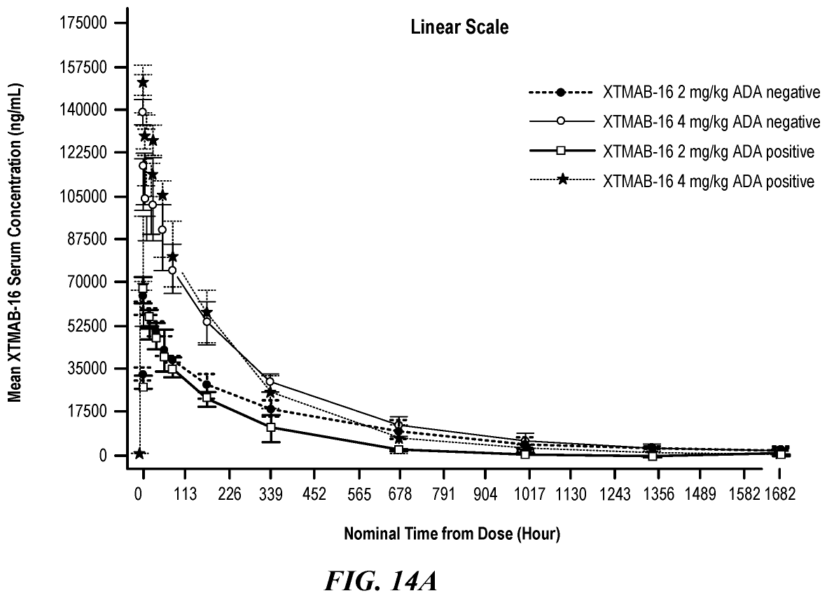

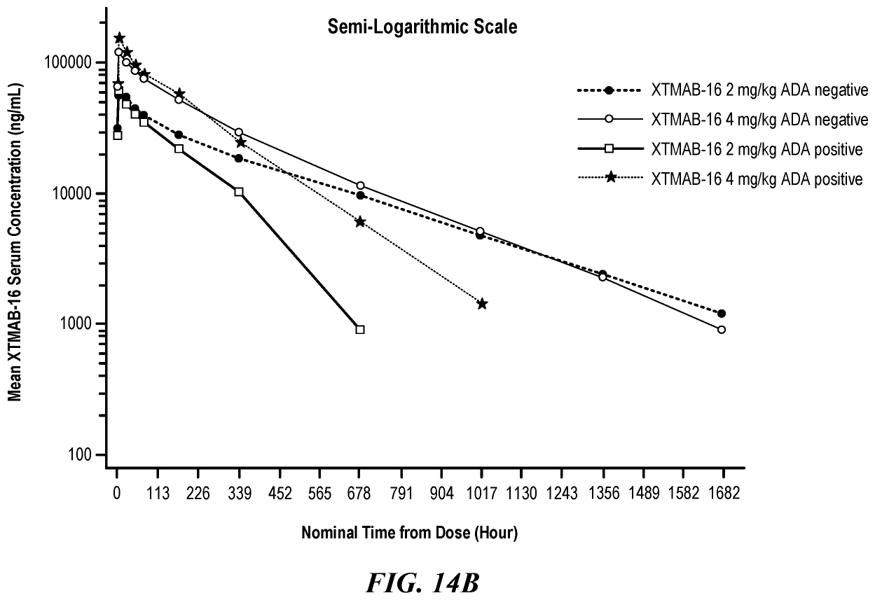

For serum concentration versus time profiles by treatment and ADA incidence, in both XTMAB-16 groups the mean profiles of subjects showing positive ADA/Nab response displayed a rapid elimination from the systemic circulation as compared to the profiles of subjects showing negative ADA/Nab response (FIGS. 14A and 14B).

The geometric mean Cmax (geometric % CV) of XTMAB-16 was 63.52 μg/mL (15%) and 129.1 μg/mL (18.8%) for XTMAB-16 in all subjects administered a single dose of 2 mg/kg and 4 mg/kg, respectively. The corresponding geometric mean (geometric % CV) for systemic XTMAB-16 exposure (AUC0-last) was 483.6 day*μg/mL (44.3%) and 1203 day*μg/mL (20.3%) for 2 mg/kg and 4 mg/kg, respectively. Moreover, the AUC0-∞ was comparable to the AUC0-last for both the cohorts Table 22.

TABLE 22
Study XTMAB-16-101: Summary of XTMAB-16 Serum Pharmacokinetic Parameters
by Treatment in ADA/nAb Positive and Overall Pooled Subjects

| | | Cohort 1 | | Cohort 2 | |
|---|---|---|---|---|---|
| | | XTMAB-16 2 mg/kg | | XTMAB-16 4 mg/kg | |
| Pharmacokinetic | | ADA/nAb | | ADA/nAb | |
| Parameter | Statistics | Positive | Overall | Positive | Overall |
| Cₘₐₓ (μg/mL) | n | 6 | 10 | 2 | 9 |
| | Mean | 64.17 | 64.17 | 153.9 | 131.0 |
| | SD | 11.24 | 9.940 | 1.197 | 23.42 |
| | % CV | 17.5 | 15.5 | 0.8 | 17.9 |
| | Geo. Mean | 63.43 | 63.52 | 153.9 | 129.1 |
| | Geo. % CV | 16.5 | 15.0 | 0.8 | 18.8 |
| | Min | 54.40 | 52.04 | 153.1 | 96.76 |
| | Median | 61.11 | 63.22 | 153.9 | 141.6 |
| | Max | 84.84 | 84.84 | 154.8 | 154.8 |
| tₘₐₓ (h) | n | 6 | 10 | 2 | 9 |
| | Minimum | 2.10 | 2.10 | 2.27 | 2.12 |
| | Median | 2.16 | 2.16 | 2.65 | 3.00 |
| | Maximum | 8.65 | 8.65 | 3.03 | 8.02 |
| C_{T} (μg/mL) | n | 6 | 10 | 2 | 9 |
| | Mean | 61.17 | 62.18 | 151.3 | 128.9 |
| | SD | 5.611 | 6.757 | 2.492 | 22.39 |
| | % CV | 9.2 | 10.9 | 1.6 | 17.4 |
| | Geo. Mean | 60.96 | 61.84 | 151.3 | 127.1 |
| | Geo. % CV | 9.2 | 11.0 | 1.6 | 18.2 |
| | Min | 54.40 | 52.04 | 149.6 | 95.66 |
| | Median | 61.11 | 63.22 | 151.3 | 141.4 |
| | Max | 67.12 | 72.80 | 153.1 | 153.1 |
| AUC₀₋ₗₐₛₜ | n | 6 | 9 | 2 | 9 |
| (day*μg/mL) | Mean | 389.0 | 525.2 | 1085 | 1224 |
| | SD | 84.92 | 238.0 | 291.4 | 235.9 |
| | % CV | 21.8 | 45.3 | 26.9 | 19.3 |
| | Geo. Mean | 380.7 | 483.6 | 1065 | 1203 |
| | Geo. % CV | 23.7 | 44.3 | 27.7 | 20.3 |
| | Min | 254.5 | 254.5 | 878.7 | 878.7 |
| | Median | 394.8 | 425.8 | 1085 | 1291 |
| | Max | 507.0 | 1018 | 1291 | 1577 |
| AUC_{0-∞} | n | 5 | 8 | 2 | 9 |
| (day*μg/mL) | Mean | 464.1 | 601.5 | 1110 | 1249 |
| | SD | 31.64 | 231.8 | 304.3 | 245.0 |
| | % CV | 6.8 | 38.5 | 27.4 | 19.6 |
| | Geo. Mean | 463.3 | 570.6 | 1089 | 1226 |
| | Geo. % CV | 6.6 | 33.9 | 28.3 | 20.6 |
| | Min | 431.5 | 431.5 | 894.8 | 894.8 |
| | Median | 455.8 | 489.0 | 1110 | 1325 |
| | Max | 516.9 | 1100 | 1325 | 1634 |
| λ_{Z} (/h) | n | 5 | 8 | 2 | 9 |
| | Mean | 0.004903 | 0.003981 | 0.004648 | 0.003199 |
| | SD | 0.0006041 | 0.001434 | 0.001662 | 0.001277 |
| | % CV | 12.3 | 36.0 | 35.8 | 39.9 |
| | Geo. Mean | 0.004873 | 0.003693 | 0.004497 | 0.002998 |
| | Geo. % CV | 12.6 | 47.1 | 37.8 | 39.1 |
| | Min | 0.004080 | 0.001507 | 0.003472 | 0.001868 |
| | Median | 0.004740 | 0.004390 | 0.004648 | 0.002862 |
| | Max | 0.005562 | 0.005562 | 0.005823 | 0.005823 |
| t_{1/2} (day) | n | 5 | 8 | 2 | 9 |
| | Mean | 5.965 | 8.644 | 6.639 | 10.23 |
| | SD | 0.7593 | 4.726 | 2.374 | 3.617 |
| | % CV | 12.7 | 54.7 | 35.8 | 35.4 |
| | Geo. Mean | 5.927 | 7.821 | 6.423 | 9.634 |
| | Geo. % CV | 12.6 | 47.1 | 37.8 | 39.1 |
| | Min | 5.192 | 5.192 | 4.960 | 4.960 |
| | Median | 6.093 | 6.611 | 6.639 | 10.09 |
| | Max | 7.078 | 19.16 | 8.317 | 15.46 |
| CL (L/day) | n | 5 | 8 | 2 | 9 |
| | Mean | 0.2806 | 0.2498 | 0.3055 | 0.2449 |
| | SD | 0.02756 | 0.04883 | 0.06872 | 0.06064 |
| | % CV | 9.8 | 19.6 | 22.5 | 24.8 |
| | Geo. Mean | 0.2794 | 0.2454 | 0.3016 | 0.2385 |
| | Geo. % CV | 10.4 | 20.7 | 23.0 | 24.6 |
| | Min | 0.2338 | 0.1767 | 0.2569 | 0.1583 |
| | Median | 0.2857 | 0.2578 | 0.3055 | 0.2247 |
| | Max | 0.3009 | 0.3009 | 0.3541 | 0.3541 |
| V_{z} (L) | n | 5 | 8 | 2 | 9 |
| | Mean | 2.397 | 2.864 | 2.808 | 3.435 |
| | SD | 0.2101 | 0.8861 | 0.3880 | 0.9878 |
| | % CV | 8.8 | 30.9 | 13.8 | 28.8 |
| | Geo. Mean | 2.389 | 2.769 | 2.795 | 3.315 |
| | Geo. % CV | 8.9 | 26.8 | 13.9 | 28.7 |
| | Min | 2.110 | 2.110 | 2.534 | 2.397 |
| | Median | 2.388 | 2.589 | 2.808 | 3.085 |
| | Max | 2.668 | 4.883 | 3.082 | 5.012 |
| Vₛₛ (L) | n | 5 | 8 | 2 | 9 |
| | Mean | 2.461 | 2.891 | 2.700 | 3.100 |
| | SD | 0.2269 | 0.7701 | 0.1775 | 0.5972 |
| | % CV | 9.2 | 26.6 | 6.6 | 19.3 |
| | Geo. Mean | 2.453 | 2.815 | 2.698 | 3.051 |
| | Geo. % CV | 9.4 | 24.2 | 6.6 | 18.8 |
| | Min | 2.136 | 2.136 | 2.575 | 2.465 |
| | Median | 2.465 | 2.649 | 2.700 | 2.997 |
| | Max | 2.765 | 4.552 | 2.826 | 4.134 |
| MRT (day) | n | 5 | 8 | 2 | 9 |
| | Mean | 8.823 | 12.51 | 9.137 | 13.32 |
| | SD | 1.007 | 6.214 | 2.637 | 3.794 |
| | % CV | 11.4 | 49.7 | 28.9 | 28.5 |
| | Geo. Mean | 8.778 | 11.47 | 8.945 | 12.79 |
| | Geo. % CV | 11.3 | 44.1 | 29.9 | 31.7 |
| | Min | 7.581 | 7.581 | 7.273 | 7.273 |
| | Median | 8.629 | 9.744 | 9.137 | 12.59 |
| | Max | 10.30 | 25.77 | 11.00 | 18.40 |
| DNCₘₐₓ | n | 6 | 10 | 2 | 9 |
| (μg/mL/mg) | Mean | 0.4785 | 0.4584 | 0.4692 | 0.4444 |
| | SD | 0.06444 | 0.06213 | 0.02747 | 0.05598 |
| | % CV | 13.5 | 13.6 | 5.9 | 12.6 |
| | Geo. Mean | 0.4750 | 0.4547 | 0.4688 | 0.4413 |
| | Geo. % CV | 13.2 | 13.5 | 5.9 | 12.8 |
| | Min | 0.4065 | 0.3592 | 0.4497 | 0.3626 |
| | Median | 0.4701 | 0.4487 | 0.4692 | 0.4497 |
| | Max | 0.5827 | 0.5827 | 0.4886 | 0.5230 |
| DNAUC₀₋ₗₐₛₜ | n | 6 | 9 | 2 | 9 |
| (day*μg/mL/mg) | Mean | 2.961 | 3.608 | 3.283 | 4.223 |
| | SD | 0.8226 | 1.185 | 0.7202 | 1.031 |
| | % CV | 27.8 | 32.9 | 21.9 | 24.4 |
| | Geo. Mean | 2.851 | 3.413 | 3.243 | 4.113 |
| | Geo. % CV | 32.3 | 38.2 | 22.4 | 24.8 |
| | Min | 1.631 | 1.631 | 2.774 | 2.774 |
| | Median | 3.037 | 3.482 | 3.283 | 4.296 |
| | Max | 4.001 | 5.235 | 3.792 | 6.269 |
| DNAUC_{0-∞} | n | 5 | 8 | 2 | 9 |
| (day*μg/mL/mg) | Mean | 3.595 | 4.153 | 3.359 | 4.302 |
| | SD | 0.3947 | 0.8800 | 0.7556 | 1.033 |
| | % CV | 11 | 21.2 | 22.5 | 24.0 |
| | Geo. Mean | 3.579 | 4.076 | 3.316 | 4.193 |
| | Geo. % CV | 10.4 | 20.7 | 23.0 | 24.6 |
| | Min | 3.323 | 3.323 | 2.824 | 2.824 |
| | Median | 3.501 | 3.913 | 3.359 | 4.451 |
| | Max | 4.277 | 5.661 | 3.893 | 6.317 |

Abbreviations: AUC₀₋ₗₐₛₜ = AUC from time 0 to the time of last quantifiable concentration; AUC_{0-∞} = AUC from time 0 extrapolated to infinite time; CL = clearance; Cₘₐₓ = maximum concentration; CT = concentration at the end of infusion time (ie, 2 hour); CV = coefficient of variation; DNAUC₀₋ₗₐₛₜ = dose normalized AUC₀₋ₗₐₛₜ; DNAUC_{0-∞} = dose normalized AUC_{0-∞}; DNCₘₐₓ = dose normalized Cₘₐₓ; Geo = geometric; Max = maximum; Min = minimum; N = number of subjects in the pharmacokinetic population in respective treatment; n = number of observations; SD = standard deviation; tₘₐₓ = time of Cₘₐₓ; t_{1/2} = terminal half-life; V_{Z} = volume of distribution at terminal phase; Vₛₛ = volume of distribution at steady state; MRT = mean residence time; λz = terminal elimination rate constant

Statistical analysis of XTMAB-16 dose proportionality using the power model is presented in Table 28. In this analysis, the slope for Cₘₐₓ (1.02) was close to 1.00 and the 95% Cis [0.79, 1.26] were almost contained within the dose-proportional interval [0.8, 1.25]. However, the slope values for the PK parameters AUC₀₋ₗₐₛₜ (1.31), and AUC₀-∞ (1.10) were greater than 1.00 and the corresponding upper 95% CI from 2 to 4 mg/kg shows greater than usual dose-proportional interval [0.8, 1.25] hence showing a greater than dose-proportional increase in the AUCs.

Based on the dose normalized PK parameters, dose normalized Cmax (DNCmax) and dose normalized AUC0-∞ (DNAUC0-∞) between the 2 cohorts were nearly in good agreement. However, there is some difference in DNAUC0-last in the 2 mg/kg group compared to the 4 mg/kg group.

TABLE 23
Study XTMAB-16-101: Statistical Analysis
to Assess Dose Proportionality of Serum
Pharmacokinetic Parameters of XTMAB-16

| Parameter (Unit) | n | μ | β (SE) | 95% CI for β |
|---|---|---|---|---|
| Cₘₐₓ (μg/mL) | 19 | 3.442 | 1.023 (0.111) | 0.79; 1.26 |
| AUC₀₋ₗₐₛₜ (day*μg/mL) | 18 | 5.270 | 1.315 (0.225) | 0.84; 1.79 |
| AUC_{0-∞} (day*μg/mL) | 17 | 5.581 | 1.104 (0.189) | 0.70; 1.51 |

Abbreviations: N = Number of observations; μ = Intercept; β = Slope; SE = Standard error of slope; CI = confidence interval.
Dose proportionality was assessed using POWER model.
Model: The linear model fitted is log (Y) = μ + β * log (dose), where Y is the PK parameter.

### Population Pharmacokinetic Analysis

Population PK analysis and simulations of XTMAB-16 in non-sarcoidosis adult subjects were performed based on data collected in the Study XTMAB-16-101.

The PK analysis dataset included 277 PK observations from 19 subjects. Number of subjects, measurable observations, and below the limit of quantitation (BLQ) observations across studies are summarized by study in Table 24. Densely sampled concentration-time profiles were available in all 19 subjects on 2 mg/kg and 4 mg/kg.

No serum XTMAB-16 samples with a quantifiable/reported concentration value were excluded from the analysis. No individual subjects were excluded from the analysis.

TABLE 24
Plasma Concentrations in the PK Analysis Dataset

| | Total | | | Post- |
|---|---|---|---|---|
| Subjects | Observations | Measurable | | Treatment |
| (No. of | (No. of | PK | Predose | BLQ (% of |
| Excluded | Excluded | Observations | BLQ (% | Postdose |
| Subjects) | Observations) | (% of Total) | of Total) | Observations) |
| 19 (0) | 277 (0) | 266 (81.6%) | 19 (6.9%) | 32 (12.4%) |

Abbreviations: BLQ = below the limit of quantitation; PK = pharmacokinetic.
Source: Modeling and Simulation Report

An NCA was conducted to provide initial estimates for XMTAB-16 population PK analysis. Actual time was used in the analysis. BLQ values were set to 0 before Tₘₐₓ and set to missing after Tₘₐₓ. The PK parameters were summarized by dose level and ADA status in Table 25. ADA positive subjects had reduced exposure in terms of AUCs compared to ADA negative individuals.

TABLE 25
Summary Statistics of Serum Pharmacokinetic Parameters
of XTMAB-16 Following Single Intravenous Administration

| | 2 mg/kg XTMAB-16 | | 4 mg/kg XTMAB-16 | |
|---|---|---|---|---|
| PK Parameters | ADA Negative | ADA Positive | ADA Negative | ADA Positive |
| Cₘₐₓ (μg/mL) | 64.2 (14.4); 4 | 64.2 (17.5); 6 | 124 (18.1); 7 | 154 (NC); 2 |
| Cₘₐₓ/D | 32.1 (14.4); 4 | 32.1 (17.5); 6 | 31.1 (18.1); 7 | 38.5 (NC); 2 |
| (kg*μg/mL/mg) | | | | |
| Cₑₒᵢ * (μg/mL) | 63.7 (14.0); 4 | 61.2 (9.2); 6 | 122 (17.4); 7 | 151 (NC); 2 |
| Tₘₐₓ (h) | 2.15 [2.15- | 2.16 [2.10- | 3.00 [2.12- | 2.65 [2.27- |
| | 3.00]; 4 | 8.65]; 6 | 8.02]; 7 | 3.03]; 2 |
| AUCₗₐₛₜ | 14900 (63.4); 4 | 9340 (21.8); 6 | 30300 (18.0); 7 | 26000 (NC); 2 |
| (h*μg/mL) | | | | |
| AUCₗₐₛₜ/D | 7440 (63.4); 4 | 4670 (21.8); 6 | 7580 (18.0); 7 | 6510 (NC); 2 |
| (h*kg*μg/mL/mg) | | | | |
| AUC_{∞} | 19900 (29.5); 3 | 11400 (7.9); 6 | 30900 (18.4); 7 | 26600 (NC); 2 |
| (h*μg/mL) | | | | |
| AUC_{∞}/D | 9970 (29.5); 3 | 5690 (7.9); 6 | 7730 (18.4); 7 | 6660 (NC); 2 |
| (h*kg*μg/mL/mg) | | | | |
| T_{1/2} (h) | 315 (41.2); 3 | 150 (15.1); 6 | 270 (29.4); 7 | 159 (NC); 2 |
| MRT_{inf} (h) | 448 (34.9); 3 | 219 (13.0); 6 | 348 (22.4); 7 | 219 (NC); 2 |
| V_{z} (mL/kg) | 44.6 (11.3); 3 | 38.0 (14.3); 6 | 50.6 (24.9); 7 | 34.1 (NC); 2 |
| Vₛₛ (mL/kg) | 44.5 (5.8); 3 | 38.7 (13.5); 6 | 45.3 (16.9); 7 | 32.9 (NC); 2 |
| CL (mL/h/kg) | 0.106 (27.6); 3 | 0.177 (7.7); 6 | 0.133 (19.7); 7 | 0.156 (NC); 2 |
| Tₗₐₛₜ (h) | 1502.07 [46.68- | 506.98 [170.02- | 1659.33 [1033.03- | 842.6 [675.65- |
| | 1703.43]; 4 | 697.05]; 6 | 1703.65]; 7 | 1009.55]; 2 |

Abbreviations: AUC_{∞} = area under the serum concentration-time curve; AUCₗₐₛₜ = area under the serum concentration-time curve from time 0 to the last measurable concentration; Cₑₒᵢ = end of infusion concentration; Cₘₐₓ = maximum serum concentration; CL = clearance; D = dose; MRT_{inf} = mean residence time to infinity; NC = not calculated; T_{1/2} = half-life; T ₘₐₓ = time of observed maximum concentration; Tₗₐₛₜ = time of last quantifiable plasma concentration; Vₛₛ = volume of distribution at steady state; V_{z} = volume of distribution at terminal phase Parameters are presented as mean (CV %); n with the exception of Tₘₐₓ and Tₗₐₛₜ presented median (min-max); n Cₑₒᵢ was set to measured serum XTMAB-16 concentration at 2-hour nominal time.
Source: Modeling and Simulation Report

Using the final base population PK model, select covariates were assessed in a univariate manner including the following:
1. Body weight, sample-level ADA, subject-level ADA, and subject-level nAb on CL
2. Body weight and subject-level ADA on central volume of distribution (V)
3. Body weight on peripheral volume of distribution (V2)
4. Weight and subject-level ADA were found to be significant influences on the PK of XTMAB-16.

The final population PK model for XTMAB-16 was a 2-compartment model with linear elimination and inter-individual variability included on CL, and central and peripheral volume of distribution. Body weight was included as a covariate on volume parameters and ADA status was included on CL. Based on the final model, the CL is 0.009 L/h, the volume of distribution at steady state (Vss) is 3.02 L, and the estimated t½ for a typical individual (with a weight of 72.4 kg) is 243.3 hours (194.0 hours if subject is ADA positive). Parameters of the final XTMAB-16 population PK model were estimated with good precision; the percentages of the relative standard error for PK parameters and the random effects were <23%. Goodness-of-fit plots suggest reasonable fit of the data. Individual- and population-predicted concentrations showed good correlation with observed data. There were no trends detected with respect to age, and minimal residual trends with respect to sex, on the PK of XTMAB-16. Due to small sample sizes the impact of race and ethnicity could not be robustly estimated; however, boxplots for ethnicity suggest overlap between Hispanic/Latino subjects and non-Hispanic/non-Latino subjects.

### Example 7: Assessing XTMAB-16 in an In Vitro Model of Sarcoidosis and Granulomatous Disease

The present example evaluates XTMAB-16 activity in an established in vitro model of granuloma formation using peripheral blood mononuclear cells from subjects with active pulmonary sarcoidosis to determine a potentially efficacious dose range. Data obtained from the first-in-human study of XTMAB-16 (NCT04971395) were used to develop a population pharmacokinetic (PPK) model to characterize the pharmacokinetics (PK) of XTMAB-16. Model simulations were performed to evaluate the sources of PK variability and to predict interstitial lung exposure based on concentrations in the in vitro granuloma model.

XTMAB-16 dose levels of 2 and 4 mg/kg, once every 2 weeks (Q2W) or once every 4 weeks (Q4W) for up to 12 weeks, were supported by data from the nonclinical, in vitro secondary pharmacology; the Phase 1 clinical study; and the PPK model developed to guide dose level and frequency assumptions. XTMAB-16 inhibited granuloma formation and suppressed interleukin-1 (IL-1 secretion in the in vitro granuloma model with a half maximal inhibitory concentration (IC50) of 5.2 and 3.5 μg/mL, respectively. Interstitial lung concentrations on average, following 2 or 4 mg/kg administered Q2W or Q4W, are anticipated to exceed the in vitro IC50 concentrations.

The data presented herein provide a rationale for dose selection of XTMAB-16 for subjects with pulmonary sarcoidosis in Example 8.

### Materials and Methods

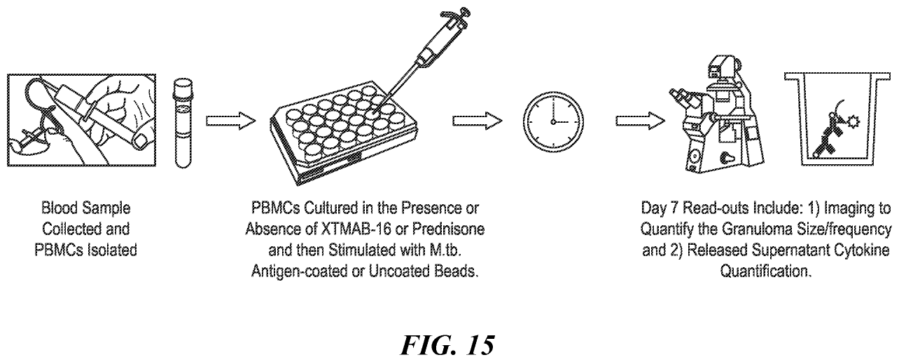

The in vitro sarcoidosis model of granuloma formation was generated to closely replicate the molecular features of granulomas in diseased sarcoidosis tissues (8-13). Peripheral blood mononuclear cells (PBMCs) from subjects with active pulmonary sarcoidosis with lung and/or lymph node involvement who were naïve to tuberculosis (TB) (negative purified protein derivative [PPD] skin test and/or Quantiferon Gold test; N=10)), non-smokers, and not treated with potent immune suppressants (e.g., methotrexate, azathioprine, prednisone, anti-TNF antibodies) within 3 months, were isolated and cultured in standard 24-well plates were isolated and cultured. PMBCs were exposed to either uncoated bead (UNC) or Mycobacterium tuberculosis (M. tb) antigen PPD-coated beads for 7 days. Sarcoid PBMCs were cultured in the presence or absence of inhibitor pre-treatments (XTMAB-16 or prednisone (in a dose-range demonstrated to suppress cultured human PBMC inflammatory responses in vitro in prior studies for 30 minutes followed by exposure to the PPD-coated beads (FIG. 15). Cells were imaged 7 days post-treatment. Quantitative analysis of Granuloma size/frequency was analyzed (performed using MIPAR image-processing software. Area fraction, defined as the cumulative granuloma area as a percentage of the total area of the image, was calculated for each treatment group based on at least 4 independent images. Following imaging, supernatant samples were collected and quantified for extracellular cytokine release using an enzyme-linked immunosorbent assay.

Granuloma formation following pre-treatment with XTMAB-16 (1, 5, 10, 20, and 40 μg/mL) or prednisone (1 and 10 μM; 0.358 and 3.58 μg/mL respectively) (30 minutes prior to PPD-coated bead exposure) was compared to that following treatment of PPD-coated beads alone. UNC served as comparable negative controls.

Model studies are under the ClinicalTrials.gov identifier, NCT01857401, the entirety of which is herein incorporated by reference.

### Phase 1 XTMAB-16 Study

The Phase 1 Study of XTMAB-16, XTMAB-16-101 (NCT04971395), was a randomized, double-blind, placebo-controlled, first-in-human study to evaluate the safety, tolerability, PK, and immunogenicity profile of single IV infusions of XTMAB-16 in non-sarcoidosis non-smoking adults. XTMAB-16 was administered via IV infusion over a 2-hour period at doses of 2 or 4 mg/kg. Based on the established no-observed-adverse-effect level (NOAEL) of the XTMAB-16 in the nonclinical program (40 mg/kg), the maximum clinical starting dose was 4 mg/kg.

Inclusion criteria included non-sarcoidosis adult male or female; aged 18 to 45 years, inclusive; with a weight of between 45 and 100 kg and a body mass index (BMI) between 18.0 and 30.0 kg/m², inclusive; and with clinically acceptable clinical laboratory values and electrocardiogram results. Exclusion criteria included receipt of any investigational compound within 90 days prior to dosing and any significant illness. A total of 25 non-sarcoidosis adult subjects were enrolled and assigned into two treatment cohorts (2 or 4 mg/kg) or placebo. The mean age (min, max) of the subjects was 32 years (19, 45); the majority were female (N=16, 64%), black or African American (N=20, 80%), and not Hispanic or Latino (N=19, 76%); with a mean BMI (min, max) of 25.84 (20.9, 29.8) (Table 26).

TABLE 26
Demographic Characteristics: Safety Set

| | | Cohort 1 | | Cohort 2 | | | | | |
|---|---|---|---|---|---|---|---|---|---|
| | | XTMAB-16 | | XTMAB-16 | | Pooled | | | |
| | | 2 mg/kg | | 4 mg/kg | | Placebo | | Overall | |
| Parameters | Statistics | (N = 10) | | (N = 9) | | (N = 6) | | (N = 25) | |
| Age (Years) | n | 10 | | 9 | | 6 | | 25 | |
| | Mean | 33.0 | | 30.6 | | 31.7 | | 31.8 | |
| | SD | 5.64 | | 6.86 | | 3.44 | | 5.59 | |
| | Median | 32.0 | | 30.0 | | 31.0 | | 32.0 | |
| | Min, Max | 26, 45 | | 19, 42 | | 28, 36 | | 19, 45 | |
| Sex, n (%) | Male | 3 | (30.0) | 3 | (33.3) | 3 | (50.0) | 9 | (36.0) |
| | Female | 7 | (70.0) | 6 | (66.7) | 3 | (50.0) | 16 | (64.0) |
| Race, n (%) | American Indian or | 1 | (10.0) | 0 | (0.0) | 0 | (0.0) | 1 | (4.0) |
| | Alaska Native | | | | | | | | |
| | Asian | 0 | (0.0) | 0 | (0.0) | 0 | (0.0) | 0 | (0.0) |
| | Black or African | 8 | (80.0) | 8 | (88.9) | 4 | (66.7) | 20 | (80.0) |
| | American | | | | | | | | |
| | Native Hawaiian or | 0 | (0.0) | 0 | (0.0) | 0 | (0.0) | 0 | (0.0) |
| | Other Pacific Islander | | | | | | | | |
| | White | 1 | (10.0) | 1 | (11.1) | 2 | (33.3) | 4 | (16.0) |
| | Other | 0 | (0.0) | 0 | (0.0) | 0 | (0.0) | 0 | (0.0) |
| Ethnicity, | Hispanic or Latino | 2 | (20.0) | 2 | (22.2) | 2 | (33.3) | 6 | (24.0) |
| n (%) | Not Hispanic or Latino | 8 | (80.0) | 7 | (77.8) | 4 | (66.7) | 19 | (76.0) |
| | Unknown | 0 | (0.0) | 0 | (0.0) | 0 | (0.0) | 0 | (0.0) |

| Height (cm) | n | 10 | 9 | 6 | 25 |
|---|---|---|---|---|---|
| | Mean | 167.1 | 167.9 | 170.2 | 168.1 |
| | SD | 10.42 | 9.12 | 8.84 | 9.28 |
| | Median | 162.0 | 165.0 | 167.5 | 165.0 |
| | Min, Max | 158, 185 | 158, 185 | 161, 186 | 158, 186 |
| Weight (kg) | n | 10 | 9 | 6 | 25 |
| | Mean | 70.68 | 74.06 | 76.38 | 73.26 |
| | SD | 11.783 | 12.134 | 11.566 | 11.596 |
| | Median | 69.05 | 72.60 | 73.40 | 72.40 |
| | Min, Max | 53.2, 97.2 | 54.3, 91.8 | 67.3, 99.0 | 53.2, 99.0 |
| Body Mass | n | 10 | 9 | 6 | 25 |
| Index (kg/m²) | Mean | 25.22 | 26.22 | 26.30 | 25.84 |
| | SD | 2.649 | 3.415 | 2.173 | 2.787 |
| | Median | 25.45 | 27.70 | 25.90 | 25.90 |
| | Min, Max | 21.0, 28.4 | 20.9, 29.8 | 23.8, 28.9 | 20.9, 29.8 |

% = percentage of subjects (the denominator is N)
Abbreviations: Max = maximum; Min = minimum; N = total number of subjects in safety set; N = number of subjects in specific category; SD = standard deviation

### Pharmacokinetic Analysis

PK analysis was performed on data collected in XTMAB-16-101. For the population PK (PPK) analysis, the dataset included information on subject identifier, date and time of XTMAB-16 dosing, nominal and actual date and time of PK sample collections, concentrations of XTMAB-16 in serum, as well as data of relevant covariates, including body weight, age, height, BMI, dose, subject-level anti-drug antibody (ADA), sample-level ADA, sex, race, and ethnicity. For the purposes of this analysis, below the limit of quantitation (BLQ) samples were set to missing.

PPK model development was performed using Phoenix® NLME (v 8.4, Certara). A two-compartment model was used as the starting point for this modeling exercise based on visual inspection of concentration-time profiles.

Covariate Analysis: Following the selection of a base PPK model, the relationships between covariates and PK parameters of XTMAB-16 were first explored graphically to obtain information on covariates likely to affect the parameters of interest, and to guide covariate analyses. Correlations between covariates were considered during the covariate selection process (i.e., where two covariates assumed or demonstrated graphically to correlation, only one of the two would be selected in a formal covariate model testing strategy).

Model Assessment: Model evaluation was based on standard model diagnostics and goodness-of-fit criteria (e.g., accuracy of parameter estimation [i.e., 95% confidence interval excluding 0], successful model convergence) and by looking at pertinent graphical representations of goodness-of-fit (e.g., fitted and observed concentrations versus time, conditionally weighted residuals versus time).

An internal qualification of the final model was to be performed through a prediction-corrected visual predictive check (pcVPC). A pcVPC was constructed using the final model estimates and involved simulating 1000 replicates of concentration-time profiles, followed by normalizing based on the typical population prediction to help account for differences in study design.

Following a successful evaluation of the final PPK model, individual estimates of PK and exposure parameters were derived by posterior Bayesian estimation and compared with PK parameters previously determined by non-compartmental analysis.

Simulations: Biweekly (Q2W) and once every 4-week (Q4W) regimens were simulated at 2 and 4 mg/kg over the course of 16 weeks. A total of 6 doses were administered in the Q2W regimen and 3 doses were administered in the Q4W regimen in the first 12 weeks. Monte-Carlo simulations were performed whereby each virtual subject's (N=500) PPK model parameters were obtained from sampling via the final PPK variance-covariance matrix. To account for body weight, body weight was assigned randomly from a uniform distribution of 50-100 kg. The infusion duration of XTMAB-16 was assumed to be 2 hours for each virtual subject. The ADA status was included as a predictor in the simulation.

To account for concentrations in lung tissue, interstitial lung concentration of XTMAB-16 was estimated using the antibody biodistribution coefficient for lung tissue (14.9%). Exposure parameters for a typical individual weighing 70 kg and with an ADA negative status following the first dose and the last dose in the lung and in serum were then calculated and presented in tabular format for illustrative purposes.

For all cohorts, 2 subjects (1 assigned to XTMAB-16 and 1 assigned to placebo; sentinel cohort) were dosed at least 48 hours before other subjects in the same cohort. After blinded review of available safety and laboratory data of the sentinel cohort, if no notable safety signals were identified, the remaining subjects within the same cohort were dosed. Subjects within a cohort were to be dosed at least 1 hour apart.

The subjects of the 4 mg/kg cohort received XTMAB-16 after completion of the 2 mg/kg cohort and at least 14 to 21 days of clinical assessment of the 2 mg/kg dose cohort subjects.

After a screening period of up to 28 days, subjects were admitted to the clinical research unit (CRU) on Day −2 for COVID-19 testing and baseline procedures. After eligibility was confirmed, subjects were randomly assigned to treatment (1:1 XTMAB-16 to placebo for the sentinel cohort, 3:1 for the remaining subjects) and administered study drug on Day 1. Subjects remained in the CRU through Day 8 for PK, biomarkers, anti-drug antibodies (ADA), and safety assessments. Subjects were discharged on Day 8 once all procedures for that day were completed. Subjects returned to the CRU on Days 15, 29, 43, 57, and 71.

Safety assessments included adverse events, safety laboratory assessments, vital signs, electrocardiogram, and physical examination. PK collection and analysis were also completed in a similar manner during the course of the study.

### Results

Activity of XTMAB-16 in the In Vitro Granuloma Model of Sarcoidosis: Demographic characteristics of the subjects included in the in vitro study are shown in Table 27. The designation of high or low responder indicates the response to PPD-coated beads in terms of in vitro granuloma formation.

TABLE 27
Demographics of Subjects Included in the In Vitro Study

| | Age | | | | Scadding | Extra- |
|---|---|---|---|---|---|---|
| Groups | (years) | Gender | Race | H/NH | CXR Stage | pulmonary |
| Sarcoidosis High | 69 | M | W | NH | III/IV | + |
| Responders | 61 | F | W | NH | II | − |
| (N = 6) | 51 | M | W | NH | II | + |
| | 63 | F | W | NH | II | − |
| | 65 | F | B | NH | III/IV | + |
| | 75 | F | W | NH | I | + |
| Mean (SEM) or | 64.0 | 2/4 | 5/1/0 | 0/6 | — | — |
| Ratio | (3.3) | (M/F) | (W/B/O) | (H/NH) | | |
| Sarcoidosis Low | 61 | M | W | NH | III | |
| Responders | 45 | F | B | NH | III | |
| (N = 5) | 63 | M | B | NH | IV | |
| | 52 | M | W | NH | I | |
| | 66 | F | B | NH | II | |
| Mean (SEM) or | 57.4 | 3/2 | 2/3/0 | 0/4 | — | — |
| Ratio | (3.9) | (M/F) | (W/B/O) | (H/NH) | | |

Abbreviations:
B = Black;
CXR = chest X-ray;
F = female;
H = Hispanic;
M = male;
NH = non-Hispanic;
O = other;
SEM = standard error of the mean;
W = White

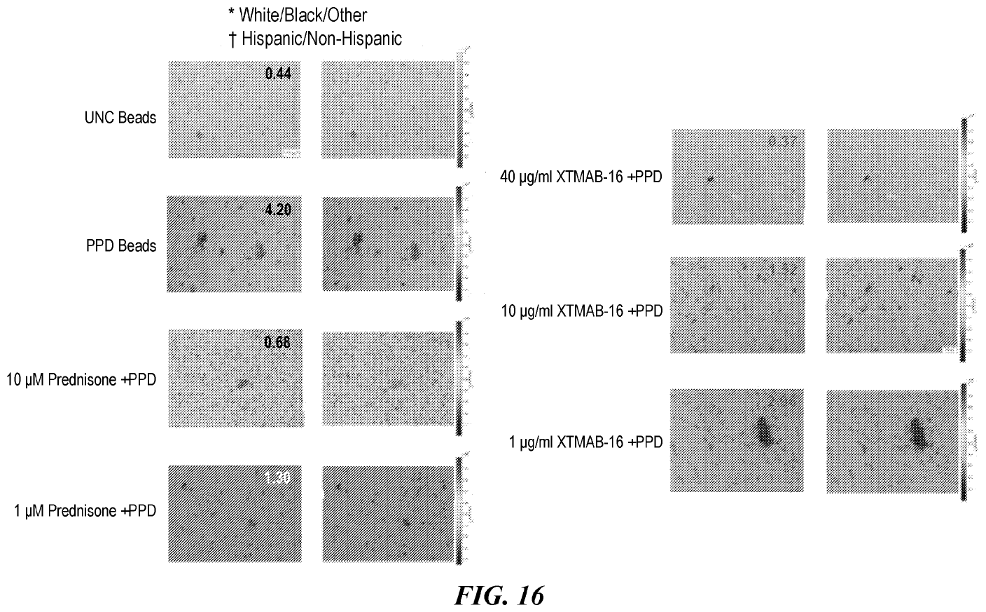

Representative photomicrographs of granuloma-like cell aggregates forming 7 days after high responder sarcoidosis PBMCs were incubated with either UNC or PPD-coated beads following XTMAB-16 or prednisone pre-treatment are shown in FIG. 16. XTMAB-16 pre-treatment significantly attenuated granuloma formation in the in vitro model in a dose-dependent fashion within the range of 1 to 40 μg/mL. The effect of XTMAB-16 at the higher doses in the in vitro granuloma model was comparable to that of prednisone (1 to 10 μM; 0.358 to 3.58 μg/mL) pre-treatment.

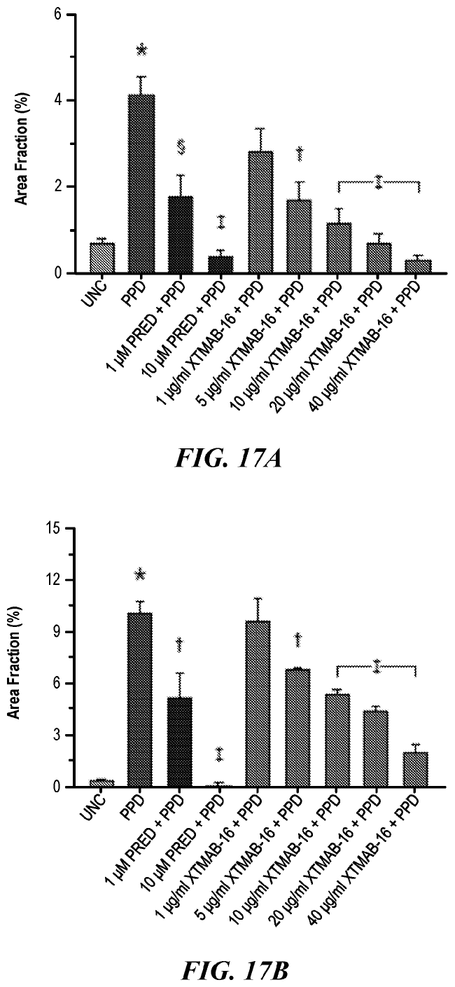

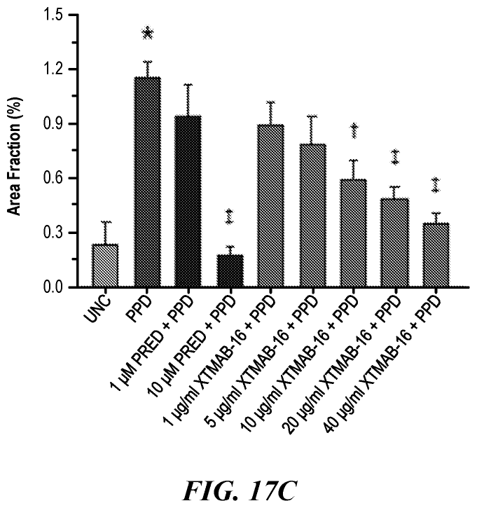

XTMAB-16 pre-treatment for 30 minutes had a significant dose-response effect in the in vitro granuloma model to suppress granuloma formation 7 days following treatment with PPD as calculated by the area fraction (FIGS. 17A-17C). For comparison, similar responses to prednisone pre-treatments were demonstrated.

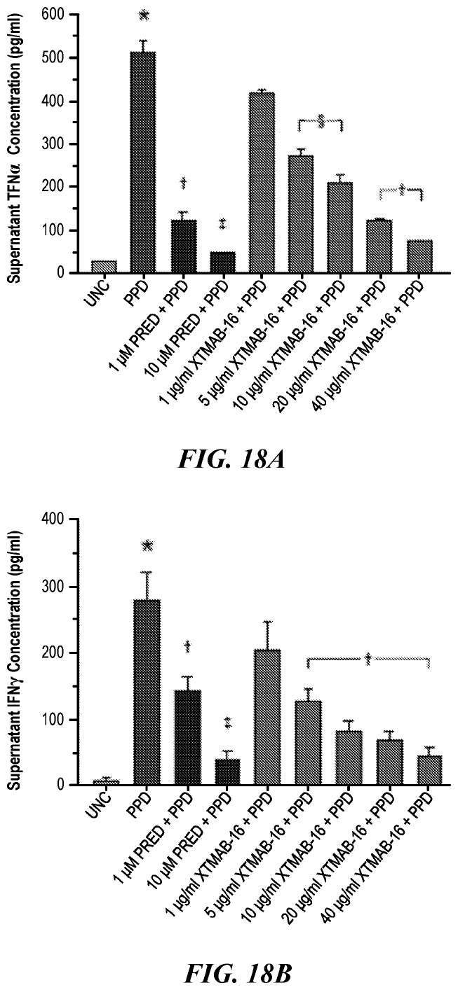

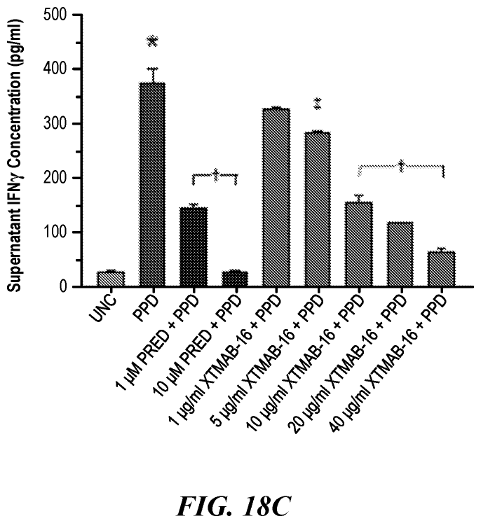

XTMAB-16 pre-treatment for 30 minutes had a significant dose-response effect in the in vitro granuloma model to suppress granuloma cytokine release 7 days following treatment with PPD (FIGS. 18A-18C). For comparison, similar responses to prednisone pre-treatments were demonstrated.

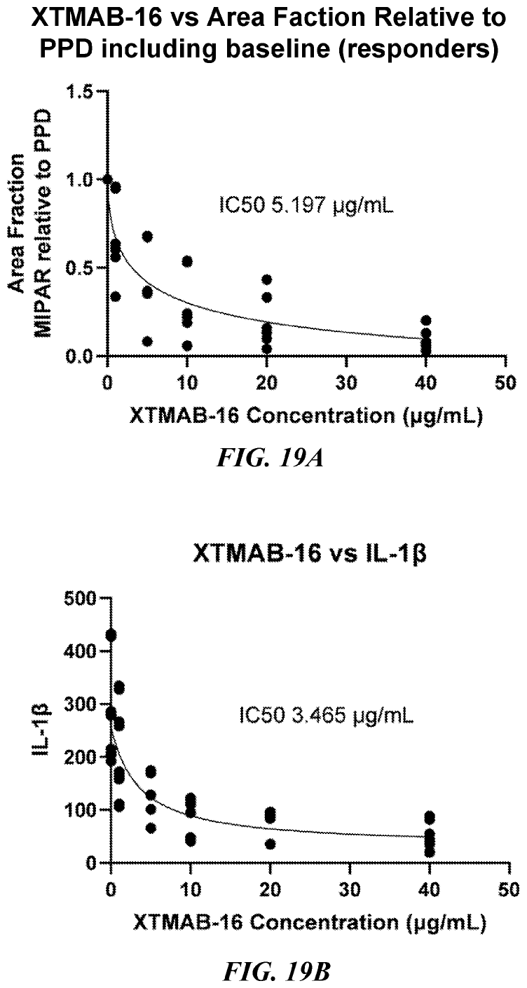

A separate statistical analysis was performed whereby all subject samples were evaluated for XTMAB-16 half maximal inhibitory concentration (IC50) against the MIPAR area fraction and the interleukin (IL)-1 concentration in the in vitro granuloma assay supernatant (FIGS. 19A and 19B). The 95% CI for the IC50 was not estimated because the software was unable to calculate a complete confidence interval. The IC50 for decrease in area fraction was 5.197 μg/mL and for decrease in IL-1613 was 3.465 μg/mL.

In vitro granuloma responses were variable between subjects. Despite this variability, there was a consistent dose-response to XTMAB-16 in vitro, reflected by objective imaging to assess granuloma area. Results were comparable to prednisone. IL-1b (rather than TNFα or IFNγ) best reflects granuloma response in this in vitro model and, therefore, may be a useful biomarker for future clinical studies in sarcoidosis. Based on the in vitro dose-response to XTMB-16, PPK modeling and simulation were performed using first-in-human study data to inform ongoing clinical development of XTMAB-16 for the treatment of sarcoidosis.

### PPK Modeling

PK Measurement: The PK analysis dataset included 277 PK observations from 19 subjects. Measurable PK observations were 226 (81.6%); pre-dose BLQ samples were 19 (6.9%) and post-treatment BLQ samples were 32 (12.4%). BLQ observations after administration of the first dose (“post-treatment BLQ”) account for 12.4% of all post-dose observations. No serum XTMAB-16 samples with a quantifiable/reported concentration value were excluded from the analysis. No individual subjects were excluded from the analysis.

Subject Characteristics: Baseline values of continuous and categorical covariates are summarized in Table 28. Descriptive statistics of ADA samples are presented in Table 29.

TABLE 28
Summary of Continuous and Categorical Covariates

| | |
|---|---|
| Covariate | Study XTMAB16-101 (N = 19) |

Continuous Covariates, Mean (SD); Median [min, max]

| | |
|---|---|
| Weight (kg) | 72.3 (11.7); 72.4 [53.2, 97.2] |
| Age (years) | 31.8 (6.2); 32 [19, 45] |
| BMI (kg/m²) | 25.7 (3.0); 25.9 [20.9, 29.8] |
| Height (cm) | 167.5 (9.6); 163 [158, 185] |

Categorical Covariates, n (%)

| | | |
|---|---|---|
| Sex, male | 6 | (31.6) |
| Caucasian race | 2 | (10.5) |
| Black or African American | 16 | (84.2) |
| American Indian or Alaska Native | 1 | (5.3) |
| ADA positive | 8 | (42.1) |
| ADA negative | 11 | (57.9) |
| Subject level neutralizing ADA positive | 8 | (42.1) |
| Subject level neutralizing ADA negative | 11 | (57.9) |

Note:
A subject with at least one positive ADA sample is considered to have a positive ADA status.
Abbreviations:
ADA = anti-drug antibody;
BMI = body mass index;
max = maximum;
min = minimum;
N = number of subjects with available information;
N = number of subjects in specific category;
SD = standard deviation

TABLE 29
Summary of ADA Samples

| | N (% of Total) | |
|---|---|---|
| | Study XTMAB16-101 | |
| Samples | (N = 19) | |
| Total number of samples | 277 | (100) |
| Total number of pre-dose samples | 19 | (6.9) |
| Total number of post-dose samples | 258 | (93.1) |
| Number of ADA samples | 18 | (6.1) |
| Number of neutralizing positive ADA samples | 18 | (6.1) |

Abbreviations:
ADA = anti-drug antibody;
N = number of subjects with available information

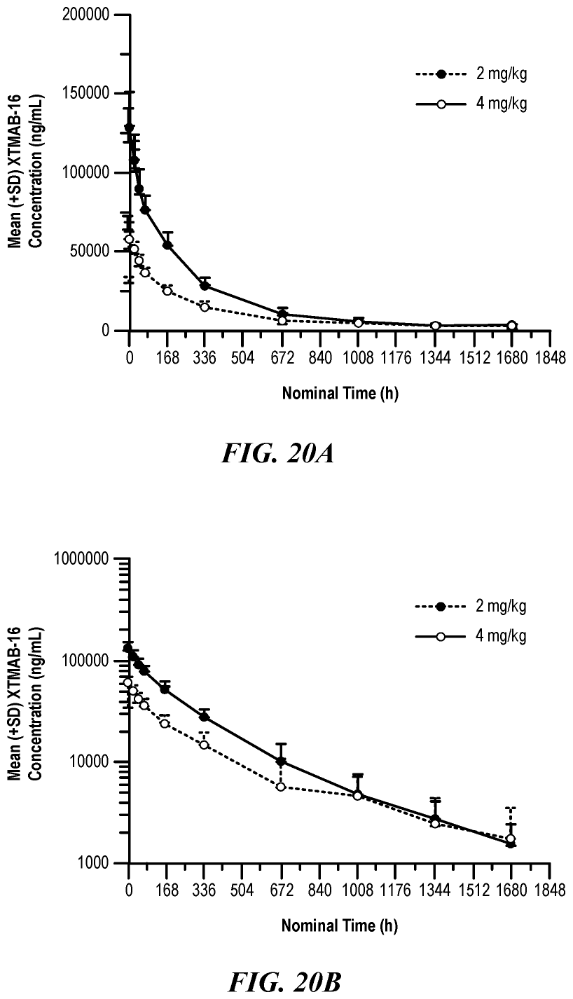

PPK Analysis: The arithmetic mean (+ standard deviation) serum drug concentration-time profiles of the study population are shown in FIGS. 20A and 20B. The shape of the elimination phase in the semi-log plots may suggest two-compartment kinetics, while the XTMAB-16 concentration versus time profiles may suggest a dose-proportional exposure with increasing doses.

Model Development and Qualification: A two-compartment model was used as an initial structural model. Inter-individual variability (IIV) was evaluated on all parameters. Because of the lack of improvement with IIV on inter-compartmental clearance (Q), only IIV on clearance (CL), central volume of distribution (V), and peripheral volume of distribution (V2) were supported and retained in the model. A combined additive and proportional error model was selected to describe the residual error. Weight and subject-level ADA were found influence the PK of XTMAB-16. The parameter estimates for the final PPK model are presented in Table 30. Based on the final model, the CL was 0.009 L/h, the volume of distribution at steady state was 3.02 L, and the estimated half-life for a typical individual (with a weight of 72.4 kg) was 243.3 hours (194.0 hours if subject is ADA positive). Parameters of the final XTMAB-16 PPK model were estimated with good precision; the percentages of the relative standard error for PK parameters and the random effects were <23%.

TABLE 30
Final Model Parameter Estimates

| Parameters | Estimates | % RSE | 95% CI |
|---|---|---|---|
| CL (L/h) | 0.00902 × 1.274 if | 6.2 | 0.008-0.010 |
| | subject is ADA positive | | |
| V (L) | 2.30 × (weight/72.4)^{0.426} | 2.2 | 2.20-2.40 |
| | 72.4 | | |
| V2 (L) | 0.715 x (weight/72.4)^{0.426} | 13.1 | 0.53-0.90 |
| | 72.4 | | |
| Q (L/h) | 0.0119 | 22.5 | 0.007-0.017 |

| Random Effects^{a} | Estimates | % RSE | Shrinkage (%) |
|---|---|---|---|
| IIV on CL | 0.0246 | 6.7 | 7.1 |
| IIV on V | 0.00936 | 2.7 | 4.2 |
| IIV on V2 | 0.203 | 16.8 | 17.7 |

| Residual Error | Estimates | % CV |
|---|---|---|
| Proportional error (%) | 6.89 | 9.6 |
| Additive error (ng/ml) | 819 | 57.5 |

^{a}Expressed as variance
Abbreviations:
ADA = anti-drug antibody;
CI = confidence interval;
CL = clearance;
CV = coefficient of variation;
IIV = inter-individual variability;
Q = inter-compartmental clearance;
RSE = relative standard error;
V = central volume of distribution;
V2 = peripheral volume of distribution

The final model resolved residual covariate trends with respect to body weight and sex on the central volume of distribution; however, a small residual trend in the peripheral volume remained with respect to sex. Addition of a covariate on sex, however, did not result in a significant drop in objective function value (Δ−2LL=5.01), and the sex covariate was not included in the final model. Due to small sample sizes, the impact of race and ethnicity was not robustly estimated.

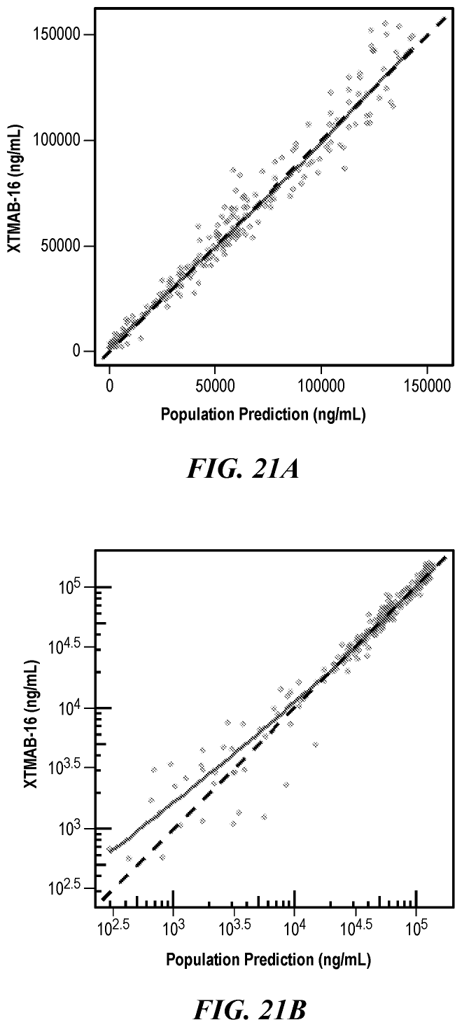

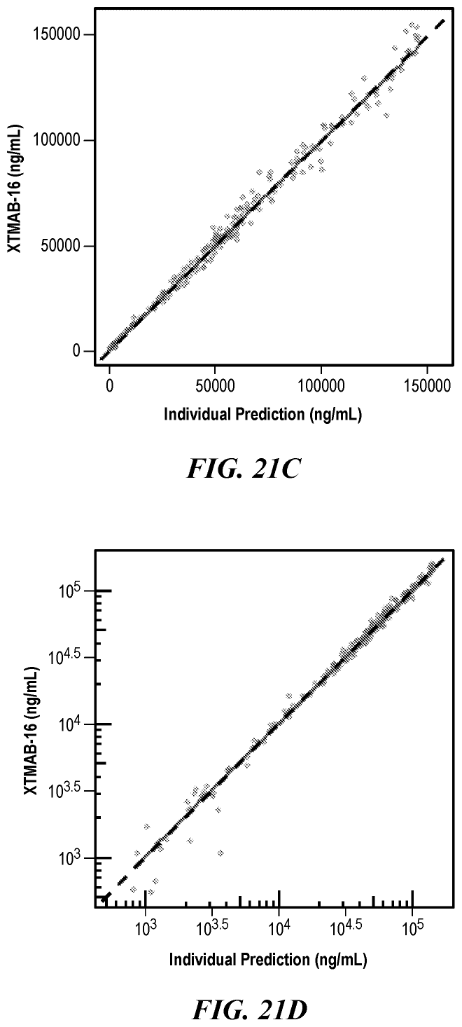

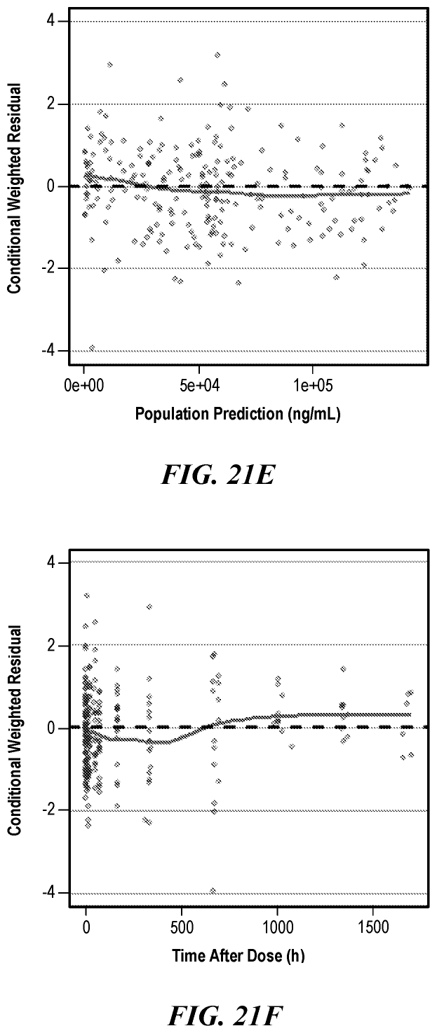

Goodness-of-fit plots suggested reasonable fit of the data (FIGS. 21A-21F). Individual- and population-predicted concentrations showed good correlation with observed data (FIGS. 21A-21D). The CWRES were evenly distributed around 0 and showed no trends over time or by concentration, thus indicating a lack of bias in the model (FIGS. 21E and 21F).

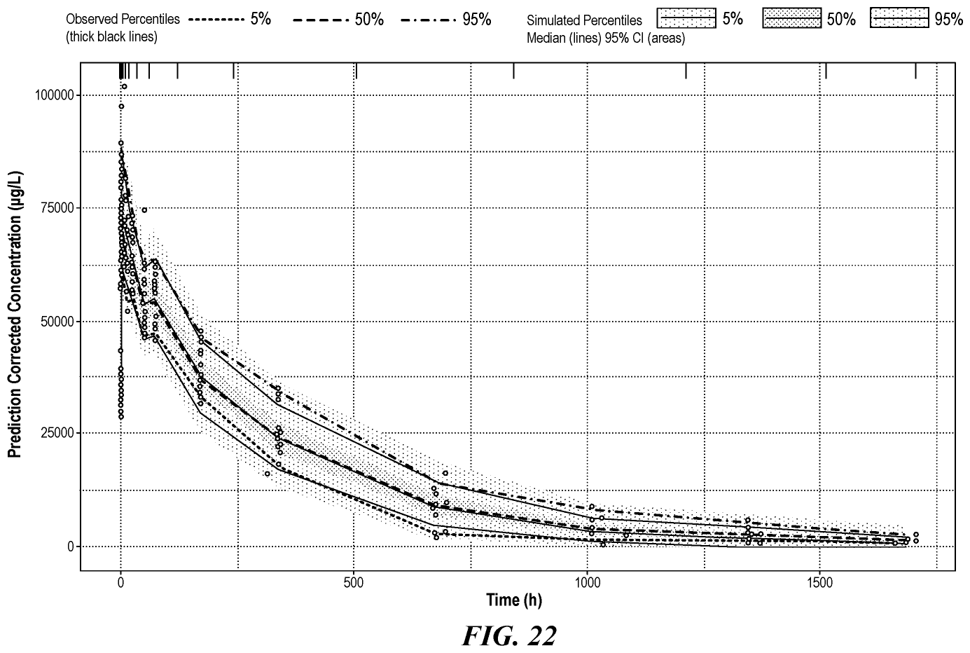

A pcVPC was performed to qualify the model, whereby 1000 replicates of the observed subjects were simulated. A pcVPC plot (linear-log) for the final XTMAB-16 PK model concentrations is presented in FIG. 22. The final PPK model was able to predict the observed median and 5th and 95th percentiles of observed XTMAB-16 concentrations with good accuracy. Model evaluation by pcVPC showed suitable predictive performance of the model, which captured both the central tendency and variability in observed concentrations.

Simulations: Simulated XMTAB-16 serum and lung exposure increased with increasing doses. For a given mg/kg dose, the Q2W dosing regimen produced a higher trough concentration (C_{trough}) and average concentration (C_{avg}) compared to the Q4W dosing regimen (Tables 31 and Table 32). PK parameters for simulated serum XTMAB-16 concentrations following single administration are shown in Table 33.

TABLE 31
Simulated XTMAB-16 Exposure in Serum
at Steady State (ADA Negative)

| Dosing Regimen | | | C_{trough} (μg/mL) | C_{avg} (μg/mL) |
|---|---|---|---|---|
| Single Dose | Q2W | 2 mg/kg | 16.1 | — |
| | Q2W | 4 mg/kg | 32.3 | — |
| | Q4W | 2 mg/kg | 6.11 | — |
| | Q4W | 4 mg/kg | 12.2 | — |
| Repeated | Q2W | 2 mg/kg | 25.9 | 46.1 |
| Dose | Q2W | 4 mg/kg | 51.8 | 92.1 |
| | Q4W | 2 mg/kg | 7.11 | 23.0 |
| | Q4W | 4 mg/kg | 14.2 | 46.1 |

Abbreviations:
ADA = anti-drug antibody;
C_{avg} = average concentration;
C_{trough} = trough concentration;
Q2W = every 2 weeks;
Q4W = every 4 weeks

TABLE 32
Simulated XTMAB-16 Exposure in Lung
at Steady State (ADA Negative)

| Dosing Regimen | | | C_{trough} (μg/mL) | C_{avg} (μg/mL) |
|---|---|---|---|---|
| Single Dose | Q2W | 2 mg/kg | 2.40 | — |
| | Q2W | 4 mg/kg | 4.81 | — |
| | Q4W | 2 mg/kg | 0.91 | — |
| | Q4W | 4 mg/kg | 1.82 | — |
| Repeated | Q2W | 2 mg/kg | 3.86 | 2.40 |
| Dose | Q2W | 4 mg/kg | 7.71 | 4.81 |
| | Q4W | 2 mg/kg | 1.06 | 0.91 |
| | Q4W | 4 mg/kg | 2.12 | 1.82 |

Abbreviations:
ADA = anti-drug antibody;
C_{avg} = average concentration;
C_{trough} = trough concentration;
Q2W = every 2 weeks;
Q4W = every 4 weeks

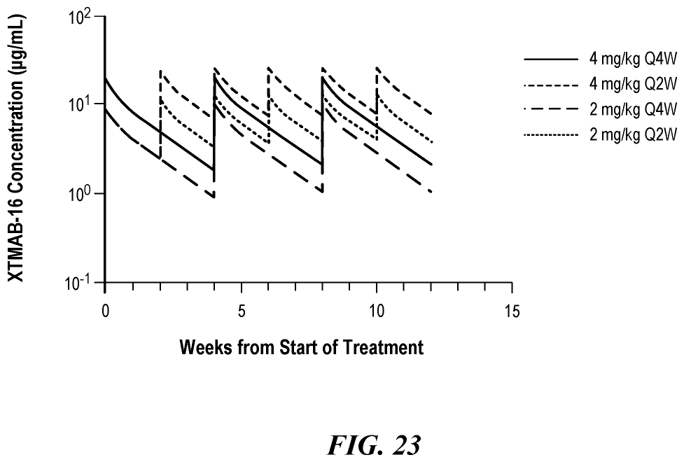

After accounting for the anticipated biodistribution for an mAb from plasma to lung tissue, simulations in adult subjects suggested that 4 mg/kg Q2W would largely achieve C_{trough} values in lung that exceed the IC50 that demonstrated reduction in granuloma growth in the in vitro sarcoidosis model (granuloma area fraction IC50=5.197 μg/mL; IL-1μ IC50=3.465 μg/mL); whereas doses of 2 mg/kg Q2W or Q4W or 4 mg/kg Q4W would fall below the in vitro IC50 (FIG. 23). The C_{avg} predicted lung exposure was similar to the granuloma IC50 for the 4 mg/kg Q2W regimen with the remaining regimens falling below the IC50. Thus, the proposed clinical dose regimens were predicted to provide for a range of XTMAB-16 PK lung exposure in the steep portion of the exposure-response curve.

TABLE 33
Pharmacokinetic Parameters for Simulated Serum XTMAB-16 Concentrations
Following Single Administration (ADA Negative)

| | Cₘₐₓ | AUC_{inf} | Cₑₒᵢ | C_{trough} | Vz | Vss | CL | Tₘₐₓ | MRT_{inf} | T_{1/2} |
|---|---|---|---|---|---|---|---|---|---|---|
| | (μg/mL) | (h*μg/mL) | (μg/mL) | (μg/mL) | (mL/kg) | (mL/kg) | (mL/h/kg) | (h) | (h) | (h) |
| 2 mg/kg | 61.1 | 15400 | 61.1 | 16.1 | 43.9 | 42.1 | 0.130 | 2.00 | 324 | 234 |
| Q2W | | | | | | | | | | |
| 2 mg/kg | 61.1 | 15500 | 61.1 | 6.11 | 44.2 | 42.4 | 0.129 | 2.00 | 329 | 238 |
| Q4W | | | | | | | | | | |
| 4 mg/kg | 122 | 30800 | 122 | 32.3 | 43.9 | 42.1 | 0.130 | 2.00 | 324 | 234 |
| Q2W | | | | | | | | | | |
| 4 mg/kg | 122 | 31000 | 122 | 12.2 | 44.2 | 42.4 | 0.129 | 2.00 | 329 | 238 |
| Q4W | | | | | | | | | | |

Cₑₒᵢ was set to simulated serum XTMAB-16 concentration at 2-hour nominal time.
Abbreviations:
ADA = anti-drug antibody;
AUC_{inf} = area under the concentration-time curve from time 0 extrapolated to infinity;
Cₑₒᵢ = end of infusion concentration;
CL = clearance;
Cₘₐₓ = maximum concentration;
C_{trough} = trough concentration;
MRT_{inf} = mean residence time;
Q2W = every 2 weeks;
Q4W = every 4 weeks;
T_{1/2} = half-life;
Tₘₐₓ = time to maximum concentration;
Vss = volume of distribution at steady state;
Vz = apparent volume of distribution during the terminal phase

### Example 9: Assessing XTMAB-16 in Subjects with Pulmonary Sarcoidosis

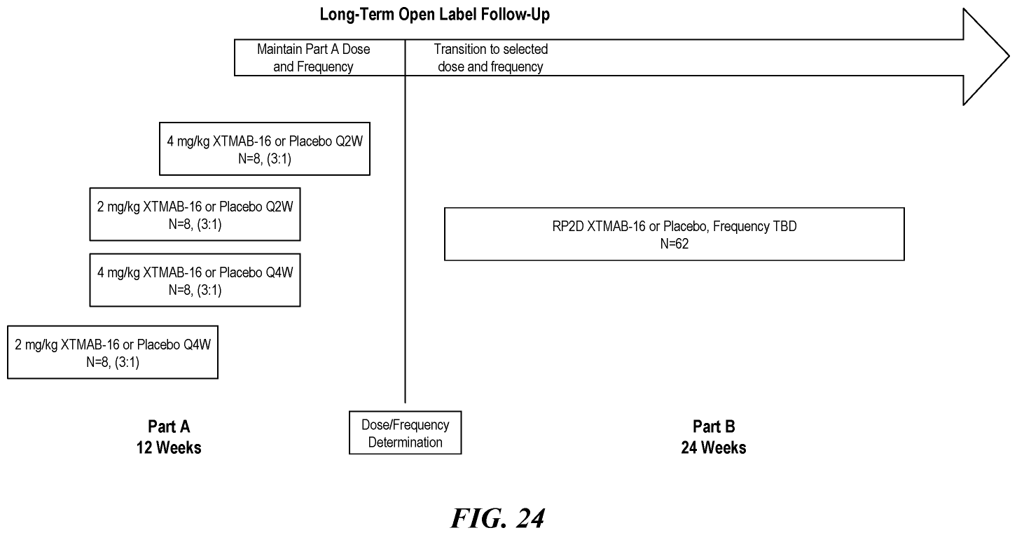

The present example will be conducted to as a Multiple Ascending Dose (MAD)/Proof of Concept (POC) Study (hereafter referred to as Phase 2 Program) of XTMAB-16 in subjects with pulmonary sarcoidosis with or without extrapulmonary involvement. The study is comprised of two parts: Part A will be a randomized double-blind placebo-controlled multiple dose-escalating study, and Part B will be a randomized double-blind placebo-controlled POC study (FIG. 24).

The objective of Part A is to evaluate the safety and tolerability of MADs of XTMAB-16, and to determine the recommended Phase 2 dose and frequency for Part B for XTMAB-16 administration in subjects with pulmonary sarcoidosis with or without extrapulmonary manifestations. Part A objectives and endpoints are outlined in Table 34-Table 36.

The objective of Part B is to confirm preliminary efficacy of XTMAB-16 as measured by the ability to reduce background oral corticosteroid use in subjects with pulmonary sarcoidosis with or without extrapulmonary manifestations. Part B objectives and endpoints are outlined in Table 37.

The rationale for this study is to conduct dose range finding to identify the recommended Phase 2 dose (RP2D) for Part B and characterize a variety of clinical and biomarker outcome measures to demonstrate efficacy and POC for XTMAB-16 in this subject population.

TABLE 34
Part A (MAD) Primary Objectives and Endpoints

| | |
|---|---|
| Objectives | Endpoints |

Primary

| | |
|---|---|
| Safety | Safety Endpoints: |
| To evaluate the safety and tolerability | Rate of Adverse Events (AEs), including |
| of multiple ascending doses of | Serious Adverse Events (SAEs), Dose |
| XTMAB-16 in subjects with pulmonary | Limiting Toxicities (DLTs), and Adverse |
| sarcoidosis with or without | Events of Special Interests (AESIs) |
| extrapulmonary manifestations | throughout the study duration |
| Determine recommended phase 2 | Endpoints: |
| dose | Pharmacokinetics (PK) profile including: |
| To determine the recommended | C_{trough} |
| Phase 2 dose level and frequency for | Cₘₐₓ |
| XTMAB-16 in subjects with pulmonary | C_{average} |
| sarcoidosis with or without | Area Under the Curve (AUC) |
| extrapulmonary manifestations for | Pharmacodynamics (PD) markers, |
| Part B of the study | including: |
| | Angiotensin converting enzyme |
| | (ACE) |
| | Soluble IL-2 receptor (sIL 2R) |
| | C-reactive protein (CRP) |
| | Interleukin-1b (IL-1b) |
| | Calcitriol (vitamin D 1, 25) |
| | Occurrence and persistence of anti-drug |
| | antibodies (ADA) |
| | Observed reduction in dose of |
| | corticosteroid |
| | Observed change in quality of life (QoL) |
| | and Pulmonary endpoints |
| | Mean percent change in forced vital |
| | capacity (FVC)% from Baseline to |
| | Week 12 |
| | Change in King's Sarcoidosis |
| | Questionnaire (KSQ) - Lung Score |
| | KSQ - General Health Status Module |
| | Leicester Cough Questionnaire (LCQ) |
| | Steroid Toxicity Questionnaire (STQ) |
| | Observed DLT, rate of AESI throughout |
| | the study duration between dose levels |
| | and frequencies |

TABLE 35
Part A (MAD) Secondary Objectives and Endpoints

| | |
|---|---|
| Objectives | Endpoints |

Secondary

| | |
|---|---|
| Efficacy: | Efficacy Endpoints: |
| To observe the potential therapeutic | Proportion of subjects who achieve the |
| effect of XTMAB-16 as measured by | targeted tapered dose of corticosteroid |
| the ability to reduce background oral | (prednisone 5 mg/day or equivalent) by |
| corticosteroid use | Week 12 |
| | Proportion of subjects who achieve at |
| | least 50% reduction in dose of |
| | corticosteroid by Week 12 |
| Pharmacokinetic and | Pharmacokinetic Endpoints: |
| Pharmacodynamics: | Clearance and volume parameters and |
| To evaluate the PK profile of XTMAB- | half-life in the intended subject |
| 16 and the effects on immunogenicity | population |
| and biomarkers in subjects with | Dose proportionality |
| pulmonary sarcoidosis with or without | Accumulation ratio repeat dosing |
| extrapulmonary manifestations | Area under the effect-time curve from |
| | time 0 (predose) to the last quantifiable |
| | effect concentration time point, t (AUEC₀₋ₜ) |
| | Maximum observed effect (Eₘₐₓ, obs) |
| | Time to reach E_{max, obs} (tEₘₐₓ, obs) |
| | Change in Biomarker Endpoints |
| | Absolute and percent change in |
| | biomarkers from Baseline to End of |
| | Infusion (EOI), Week 2, 4, 8, and 12 |
| | Interleukin-6 (IL-6) |
| | Soluble Tumor Necrosis Factor α |
| | (sTNFα) |
| | Immunogenicity |
| | Number and percentage of subjects by |
| | cohort who test positive for XTMAB-16 |
| | ADA at Baseline, Week 4, Week 8, and |
| | Week 12 and transient & persistent |
| | positive status at follow up assessments |
| | Number and percentage of subjects by |
| | cohort who test positive for XTMAB-16 |
| | nAb at Baseline, Week 4, Week 8, and |
| | Week 12 and transient & persistent |
| | positive status at follow up assessments |

TABLE 36
Part A (MAD) Exploratory Objectives and Endpoints

| | |
|---|---|
| Objectives | Endpoints |

Exploratory

| | |
|---|---|
| To observe the | Change in Quality-of-Life Measurements |
| potential therapeutic | from Baseline to Week 12 |
| effect of XTMAB-16 | Patient Global Assessment (PGA) |
| as measured by | Fatigue Assessment Scale (FAS) |
| Quality of Life, | Modified Medical Research Council |
| Pulmonary, and | (mMRC) Dyspnea Scale |
| Imaging endpoints | Short Form-36 (SF-36) |
| | Pulmonary Endpoints at Baseline to |
| | Week 12 |
| | Change in 6 Minute Walk Distance |
| | (6MWD) |
| | Imaging Endpoints at Baseline to Week 12 |
| | Change in high-resolution computed |
| | tomography (HRCT) |

Abbreviations: 6 MWD=6 minute walk distance; ACE=angiotensin converting enzyme; ADA=anti-drug antibodies; AE=adverse event; AESI=adverse events of special interest; AUC=area under the curve; AUEC=area under the effect curve; C_{average}=average concentration; Cₘₐₓ=Maximum observed concentration; CRP=C-reactive protein; C_{trough}=trough concentration; DLT=dose limiting toxicities; Eₘₐₓ,obs=maximum observed effect; EOI=end of infusion; FAS=fatigue assessment scale; FVC=forced vital capacity; HRCT=high-resolution computed tomography; IL-1b=interleukin 1b; IL-6=interleukin-6; KSQ=King's Sarcoidosis Questionnaire; LCQ=Leicester Cough Questionnaire; MAD=multiple ascending dose; mg=milligram; mMRC=Modified Medical Research Council; nAb=neutralizing antibodies; PD=pharmacodynamics; PGA=Patient Global Assessment;

PK=pharmacokinetics; QoL=quality of life; SAE=serious adverse event; SF-36=Short Form-36; sIL 2R=soluble interleukin-2 receptor; sTNFα=soluble tumor necrosis factor α; STQ=steroid toxicity questionnaire;
tEₘₐₓ,obs=time to reach maximum observed effect.

TABLE 37
Part B (POC) Primary, Secondary, and Exploratory Objectives and Endpoints

| | |
|---|---|
| Objectives | Endpoints |

Primary

| | |
|---|---|
| Efficacy | Efficacy Endpoints: |
| To establish efficacy of | Proportion of subjects who achieve the targeted |
| XTMAB-16 as measured by the | tapered dose of corticosteroid (prednisone 5 |
| ability to reduce background | mg/day or equivalent) by Week 12 |
| oral corticosteroid use in | |
| subjects with pulmonary | |
| sarcoidosis with or without | |
| extrapulmonary manifestations | |

Secondary

| | |
|---|---|
| Safety | Safety Endpoints: |
| To evaluate continued safety | Rate of Adverse Events (AEs), including Serious |
| and tolerability of XTMAB-16 in | Adverse Events (SAEs) throughout the study |
| subjects with pulmonary | duration |
| sarcoidosis with or without | |
| extrapulmonary manifestations | |
| Efficacy | Efficacy Endpoints: |
| To evaluate efficacy of XTMAB- | Proportion of subjects who achieve at least 50% |
| 16 as measured by the ability to | reduction in dose of corticosteroid by Week 12 |
| reduce background oral | Proportion of subjects able to maintain steroid |
| corticosteroid use and maintain | reduction through Week 24 |
| reduction in subjects with | |
| pulmonary sarcoidosis with or | |
| without extrapulmonary | |
| manifestations | |
| Pharmacokinetics: | Pharmacokinetic Endpoints: |
| To evaluate continued PK and | Clearance and volume parameters and half-life |
| PD of XTMAB-16 in subjects | in the intended subject population. |
| with sarcoidosis with or without | First dose and steady-state AUC, Cₘₐₓ, C_{trough} |
| extrapulmonary manifestations | and C_{average}. |
| Pharmacodynamics: | Change in Biomarkers at Baseline to Week 12 |
| To evaluate the effect of | and 24: |
| XTMAB-16 on immunogenicity | Absolute and percent change in biomarkers |
| and biomarkers in subjects with | ACE |
| pulmonary sarcoidosis with or | Interleukin-6 (IL-6) |
| without extrapulmonary | Soluble Interleukin-2 Receptor (sIL 2R) |
| manifestations | Soluble tumor necrosis factor α (sTNFα) |
| | CRP |
| | Calcitriol (Vitamin D 1, 25) |
| | Interleukin-1b (IL-1b) |
| | Immunogenicity |
| | Number and percentage of subjects by cohort |
| | who test positive for XTMAB-16 ADA at Baseline |
| | to Week 24 and transient & persistent positive |
| | status at follow up assessments. |
| | Number and percentage of subjects by cohort |
| | who test positive for XTMAB-16 nAb at Baseline |
| | to Week 24 and transient & persistent positive |
| | status at follow up assessments |

Exploratory

| | |
|---|---|
| To evaluate the potential | Change in Quality-of-Life Measurements from |
| therapeutic effect of XTMAB-16 | Baseline to Week 12 and Week 24 |
| as measured by Quality of Life, | King's Sarcoidosis Questionnaire (KSQ) General |
| Pulmonary, and Imaging | Health Status Module |
| endpoints | KSQ - Lung Module |
| | Leicester Cough Questionnaire (LCQ) |
| | Patient Global Assessment (PGA) |
| | Fatigue Assessment Scale (FAS) |
| | Modified Medical Research Council (mMRC) |
| | Dyspnea Scale |
| | Short Form - 36 (SF-36) |
| | Steroid Toxicity Questionnaire (STQ) |
| | Pulmonary Endpoints at Baseline to Week 12 |
| | and Week 24 |
| | Mean percent change in forced vital capacity |
| | (FVC)% from Baseline to Week 12 |
| | Change in KSQ-Lung Score |
| | Change in 6 Minute Walk Distance (6MWD) |
| | Imaging Endpoints at Baseline to Week 12 and |
| | Week 24 |
| | Change in high-resolution computed tomography |
| | (HRCT) |

Abbreviations: 6MWD = 6-minute walk distance; ACE = angiotensin converting enzyme; ADA = anti-drug antibodies; AE = adverse event; AUC = area under the concentration-time curve; C_{average} = average concentration; Cₘₐₓ = maximum observed concentration; CRP = C--reactive protein; C_{trough} = trough concentration; FAS = fatigue assessment scale; FVC = forced vital capacity; HRCT = high-resolution computed tomography; IL-1b = interleukin-1b; IL-6 = interleukin-6; KSQ = King's Sarcoidosis Questionnaire; LCQ = Leicester Cough Questionnaire; mg = milligram; mMRC = Modified Medical Research Council; nAb = neutralizing antibodies; PD = pharmacodynamics; PGA = patient global assessment; PK = pharmacokinetics; POC = proof of concept; SAE = serious adverse event; SF-36 = Short Form-36; sIL 2R = soluble interleukin-2 receptor; sTNFα = soluble tumor necrosis factor α; STQ = steroid toxicity questionnaire.

### Overall Design

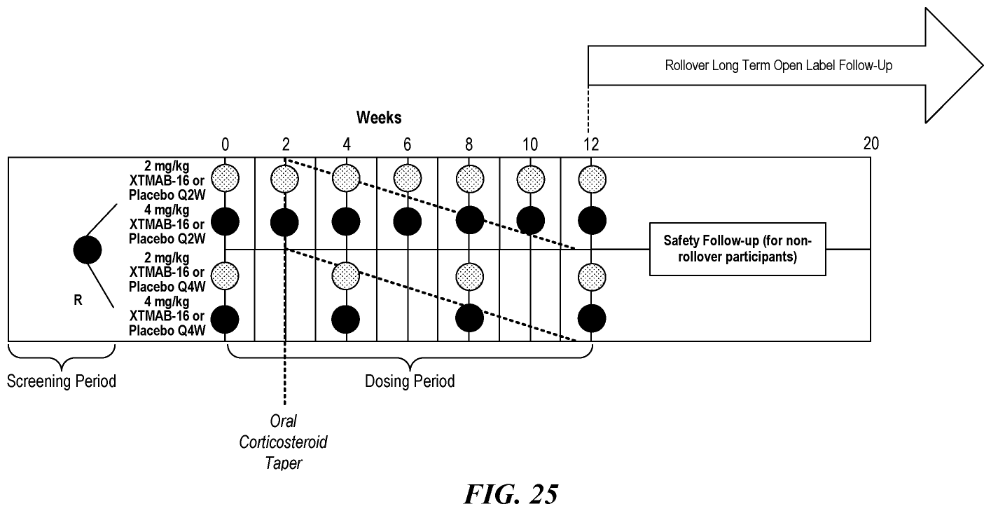

Example 9 comprises a Multiple Ascending Dose (MAD)/POC Study of XTMAB-16 to evaluate the safety, tolerability, immunogenicity, pharmacokinetics, and preliminary efficacy of XTMAB-16 in subjects with pulmonary sarcoidosis with or without extrapulmonary involvement. Part A will consist of a screening period of up to 42 days and a 12-week randomized double blind placebo-controlled treatment period of MAD regimens (FIG. 25), in which subjects will be randomized to XTMAB-16 or placebo in a 3:1 ratio per cohort (6 active, 2 placebo). A recommended phase 2 dose regimen (RP2D) will be determined from the results of Part A and Part B will commence as a double-blind placebo controlled POC study.

Subjects who complete Part A will maintain their assigned dose regimen in a rollover long-term open label safety study until all doses have been evaluated by the Data Safety Monitoring Committee (DSMC). Once the RP2D for Part B has been selected, subjects on treatment in the rollover study will transition to the RP2D.

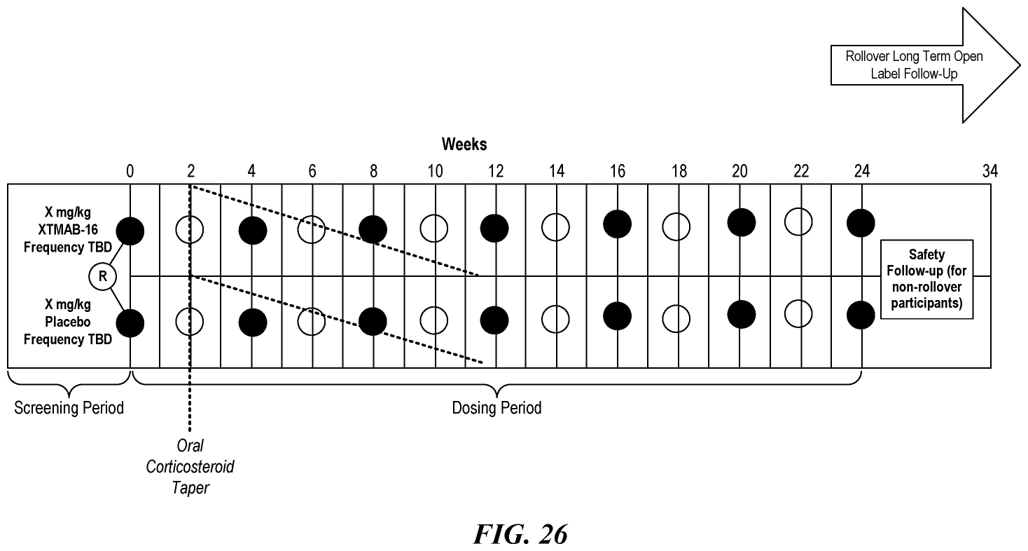

Part B will include a double-blind placebo-controlled POC study (FIG. 26), with a 1:1 XTMAB-16 to placebo allocation ratio for 24 weeks of the RP2D determined in Part A. At the completion of Part B, all subjects who have completed up to 24 weeks of treatment and evaluations will be given the opportunity to rollover into the ongoing long-term open label safety study.

### Number of Subjects

Up to 32 subjects will be enrolled in Part A to receive either XTMAB-16 or placebo in a MAD fashion in planned cohorts for 12 weeks: 2 mg/kg every four weeks (Q4W) for 12 weeks (n=6 active, 2 placebo), 4 mg/kg Q4W for 12 weeks (n=6 active, 2 placebo), 2 mg/kg every 2 weeks (Q2W) for 12 weeks (n=6 active, 2 placebo), 4 mg/kg Q2W for 12 weeks (n=6 active, 2 placebo).

Approximately 62 subjects will be enrolled in Part B to receive XTMAB-16 or placebo (1:1) in a randomized double-blind placebo-controlled manner for 24 weeks. Baseline dose of prednisone or equivalent will be used as a stratification factor during the randomization.

All subjects who have completed study participation in either Part A or B will be offered the opportunity to roll into an open label long-term safety study.

### Summary of Key Eligibility Criteria: Inclusion Criteria

1. Subject between 18 and 80 years (inclusive) of age.
2. Weighs between 45 and 160 kg (99 to 353 lbs.) at Screening.
3. Diagnosis of pulmonary sarcoidosis (at least 6 months before Screening) using the 2020 American Thoracic Society (ATS) Clinical Practice Guideline, the European Respiratory Society (ERS) or the World Association of Sarcoidosis and Other Granulomatous Disorders (WASOG) criteria including a compatible clinical and radiologic presentation with other causes of granulomatous disease ruled out (cutaneous and ocular involvement permitted).
4. Modified Medical Research Conference (mMRC) Dyspnea Scale of ≥1.
5. Receiving treatment of 7.5 to 25 mg/day of oral prednisone, or equivalent, during the screening period and, at the determination of the investigator, is capable of undergoing the protocol specific corticosteroid taper regimen.
6. Receiving treatment with methotrexate, azathioprine, mycophenolate, leflunomide, chloroquine, or hydroxychloroquine for at least 3 months before Screening that has been at a stable dose for 4 weeks before Screening. All efforts should be made to maintain stable background therapy at the Screening dose through the intervention period at the Investigator's discretion.
7. PART A only: Willing to refrain from consumption of grapefruit or grapefruit juice [pomelos, exotic citrus fruits, or grapefruit hybrids] from screening visit until after the final dose.
8. Polymerase chain reaction (PCR) test or rapid antigen test negative for severe acute respiratory syndrome coronavirus 2 (SARS-CoV-2) at Screening.
9. Able to provide written informed consent.
10. In the opinion of the Investigator, the subject is capable of understanding and complying with protocol requirements.

### Summary of Key Eligibility Criteria: Exclusion Criteria

1. Pregnant or breastfeeding women or women who are planning to become pregnant during the study.
2. PART A ONLY: Subjects>65 years of age.
3. PART A ONLY: Known potentially significant fibrotic disease and/or active inflammation contained solely in the hilar region as shown by high-resolution computed tomography (HRCT), confirmed by a central reader. Subjects with current active inflammation in the hilar region with concurrent inflammation outside the hilar region may be included. For subjects with disease onset of <2 years, a historical computed tomography (CT) within 6 months prior to screening confirmed by a central read is acceptable. For subjects with disease onset of >2 years and without a CT within 6 months prior to screening, a CT will be performed at Screening.
   Note: For all subjects, regardless of their time of disease onset, if a historical HRCT is to be submitted for diagnosis confirmation, that HRCT must have been performed within 6 months of screening. If their last HRCT was from >6 months prior to screening, then they will need to have an HRCT performed during screening for diagnosis confirmation.
   Note: Significant fibrotic disease is defined as >20% fibrosis on HRCT.
4. PART A ONLY: Any prior Tumor Necrosis Factor α (TNFα) inhibitor therapy.
5. Clinically significant extra-pulmonary sarcoidosis requiring systemic therapy as determined by the investigator.
6. PART B ONLY: Any therapy with an anti-TNFα monoclonal antibody (e.g., infliximab, adalimumab, golimumab and their biosimilars) within 6 months.
7. Baseline percent predicted FVC of <50%.
8. Prior treatment with rituximab or repository corticotropin injection within the previous 12 months.
9. Clinically significant Central Nervous System (CNS) sarcoidosis requiring therapy, except history of isolated seventh cranial nerve palsy or evidence of demyelinating neurologic disease.
10. Advanced congestive heart failure (New York Heart Association [NYHA] 3 or 4).
11. Current disease presentation consistent with Lofgren's syndrome (i.e., presence of the triad of erythema nodosum, bilateral hilar lymphadenopathy on chest X-ray, and joint pain).
12. Clinically significant pulmonary hypertension requiring treatment.
   Note: Clinically significant pulmonary hypertension requiring treatment would be defined as treatment with, i.e., prostacyclins, phosphodiesterase 5 inhibitors, and endothelin receptor antagonists.
13. Known hypersensitivity to any component of the formulation of XTMAB-16.
14. Live or messenger ribonucleic acid (mRNA) vaccination within 2 weeks before Day 1 or inoculation with a live or mRNA vaccine is planned during study participation.
15. Evidence of active or latent tuberculosis (TB) by interferon-gamma release assay (IGRA) or invasive fungal infections at Screening.
16. Known positive history of malignancy other than non-melanomatous skin cancer in the last 2 years, including in-situ carcinoma of the uterine cervix completely cured by radical surgery.
17. Positive test result for hepatitis B surface antigen (HBsAg), hepatitis C virus (HCV) antibody, coronavirus disease (COVID-19), TB, or a known history of human immunodeficiency virus (HIV) infection at Screening.
18. Women of childbearing potential who are sexually active with a non-sterilized male partner and are not willing to adhere to adequate birth control measures from the time of signing the informed consent, throughout the duration of the study, and for 90 days after 5 half-lives have elapsed since the last dose of study drug.
19. Male subjects who are non-sterilized and sexually active with a female partner of childbearing potential and are not willing to use adequate contraception from the time of signing the informed consent throughout the duration of the study, and for 90 days after 5 half-lives have elapsed since last dose of study drug.
20. Clinically significant hepatic or renal disease, including uncontrolled diabetes at the discretion of the investigator.
21. Any severe prior reaction to any type of biologics or human blood product such as albumin, immunoglobulin G (IgG) etc.
22. Concurrent emphysema.
23. Known hypercalcemia due to non-sarcoidosis conditions such as untreated hyperparathyroidism, at the discretion of the investigator.
24. Abnormal electrocardiogram (ECG): ventricular arrhythmias (non-sustained ventricular tachycardia (VT), multifocal or frequent premature ventricular contractions, bundle branch block, axis deviation, or abnormal Q waves). In the case of a QTcF (corrected OT interval by Fredericia) interval>450 ms (men) or >480 ms (women; subjects with bundle branch block) or PR interval outside the range of 120 to 220 ms, the assessment may be repeated once for eligibility determination at Screening or Baseline.
25. Donation or loss of 450 mL or more of his or her blood volume (including plasmapheresis) or transfusion of any blood product within 90 days prior to dosing.
26. Known uncontrolled hypertension.
   Note: Uncontrolled hypertension is noted as blood pressure≥160/100 mmHg despite anti hypertensive therapy within 3 months of randomization.
27. Clinical signs and symptoms consistent with COVID-19, e.g., fever, dry cough, dyspnea, sore throat, fatigue, new smell or taste disorder or confirmed infection by appropriate laboratory test within the last 4 weeks prior to Screening.
28. In the opinion of the investigator, inability to tolerate corticosteroid taper.
29. Concurrent systemic steroid use for non-sarcoidosis conditions.
30. Concurrent known auto-immune disease requiring treatment.
31. Participation in another clinical trial of an investigational agent within 3 months (small molecule)/6 months (biologics) or 5 half-lives (if known) of the agent, whichever is longer.
32. Any condition that required hospitalization within the 3 months prior to Day 1 or is likely to require so during the study.
33. Clinically significant abnormalities in the Screening physical exam, medical history, vital signs, ECG, or clinical laboratory tests that are not known to be due to concurrent sarcoidosis, and in the opinion of the Investigator and Medical Monitor should preclude the subject's participation in the clinical study.

### Lifestyle Considerations:

During this study, subjects will be asked to:
1. Refrain from consumption of red wine and Seville oranges from screening visit until after the final dose. In addition, subjects should also avoid consuming grapefruit or grapefruit juice (pomelos, exotic citrus fruits, or grapefruit hybrids) and any drug or diet that may interact with drug metabolizing enzymes activity from screening visit until after the final dose. This is essential as grapefruit enhances cortisol availability and will interfere with corticosteroid tapering essential for the study endpoint.
2. Minimize interactions with household contacts who may be immunocompromised.
3. Be vaccinated for COVID-19 at least 2 weeks prior to participation, defined as the initial 2-vaccine course of the Moderna or Pfizer vaccines, or a single J&J vaccine.

### Screen Failures

Screen failures are defined as subjects who consent to participate in the clinical trial but are not subsequently randomized or assigned to the study intervention or entered in the study.

A minimal set of screen failure information is required to ensure transparent reporting of screen failure subjects, to meet the Consolidated Standards of Reporting Trials (CONSORT) publishing requirements and to respond to queries from regulatory authorities. Minimal information includes demographics, screen failure details, and eligibility criteria.

Individuals who do not meet the criteria for participation in this trial (screen failure) may be rescreened once at the discretion of the Investigator in consultation with the medical monitor. Rescreened subjects should be assigned the same subject number as for the initial screening.

### Treatment Failures

If the subject experiences the following, they will be considered a treatment failure:
1. Addition of background second-line antimetabolite therapy. Note, antimetabolite therapy dose may be adjusted to avoid toxicity.
2. Greater than 1 rescue period of corticosteroids.
3. Breakthrough extrapulmonary disease requiring systemic treatment (topical and ophthalmic therapy for concurrent cutaneous and ocular disease are allowed).
4. Inability to reach expected taper dose of 5 mg by Week 12.
5. Inability to maintain taper dose or lower by Week 24 (Part B).
6. Inability to taper steroids to targeted maintenance dose by Week 12 or Week 24 after rescue period.
7. Rescue period requiring treatment longer than 2 weeks.
8. Permanent drug discontinuation due to at least two Grade 3 or one Grade 4 toxicity considered by the Investigator to be associated with XTMAB-16 treatment (discontinuation of study drug will be considered treatment failure for analysis purposes).

### Study Intervention(s) and Concomitant Therapy

Study interventions will be conducted as outlined in Table 38:

TABLE 38
Study Treatment Formulation and Preparation

| Intervention | | |
|---|---|---|
| Label | XTMAB-16 | Placebo |
| Dosage | Drug product is formulated as | Placebo is formulated as 100 mg |
| Formulation | 100 mg XTMAB-16 in a 20 mL | placebo in a 20 mL vial. |
| | vial. Excipients (per vial) | Excipients (per vial) include |
| | include 500 mg sucrose, 0.5 | contains 500 mg sucrose, 0.5 |
| | mg polysorbate 80, 5.52 mg | mg polysorbate 80, 5.52 mg |
| | monobasic sodium phosphate, | monobasic sodium phosphate |
| | monohydrate, and 1.78 mg | monohydrate, and 1.78 mg |
| | dibasic sodium phosphate, | dibasic sodium phosphate |
| | dihydrate. No preservatives are | dihydrate. No preservatives are |
| | present. | present. |
| | Each single-use glass vial of | Each single-use glass vial of |
| | XTMAB-16 is reconstituted with | placebo is reconstituted with 10 |
| | 10 mL of SWFI. | mL of SWFI. |
| Unit Dose | 2 mg/kg, 4 mg/kg | 2 mg/kg, 4 mg/kg |
| Strengths/Dosage | | |
| levels | | |
| Route of | IV infusion | IV infusion |
| Administration | | |
| Time of | 2 hours | 2 hours |
| Administration | | |

Abbreviations: IV = intravenous; mg = milligram; SWFI = sterile water for injection.
Source: Pharmacy Manual

### Dosing and Administration: Part A Dose Escalation:

During Part A, multiple doses and frequencies will be explored in an ascending fashion (FIGS. 10 and 13). To identify the recommended Phase 2 dose for Part B, the dose level groups will be enrolled both sequentially and in parallel by dose density as described below, and the cumulative safety data will be reviewed on an ongoing basis by the DSMC,

Part A will consist of 32 subjects in 4 cohorts of 8 subjects each, randomized in a 3:1 fashion (6 active, 2 placebo). First cohort at 2 mg/kg Q4W dose group will start enrolling the subjects initially. After the completion of a safety assessment of at least 4 subjects at this dose group who have completed 3 doses, it will be considered safe to proceed with dosing in the second and third cohort at 4 mg/kg Q4W and 2 mg/kg Q2W in parallel, as the dose density of these regimens are considered equal.

After the completion of a safety assessment of at least 4 subjects in both 4 mg/kg Q4W and 2 mg/kg Q2W dose groups (completion of 3 doses in the Q4W cohort and 6 doses of the Q2W cohort), it will be considered safe to proceed with dosing in the fourth cohort at 4 mg/kg Q2W.

The DSMC may make independent decisions about the safety of both 4 mg/kg Q4W and 2 mg/kg Q2W dose groups in the second and third cohort to decide if it is safe to go to the next dose group.

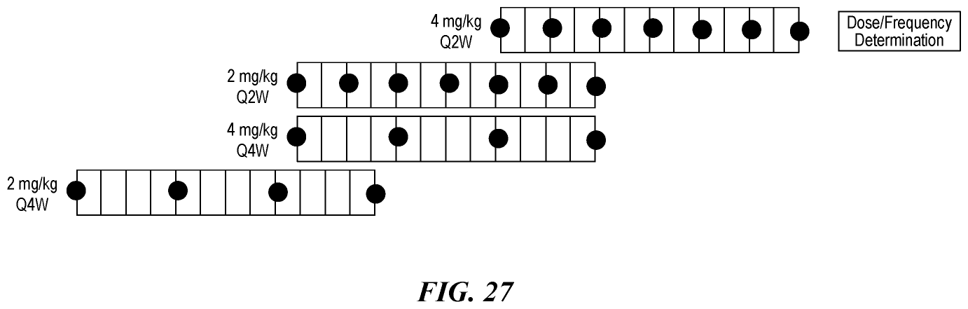

The escalation scheme will be conducted according to the schema illustrated in FIG. 27.

Enrollment will not pause between cohorts unless the minimum follow-up in a cohort has not been achieved and will enroll continuously while regular safety reviews occur. Following regular safety data reviews, at the discretion of the DSMC, dose escalation and enrollment in all dose groups may be paused for a complete safety evaluation if any safety signals or DLTs are identified. Criteria for a DLT are as follows: any prolonged Grade 2 (defined as continuous at Grade 2 (or higher) for more than 3 weeks) or any Grade 3 or higher adverse event that is considered related to study drug and is not attributable to worsening of concurrent illness.

Stopping criteria for a safety signal that may warrant the DSMC to request additional safety review during the dose escalation phase are as follows:
1. Any SAE assessed as related to study drug
2. Any signs of serious infection such as TB, bacterial sepsis or invasive fungal infection, or lymphoma diagnosis
3. Grade 3 hypersensitivity or anaphylaxis

The completion of extended DSMC safety review may result in resuming enrollment, proceeding with dose escalation, expanding the current dose level cohort, or terminating enrollment. Dose escalation will continue in this manner until the highest planned dose level is complete or a DSMC decision to discontinue enrollment due to safety. An unscheduled PK sample, ECG or clinical laboratory data analysis may be performed to support the safety assessment, if needed, in the event of a safety signal of concern.

### Dose Selection: Part B (POC)

At the completion and review of data by the DSMC from Part A of this study, a dose level and frequency will be determined for the Part B POC portion of the study. Dose determination will be guided by observed meaningful relevant PK parameters (C_{trough}, AUC), observed relationship of PK and in vitro measures of pharmacologic activity, observed change in clinically meaningful biomarkers (ACE, sIL 2R, CRP, IL-1b), observed clinical benefit (ability to reduce background oral corticosteroid use, change in QoL and Pulmonary endpoints), and observed safety signals and DLTs per cohort. An exploratory efficacy and/or PK data analysis may be performed to select the recommended Phase 2 dose for Part B, if needed.

### Oral Corticosteroid Taper Protocol: Part A (MAD)

During Part A of the study, two weeks after completion of the first dose and prior to the second administered dose for subjects assigned to Q2W dosing schedule, subjects will begin tapering corticosteroids from their starting dose to a target dose of 5 mg/day of prednisone, or equivalent, or less, to be completed on or before Week 12. The corticosteroid will be tapered depending on the starting dose as shown in Table 39. The subject's corticosteroid dosage may be further titrated below 5 mg/day or discontinued when feasible.

In cases where the subject has had >5% absolute worsening of FVC % (at 2 visits at least 7 days apart), subjects may have their oral corticosteroid dose increased at the investigator's discretion during the rescue therapy period of two weeks. Symptomatic flare will be confirmed by a change of score by ≥4-point fall of King's Sarcoidosis Questionnaire Lung Module (KSQ-Lung) and/or a decrease in Leicester Cough Questionnaire (LCQ) score by ≥1 point, or the investigator determines a rescue dose of corticosteroids is warranted due to worsening cough, shortness of breath, exercise intolerance, or worsening symptoms. Subjects should be evaluated in clinic when possible or via telehealth.

If an increase in steroid dose is not clinically indicated, steroid rescue therapy is not mandated in the event of worsening cough or worsening dyspnea or change in FVC. Subjects who require an increase in their steroid dose at any time in the study should continue to receive study drug and follow through to the end of the study. Subject will document steroid doses in a diary, which will be reviewed at scheduled telephone contacts and site visits.

For subjects who require a corticosteroid rescue therapy between Baseline and Week 12, a taper may continue after the acute event resolves at the investigator's discretion. The steroid taper must be completed by Week 12, regardless of rescue therapy, to be considered a treatment success. Inability to taper after rescue therapy will constitute a treatment failure. Subjects will be allowed up to one corticosteroid rescue therapy between Baseline and Week 12 to be considered responsive to treatment, however, subjects may be given additional corticosteroid rescue therapy if determined necessary by the Investigator.

TABLE 39
Corticosteroid Taper Protocol by Start Dose Part A (MAD)
Part A (MAD) Corticosteroid Taper Protocol by Start Dose^{a}

| | | Week | | | | | | | | | | |
|---|---|---|---|---|---|---|---|---|---|---|---|---|
| | | 2 | 3 | 4 | 5 | 6 | 7 | 8 | 9 | 10 | 11 | 12 |
| 25 | mg | 20 | 17.5 | 17.5 | 15 | 15 | 12.5 | 12.5 | 10 | 7.5 | 5 | |
| 20 | mg | 17.5 | 17.5 | 15 | 15 | 12.5 | 12.5 | 10 | 7.5 | 5 | | |
| 15 | mg | 12.5 | 12.5 | 10 | 7.5 | 5 | | | | | | |
| 10 | mg | 7.5 | 5 | | | | | | | | | |
| 7.5 | mg | 5 | | | | | | | | | | |

Abbreviations: MAD = multiple ascending dose; mg = milligram.
^{a}The doses reflected in the table are prednisone doses.

### Oral Corticosteroid Taper Protocol: Part B (POC)

During Part B of the study, two weeks after completion of the first dose and prior to the second administered, subjects will begin tapering corticosteroids from their starting dose to a target dose of 5 mg/day of prednisone, or equivalent, or less, to be completed on or before Week 12 and maintained through Week 24 (Table 40). The corticosteroid will be tapered depending on the starting dose, and at the investigator's discretion. At the investigator's discretion, subject's corticosteroid dosage may be further titrated below 5 mg/day or discontinued when feasible.

In cases where the subject has had >5% absolute worsening of FVC % (at 2 visits at least 7 days apart), subjects may have their corticosteroid dose increased at the investigator's discretion during the rescue therapy period of two weeks. Symptomatic flare will be confirmed by a change of score by ≥4-point fall of KSQ-Lung and/or a decrease in LCQ score by ≥1 point, or the investigator determines a rescue dose of corticosteroids is warranted due to worsening cough, shortness of breath, exercise intolerance, or worsening symptoms. Subjects should be evaluated in clinic or via telehealth.

If an increase in steroid dose is not clinically indicated, steroid rescue therapy is not mandated in the event of worsening cough or worsening dyspnea or change in FVC. Subjects who require an increase in their steroid dose at any time in the study should continue to receive blinded investigational product and follow through to the end of the study. Subjects will document steroid doses in a diary, which will be reviewed at relevant scheduled telephone contacts and site visits.

For subjects who require a corticosteroid rescue therapy between Baseline and Week 12, a taper may continue after the acute event resolves at the investigator's discretion. The steroid taper must be completed by Week 12 and maintained through Week 24, regardless of rescue therapy, to be considered a treatment success. Inability to taper after rescue therapy will constitute a treatment failure. Subjects will be allowed up to one corticosteroid rescue therapy to be considered responsive to treatment, however, subjects may be given additional corticosteroid rescue therapy if determined necessary.

TABLE 40
Corticosteroid Taper Protocol by Start Dose Part B (POC)
Part B POC Corticosteroid Taper Protocol by Start Dose^{a}

| | | Week | | | | | | | | | | | |
|---|---|---|---|---|---|---|---|---|---|---|---|---|---|
| | | 2 | 3 | 4 | 5 | 6 | 7 | 8 | 9 | 10 | 11 | 12 | 12-24 |
| 25 | mg | 20 | 17.5 | 17.5 | 15 | 15 | 12.5 | 12.5 | 10 | 7.5 | 5 | | |
| 20 | mg | 17.5 | 17.5 | 15 | 15 | 12.5 | 12.5 | 10 | 7.5 | 5 | | | |
| 15 | mg | 12.5 | 12.5 | 10 | 7.5 | 5 | | | | | | | |
| 10 | mg | 7.5 | 5 | | | | | | | | | | |
| 7.5 | mg | 5 | | | | | | | | | | | |

Abbreviations: mg = milligram; POC = proof of concept.
^{a}The doses reflected in the table are prednisone doses.

### Formulation of XTMAB-16

The XTMAB-16 for injection (drug product) manufacturing process is comprised of formulation, filtration, fill, semi stoppering, lyophilization, full-stoppering, and seal steps. The drug product is sterilized by filtration prior to aseptic filling. No impurities are introduced during drug product manufacturing.

The lyophilized drug product, XTMAB-16, is supplied as a sterile, white to off-white, lyophilized powder or lyophilized cake (100 mg XTMAB-16 per vial) in 20 mL vial for reconstitution with 10 mL of sterile water for injection (SWFI) and IV infusion. The reconstituted solution concentration is 10 mg/mL. With a pH of 5.8 to 6.2. The reconstituted solution is diluted with sterile 0.9% sodium chloride injection, United States Pharmacopeia (USP) to the range between 0.4 mg/mL and 4 mg/mL for infusion.

The matching placebo has the same formulation as the drug product except it contains no XTMAB-16.

### Randomization and Blinding

In both Part A (MAD) and Part B (POC) of this study, subjects will be randomly assigned to treatment (XTMAB-16 or placebo) and treatment assignment will be double-blind; all study staff will be blind to subject treatment assignment for the duration of the study. The infusion solution containing active drug and placebo will be indistinguishable in appearance. All study assessments and causality will be performed, if possible, prior to unblinding.

### Prior and Concomitant Therapy

Prior medication history for up to a year before screening will be collected. The use of concomitant medication is allowed during the study unless specified in the inclusion or exclusion criteria or study procedures. The name of the medication, indication, dose, and duration of use will be recorded on the eCRF. If a new concomitant therapy is started due to an adverse event, the adverse event must be recorded on the eCRF.

Concomitant corticosteroid dose will be self-recorded in a subject diary and verified by site personnel at each in person or telephone visit.

Subjects will receive concomitant treatment with methotrexate, azathioprine, mycophenolate, leflunomide, chloroquine, or hydroxychloroquine for at least 3 months before screening that has been at a stable dose for 4 weeks before screening. All efforts should be made to maintain stable background therapy at the screening dose through the intervention period.

Subjects will also receive treatment with 7.5 to 25 mg/day of oral prednisone, or equivalent, during the screening period, which will be weaned during the duration of the study period.

### Study Assessments and Procedures: Efficacy Assessments

Planned time points for all efficacy assessments are provided in the SoA (Table 44-Table 51).

### Study Assessments and Procedures: Quality of Life Measures

All Quality of Life assessments are to be performed before other study procedures at each designated visit and are to be completed in the same order at each designated visit. The order of Quality of Life Measurements will be outlined in the electronic Clinical Outcome Assessments (eCOA) Site Manual.

### Study Assessments and Procedures: King's Sarcoidosis Questionnaire

The King's Sarcoidosis Questionnaire (KSQ) is an online 29-item questionnaire to be completed by sarcoidosis subjects. The KSQ is split into 5 sections: general health status, lungs, medication, skin, and eyes. Results are given as a number between 1 to 100 with higher numbers indicating better health. The questionnaire takes approximately 10 minutes to complete. The minimal clinically important difference (MCID) for KSQ remains unclear in sarcoidosis.

### Study Assessments and Procedures: Leicester Cough Questionnaire

The Leicester Cough Questionnaire (LCQ) is a 19-item self-completed quality of life measure of chronic cough which is responsive to change. Items on the scale are divided into 3 domains, physical, psychological, and social. Items are scored on a 7-point Likert scale. A total score (range 3 to 21) is also calculated by adding the domain scores together, with higher scores indicating better quality of life. The questionnaire takes 5 to 10 minutes to complete. The MCID for chronic cough is 1.3.

### Study Assessments and Procedures: Short Form-36

The Short Form-36 (SF-36) is a 36-item, subject-reported survey of subject health. The SF-36 includes one multi-item scale that assesses eight health concepts: 1) limitations in physical activities because of health problems; 2) limitations in social activities because of physical or emotional problems; 3) limitations in usual role activities because of physical health problems; 4) bodily pain; 5) general mental health (psychological distress and well-being); 6) limitations in usual role activities because of emotional problems; 7) vitality (energy and fatigue); and 8) general health perceptions.

### Study Assessments and Procedures: Fatigue Assessment Scale

The Fatigue Assessment Scale (FAS) contains 10 specific fatigue questions that have been validated in sarcoidosis subjects. On each question, one of 5 answer categories may be chosen, from never to always: 1=never; 2=sometimes (about monthly or less); 3=regularly (about a few times a month); 4=often (about weekly); 5=always (about every day). An answer to each question has to be given, even if the person does not have any complaints at the moment. Scores on questions 4 and 10 should be recoded (1=5, 2=4, 3=3, 4=2, 5=1). Subsequently, the total FAS score may be calculated by summing the scores on all questions (the recoded scores for question 4 and 10). The sum of questions 3 and 6 to 9 indicates mental fatigue, and the sum of the questions 1, 2, 4, 5 and 10 indicates physical fatigue. The minimal score is 10, and the maximal score is 50. Scores of ≥22 are considered to represent substantial fatigue. A change in the FAS score of 4 points is considered to be the MCID. The FAS takes approximately 5 to 10 minutes to complete.

### Study Assessments and Procedures: Patient Global Assessment

The patient global assessment (PGA) is a subject-reported outcome instrument used to gauge the subject's well-being and is scored using a visual analogue scale (VAS). The MCID in sarcoidosis remains unclear in randomized controlled trials. A previously reported MCID for PGA in sarcoidosis was <7 cm, 7 to 10 cm.

### Study Assessments and Procedures: Steroid Toxicity Questionnaire

Glucocorticoids (GCs) are associated with clinically important toxicities in sarcoidosis subjects, associated with both the cumulative dose and duration of treatment. The Steroid Toxicity Questionnaire (STQ) is a simple 12 question tool used to characterize the toxicity associated with steroid use using a 4-point scale. The MCID remains unclear in sarcoidosis.

### Study Assessments and Procedures: Modified Medical Research Council Dyspnea Scale

The modified Medical Research Council (mMRC) Dyspnea Scale is a five-level rating scale based on the subject's perception of dyspnea in daily activities, ranging from none (Grade 0) to very severe dyspnea (Grade 4). The score is the number that best fits the subject's level of activity.

### Study Assessments and Procedures: Pulmonary Function Tests

Pulmonary function tests (PFTs), including FVC, forced expiratory volume in 1 second (FEV1), and FEV1/FVC ratio, are to be determined by spirometry at the time points designated in SoA (Table 44-Table 51). Spirometry testing will be performed according to the ATS/ERS guidelines. The spirometer (SpiroSphere) and associated equipment be supplied to each site for the study. The same spirometry equipment must be used for all assessments performed for a subject. All subjects at all visits will be evaluated throughout the entire trial. Where possible the same technician should perform all maneuvers for an individual subject. All staff conducting the spirometry tests must have received appropriate training and pass two proficiency tests which must be documented. Spirometry ideally is to be performed with the subject in a seated position for at least 2 minutes with a tight seal around the mouth and wearing nose-clips or manual occlusion of the nostrils; the subject is to remain seated with head slightly elevated throughout the test procedure. If the testing is undertaken with the subject in another position, this must be documented in the report. It is recommended that the test be performed at least 1 hour after a light meal, avoid smoking and/or vaping and/or water pipe use within 1 hour before testing, avoid consuming intoxicants within 8 hours before testing, avoid wearing clothing that substantially restricts full chest and abdominal expansion and refrain from vigorous exercise within 1 hour of testing. If treated with bronchodilators, subjects should withhold medications prior to spirometry according to guidelines listed in Table 47. Spirometry is to be performed in the same manner across all assessment time points.

TABLE 41
Guidelines for Withholding Bronchodilator Medications

| Bronchodilator Medication | Withholding time |
|---|---|
| SABA (e.g., albuterol, salbutamol) | 4 to 6 hours |
| SAMA (e.g., ipratropium bromide) | 12 hours |
| LABA (e.g., formoterol or salmeterol) | 24 hours |
| Ultra-LABA (e.g., indacaterol, vilanterol, or | 36 hours |
| olodaterol) | |
| LAMA (e.g., tiotropium, umeclidinium, aclidinium, | 36 to 48 hours |
| or glycopyrronium) | |

Abbreviations: LABA = long-acting β₂ agonists; LAMA = long-acting muscarinic antagonists; SABA = short acting β₂ agonists; SAMA = short-acting muscarinic antagonists.

### Study Assessments and Procedures: 6 Minute Walk Distance

The 6-minute walk distance (6 MWD) will record the distance walked in six minutes, the level of oxygenation using a pulse oximeter and the level of dyspnea using a ten point dyspnea scale (Borg score). A standardized protocol for the six-minute walk will be done for all sites and must be performed in the same manner across all timepoints.

### Study Assessments and Procedures: High-Resolution Computed Tomography

High-resolution computed tomography (HRCT) is the modality of choice for evaluating the features of thoracic sarcoidosis. It depicts the characteristic features of the parenchymal nodules and lesions, their distribution, the associated changes, and atypical features accurately. It also helps in guiding the appropriate therapy by differentiating active lesions from irreversible fibrosis.

### Safety and Other Assessments

Planned timepoints for all safety and other assessments are provided in the SoA (Table 44-Table 51).

### Safety and Other Assessments: Physical Examination

A complete physical examination will include assessments of general appearance, skin; head/neck; pulmonary, cardiovascular, gastrointestinal, external genitourinary (optional), lymphatic, and musculoskeletal systems; extremities; eyes (inspection and vision control); nose; throat; and neurologic status. An abbreviated physical examination will include assessments of general appearance; head, ears, eyes, nose, and throat; cardiovascular; respiratory; and gastrointestinal systems (including the abdomen). A targeted physical examination may be conducted at any time at the Investigator's discretion and should be limited to systems of clinical relevance (i.e., cardiovascular, respiratory, lymph nodes, liver).

### Safety and Other Assessments: Height and Weight

Height (cm) will be measured only at Screening. Weight will be measured in kilograms at every visit according to the SoA (Table 44-Table 51).

### Safety and Other Assessments: Vital Signs

Oral, tympanic, or temporal body temperature, respiratory rate, pulse, and systolic and diastolic blood pressure (BP) will be assessed. The method of body temperature assessment chosen should be recorded and consistent throughout the study. If oral temperatures (° C.) are taken, subjects will have had no recent hot or cold beverages or smoking.

During the infusion, systolic/diastolic blood pressure and pulse should be measured in a semi-supine/semi-reclined position. For all other readings, blood pressure and pulse will be taken in a supine position after 10 to 15 minutes rest. During the infusion, vital signs should be collected per the study site's standard of care intervals.

Respiratory rate (breaths/min) will be taken after at least 5 minutes rest; breaths are counted for 30 seconds and multiplied by 2. Respiration rate will be taken once during the ECG. During the infusion, respiration rate will be taken in a semi-supine/semi-reclined position.

### Safety and Other Assessments: Electrocardiograms

Standard 12-lead ECGs will be recorded as the subject has been resting in the supine position for at least 10 minutes. ECGs will be collected in triplicate at the timepoints noted the SoA (Table 44-Table 51) with approximately 1 minute between each ECG; all other ECGs will be collected as single recordings. Additional ECGs will be performed at the discretion of the Investigator.

The Investigator will be responsible for reviewing the ECG to assess whether the ECG is within the reference limits and to determine clinical significance of the results. If necessary, the Investigator has the right to override the interpretation and intervals as measured by the ECG machine. Respiration rate will be taken once during the ECG. The ECG will be assessed for the following measures: P wave, QRS complex, U wave, QRS duration, QT interval, T wave, ST segment, RR interval, PR interval, and qualitative results.

### Safety and Other Assessments: Clinical Laboratory Evaluation

Clinical laboratory assessments will be collected under appropriate conditions defined by the clinical unit's standard of practice at the time points specified in the SoA (Table 44-Table 51).

The central lab will be used to perform all clinical laboratory tests excluding urine pregnancy dipstick, which will be assessed by the site staff. The Investigator should take immediate action for any safety concerns based on laboratory results.

Record any clinically relevant changes occurring during the study in the EDC system. The laboratory reports must be filed with the source documents. Clinically significant abnormal laboratory findings are those that are not associated with an underlying disease, unless judged by the Investigator to be more severe than expected for the subject's condition.

All laboratory tests with values considered clinically significantly abnormal during participation in the study, including the subject's last EOS visit, should be repeated until the values return to normal or Baseline or are no longer considered clinically significant or follow-up is no longer needed per the judgment of the Investigator or Medical Monitor.

If such values do not return to normal/Baseline within a period of time judged reasonable by the Investigator, the etiology should be identified, and the Sponsor notified.

### Safety and Other Assessments: Computed Tomography Scan

Images of the Computed Tomography (CT) scan will be centrally read and assessed by a three-point Likert scale (worse to better) by the central reader.

### Pharmacokinetics

Whole blood samples will be collected for measurement of serum concentrations of XTMAB-16. Instructions for the collection and handling of biological samples will be provided by the Sponsor in a separate laboratory manual. The actual date and time (24-hour clock) of each sample will be recorded in the eCRF. Samples will be used to evaluate the serum concentration and PK of XTMAB-16. Each serum sample will be divided into 2 aliquots (1 each for primary and back-up).

Measurement of concentrations of XTMAB-16 will be performed using a validated assay method under the supervision of the Sponsor or qualified designee. The analytical methods used to measure concentrations of XTMAB-16 will be described in a separate bioanalytical report. Only samples that are within the window of sample stability will be analyzed

While PK samples must be collected from subjects assigned to the placebo arm to maintain the blinding of treatment assignment, PK assay results for these subjects are not needed for the safety conduct or proper interpretation of this trial and most samples will therefore not be analyzed. Personnel responsible for performing PK analysis will be unblinded to subjects' treatment assignments to identify appropriate PK samples to be analyzed. Samples from subjects assigned to placebo may be analyzed upon request (i.e., to evaluate a possible error in dosing).

Any unfavorable and unintended sign (including an abnormal laboratory finding), symptom, or disease temporally associated with the use of a medicinal or investigational product, whether or not related to the medicinal or investigational product.

### Clinical Laboratory Tests

Additional tests may be performed at any time during the study. Nonlimiting examples of such are outlined in Table 42.

TABLE 42
Clinical Laboratory Parameters

| Hematology | Blood Chemistry | Serology | Urinalysis |
|---|---|---|---|
| Hematocrit | Albumin | Hepatitis B | Protein Glucose |
| Hemoglobin Platelet | Alkaline phosphatase | surface | Red blood cells |
| count | Alanine aminotransferase | antigen | White blood cells |
| Red blood cell | Aspartate | (hBsAg) | Ketone bodies pH |
| count White blood | aminotransferase Bilirubin | Hepatitis C | At discretion of |
| cell count with | (total and conjugated) | antibody HIV | Investigator |
| differential: | Calcium Chloride | antibody (HIV1 | based on |
| Neutrophils | Creatinine | and HIV2) | urinalysis results: |
| Eosinophils | Creatine phosphokinase | Tuberculosis | Microbiology |
| Basophils | Gamma-glutamyl | | Urine microscopy |
| Monocytes | transferase | | |
| Lymphocytes | Glucose Potassium | | |
| Activated partial | Sodium | | |
| thromboplastin time | Total cholesterol Total | | |
| | protein Triglycerides | | |
| | Tryptase^{a} | | |
| | WOCBP Only (per SoA): | | |
| | Follicle stimulating | | |
| | hormone | | |

Abbreviations: HIV = human immunodeficiency virus; SoA = schedule of activities; WOCBP = women of childbearing potential.
^{a}Tryptase will be assessed only at Baseline (for all subjects) and in the event a subject experiences anaphylaxis within 4 hours after end of infusion (sample to be collected as soon as is reasonable after treatment).

### Adverse Event of Special Interest (AESI)

An AE (serious or non-serious) of scientific and medical concern specific to the Sponsor's study drug or program, for which ongoing monitoring and rapid communication by the Investigator to the Sponsor may be appropriate. An AESI might require further investigation in order to characterize and understand it. Depending on the nature of the AESI, rapid communication between the Sponsor and Regulatory Authorities might also be warranted.

### Biomarkers

Whole blood samples will be collected for measurement of exploratory circulating biomarkers according to Table 60. Instructions for the collection and handling of biological samples will be provided by the Sponsor in a separate laboratory manual. The actual date and time (24-hour clock time) of each sample will be recorded in the eCRF. Data collected from analyses of circulating biomarkers may be used to correlate exposure to safety or efficacy aspects related to concerns arising during or after the study.

Measurement of circulating biomarkers will be performed using a fit-for-purpose qualified assay method under the supervision of the Sponsor or qualified designee. The analytical methods used to measure these biomarker endpoints will be described in separate bioanalytical reports. Only samples that are within the window of sample stability will be analyzed.

Circulating biomarkers that will be evaluated include ACE, interleukin-6 (IL-6), sIL 2R, soluble tumor necrosis factor α (sTNFα), C-reactive protein (CRP), calcitriol and IL-1b. ACE is a common biomarker used in the diagnosis of sarcoidosis. sIL 2R, a surrogate marker for T cell activation, may be elevated in some sarcoidosis subjects, and has been shown to have a high and superior sensitivity and specificity, compared to ACE, for diagnosing sarcoidosis in subjects. IL-6 is commonly elevated in subjects with sarcoidosis and may be involved in the initiation and maintenance of alveolitis by activating and causing the proliferation of T cells. TNFα induces IL-6 through the nuclear factor k B pathway and, thus, inhibition of TNFα through XTMAB-16 is likely to be reflected in a change in IL-6 circulating levels. Acute tissue injury in the lung, initiated by IL-1b converts to fibrotic changes. IL-1b may be a target for therapeutic intervention in diseases associated with fibrosis and tissue remodeling, like sarcoidosis.

### Immunogenicity

Antibodies to XTMAB-16 will be evaluated in serum samples collected from all subjects according to Table 60. Additionally, serum samples should also be collected at safety follow-up visits from subjects who discontinue from the study or choose to not rollover into the long term open label safety study. Persistence of ADA status will be measured as defined below.

### Transient ADA Response:

(i) Treatment-induced ADA detected only at one sampling time point during the treatment or follow-up observation period (excluding the last sampling time point, which ought to be considered persistent unless shown to be undetectable at a later time), or
(ii) Treatment-induced ADA detected at two or more sampling time points during the treatment (including follow-up period if any), where the first and last ADA-positive samples (irrespective of any negative samples in between) are separated by a period less than 16 weeks, and the subject's last sampling time point is ADA-negative.

### Persistent ADA Response:

(i) Treatment-induced ADA detected at two or more sampling time points during the treatment (including follow-up period if any), where the first and last ADA-positive samples (irrespective of any negative samples in between) are separated by a period of 16 weeks or longer, or
(ii) Treatment-induced ADA incidence only in the last sampling time point of the treatment study period or at a sampling time point with less than 16 weeks before an ADA-negative last sample.

Instructions for the collection and handling of biological samples will be provided by the Sponsor in a separate laboratory manual. These samples will be tested by the Sponsor or Sponsor's designee. Each serum sample will be divided into 2 aliquots (1 each for primary and a back-up).

Serum samples will be tested in a multi-tiered approach. A validated screening assay for antibodies binding to XTMAB-16 will be initially used to assess serum samples. Samples that are determined putative positive for ADA in the screening assay will then be subjected to a confirmatory assay to demonstrate that antibodies are specific to XTMAB-16. Samples that are identified as positive in the confirmatory assay will be further characterized in a validated tier assay and titer of confirmed positive samples will be reported. Other analyses may be performed to verify the stability of antibodies to study treatment and/or to further characterize the immunogenicity of study treatment. Serum samples will be screened for antibodies binding to XTMAB-16 and the titer of confirmed positive samples will be reported.

The detection and characterization of antibodies to XTMAB-16 will be performed using a validated assay method under the supervision of the Sponsor or qualified designee. The analytical methods used to measure immunogenicity will be described in a separate bioanalytical report. Only samples which are within the window of sample stability will be analyzed.

Samples that are confirmed positive for antibodies binding to XTMAB-16 with a positive titer may be further characterized and/or evaluated for their ability to neutralize the activity of the study treatment using a validated assay method.

### Summary of Statistical Considerations

Summary statistics for continuous variables will consist of number of non-missing observations (n), mean, standard deviation (SD), minimum, median, and maximum, unless specified otherwise. For categorical variables, the number and percentage of subjects in each category will be presented. When appropriate, 95% confidence intervals (CI) will be provided. Primary analysis set for Part A is SAS and for Part B is FAS, unless specified otherwise.

The data will be summarized in tabular form by treatment group combined across cohort in Part A and by treatment group in Part B. In Part A the 2 placebo subjects in each cohort will be combined into a placebo group of 8 subjects.

Baseline will be defined as the last non-missing value (including scheduled and unscheduled assessments) prior to the subject receiving study treatment.

Baseline for corticosteroid (prednisone or equivalent) dose will be the dosing value established at the time of receiving the study treatment on day 1.

### General Calculation or Derivation of Efficacy Variables

Percentage Reduction from Baseline in Prednisone Dose:

The percentage reduction from baseline in prednisone, or equivalent, dose for a given timepoint, defined as: {(Baseline dose−timepoint dose)/baseline dose}*100%

Proportion of Subjects with Prednisone Dose≤5 mg/day:

For each treatment group for a particular timepoint, the number of subjects with prednisone, or equivalent, dose≤5 mg/day will be calculated. The proportion of such subjects will be calculated for each treatment group for a given timepoint as: (Number of subjects with prednisone, or equivalent, dose≤5 mg/day at given timepoint/number of subjects in treatment group)

Proportion of Subjects with ≥50% Reduction from Baseline in the Prednisone Dose:

For an individual subject, if the above calculation results in a value of 50% or greater, that subject will be classified as having at least a 50% reduction in daily prednisone, or equivalent, dose. The proportion of such subjects will be calculated for each treatment group as: (Number of subjects with ≥50% reduction at given timepoint/number of subjects in treatment group).

### Analysis of the Primary Endpoints: Part A (MAD)

There is no primary efficacy endpoint for Part A of the study.

To observe the primary safety endpoint, the frequencies of treatment-emergent adverse events (TEAEs) and the number of subjects with TEAEs will be summarized overall and for applicable categories (e.g., seriousness, treatment-related, severity) by treatment group across cohorts in Part A in the SAS population. Observed DLT, rate of AESI will also be summarized by dose levels and frequencies.

PK, PD, ADA, reduction in dose of corticosteroid, QoL and pulmonary data will be observed and summarized descriptively to determine the recommended Phase 2 dose for Part B.

For PK endpoint, plasma concentrations of XTMAB-16 will be summarized in tabular form for at least minimal key PK parameters of interest (C_{trough} and AUC over the dosing interval). PK methods and the analysis will be described in a standalone PK analysis plan

For PD endpoint, biomarkers at each biomarker sampling time point will be summarized with absolute and percentage change descriptively for ACE, sIL 2R, CRP, IL-1b and Calcitriol.

Reduction in dose of corticosteroid will be summarized with absolute and percentage change descriptively over the time.

Quality of life and pulmonary data will be summarized with absolute and percentage change descriptively over the time from baseline for mean percent change in FVC %, KSQ General, KSQ-L scores, LSQ and STQ scores.

### Analysis of the Primary Endpoints: Part B (POC)

For the primary efficacy endpoint [targeted tapered dose of corticosteroid (maximum dose of prednisone 5 mg/day or equivalent) responders] for Part B of the study, a 1-sided 95% confidence interval (CI) approach for the difference of proportion of subjects having targeted tapered dose of corticosteroid (using the point estimate of the difference between XTMAB-16 vs Placebo) by Week 12 will be used to test for the superiority of XTMAB-16 in the FAS population. The proportion and their 95% CI using Clopper-Pearson test will also be displayed. Cochran Mantel Haenszel test (CMH) stratified for the stratification factor (baseline dose of prednisone or equivalent) will also be performed. This analysis will be performed on FAS population.

The categories of response are defined as follows:
Responder: For subjects who require a corticosteroid rescue therapy between Baseline and Week 12, a taper may continue after the acute event resolves at the investigator's discretion. The steroid taper must be completed by Week 12 regardless of corticosteroid rescue therapy, to be considered as a responder.
Non-Responder: Inability to taper after rescue therapy by Week 12 will be considered as a non-responder.

### Estimand Framework

An addendum to International Council for Harmonization (ICH) E9 (R1) introduces the concept of an estimand which translates the trial objective into a precise definition of the treatment effect that is to be estimated. For Part B primary endpoint, the description of the estimand includes four attributes: the population, the variable (or endpoint) to be obtained for each subject, the specification of how to account for intercurrent events (ICE) and the population-level summary for the variable. These attributes are described in Table 43.

TABLE 43
Estimand Framework

| | Difference in Proportion |
|---|---|
| Population | Full Analysis Set (FAS) Population |
| Variable/ | Programmatic determination of responders/difference in |
| Endpoint | responder's proportion between XTMAB-16 vs Placebo |
| Handling of | Composite estimand as the following ICEs are |
| Intercurrent | incorporated into the variable/endpoint: |
| Events (ICE) | Receipt of more than one corticosteroid rescue therapy |
| | (failure) |
| | Extrapulmonary disease requiring systemic treatment |
| | other than topical cutaneous and ophthalmic treatment |
| | (failure) |
| | Addition of background sarcoidosis concomitant |
| | medications noted in Inclusion criterion 6 (failure) |
| | Discontinuation of study drug due to any reason |
| | (failure) |
| Population- | Comparison between treatment groups in proportion of |
| level summary | responders using Cochran-Mantel Haenszel (CMH) test |

The categories of response in Estimand framework are defined as follows:
Responder: For subjects who require a corticosteroid rescue therapy between Baseline and Week 12, a taper may continue after the acute event resolves at the investigator's discretion. The steroid taper must be completed by Week 12 regardless of corticosteroid rescue therapy, to be considered as a responder.
Non-Responders: Inability to steroid taper by Week 12 will be considered as a non-responder, if the subjects have the following events reported:
i. Receipt of more than one corticosteroid rescue therapy
ii. Extrapulmonary disease requiring systemic treatment other than topical cutaneous and ophthalmic treatment
iii. Addition of background sarcoidosis concomitant medications noted in Inclusion criterion 6
iv. Discontinuation of study drug due to any reason

### Analysis of the Secondary Endpoint(s): Part A (MAD)

Part A will be summarized for Efficacy, PK, Biomarker and Immunogenicity secondary endpoints.

### (i) Efficacy:

Number and proportion of subjects who achieve the targeted tapered dose of corticosteroid (maximum dose of prednisone 5 mg/day or equivalent) and subjects who achieve at least 50% reduction in dose of corticosteroid by Week 12 will be summarized by treatment group and by combining active treatment groups in Part A. The proportion and their 95% CI using Clopper-Pearson test will also be displayed.

Absolute and change from baseline for reduction in dose of corticosteroid will also be summarized descriptively over the time by treatment group and by combining active treatment groups in Part A.

The efficacy analysis will be performed on FAS population.

### (ii) Pharmacokinetics:

Plasma concentrations of XTMAB-16 will be analyzed by either noncompartmental and/or population PK methods and the analysis will be described in a standalone PK analysis plan. At a minimum, key PK parameters of interest (C_{trough} and AUC over the dosing interval) will be estimated and descriptively summarized. Additional evaluations may include an assessment of dose proportionality and estimated time to achieve steady-state.

Consideration of stratification by key covariates of interested (e.g., ADA confirmed positive status) may be performed as data permits.

(iii) Biomarker:

Biomarkers at each biomarker sampling time point will be summarized with descriptive statistics (N, mean, SD, coefficient of variation (CV) %, geometric mean, geometric CV %, median, minimum, and maximum) and listed over the time by treatment group. Biomarker-Time Profile plots for individual and combined mean (+/−SD) will be generated as appropriate.

Absolute and percent change in biomarkers from Baseline to EOI, Week 2, 4, 8, and 12 for the following parameters: IL-6, sTNFα.

### (iv) Immunogenicity:

Anti-drug antibodies and nAb at each sampling time point will be summarized with descriptive statistics (N, mean, SD, CV, geometric mean, geometric CV, median, minimum, and maximum) by treatment group.

Number and percentage of subjects who test positive for XTMAB-16 ADA at Baseline, Week 4, Week 8, and Week 12 and number and percentage who test positive for XTMAB-16 nAb at Baseline, Week 4, Week 8, and Week 12 by treatment group will be displayed. The transient and persistent positive status will also be summarized by number and percentages for follow-up assessments.

### Analysis of the Secondary Endpoint(s): Part B (POC)

Part B will be summarized for Safety, Efficacy, PK, Biomarker and Immunogenicity secondary endpoints.

### (i) Efficacy:

Statistical analysis for difference in proportion of subjects who achieve at least 50% reduction by Week 12 and difference in proportion of subjects able to maintain steroid reduction at Week 24 will be performed similar to primary endpoint analysis for Part B by treatment group. The proportion and their 95% CI using Clopper-Pearson test will also be displayed.

Absolute and change from baseline for reduction in dose of corticosteroid will also be summarized descriptively over the time. Number and percentage for targeted tapered dose of corticosteroid (maximum prednisone 5 mg/day or equivalent) over the timepoint 12 weeks onwards will also be summarized by treatment group.

The analysis will be performed on FAS population.

A sensitivity analysis may be performed excluding treatment discontinuation before 12-week taper assessment for primary efficacy endpoint.

### (ii) Pharmacokinetics:

Plasma concentrations of XTMAB-16 will be analyzed by either noncompartmental and/or population PK methods and the analysis will be described in a standalone PK analysis plan. At a minimum, key PK parameters of interest (C_{trough} and AUC over the dosing interval) will be estimated and descriptively summarized. Additional evaluations may include an assessment of dose proportionality and estimated time to achieve steady-state.

Consideration of stratification by key covariates of interested (e.g., ADA confirmed positive status) may be performed as data permits.

(iii) Biomarker:

Biomarkers at each biomarker sampling time point will be summarized with descriptive statistics (N, mean, SD, CV %, geometric mean, geometric CV %, median, minimum, and maximum) and listed over the time. Biomarker-Time Profile plots for individual and combined mean (±SD) will be generated as appropriate.

Absolute and percent change in biomarkers from Baseline to Week 12 and Week 24 for the following parameters: ACE, IL-6, sIL 2R, sTNFα, CRP, Calcitriol (vitamin D 1, 25), IL-1b.

### (iv) Immunogenicity:

Anti-drug antibodies and nAb at each sampling time point will be summarized with descriptive statistics (N, mean, SD, CV, geometric mean, geometric CV, median, minimum, and maximum).

Number and percentage of subjects who test positive for XTMAB-16 ADA at Baseline to Week 24 and number and count and percentage who test positive for XTMAB-16 nAb at Baseline to Week 24 will be displayed. The transient and persistent positive status will also be summarized by count and percentages for follow-up assessments.

### Analysis of the Exploratory Endpoint(s): Part A (MAD)

Part A will be summarized for Quality of Life, Pulmonary, and Imaging exploratory endpoints. The analysis will be performed on SAS population by treatment group.

### (i) Quality of Life (QoL):

Descriptive summary for absolute and change from baseline QoL endpoints will be summarized over the time by treatment group and by combining active treatment groups in Part A.

The list of QoL assessments are as follows. Please refer to the eCOA site manual for the order of assessments: PGA, Fatigue Assessment Scale (FAS), mMRC Dyspnea Scale, SF-36.

### (ii) Pulmonary and Imaging:

Descriptive summary of pulmonary and imaging endpoints will be summarized over the time. Change from baseline will also be summarized by treatment group and by combining active treatment groups in Part A. Assessments are as follows: 6 MWD, HRCT.

### Analysis of the Exploratory Endpoint(s): Part B (POC)

Part B will be summarized for Quality of Life, Pulmonary, and Imaging exploratory endpoints. The analysis will be performed on FAS population by treatment.

### (i) Quality of Life (QoL):

Descriptive summary for absolute and change from baseline QoL endpoints will be summarized over the time.

The list of QoL assessments are as follows. KSQ—General, KSQ—Lung Module, LCQ, PGA, Fatigue Assessment Scale (FAS), mMRC Dyspnea Scale, SF-36, STQ.

### (ii) Pulmonary and Imaging:

Descriptive summary of pulmonary and imaging endpoints will be summarized over the time. Change from baseline also be summarized. Here is the list of assessments: FVC %, KSQ-Lung Score, 6 MWD, HRCT.

### Physical Examination and Vital Signs

Physical examination will be summarized descriptively by visit. Vital signs observed values and change from baseline by parameter will be summarized by subject, over time. Clinically notable vital signs (meeting predefined criteria as specified in the SAP) by visit subjects with notable vital signs data will be listed.

### Electrocardiogram

Electrocardiogram (ECG) observed values and change from baseline by parameter (unit) will be summarized over time. Echocardiogram data will be summarized descriptively. Echocardiogram data will also be summarized descriptively for the overall worst post-baseline value.

### Clinical Laboratory Evaluation

Descriptive statistics for clinical laboratory values will be presented for each clinical laboratory assessment. Observed values and change from baseline in hematology, blood chemistry and urinalysis assessments will be summarized over time. If the test results are reported in categorical format, the results will be summarized by subject counts and percentage for each category.

Each hematology, blood chemistry and urinalysis parameter will be classed as low, normal, high, missing based on the reference ranges. Shift tables in relation to the normal range from baseline over time will be presented. Clinically notable laboratory values will be submitted based on meeting predefined criteria specified in the SAP by visit, if any.

Listings of individual subjects' laboratory data will be generated. Values meeting predefined criteria for being clinically notable will be flagged within the listings as required.

### Baseline Descriptive Statistics

Demographics and Baseline Characteristics, Medical History, and Prior and Concomitant Medications summaries and analyses will be based upon the SAS population for Part A and ITT for Part B. Medical history data will be listed by subject including visit, description of the disease/procedure, MedDRA and Prior and concomitant medication will be coded according to the World Health Organization Drug Dictionary (WHO-DD) latest versions.

Subject disposition, protocol deviation is based on all subjects who have signed the ICF for both Part A and Part B.

Summary statistics for continuous variables will consist of number of non-missing observations (n), mean, SD, minimum, median, and maximum, unless specified otherwise. For categorical variables, the number and percentage of subjects in each category will be presented. Listing will also be provided for demographics and baseline characteristics, disposition, protocol deviations and randomization list (the treatment to which the subject has been randomized).

### Planned Interim Analyses

Part B interim analyses: No formal interim analysis is planned for this study. Ad-hoc review plan will be defined as per DSMC feedback, if needed

Sub-group Analyses: No subgroup analysis is planned for this study.

### Sample Size Determination: Part A (MAD)

Thirty two (32) subjects will be enrolled in Part A to receive XTMAB-16 or placebo in a MAD fashion in four cohorts for 12 weeks:
Cohort 1: 2 mg/kg Q4W for 12 weeks (n=6 active, 2 placebo)
Cohort 2: 4 mg/kg Q4W for 12 weeks (n=6 active, 2 placebo)
Cohort 3: 2 mg/kg Q2W for 12 weeks (n=6 active, 2 placebo)
Cohort 4: 4 mg/kg Q2W for 12 weeks (n=6 active, 2 placebo)

Each of the cohorts will enroll 8 subjects. The DSMC will perform periodical reviews of safety, tolerability, PK, and immunogenicity from the study according to the DSMC Charter to provide direction on whether to escalate, de-escalate the dose and frequency of XTMAB-16.

### Sample Size Determination: Part B (POC)

For Part B a total of 62 subjects will be randomized 1:1 to XTMAB-16:placebo for 24 weeks to demonstrate a statistically significant difference in favor of XTMAB-16. Assuming that the proportion of subjects in the placebo arm who achieve the targeted tapered dose of corticosteroid (maximum dose of prednisone 5 mg/day or equivalent) by Week 12 is about 15% and the expected proportion in the XTMAB-16 arm is 45%, i.e., the treatment difference is about 30%, a total of 56 subjects are required to demonstrate such a benefit at Week 12 using a one-sided α=0.05 and 80% power to test the proportion using CMH test with stratification factor (baseline dose of prednisone or equivalent). Considering about 10% dropouts, a total of 62 subjects randomized is adequate for this POC part of the study.

### Schedule of Activities (SoA)

Part A (MAD) of the study is a standalone 12-week sequential multiple doses escalating study. No formal statistical hypothesis will be tested in Part A of the study, due to the limited sample size in each treatment group.

Part B (POC) is a standalone double-blind placebo-controlled proof of concept study. To confirm preliminary efficacy of XTMAB-16 at Week 12, the following hypotheses will be tested in Part B of the study:

$${H}_{0} \text{:} {p}_{x} - {p}_{p} = 0 ,$$

$${H}_{1} \text{:} {p}_{x} - {p}_{p} > 0$$

pₓ and pₚ represent the proportion of subjects in the XTMAB-16 arm and placebo arm respectively, who achieve the targeted tapered dose of corticosteroid (dose of prednisone≤5 mg/day, or equivalent) by Week 12.

TABLE 44
Schedule of Activities 1 of 4 - Part A (MAD)

| | Study Period | | | | | | | | | |
|---|---|---|---|---|---|---|---|---|---|---|
| | | Study Day/Week | | | | | | | | |
| | | W 0 | | | | | | | | |
| | | (D 1) | | | | | | | W 12 | |
| | SCR^{a} | BL | W 1 | W 2 | W 4 | W 6 | W 8 | W 10 | EOT^{b} | Unsch |
| | Window | | | | | | | | | |
| | Day −42 | | | | | | | | | |
| | to 0 | | ±1 day | +/−3 days | | | | | | |
| Informed consent | X | | | | | | | | | |
| Demography | X | | | | | | | | | |
| Eligibility criteria (inclusion/exclusion) | X | X | | | | | | | | |
| Eligibility Confirmation & Randomization | | X | | | | | | | | |
| XTMAB-16 Infusion (Q4W Group)^{c} | | X | | | X | | X | | X | |
| XTMAB-16 Infusion (Q2W Group)^{c} | | X | | X | X | X | X | X | X | |
| Medical history | X | X | | | | | | | | |
| Physical Examination^{d} and Vital Signs | X | X | X | X | X | X | X | X | X | X |
| Oral Corticosteroid Diary Review | | X | X | X | X | X | X | X | X | X |
| Pregnancy Test^{e} | X | X | | X | X | X | X | X | X | X |
| FSH level test | X | | | | | | | | | |
| Blood Chemistry, Hematology^{f}, and | X | X | X | X | X | X | X | X | X | X |
| Urinalysis | | | | | | | | | | |
| 12-lead ECG^{g} | X | X | | | X | | X | | X | |
| Serology (HIV, HBsAg, HCV) | X | | | | | | | | | |
| QuantiFERON Gold TB | X | | | | | | | | X | |
| IgG | X | | | | | | | | | |
| Activated partial thromboplastin time | X | X | X | X | X | X | X | X | X | |
| COVID-19^{h} | X | X | X | X | X | X | X | X | X | X |
| Spirometry (FVC) | X | X | X | X | X | | X | | X | |
| 6MWD | X | X | | | X | | X | | X | |
| HRCTⁱ | X | X^{j} | | | | | | | X | |

TABLE 45
Schedule of Activities 2 of 4 - Part A (MAD)

| | Study Period | | | | | | | | | |
|---|---|---|---|---|---|---|---|---|---|---|
| | | Study Day/Week | | | | | | | | |
| | | W 0 | | | | | | | | |
| | | (D 1) | | | | | | | W 12 | |
| | SCR^{a} | BL | W 1 | W 2 | W 4 | W6 | W 8 | W 10 | EOT^{b} | Unsch |
| | Window | | | | | | | | | |
| | Day −42 | | ±1 | | | | | | | |
| | to 0 | | day | +/−3 days | | | | | | |
| mMRC | X | X | X | X | X | X | X | X | X | |
| Quality of Life Measurements^{k} | X | X | X | X | X | X | X | X | X | |
| PD Biomarkers (ACE, IL-6, sIL 2R, | | Per Table 60 | | | | | | | | |
| sTNFα, CRP, Calcitriol, IL-1b)^{l} | | | | | | | | | | |
| Pharmacokinetics^{l} | | Per Table 60 | | | | | | | | |
| Anti-Drug Antibodies (ADA and nAb)^{l} | | Per Table 60 | | | | | | | | |
| AE and SAE Assessment | | X | X | X | X | X | X | X | X | X |
| Prior/Concomitant Medications | X | X | X | X | X | X | X | X | X | X |
| Optional Rollover to Open Label Safety | | | | | | | | | X | |

TABLE 46
Schedule of Activities 3 of 4 - Part A (MAD)

| | Study Period | | | | |
|---|---|---|---|---|---|
| | | Safety Follow-Up (For | | | |
| | | non-rollover subjects) | | | |
| | Study Day/Week | | | | |
| | W 12 (D 0)^{b} | W 14 | W 16 | W 18 | W 20 |
| | Window | | | | |
| | Per Table 60 | +/−5 Days | | | |
| Physical Examination^{d} and Vital Signs | X | X | X | X | X |
| Oral Corticosteroid Diary Review | X | X | X | X | X |
| Pregnancy Test^{e} | X | X | X | X | X |
| Blood Chemistry, Hematology^{f}, and Urinalysis | X | X | X | X | X |
| QuantiFERON-TB Gold | | | | | X |
| Activated partial thromboplastin time | X | X | X | X | X |
| COVID-19^{h} | X | X | X | X | X |
| Spirometry (FVC) | | | | | X |
| mMRC | | | | | X |

TABLE 47
Schedule of Activities 4 of 4 - Part A (MAD)

| | | Safety Follow-Up (For | | | |
|---|---|---|---|---|---|
| Study Period | | non-rollover subjects) | | | |
| Study Day/Week | W 12 (D 0)^{b} | W 14 | W 16 | W 18 | W 20 |
| Window | Per Table 60 | +/− 5 Days | | | |
| Quality of Life Measurements^{k} | | | | | X |
| PD Biomarkers (ACE, IL-6, sIL 2R, | Per Table 60 | | | | |
| sTNFα, CRP, Calcitriol, IL-1b)^{l} | | | | | |
| Pharmacokinetics^{l} | Per Table 60 | | | | |
| Anti-Drug Antibodies (ADA and nAb)^{l} | Per Table 60 | | | | |
| AE and SAE Assessment | X | X | X | X | X |
| Prior/Concomitant Medications | X | X | X | X | X |

Abbreviations: 6 MWD=6 minute walk distance; ACE=angiotensin converting enzyme; ADA=anti-drug antibodies; AE=adverse event; BL=baseline; COVID-19=coronavirus disease; CRP=C-reactive protein; CT=computed tomography; D=day; ECG=electrocardiogram; EOT=end of treatment; FSH=Follicle stimulating hormone; FVC=forced vital capacity; HBsAg=Hepatitis B surface antigen; HCV=Hepatitis C virus; HIV=Human immunodeficiency virus; HRCT=high-resolution computed tomography; IgG=Immunoglobulin G; IL-1b=interleukin-1 b; IL-6=interleukin-6; IRR=infusion-related reaction; MAD=multiple ascending dose; mMRC=Modified Medical Research Council; nAb=neutralizing antibodies; PCR=polymerase chain reaction; PD=pharmacodynamic(s); Q2W=every 2 weeks; Q4W=every four weeks; SAE=serious adverse event; SCR=screening; sIL 2R=soluble interleukin-2 receptor; sTNFα=soluble tumor necrosis factor α; TB=tuberculosis; Unsch=unscheduled; W=week.
a The screening period is up to 42 days from the time of informed consent to enrollment.
b If the subject completes the W12 EOT assessments, the W12 D0 assessments do not need to be repeated.
c To assess for infusion site reactions, the infusion site should be examined at 0.5 and 1.5 hours (±10 minutes) after the end of infusion. The subject should be monitored for IRRs and anaphylaxis during this time.
d A complete physical examination will be done during screening, EOT, and on all dosing days. Abbreviated physical examination is done on visits when there is no dosing.
   A targeted physical examination may be conducted at any time at the Investigator's discretion. Height is measure at screening; weight is measured at every visit.
e Serum pregnancy test is done at screening, urine pregnancy test is done thereafter.
f Tryptase levels will be assessed at Baseline for all subjects. For any subject who experiences anaphylaxis within 4 hours after the end of infusion, an additional sample for assessment of tryptase levels should be collected as soon as is reasonable.
g Standard 12-lead ECGs will be recorded. ECGs will be collected in triplicate during screening and baseline with approximately 1 minute between each ECG; all other ECGs will be collected as single recordings.
h COVID-19 tests will be completed by rapid antigen testing or PCR test done locally.
i Known potentially significant fibrotic disease and/or active inflammation contained solely in the hilar region as shown by HRCT, confirmed by a central reader. Subjects with current active inflammation in the hilar region with concurrent inflammation outside the hilar region may be included. For subjects with disease onset of <2 years, a historical CT within 6 months prior to screening confirmed by central read is acceptable. For subjects with disease onset of >2 years and without CT within 6 months prior to screening, a CT will be performed at Screening.
j HRCT scan to be completed at baseline±10 days.
k All quality of life assessments are to be performed before other study procedures at each designated visit and are to be completed in the same order at each designated visit.
l Refer to Table 60 for pharmacokinetic, anti-drug antibody, and biomarker collection time points.

TABLE 48
Schedule of Activities 1 of 4 - Part B (POC)

| | Study Period | | | | | | | | | | | | | | |
|---|---|---|---|---|---|---|---|---|---|---|---|---|---|---|---|
| | | Intervention Period | | | | | | | | | | | | | |
| | | Study Week | | | | | | | | | | | | | |
| | | W 0 | | | | | | | | | | | | | |
| | | Base- | | | | | | | | | | | | W 24/ | |
| | SCR^{a} | line | W 2 | W 4 | W 6 | W 8 | W 10 | W 12 | W 14 | W 16 | W 18 | W 20 | W 22 | EOT^{b} | Unsch |
| | Window | | | | | | | | | | | | | | |
| | Day −42 | | | | | | | | | | | | | | |
| | to 0 | D 1 | ±3 days | | | | | | | | | | | | |
| Informed consent | X | | | | | | | | | | | | | | |
| Demography | X | | | | | | | | | | | | | | |
| Eligibility criteria | X | X | | | | | | | | | | | | | |
| (inclusion/ | | | | | | | | | | | | | | | |
| exclusion) | | | | | | | | | | | | | | | |
| Eligibility | | X | | | | | | | | | | | | | |
| Confirmation & | | | | | | | | | | | | | | | |
| Randomization | | | | | | | | | | | | | | | |
| XTMAB-16 | | X | | X | | X | | X | | X | | X | | X | |
| Infusion (Q4W | | | | | | | | | | | | | | | |
| Group)^{c} | | | | | | | | | | | | | | | |
| XTMAB-16 | | X | X | X | X | X | X | X | X | X | X | X | X | X | |
| Infusion (Q2W | | | | | | | | | | | | | | | |
| Group)^{c} | | | | | | | | | | | | | | | |
| Medical history | X | X | | | | | | | | | | | | | |
| Physical | X | X | X | X | X | X | X | X | X | X | X | X | X | X | X |
| Examination^{d} and | | | | | | | | | | | | | | | |
| Vital Signs | | | | | | | | | | | | | | | |
| Oral Corticosteroid | | X | X | X | X | X | X | X | X | X | X | X | X | X | X |
| Diary Review | | | | | | | | | | | | | | | |
| Pregnancy Test^{e} | X | X | X | X | | X | | X | | X | | X | | X | X |
| FSH level test | X | | | | | | | | | | | | | | |
| Blood Chemistry, | X | X | X | X | | X | | X | | X | | X | | X | X |
| Hematology^{f}, and | | | | | | | | | | | | | | | |
| Urinalysis | | | | | | | | | | | | | | | |
| 12-lead ECG^{g} | X | X | | X | | | | X | | | | | | X | |
| Serology (HIV, | | | | | | | | | | | | | | | |
| HBsAg, HCV) | | | | | | | | | | | | | | | |
| QuantiFERON-TB | X | | | | | | | | | | | | | X | |
| Gold | | | | | | | | | | | | | | | |
| IgG | X | | | | | | | | | | | | | | |
| Activated partial | X | X | X | X | | X | | X | | X | | X | | X | X |
| thromboplastin | | | | | | | | | | | | | | | |
| time | | | | | | | | | | | | | | | |
| COVID-19^{h} | X | X | X | X | X | X | X | X | X | X | X | X | X | X | X |
| Spirometry (FVC) | X | X | | X | | X | | X | | X | | X | | X | |
| 6MWD | | X | | | | | | X | | | | | | X | |

TABLE 49
Schedule of Activities 2 of 4 - Part B (POC)

| | Study Period | | | | | | | | | | | | | | |
|---|---|---|---|---|---|---|---|---|---|---|---|---|---|---|---|
| | | Intervention Period | | | | | | | | | | | | | |
| | | Study Week | | | | | | | | | | | | | |
| | | W 0 | | | | | | | | | | | | | |
| | | Base- | | | | | | | | | | | | W 24/ | |
| | SCR^{a} | line | W 2 | W 4 | W 6 | W 8 | W 10 | W 12 | W 14 | W 16 | W 18 | W 20 | W 22 | EOT^{b} | Unsch |
| | Window | | | | | | | | | | | | | | |
| | Day −42 | | | | | | | | | | | | | | |
| | to 0 | D 1 | ±3 days | | | | | | | | | | | | |
| HRCT | | Xⁱ | | | | | | X | | | | | | X | |
| mMRC | X | X | | X | | X | | X | | X | | X | | X | |
| Quality of Life | X | X | | X | | X | | X | | X | | X | | X | |
| Measurements^{j} | | | | | | | | | | | | | | | |
| PD Biomarkers | Per Table 60 | | | | | | | | | | | | | | X |
| (ACE, IL-6, sIL 2R, | | | | | | | | | | | | | | | |
| sTNFα, | | | | | | | | | | | | | | | |
| CRP, Calcitriol, and | | | | | | | | | | | | | | | |
| IL-1b)^{k} | | | | | | | | | | | | | | | |
| Pharmacokinetics^{k} | Per Table 60 | | | | | | | | | | | | | | X |
| Anti-Drug | Per Table 60 | | | | | | | | | | | | | | X |
| Antibodies (ADA | | | | | | | | | | | | | | | |
| and nAb)^{k} | | | | | | | | | | | | | | | |
| AE and SAE | | X | X | X | X | X | X | X | X | X | X | X | X | X | X |
| Assessment | | | | | | | | | | | | | | | |
| Prior/Concomitant | X | X | X | X | X | X | X | X | X | X | X | X | X | X | X |
| Medications | | | | | | | | | | | | | | | |
| Optional Rollover to | | | | | | | | | | | | | | X | |
| Open Label Safety | | | | | | | | | | | | | | | |

TABLE 50
Schedule of Activities 3 of 4 - Part B (POC)

| | Study Period | | | | | |
|---|---|---|---|---|---|---|
| | | Safety Follow-Up (For non- | | | | |
| | | rollover subjects) | | | | |
| | Study Day/Week | | | | | |
| | W 24 (D 0)^{b} | W 26 | W 28 | W 30 | W 32 | W 34 |
| | Window | | | | | |
| | Per Table 60 | +/−5 Days | | | | |
| Physical Examination^{d} and Vital Signs | X | X | X | X | X | X |
| Oral Corticosteroid Diary Review | X | X | X | X | X | X |
| Pregnancy Test^{e} | X | X | X | X | X | X |
| Blood Chemistry, Hematology^{f}, and Urinalysis | X | X | X | X | X | X |
| QuantiFERON-TB Gold | | | | | | X |
| Activated partial thromboplastin time | X | X | X | X | X | X |
| COVID-19^{h} | X | X | X | X | X | X |
| Spirometry (FVC) | | | | | | X |
| mMRC | | | | | | X |
| Quality of Life Measurements^{j} | | | | | | X |

TABLE 51
Schedule of Activities 4 of 4 - Part B (POC)

| | Study Period | | | | | |
|---|---|---|---|---|---|---|
| | | Safety Follow-Up (For | | | | |
| | | non-rollover subjects) | | | | |
| | Study Day/Week | | | | | |
| | W 24 (D 0)^{b} | W 26 | W 28 | W 30 | W 32 | W 34 |
| | Window | | | | | |
| | Per Table 60 | +/−5 Days | | | | |
| PD Biomarkers (ACE, IL-6, sIL 2R, sTNFα, | Per Table 60 | | | | | |
| CRP, Calcitriol, IL-1b)^{k} | | | | | | |
| Pharmacokinetics^{k} | Per Table 60 | | | | | |
| Anti-Drug Antibodies (ADA and nAb)^{k} | Per Table 60 | | | | | |
| AE and SAE Assessment | X | X | X | X | X | X |
| Prior/Concomitant Medications | X | X | X | X | X | X |

Abbreviations: 6 MWD=6 minute walk distance; ACE=angiotensin converting enzyme; ADA=anti-drug antibodies; AE=adverse event; COVID-19=coronavirus disease; CRP=C-reactive protein; D=day; ECG=electrocardiogram; EOT=end of treatment; FSH=follicle stimulating hormone; FVC=forced vital capacity; HBsAg=Hepatitis B surface antigen; HCV=Hepatitis C virus; HIV=Human immunodeficiency virus; HRCT=high-resolution computed tomography; IgG=Immunoglobulin G; IL-1b=interleukin 1b; IL-6=interleukin-6; IRR=infusion-related reaction; mMRC=Modified Medical Research Council; nAb=neutralizing antibodies; PCR=polymerase chain reaction; PD=pharmacodynamics; Q2W=every two weeks; Q4W=every four weeks; SAE=serious adverse event; SCR=screening; sIL 2R=soluble interleukin-2 receptor; sTNFα=soluble tumor necrosis factor α; TB=tuberculosis; Unsch=unscheduled; W=week.
a The screening period is up to 42 days from the time of informed consent to enrollment.
b If the subject completes the W24 EOT assessments, the W24 D0 assessments do not need to be repeated.
c To assess for infusion site reactions, the infusion site should be examined at 0.5 and 1.5 hours (±10 minutes) after the end of infusion. The subject should be monitored for IRRs and anaphylaxis during this time.
d A complete physical examination will be done during screening, EOT, and on all dosing days. Abbreviated physical examination is done on visits when there is no dosing.
   Targeted physical examination may be conducted at any time at the Investigator's discretion. Height is measure at screening; weight is measured at every visit.
e Serum pregnancy test is done at screening, urine pregnancy test is done thereafter.
f Tryptase levels will be assessed at Baseline for all subjects. For any subject who experiences anaphylaxis within 4 hours after the end of infusion, an additional sample for assessment of tryptase levels should be collected as soon as is reasonable.
g Standard 12-lead ECGs will be recorded. ECGs will be collected as single recordings.
h COVID-19 tests will be completed by rapid antigen testing or PCR test done locally.
i HRCT scan to be completed at baseline±10 days.
j All quality of life assessments are to be performed before other study procedures at each designated visit and are to be completed in the same order at each designated visit.
k Refer to Table 60 for pharmacokinetic, anti-drug antibody, and biomarker collection time points.

TABLE 52
Pharmacokinetic, Anti-drug Antibody, and Biomarker Collection Time Points

| | | | | |
|---|---|---|---|---|
| | | | ADA and | Biomarker |
| | | PK Sample | nAb Sample | Sample |
| Week/Day | Hour | Serum | Serum | Serum |

Part A Q2W regimen

| | | | | | |
|---|---|---|---|---|---|
| All | Week 0/ | Predose (Hour 0)^{a} | X | X | X |
| subjects | Day 1 | EOI | | (Predose) | (Predose) |
| | | 4 hours post SOI | | | |
| | | 6 hours post SOI^{b} | | | |
| | Week 1 | 168 post SOI^{c} | X | | |
| | Week 2 | Predose^{a} | X | X | X |
| | Week 4 | Predose^{a} | X | X | X |
| | Week 6 | Predose^{a} | X | X | X |
| | Week 8 | Predose^{a} | X | X | X |
| | Week 10 | Predose^{a} | X | X | X |
| | Week 12^{d} | Predose^{a} | X | X | X |
| Non- | Week 14 | With labs | X | X | X |
| rollover | Week 16 | With labs | X | X | X |
| subjects | Week 18 | With labs | X | X | X |
| | Week 20 | With labs | | X | X |

Part A Q4W regimen

| | | | | | |
|---|---|---|---|---|---|
| All | Week 0/ | Predose (Hour 0)^{a} | X | X | X |
| subjects | Day 1 | EOI | | (Predose) | (Predose) |
| | | 4 post SOI | | | |
| | | 6 post SOI^{b} | | | |
| | Week 1 | 168 post SOI^{c} | X | | |
| | Week 2 | With labs | X | X | X |
| | Week 4 | Predose^{a} | X | X | X |
| | Week 6 | With labs | X | X | X |
| | Week 8 | Predose^{a} | X | X | X |
| | Week 10 | With labs | X | X | X |
| | Week 12 | Predose^{a} | X | X | X |
| | Week 14 | With labs | X | X | X |
| | Week 16 | With labs | X | X | X |
| | Week 18 | With labs | X | X | X |
| | Week 20 | With labs | | X | X |

Part B

| | | | | ADA and | Biomarker |
|---|---|---|---|---|---|
| | | | PK Sample | nAb Sample | Sample |
| Week/Day | | | Serum | Serum | Serum |
| All Subjects | Week 0 | Pre-Dose depending on | | | Predose |
| | Week 2 | the results of Part A | | | |
| | Week 4 | | | | |
| | Week 6 | | | | |
| | Week 8 | | | | |
| | Week 10 | | | | |
| | Week 12 | | | | Predose |
| | Week 14 | | | | |
| | Week 16 | | | | |
| | Week 18 | | | | |
| | Week 20 | | | | |
| | Week 22 | | | | |
| | Week 24^{d} | | | | Predose |
| Non-rollover | Week 26 | | With labs | | |
| subjects | Week 28 | | With labs | | |
| | Week 30 | | With labs | | |
| | Week 32 | | | With labs | |
| | Week 34 | | | With labs | With labs |

Abbreviations: ADA = anti-drug antibodies; EOI = end of infusion; EOT = end of treatment; nAb = neutralizing antibodies; PK = pharmacokinetics; Q2W = every two weeks; Q4W = every four weeks; SOI = start of infusion.
^{a}Sampling window is −30 minutes.
^{b}Sampling window is +5 minutes.
^{c}Sampling window is +3 hour.
^{d}Week 12 or 24 Visits in Parts A and B, respectively, is the EOT Visit and not the Safety Follow-Up visit for non-rollover subjects.

### Scientific Rationale for Study Design

Part A of this study will evaluate safety, pharmacokinetics, pharmacodynamics, effect on inflammatory biomarkers, and potential for efficacy of four distinct multiple dose regimens (mg/kg dose and frequency of administration) (Table 61), which vary in predicted average concentration and trough concentration of XTMAB-16, to enable identification of the optimal regimen for subjects with sarcoidosis with or without pulmonary manifestations.

Within Part B, the study will confirm the preliminary efficacy of XTMAB-16 as measured by the ability to reduce background oral corticosteroid use in subjects with pulmonary sarcoidosis with or without extrapulmonary manifestations, as well as evaluate multiple clinically relevant endpoints.

Dose justification is based, in part, on the results of Example 8.

TABLE 53
Stimulated Repeat Dose Conditions

| ADA | | | | | | | | | |
|---|---|---|---|---|---|---|---|---|---|
| negative | Cₘₐₓ | Ceoi | C_{trough} | Vₛₛ | Tₘₐₓ | MRT_{inf} | T_{1/2} | AUCₜₐᵤ | C_{avg} |
| Profile | (ug/mL) | (ug/ml) | (ug/mL) | (mL/kg) | (h) | (h) | (h) | (h*ug/mL) | (ug/mL) |
| 2 mg/kg | 86.7 | 86.7 | 25.9 | 42.1 | 2.00 | 326 | 235 | 15500 | 46.1 |
| Q2W | | | | | | | | | |
| Multiple | | | | | | | | | |
| Doses | | | | | | | | | |
| 2 mg/kg | 68.0 | 68.0 | 7.11 | 42.5 | 2.00 | 329 | 238 | 15500 | 23.0 |
| Q4W | | | | | | | | | |
| Multiple | | | | | | | | | |
| Doses | | | | | | | | | |
| 4 mg/kg | 173 | 173 | 51.8 | 42.1 | 2.00 | 326 | 235 | 31000 | 92.1 |
| Q2W | | | | | | | | | |
| Multiple | | | | | | | | | |
| Doses | | | | | | | | | |
| 4 mg/kg | 136 | 136 | 14.2 | 42.5 | 2.00 | 329 | 238 | 31000 | 46.1 |
| Q4W | | | | | | | | | |
| Multiple | | | | | | | | | |
| Doses | | | | | | | | | |

Abbreviations:
ADA = anti-drug antibodies;
AUCₜₐᵤ = area under the curve;
C_{avg} = average concentration;
Ceoi = end of infusion concentration;
Cₘₐₓ = maximum concentration;
C_{trough} = trough concentration;
kg = kilogram;
mg = milligram;
MRT = mean residence time;
Q2W = every 2 weeks;
Q4W = every four weeks;
t_{1/2} = half-life;
Tₘₐₓ = time to maximum observed concentration;
Vₛₛ = apparent volume of distribution at steady state.
Ceoi was set to simulated serum XTMAB-16 concentration at 2-hour nominal time.
Tau = 336 hours for Q2W dosing,
Tau = 672 hours for Q4W dosing

### End of Study Definition

A subject will be considered to have completed the study if he or she has completed all phases of the study including the last visit or the last scheduled procedure shown in the Schedule of Activities (SoA) (Table 52-Table 59).

All subjects who complete Part A or Part B will be offered participation in a long-term open label follow-up study at the dose and frequency established in Part A.

Part A End of Treatment (EOT) will be considered the last dose at Week 12. Subjects who do not choose to participate in the open label extension will complete safety follow up visits to Week 20 as outlined in the SoA, (Table 52-Table 59).

Part B EOT will be considered the last dose at Week 24. Subjects who do not choose to participate in the open label extension will complete safety follow up visits to Week 34 as outlined in the SoA (Table 44-Table 51).

The end of the study will be defined as the date of the last visit of the last subject in the study or last scheduled procedure shown in the SoA for the last subject in the trial globally.

The teachings of the present technology provided herein may be applied to other methods, not necessarily the method described above. The elements and acts of the various embodiments described above may be combined to provide further embodiments.

All of the above patents and applications and other references, including any that may be listed in accompanying filing papers, are incorporated herein by reference. Aspects of the present technology may be modified, if necessary, to employ the methods, functions, and concepts of the various references described above to provide yet further embodiments of the present technology.

These and other changes may be made to the present technology in light of the above detailed description. While the above description details certain embodiments of the present technology and describes the best mode contemplated, no matter how detailed the above appears in text, the present technology may be practiced in many ways. Details of the methods may vary considerably in its implementation details, while still being encompassed by the present technology.

As noted above, the particular terminology used when describing certain features or aspects of the present technology should not be taken to imply that the terminology is being redefined herein to be restricted to any specific characteristics, features, or aspects of the present technology with which that terminology is associated. In general, the terms used in the following claims should not be construed to limit the present technology to the specific embodiments of the present technology, unless the above detailed description section explicitly defines such terms. Accordingly, the actual scope of the present technology encompasses not only the embodiments of the present technology, but also all equivalent ways of practicing or implementing the present technology under the claims.

From the foregoing, it will be appreciated that specific embodiments of the present technology have been described herein for purposes of illustration, but that various modifications may be made without deviating from the scope of the present technology. Accordingly, the present technology is not limited except as by the appended claims.

### Additional Embodiments

The present technology includes, but is not limited to, the following specific embodiments set forth herein below in paragraphs [0753]-[1031]:
1. A method of treating or reducing sarcoidosis in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every four weeks for a first period of time.
2. A method of treating or reducing sarcoidosis in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every two weeks for a first period of time.
3. A method of reducing granuloma size or granuloma formation in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every four weeks for a first period of time.
4. A method of reducing granuloma size or granuloma formation in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every two weeks for a first period of time.
5. The method of any one of embodiments 1-4, wherein the first period of time is about 6 weeks to about 24 weeks.
6. The method of embodiment 5, wherein the first period of time is about 8 weeks.
7. The method of embodiment 5, wherein the first period of time is about 12 weeks.
8. The method of embodiment 5, wherein the first period of time is about 24 weeks.
9. The method of any one of embodiments 1-8, wherein the first dose is about 2 mg/kg of the composition.
10. The method of any one of embodiments 1-8, wherein the first dose is about 4 mg/kg of the composition.
11. The method of any one of embodiments 1-10, wherein the anti-TNFα antibody comprises a complementarity determining region (CDR) 100% identical to SEQ ID NO: 3, 4, 5, 6, 7, 8, 10, 12, 13, 14, or 15, or YA.
12. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time, and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to SEQ ID NO: YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
13. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
14. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every four weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
15. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
16. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
17. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
18. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every four weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
19. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
20. The method of any one of embodiments 1-19, further comprising administering to the subject a second dose of the composition for a second period of time.
21. The method of embodiment 20, wherein the second period of time is about 6 weeks to about 16 weeks.
22. The method of embodiment 21, wherein the second period of time is about 8 weeks.
23. The method of embodiment 21, wherein the second period of time is about 12 weeks.
24. The method of any one of embodiments 20-23, wherein the second dose comprises about 1 mg/kg to about 5 mg/kg.
25. The method of embodiment 24, wherein the second dose is about 2 mg/kg.
26. The method of embodiment 24, wherein the second dose is about 4 mg/kg.
27. The method of embodiment 24, wherein the second dose is about two times greater than the first dose.
28. The method of embodiment 27, wherein the administration frequency of the second dose comprises the same administration frequency of the first dose.
29. The method of embodiment 27, wherein the administration frequency of the second dose consists of the same administration frequency of the first dose.
30. The method of embodiment 28 or 29, wherein the first dose and the second dose are each administered about once every four weeks.
31. The method of embodiment 20, wherein the second dose is about the same dose as the first dose.
32. The method of embodiment 20, wherein the second dose is the same dose as the first dose.
33. The method of embodiment 31 or 32, wherein the first dose and the second dose are about 2 mg/kg.
34. The method of embodiment 31 or 32, wherein the administration frequency of the second dose is about two times greater than the administration frequency of the first dose.
35. The method of embodiment 33 or 34, wherein the first dose is administered about once every four weeks and the second dose is administered about once every two weeks.
36. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.
37. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.
38. The method of embodiment 36 or 37, wherein the first period of time is about 6 weeks to about 12 weeks.
39. The method of embodiment 38, wherein the first period of time is about 8 weeks.
40. The method of embodiment 38, wherein the first period of time is about 12 weeks.
41. The method of any one of embodiments 36-40, wherein the second period of time is about 6 weeks to about 12 weeks.
42. The method of embodiment 41, wherein the second period of time is about 8 weeks.
43. The method of embodiment 41, wherein the second period of time is about 12 weeks.
44. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%,
45. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time; and
the second dose comprising (i) about 2 mg/kg of the composition administered once every two weeks, or (iii) about 4 mg/kg of the composition administered once every four weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
46. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time;
the second dose comprising (i) about 2 mg/kg of the composition administered once every two weeks, or (iii) about 4 mg/kg of the composition administered once every four weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
47. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time;
the second dose comprising (i) about 2 mg/kg of the composition administered once every two weeks, or (iii) about 4 mg/kg of the composition administered once every four weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
48. The method of any one of embodiments 20-47, further comprising administering (c) a third dose of about 4 mg/kg of the composition about once every two weeks, for a third period of time.
49. The method of embodiment 48, wherein the third period of time is at least about 24 weeks.
50. The method of any one of embodiments 1-49, wherein the anti-TNFα antibody comprises a variable heavy (VH) domain having an amino acid sequence at least about 80%, 85%, 90%, 95%, 99%, or 100% identical to SEQ ID NO: 16.
51. The method of any one of embodiments 1-49, wherein the anti-TNFα antibody comprises a variable light (VL) domain having an amino acid sequence at least about 80%, 85%, 90%, 95%, 99%, or 100% identical to SEQ ID NO: 9.
52. The method of any one of embodiments 1-51, wherein the anti-TNFα antibody comprises an asparagine-linked glycosylation site within a constant Fc region on at least one of the heavy chains.
53. The method of embodiment 52, wherein the anti-TNFα antibody comprises an asparagine-linked glycosylation site within the constant Fc region on both of the heavy chains.
54. The method of any one of embodiments 50-53, wherein the anti-TNFα antibody is sialylated at Asn 297.
55. The method of any one of embodiments 1-54, wherein the anti-TNFα antibody comprises no N-Glycolylneuraminic acid (Neu5Gc).
56. The method of any one of embodiments 1-55, wherein the anti-TNFα antibody is an immunoglobulin G (IgG) antibody.
57. The method of claim 43, wherein the IgG antibody is an IgG1 antibody.
58. The method of embodiment 57, wherein the IgG1 antibody comprises a kappa isotype.
59. The method of any one of embodiments 1-58, wherein the anti-TNFα antibody is a chimeric antibody.
60. The method of any one of embodiments 1-59, wherein the anti-TNFα antibody comprises a C-terminal lysine on at least one of the heavy chains.
61. The method of embodiment 60, wherein the anti-TNFα antibody comprises a C-terminal lysine on both of the heavy chains.
62. The method of embodiment 60 or 61, wherein the C-terminal lysine is not clipped.
63. The method of any one of embodiments 1-62, wherein the composition is administered by an infusion.
64. The method of embodiment 63, wherein the infusion is an intravenous (IV) infusion.
65. The method of embodiment 63 or 64, wherein the infusion is administered over a period of at least about 2 hours.
66. The method of embodiment 63 or 64, wherein the infusion is administered over a period of about 2 hours.
67. The method of any one of embodiments 1-66, wherein the subject receives a cumulative dose of about 6 mg/kg of the composition during the first period of time.
68. The method of any one of embodiments 1-66, wherein the subject receives a cumulative dose of about 8 mg/kg of the composition during the first period of time.
69. The method of any one of embodiments 1-66, wherein the subject receives a cumulative dose of about 16 mg/kg of the composition during the first period of time.
70. The method of any one of embodiments 41-69, wherein the subject receives a cumulative dose of about 10 mg/kg to about 16 mg/kg of the composition during the second period of time.
71. The method of embodiment 70, wherein the subject receives a cumulative dose of about 10 mg/kg of the composition during the second period of time.
72. The method of embodiment 70, wherein the subject receives a cumulative dose of about 12 mg/kg of the composition during the second period of time.
73. The method of embodiment 70, wherein the subject receives a cumulative dose of about 14 mg/kg of the composition during the second period of time.
74. The method of embodiment 70, wherein the subject receives a cumulative dose of about 16 mg/kg of the composition during the second period of time.
75. The method of any one of embodiments 48-74, wherein the subject receives a cumulative dose of about 28 mg/kg of the composition.
76. The method of any one of embodiments 1-75, wherein the subject is administered a cumulative dose of about 30 mg/kg to about 60 mg/kg of the composition.
77. The method of embodiment 76, wherein the subject is administered a cumulative dose of about 46 mg/kg of the composition.
78. The method of embodiment 76, wherein the subject is administered a cumulative dose of about 48 mg/kg of the composition.
79. The method of embodiment 76, wherein the subject is administered a cumulative dose of about 50 mg/kg of the composition.
80. The method of embodiment 76, wherein the subject is administered a cumulative dose of about 52 mg/kg of the composition.
81. The method of any one of embodiments 1-80, wherein the subject is administered at least 3 doses of the composition.
82. The method of any one of embodiments 1-81, wherein the subject is administered at least 4 doses of the composition.
83. The method of any one of embodiments 1-82, wherein the subject is administered at least 6 doses of the composition.
84. The method of any one of embodiments 1-83, wherein the subject is administered at least 7 doses of the composition.
85. The method of embodiment 81, wherein the at least 3 doses are administered over a 4-week period of time.
86. The method of embodiment 81, wherein the at least 3 doses are administered over an 8-week period of time.
87. The method of embodiment 81, wherein the at least 3 doses are administered over a 12-week period of time.
88. The method of embodiment 82, wherein the at least 4 doses are administered over a 9-week period of time.
89. The method of embodiment 82, wherein the at least 4 doses are administered over a 10-week period of time.
90. The method of embodiment 82, wherein the at least 4 doses are administered over an 11-week period of time.
91. The method of embodiment 82, wherein the at least 4 doses are administered over a 12-week period of time.
92. The method of embodiment 83, wherein the at least 6 doses are administered over a 10-week period of time.
93. The method of embodiment 83, wherein the at least 6 doses are administered over a 13-week period of time.
94. The method of embodiment 83, wherein the at least 6 doses are administered over a 16-week period of time.
95. The method of embodiment 83, wherein the at least 6 doses are administered over a 17-week period of time.
96. The method of embodiment 83, wherein the at least 6 doses are administered over a 20-week period of time.
97. The method of embodiment 84, wherein the at least 7 doses are administered over a 12-week period of time.
98. The method of embodiment 84, wherein the at least 7 doses are administered over a 15-week period of time.
99. The method of embodiment 84, wherein the at least 7 doses are administered over a 21-week period of time.
100. The method of claim 82, wherein the at least 7 doses are administered over a 24-week period of time.
101. The method of embodiment 84, wherein the at least 7 doses are administered over a 28-week period of time.
102. The method of any one of embodiments 1-101, wherein administration of the composition reduces a level of one or more transcripts selected from the group consisting of angiotensin-converting enzyme 2 (ACE2), interleukin 6 (IL6), interleukin 2 receptor (IL2R), tumor necrosis factor alpha (TNFα), C-Reactive Protein (CRP), interleukin 1B (IL1B), interleukin 12 (IL12), interleukin 18 (IL18), interferon gamma (IFNG), interleukin 8 (IL8), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JunB), mitogen-activated protein kinase (MKP1), TNF alpha induced protein 1 (TNFAIP1), TNF alpha induced protein 2 (TNFAIP2), TNF alpha induced protein 3 (TNFAIP3), syndecan 4 (SDC4), superoxide dismutase 2 (SOD2), cytochrome c oxidase subunit II (COX2), interleukin 32 (IL32), intercellular adhesion molecule 1 (ICAM-1), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), and vascular adhesion molecule 1 (VCAM-1), relative to a control.
103. The method of embodiment 102, wherein the level of the one or more transcripts is reduced by at least about 10%, 20%, 30%, 40%, 50%, 60%, 70%, 80%, 90%, or 100% relative to the control.
104. The method of any one of embodiments 1-103, wherein administration of the composition reduces a level of one or more proteins selected from the group consisting of angiotensin-converting enzyme 2 (ACE2), interleukin 6 (IL-6), interleukin 2 receptor (IL-2R), tumor necrosis factor alpha (TNF-α), C-Reactive Protein (CRP), interleukin 1B (IL-1B), interferon gamma (IFN-γ), interleukin 8 (IL-8), interleukin 12 (IL-12), interleukin 18 (IL-18), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JUNB), mitogen-activated protein kinase phosphatase 1 (MKP-1), TNF alpha-induced protein 1 (TNFAIP1), TNF alpha-induced protein 2 (TNFAIP2), TNF alpha-induced protein 3 (TNFAIP3), syndecan 4 (SDC4), superoxide dismutase 2 (SOD2), cytochrome c oxidase subunit II (COX2), interleukin 32 (IL-32), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), intercellular adhesion molecule 1 (ICAM-1), and vascular adhesion molecule 1 (VCAM-1) relative to a control.
105. The method of embodiment 104, wherein the level of the one or more proteins is reduced by at least about 10%, 20%, 30%, 40%, 50%, 60%, 70%, 80%, 90%, or 100% relative to the control.
106. The method of any one of embodiments 1-105, wherein administration of the composition increases one or more of a Forced Vital Capacity (FVC) measurement, a Forced Expiratory Volume in 1 second (FEV1) measurement, a Leicester Cough Questionnaire (LCQ) score, a Short Form 36 Health Survey (SF-36) score, a King's Sarcoidosis Questionnaire (KSQ) score, a Steroid-toxicity scale (STS) score, or a 6-minute walk test (6MWT) distance relative to a control.
107. The method of embodiment 106, wherein the FVC measurement is increased by at least about 10%, 20%, 30%, 40%, 50%, 60%, 70%, 80%, 90%, or 100% relative to the control.
108. The method of embodiment 106, wherein the FEV1 measurement is increased by at least about 10%, 20%, 30%, 40%, 50%, 60%, 70%, 80%, 90%, or 100% relative to the control.
109. The method of embodiment 106, wherein the LCQ score is increased by at least about 1, 2, 3, 4, 5, 6, 7, 8, 9, 10, 11, 12, 13, 14, 15, 16, 17, or 18 points relative to the control.
110. The method of embodiment 106, wherein the SF-36 score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.
111. The method of embodiment 106, wherein the KSQ score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.
112. The method of embodiment 106 or 111, wherein the KSQ score is a KSQ General Health Status Module or a KSQ lung score.
113. The method of embodiment 106, wherein the STS score is increased by at least 1, 2, 3, 4, 5, or 6 points relative to the control.
114. The method of embodiment 106, wherein the 6MWT distance is increased by at least 10 m, 20 m, 30 m, 40 m, 50 m, 60 m, 70 m, 80 m, 90 m, 100 m, 125 m, 150 m, 175 m, 200 m, 250 m, 300 m, 350 m, 400 m, 450 m, or 500 m relative to the control.
115. The method of any one of embodiments 1-114, wherein administration of the composition reduces one or more of a Patient Global Assessment (PGA) score, a Fatigue Assessment Scale (FAS) score, Modified Medical Research Council (mMRC) Dyspnea Scale score, a Saint George's Respiratory Questionnaire (SGRQ) score, or a Borg's CR10 dyspnea score relative to a control.
116. The method of embodiment 115, wherein the PGA score is reduced by at least about 1, 2, 3, 4, 5, 6, 7, 8, 9, or 10 points relative to the control.
117. The method of embodiment 115, wherein the FAS score is reduced by at least about 1, 2, 3, or 4 points relative to a control.
118. The method of embodiment 115, wherein the mMRC Scale score is reduced by at least about 1, 2, 3, or 4 grades relative to the control.
119. The method of embodiment 115, wherein the SGRQ score is reduced by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.
120. The method of embodiment 115, wherein the Borg's CR10 dyspnea score is reduced by at least about 1, 2, 3, 4, 5, 6, 7, 8, 9, or 10 points relative to the control.
121. The method of any one of embodiments 1-120, wherein administration of the composition reduces a need for an anti-inflammatory medication relative to a control.
122. The method of embodiment 121, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.
123. The method of embodiment 122, wherein the corticosteroid is selected from the group consisting of prednisone, prednisolone, dexamethasone, hydrocortisone, methylprednisolone, betamethasone, cortisone, fludrocortisone, and triamcinolone.
124. The method of any one of embodiments 121-123, wherein the reduced need for anti-inflammatory medication comprises a reduction in frequency of, dosage of, or duration of administration of the anti-inflammatory medication.
125. The method of any one of embodiments 1-123, wherein administration of the composition reduces a granuloma formation or a granuloma size in the subject by about 1%, 2%, 3%, 4%, 5%, 10%, 15%, 20%, 25%, 30%, 40%, 50%, 60%, 70%, 80%, 90%, or 100%, relative to a control.
126. The method of embodiment 125, wherein the granuloma is a skin granuloma, a lung granuloma, a lymph node granuloma, an ocular granuloma, a liver granuloma, a spleen granuloma, a cardiac granuloma, or a rheumatoid granuloma.
127. The method of any one of embodiments 1-126, wherein administration of the composition reduces a calcitriol level in the subject relative to a control.
128. The method of embodiment 127, wherein the calcitriol level is reduced by at least about 5%, 10%, 15%, 20%, 25%, 30%, 40%, 50%, 60%, 70%, 80%, 90%, or 100% relative to the control.
129. The method of any one of embodiments 1-128, wherein administration of the composition reduces a neopterin level in the subject relative to a control.
130. The method of embodiment 129, wherein the neopterin level is reduced by at least about 5%, 10%, 15%, 20%, 25%, 30%, 40%, 50%, 60%, 70%, 80%, 90%, or 100% relative to the control.
131. The method of any one of embodiments 1-130, wherein administration of the composition reduces a lysozyme level in the subject relative to a control.
132. The method of embodiment 131, wherein the lysozyme level is reduced by at least about 5%, 10%, 15%, 20%, 25%, 30%, 40%, 50%, 60%, 70%, 80%, 90%, or 100% relative to the control.
133. The method of any one of embodiments 1-132, wherein administration of the composition reduces a soluble IL-2R (sIL-2R) level in the subject relative to a control.
134. The method of embodiment 132, wherein the sIL-2R level is reduced by at least about 5%, 10%, 15%, 20%, 25%, 30%, 40%, 50%, 60%, 70%, 80%, 90%, or 100% relative to the control.
135. The method of any one of embodiments 1-134, wherein the subject has one or more of acute sarcoidosis, chronic sarcoidosis, progressive sarcoidosis, pulmonary sarcoidosis, remitting sarcoidosis, refractory sarcoidosis, advanced sarcoidosis, ocular sarcoidosis, cutaneous sarcoidosis, or neurosarcoidosis.
136. The method of any one of embodiments 1-135, wherein the subject has received an anti-inflammatory medication.
137. The method of any one of embodiments 1-136, wherein the subject is receiving an anti-inflammation medication.
138. The method of embodiment 136 or 137, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.
139. The method of embodiment 138, wherein the corticosteroid is selected from the group consisting of prednisone, prednisolone, dexamethasone, hydrocortisone, methylprednisolone, betamethasone, cortisone, fludrocortisone, and triamcinolone.
140. A composition comprising an anti-TNFα antibody sialylated with one or more N-acetylneuraminic acid molecules for use in a method of treating or reducing sarcoidosis in a subject in need thereof relative to a control, wherein the composition is administered as a first dose of about 0.5 mg/kg to about 6.0 mg/kg about once every four weeks for a first period of time.
141. A composition comprising an anti-TNFα antibody sialylated with one or more N-acetylneuraminic acid molecules for use in a method of treating or reducing sarcoidosis in a subject in need thereof relative to a control, wherein the composition is administered as a first dose of about 0.5 mg/kg to about 6.0 mg/kg about once every two weeks for a first period of time.
142. A composition comprising an anti-TNFα antibody sialylated with one or more N-acetylneuraminic acid molecules for use in a method of reducing granuloma size or granuloma formation in a subject in need thereof relative to a control, wherein the composition is administered as a first dose of about 0.5 mg/kg to about 6.0 mg/kg about once every four weeks for a first period of time.
143. A composition comprising an anti-TNFα antibody sialylated with one or more N-acetylneuraminic acid molecules for use in a method of reducing granuloma size or granuloma formation in a subject in need thereof relative to a control, wherein the composition is administered as a first dose of about 0.5 mg/kg to about 6.0 mg/kg about once every two weeks for a first period of time.
144. The composition of any one of embodiments 140-143, wherein the first period of time is about 6 weeks to about 24 weeks.
145. The composition of embodiment 144, wherein the first period of time is about 8 weeks.
146. The composition of embodiment 144, wherein the first period of time is about 12 weeks.
147. The composition of embodiment 144, wherein the first period of time is about 24 weeks.
148. The composition of any one of embodiments 140-147, wherein the first dose is about 2 mg/kg of the composition.
149. The composition of any one of embodiments 140-147, wherein the first dose is about 4 mg/kg of the composition.
150. The composition of any one of embodiments 140-149, wherein the anti-TNFα antibody comprises a complementarity determining region (CDR) 100% identical to SEQ ID NO: 3, 4, 5, 6, 7, 8, 10, 12, 13, 14, or 15, or YA.
151. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 2.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
152. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 2.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
153. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 4.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
154. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 4.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
155. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 2.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
156. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 2.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
157. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 4.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
158. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 4.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.
159. The composition of any one of embodiments 140-158, wherein the method further comprises administering to the subject a second dose of the composition for a second period of time
160. The composition of embodiment 159, wherein the second period of time is about 6 weeks to about 16 weeks.
161. The composition of embodiment 60, wherein the second period of time is about 8 weeks.
162. The composition of embodiment 60, wherein the second period of time is about 12 weeks.
163. The composition of any one of embodiments 159-162, wherein the second dose comprises about 1 mg/kg to about 5 mg/kg.
164. The composition of embodiment 163, wherein the second dose is about 2 mg/kg.
165. The composition of embodiment 163, wherein the second dose is about 4 mg/kg.
166. The composition of embodiment 163, wherein the second dose is about two times greater than the first dose.
167. The composition of embodiment 166, wherein the administration frequency of the second dose comprises the same administration frequency of the first dose.
168. The composition of embodiment 166, wherein the administration frequency of the second dose consists of the same administration frequency of the first dose.
169. The composition of embodiment 167 or 168, wherein the first dose and the second dose are each administered about once every four weeks.
170. The composition of embodiment 159, wherein the second dose is about the same dose as the first dose.
171. The composition of embodiment 159, wherein the second dose is the same dose as the first dose.
172. The composition of embodiment 170 or 171, wherein the first dose and the second dose are about 2 mg/kg.
173. The composition of embodiment 170 or 171, wherein the administration frequency of the second dose is about two times greater than the administration frequency of the first dose.
174. The composition of embodiment 172 or 173, wherein the first dose is administered about once every four weeks and the second dose is administered about once every two weeks.
175. A composition comprising an anti-TNFα antibody sialylated with one or more N-acetylneuraminic acid molecules for use in a method of treating or reducing sarcoidosis in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.
176. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.
177. The composition of embodiment 175 or 176, wherein the first period of time is about 6 weeks to about 12 weeks.
178. The composition of embodiment 177, wherein the first period of time is about 8 weeks.
179. The composition of embodiment 177, wherein the first period of time is about 12 weeks.
180. The composition of any one of embodiments 175-179, wherein the second period of time is about 6 weeks to about 12 weeks.
181. The composition of embodiment 180, wherein the second period of time is about 8 weeks.
182. The composition of embodiment 180, wherein the second period of time is about 12 weeks.
183. A composition comprising an anti-TNFα antibody sialylated with one or more N-acetylneuraminic acid molecules for use in a method of treating or reducing sarcoidosis in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time,
   wherein the anti-TNFα antibody comprises:
   (i) a first CDR having an amino acid sequence at least about 80%, 85%, 90%, 95%, 99%, or 100% identical to SEQ ID NO: 3;
   (ii) a second CDR having an amino acid sequence at least about 80%, 85%, 90%, 95%, 99%, or 100% identical to SEQ ID NO: 4;
   (iii) a third CDR having an amino acid sequence at least about 80%, 85%, 90%, 95%, 99%, or 100% identical to SEQ ID NO: 5;
   (iv) a fourth CDR having an amino acid sequence at least about 80%, 85%, 90%, 95%, 99%, or 100% identical to SEQ ID NO: 6;
   (v) a fifth CDR having an amino acid sequence at least about 80%, 85%, 90%, 95%, 99%, or 100% identical to SEQ ID NO: 7;
   (vi) a sixth CDR having an amino acid sequence at least about 80%, 85%, 90%, 95%, 99%, or 100% identical to SEQ ID NO: 8;
   (vii) a seventh CDR having an amino acid sequence at least about 80%, 85%, 90%, 95%, 99%, or 100% identical to SEQ ID NO: 10;
   (viii) an eighth CDR having an amino acid sequence at least about 80%, 85%, 90%, 95%, 99%, or 100% identical to YA;
   (ix) a ninth CDR having an amino acid sequence at least about 80%, 85%, 90%, 95%, 99%, or 100% identical to SEQ ID NO: 12;
   (x) a tenth CDR having an amino acid sequence at least about 80%, 85%, 90%, 95%, 99%, or 100% identical to SEQ ID NO: 13;
   (xi) an eleventh CDR having an amino acid sequence at least about 80%, 85%, 90%, 95%, 99%, or 100% identical to SEQ ID NO: 14; and
   (xii) a twelfth CDR having an amino acid sequence at least about 80%, 85%, 90%, 95%, 99%, or 100% identical to SEQ ID NO: 15.
184. A composition comprising an anti-TNFα antibody sialylated with one or more N-acetylneuraminic acid molecules for use in a method of treating or reducing sarcoidosis in a subject in need thereof relative to a control, the method comprising administering to the subject a first dose and a second dose of the composition;
the first dose comprising about 2 mg/kg of the composition administered once every two weeks for a first period of time; and
the second dose comprising (i) about 2 mg/kg of the composition administered once every two weeks, or (iii) about 4 mg/kg of the composition administered once every 4 weeks, for a second period of time,
   wherein the anti-TNFα antibody comprises:
   (i) a first CDR having an amino acid sequence at least about 80%, 85%, 90%, 95%, 99%, or 100% identical to SEQ ID NO: 3;
   (ii) a second CDR having an amino acid sequence at least about 80%, 85%, 90%, 95%, 99%, or 100% identical to SEQ ID NO: 4;
   (iii) a third CDR having an amino acid sequence at least about 80%, 85%, 90%, 95%, 99%, or 100% identical to SEQ ID NO: 5;
   (iv) a fourth CDR having an amino acid sequence at least about 80%, 85%, 90%, 95%, 99%, or 100% identical to SEQ ID NO: 6;
   (v) a fifth CDR having an amino acid sequence at least about 80%, 85%, 90%, 95%, 99%, or 100% identical to SEQ ID NO: 7;
   (vi) a sixth CDR having an amino acid sequence at least about 80%, 85%, 90%, 95%, 99%, or 100% identical to SEQ ID NO: 8;
   (vii) a seventh CDR having an amino acid sequence at least about 80%, 85%, 90%, 95%, 99%, or 100% identical to SEQ ID NO: 10;
   (viii) an eighth CDR having an amino acid sequence at least about 80%, 85%, 90%, 95%, 99%, or 100% identical to YA;
   (ix) a ninth CDR having an amino acid sequence at least about 80%, 85%, 90%, 95%, 99%, or 100% identical to SEQ ID NO: 12;
   (x) a tenth CDR having an amino acid sequence at least about 80%, 85%, 90%, 95%, 99%, or 100% identical to SEQ ID NO: 13;
   (xi) an eleventh CDR having an amino acid sequence at least about 80%, 85%, 90%, 95%, 99%, or 100% identical to SEQ ID NO: 14; and
   (xii) a twelfth CDR having an amino acid sequence at least about 80%, 85%, 90%, 95%, 99%, or 100% identical to SEQ ID NO: 15.
185. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time,
wherein the anti-TNFα antibody comprises:
   (i) a first CDR having an amino acid sequence at least about 80%, 85%, 90%, 95%, 99%, or 100% identical to SEQ ID NO: 3;
   (ii) a second CDR having an amino acid sequence at least about 80%, 85%, 90%, 95%, 99%, or 100% identical to SEQ ID NO: 4;
   (iii) a third CDR having an amino acid sequence at least about 80%, 85%, 90%, 95%, 99%, or 100% identical to SEQ ID NO: 5;
   (iv) a fourth CDR having an amino acid sequence at least about 80%, 85%, 90%, 95%, 99%, or 100% identical to SEQ ID NO: 6;
   (v) a fifth CDR having an amino acid sequence at least about 80%, 85%, 90%, 95%, 99%, or 100% identical to SEQ ID NO: 7;
   (vi) a sixth CDR having an amino acid sequence at least about 80%, 85%, 90%, 95%, 99%, or 100% identical to SEQ ID NO: 8;
   (vii) a seventh CDR having an amino acid sequence at least about 80%, 85%, 90%, 95%, 99%, or 100% identical to SEQ ID NO: 10;
   (viii) an eighth CDR having an amino acid sequence at least about 80%, 85%, 90%, 95%, 99%, or 100% identical to YA;
   (ix) a ninth CDR having an amino acid sequence at least about 80%, 85%, 90%, 95%, 99%, or 100% identical to SEQ ID NO: 12;
   (x) a tenth CDR having an amino acid sequence at least about 80%, 85%, 90%, 95%, 99%, or 100% identical to SEQ ID NO: 13;
   (xi) an eleventh CDR having an amino acid sequence at least about 80%, 85%, 90%, 95%, 99%, or 100% identical to SEQ ID NO: 14; and
   (xii) a twelfth CDR having an amino acid sequence at least about 80%, 85%, 90%, 95%, 99%, or 100% identical to SEQ ID NO: 15.
186. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time,
   wherein the anti-TNFα antibody comprises:
   (i) a first CDR having an amino acid sequence at least about 80%, 85%, 90%, 95%, 99%, or 100% identical to SEQ ID NO: 3;
   (ii) a second CDR having an amino acid sequence at least about 80%, 85%, 90%, 95%, 99%, or 100% identical to SEQ ID NO: 4;
   (iii) a third CDR having an amino acid sequence at least about 80%, 85%, 90%, 95%, 99%, or 100% identical to SEQ ID NO: 5;
   (iv) a fourth CDR having an amino acid sequence at least about 80%, 85%, 90%, 95%, 99%, or 100% identical to SEQ ID NO: 6;
   (v) a fifth CDR having an amino acid sequence at least about 80%, 85%, 90%, 95%, 99%, or 100% identical to SEQ ID NO: 7;
   (vi) a sixth CDR having an amino acid sequence at least about 80%, 85%, 90%, 95%, 99%, or 100% identical to SEQ ID NO: 8;
   (vii) a seventh CDR having an amino acid sequence at least about 80%, 85%, 90%, 95%, 99%, or 100% identical to SEQ ID NO: 10;
   (viii) an eighth CDR having an amino acid sequence at least about 80%, 85%, 90%, 95%, 99%, or 100% identical to YA;
   (ix) a ninth CDR having an amino acid sequence at least about 80%, 85%, 90%, 95%, 99%, or 100% identical to SEQ ID NO: 12;
   (x) a tenth CDR having an amino acid sequence at least about 80%, 85%, 90%, 95%, 99%, or 100% identical to SEQ ID NO: 13;
   (xi) an eleventh CDR having an amino acid sequence at least about 80%, 85%, 90%, 95%, 99%, or 100% identical to SEQ ID NO: 14; and
   (xii) a twelfth CDR having an amino acid sequence at least about 80%, 85%, 90%, 95%, 99%, or 100% identical to SEQ ID NO: 15.
187. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every two weeks for a first period of time; and
the second dose comprising (i) about 2 mg/kg of the composition administered once every two weeks, or (iii) about 4 mg/kg of the composition administered once every 4 weeks, for a second period of time,
   wherein the anti-TNFα antibody comprises:
   (i) a first CDR having an amino acid sequence at least about 80%, 85%, 90%, 95%, 99%, or 100% identical to SEQ ID NO: 3;
   (ii) a second CDR having an amino acid sequence at least about 80%, 85%, 90%, 95%, 99%, or 100% identical to SEQ ID NO: 4;
   (iii) a third CDR having an amino acid sequence at least about 80%, 85%, 90%, 95%, 99%, or 100% identical to SEQ ID NO: 5;
   (iv) a fourth CDR having an amino acid sequence at least about 80%, 85%, 90%, 95%, 99%, or 100% identical to SEQ ID NO: 6;
   (v) a fifth CDR having an amino acid sequence at least about 80%, 85%, 90%, 95%, 99%, or 100% identical to SEQ ID NO: 7;
   (vi) a sixth CDR having an amino acid sequence at least about 80%, 85%, 90%, 95%, 99%, or 100% identical to SEQ ID NO: 8;
   (vii) a seventh CDR having an amino acid sequence at least about 80%, 85%, 90%, 95%, 99%, or 100% identical to SEQ ID NO: 10;
   (viii) an eighth CDR having an amino acid sequence at least about 80%, 85%, 90%, 95%, 99%, or 100% identical to YA;
   (ix) a ninth CDR having an amino acid sequence at least about 80%, 85%, 90%, 95%, 99%, or 100% identical to SEQ ID NO: 12;
   (x) a tenth CDR having an amino acid sequence at least about 80%, 85%, 90%, 95%, 99%, or 100% identical to SEQ ID NO: 13;
   (xi) an eleventh CDR having an amino acid sequence at least about 80%, 85%, 90%, 95%, 99%, or 100% identical to SEQ ID NO: 14; and
   (xii) a twelfth CDR having an amino acid sequence at least about 80%, 85%, 90%, 95%, 99%, or 100% identical to SEQ ID NO: 15.
188. The composition of any one of embodiments 160-187, further comprising administering (c) a third dose of about 4 mg/kg of the composition about once every two weeks, for a third period of time.
189. The composition of embodiment 187, wherein the third period of time is at least about 24 weeks.
190. The composition of any one of embodiments 140-189, wherein the anti-TNFα antibody comprises a variable heavy (VH) domain having an amino acid sequence at least about 80%, 85%, 90%, 95%, 99%, or 100% identical to SEQ ID NO: 16.
191. The composition of any one of embodiments 140-189, wherein the anti-TNFα antibody comprises a variable light (VL) domain having an amino acid sequence at least about 80%, 85%, 90%, 95%, 99%, or 100% identical to SEQ ID NO: 9.
192. The composition of any one of embodiments 140-191, wherein the anti-TNFα antibody comprises an asparagine-linked glycosylation site within a constant Fc region on at least one of the heavy chains.
193. The composition of embodiment 192, wherein the anti-TNFα antibody comprises an asparagine-linked glycosylation site within the constant Fc region on both of the heavy chains.
194. The composition of any one of embodiments 140-193, wherein the anti-TNFα antibody is sialylated at Asn 297.
195. The composition of any one of embodiments 140-194, wherein the anti-TNFα antibody comprises no Neu5Gc.
196. The composition of any one of embodiments 140-195, wherein the anti-TNFα antibody is an IgG antibody.
197. The composition of embodiment 196, wherein the IgG antibody is an IgG1 antibody.
198. The composition of embodiment 197, wherein the IgG1 antibody comprises a kappa isotype.
199. The composition of any one of embodiments 140-198, wherein the anti-TNFα antibody is a chimeric antibody.
200. The composition of any one of embodiments 140-199, wherein the anti-TNFα antibody comprises a C-terminal lysine on at least one of the heavy chains.
201. The composition of embodiment 200, wherein the anti-TNFα antibody comprises a C-terminal lysine on both of the heavy chains.
202. The composition of embodiment 200 or 201, wherein the C-terminal lysine is not clipped.
203. The composition of any one of embodiments 140-202, wherein the composition is administered by an infusion.
204. The composition of embodiment 203, wherein the infusion is an intravenous (IV) infusion.
205. The composition of embodiment 203 or 204, wherein the infusion is administered over a period of at least about 2 hours.
206. The composition of embodiment 203 or 204, wherein the infusion is administered over a period of at about 2 hours.
207. The composition of any one of embodiments 140-206, wherein the subject receives a cumulative dose of about 6 mg/kg of the composition during the first period of time.
208. The composition of any one of embodiments 140-206, wherein the subject receives a cumulative dose of about 8 mg/kg of the composition during the first period of time.
209. The composition of any one of embodiments 140-206, wherein the subject receives a cumulative dose of about 16 mg/kg of the composition during the first period of time.
210. The composition of any one of embodiments 181-209, wherein the subject receives a cumulative dose of about 10 mg/kg to about 16 mg/kg of the composition during the second period of time.
211. The composition of embodiment 210, wherein the subject receives a cumulative dose of about 10 mg/kg of the composition during the second period of time.
212. The composition of embodiment 210, wherein the subject receives a cumulative dose of about 12 mg/kg of the composition during the second period of time.
213. The composition of embodiment 210, wherein the subject receives a cumulative dose of about 14 mg/kg of the composition during the second period of time.
214. The composition of embodiment 210, wherein the subject receives a cumulative dose of about 16 mg/kg of the composition during the second period of time.
215. The composition of any one of embodiments 188-214, wherein the subject receives a cumulative dose of about 28 mg/kg of the composition.
216. The composition of any one of embodiments 140-215, wherein the subject is administered a cumulative dose of about 30 mg/kg to about 60 mg/kg of the composition.
217. The composition of embodiment 216, wherein the subject is administered a cumulative dose of about 46 mg/kg of the composition.
218. The composition of embodiment 216, wherein the subject is administered a cumulative dose of about 48 mg/kg of the composition.
219. The composition of embodiment 216, wherein the subject is administered a cumulative dose of about 50 mg/kg of the composition.
220. The composition of embodiment 216, wherein the subject is administered a cumulative dose of about 52 mg/kg of the composition.
221. The composition of any one of embodiments 140-220, wherein the subject is administered at least 3 doses of the composition.
222. The composition of any one of embodiments 140-221, wherein the subject is administered at least 4 doses of the composition.
223. The composition of any one of embodiments 140-222, wherein the subject is administered at least 6 doses of the composition.
224. The composition of any one of embodiments 140-223, wherein the subject is administered at least 7 doses of the composition.
225. The composition of embodiment 221, wherein the at least 3 doses are administered over a 4-week period of time.
226. The composition of embodiment 221, wherein the at least 3 doses are administered over an 8-week period of time.
227. The composition of embodiment 221, wherein the at least 3 doses are administered over a 12-week period of time.
228. The composition of embodiment 222, wherein the at least 4 doses are administered over a 9-week period of time.
229. The composition of embodiment 222, wherein the at least 4 doses are administered over a 10-week period of time.
230. The composition of embodiment 222, wherein the at least 4 doses are administered over an 11-week period of time.
231. The composition of embodiment 222, wherein the at least 4 doses are administered over a 12-week period of time.
232. The composition of embodiment 223, wherein the at least 6 doses are administered over a 10-week period of time.
233. The composition of embodiment 223, wherein the at least 6 doses are administered over a 13-week period of time.
234. The composition of embodiment 223, wherein the at least 6 doses are administered over a 16-week period of time.
235. The composition of embodiment 223, wherein the at least 6 doses are administered over a 17-week period of time.
236. The composition of embodiment 223, wherein the at least 6 doses are administered over a 20-week period of time.
237. The composition of embodiment 234, wherein the at least 7 doses are administered over a 12-week period of time.
238. The composition of embodiment 234, wherein the at least 7 doses are administered over a 15-week period of time.
239. The composition of embodiment 234, wherein the at least 7 doses are administered over a 21-week period of time.
240. The composition of embodiment 234, wherein the at least 7 doses are administered over a 24-week period of time.
241. The composition of embodiment 234, wherein the at least 7 doses are administered over a 28-week period of time.
242. The composition of any one of embodiments 140-241, wherein administration of the composition reduces a level of one or more transcripts selected from the group consisting of angiotensin-converting enzyme 2 (ACE2), interleukin 6 (IL6), interleukin 2 receptor (IL2R), tumor necrosis factor alpha (TNFα), C-Reactive Protein (CRP), interleukin 1B (IL1B), interleukin 12 (IL12), interleukin 18 (IL18), interferon gamma (IFNG), interleukin 8 (IL8), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JunB), mitogen-activated protein kinase (MKP1), TNF alpha induced protein 1 (TNFAIP1), TNF alpha induced protein 2 (TNFAIP2), TNF alpha induced protein 3 (TNFAIP3), syndecan 4 (SDC4), superoxide dismutase 2 (SOD2), cytochrome c oxidase subunit II (COX2), interleukin 32 (IL32), intercellular adhesion molecule 1 (ICAM-1), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), and vascular adhesion molecule 1 (VCAM-1), relative to a control.
243. The composition of embodiment 242, wherein the level of the one or more transcripts is reduced by at least about 10%, 20%, 30%, 40%, 50%, 60%, 70%, 80%, 90%, or 100% relative to the control.
244. The composition of any one of embodiments 140-243, wherein administration of the composition reduces a level of one or more proteins selected from the group consisting of angiotensin-converting enzyme 2 (ACE2), interleukin 6 (IL-6), interleukin 2 receptor (IL-2R), tumor necrosis factor alpha (TNF-α), C-Reactive Protein (CRP), interleukin 1B (IL-1B), interferon gamma (IFN-γ), interleukin 8 (IL-8), interleukin 12 (IL-12), interleukin 18 (IL-18), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JUNB), mitogen-activated protein kinase phosphatase 1 (MKP-1), TNF alpha-induced protein 1 (TNFAIP1), TNF alpha-induced protein 2 (TNFAIP2), TNF alpha-induced protein 3 (TNFAIP3), syndecan 4 (SDC4), superoxide dismutase 2 (SOD2), cytochrome c oxidase subunit II (COX2), interleukin 32 (IL-32), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), intercellular adhesion molecule 1 (ICAM-1), and vascular adhesion molecule 1 (VCAM-1) relative to a control.
245. The composition of embodiment 244, wherein the level of the one or more proteins is reduced by at least about 10%, 20%, 30%, 40%, 50%, 60%, 70%, 80%, 90%, or 100% relative to the control.
246. The composition of any one of embodiments 140-245, wherein administration of the composition increases one or more of a Forced Vital Capacity (FVC) measurement, a Forced Expiratory Volume in 1 second (FEV1) measurement, a Leicester Cough Questionnaire (LCQ) score, a Short Form 36 Health Survey (SF-36) score, a King's Sarcoidosis Questionnaire (KSQ) score, a Steroid-toxicity scale (STS) score, or a 6-minute walk test (6MWT) distance relative to a control.
247. The composition of embodiment 246, wherein the FVC measurement is increased by at least about 10%, 20%, 30%, 40%, 50%, 60%, 70%, 80%, 90%, or 100% relative to the control.
248. The composition of embodiment 246, wherein the FEV1 measurement is increased by at least about 10%, 20%, 30%, 40%, 50%, 60%, 70%, 80%, 90%, or 100% relative to the control.
249. The composition of embodiment 246, wherein the LCQ score is increased by at least about 1, 2, 3, 4, 5, 6, 7, 8, 9, 10, 11, 12, 13, 14, 15, 16, 17, or 18 points relative to the control.
250. The composition of embodiment 246, wherein the SF-36 score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.
251. The composition of embodiment 246, wherein the KSQ score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.
252. The composition of embodiment 246 or 251, wherein the KSQ score is a KSQ General Health Status Module or a KSQ lung score.
253. The composition of embodiment 246, wherein the STS score is increased by at least 1, 2, 3, 4, 5, or 6 points relative to the control.
254. The composition of embodiment 246, wherein the 6MWT distance is increased by at least 10 m, 20 m, 30 m, 40 m, 50 m, 60 m, 70 m, 80 m, 90 m, 100 m, 125 m, 150 m, 175 m, 200 m, 250 m, 300 m, 350 m, 400 m, 450 m, or 500 m relative to the control.
255. The composition of any one of embodiments 140-254, wherein administration of the composition reduces one or more of a Patient Global Assessment (PGA) score, a Fatigue Assessment Scale (FAS) score, Modified Medical Research Council (mMRC) Dyspnea Scale score, a Saint George's Respiratory Questionnaire (SGRQ) score, or a Borg's CR10 dyspnea score relative to a control.
256. The composition of embodiment 255, wherein the PGA score is reduced by at least about 1, 2, 3, 4, 5, 6, 7, 8, 9, or 10 points relative to the control.
257. The composition of embodiment 255, wherein the FAS score is reduced by at least about 1, 2, 3, or 4 points relative to a control.
258. The composition of embodiment 255, wherein the mMRC Scale score is reduced by at least about 1, 2, 3, or 4 grades relative to the control
259. The composition of embodiment 255, wherein the SGRQ score is reduced by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.
260. The composition of embodiment 255, wherein the Borg's CR10 dyspnea score is reduced by at least about 1, 2, 3, 4, 5, 6, 7, 8, 9, or 10 points relative to the control.
261. The composition of any one of embodiments 140-260, wherein administration of the composition reduces a need for an anti-inflammatory medication relative to a control.
262. The composition of embodiment 261, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.
263. The composition of embodiment 262, wherein the corticosteroid is selected from the group consisting of prednisone, prednisolone, dexamethasone, hydrocortisone, methylprednisolone, betamethasone, cortisone, fludrocortisone, and triamcinolone.
264. The composition of any one of embodiments 261-263, wherein the reduced need for anti-inflammatory medication comprises a reduction in frequency of, dosage of, or duration of administration of the anti-inflammatory medication.
265. The composition of any one of embodiments 140-264, wherein administration of the composition reduces a granuloma formation or a granuloma size in the subject by about 1%, 2%, 3%, 4%, 5%, 10%, 15%, 20%, 25%, 30%, 40%, 50%, 60%, 70%, 80%, 90%, or 100%, relative to a control.
266. The composition of embodiment 265, wherein the granuloma is a skin granuloma, a lung granuloma, a lymph node granuloma, an ocular granuloma, a liver granuloma, a spleen granuloma, a cardiac granuloma, or a rheumatoid granuloma.
267. The composition of any one of embodiments 140-266, wherein administration of the composition reduces a calcitriol level in the subject relative to a control.
268. The composition of embodiment 267, wherein the calcitriol level is reduced by at least about 5%, 10%, 15%, 20%, 25%, 30%, 40%, 50%, 60%, 70%, 80%, 90%, or 100% relative to the control.
269. The composition of any one of embodiments 140-268, wherein administration of the composition reduces a neopterin level in the subject relative to a control.
270. The composition of embodiment 260, wherein the neopterin level is reduced by at least about 5%, 10%, 15%, 20%, 25%, 30%, 40%, 50%, 60%, 70%, 80%, 90%, or 100% relative to the control.
271. The composition of any one of embodiments 140-270, wherein administration of the composition reduces a lysozyme level in the subject relative to a control.
272. The composition of embodiment 271, wherein the lysozyme level is reduced by at least about 5%, 10%, 15%, 20%, 25%, 30%, 40%, 50%, 60%, 70%, 80%, 90%, or 100% relative to the control.
273. The composition of any one of embodiments 140-272, wherein administration of the composition reduces a soluble IL-2R (sIL-2R) level in the subject relative to a control.
274. The composition of embodiment 273, wherein the sIL-2R level is reduced by at least about 5%, 10%, 15%, 20%, 25%, 30%, 40%, 50%, 60%, 70%, 80%, 90%, or 100% relative to the control.
275. The composition of any one of embodiments 140-274, wherein the subject has one or more of acute sarcoidosis, chronic sarcoidosis, progressive sarcoidosis, pulmonary sarcoidosis, remitting sarcoidosis, refractory sarcoidosis, advanced sarcoidosis, ocular sarcoidosis, cutaneous sarcoidosis, or neurosarcoidosis.
276. The composition of any one of embodiments 140-275, wherein the subject has received an anti-inflammatory medication.
277. The composition of any one of embodiments 140-276, wherein the subject is receiving an anti-inflammation medication.
278. The composition of embodiment 276 or 277, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.
279. The composition of embodiment 278, wherein the corticosteroid is selected from the group consisting of prednisone, prednisolone, dexamethasone, hydrocortisone, methylprednisolone, betamethasone, cortisone, fludrocortisone, and triamcinolone.

I/We claim:

1. A method of treating or reducing sarcoidosis in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every four weeks for a first period of time.

2. A method of treating or reducing sarcoidosis in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every two weeks for a first period of time.

3. A method of reducing granuloma size or granuloma formation in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every four weeks for a first period of time.

4. A method of reducing granuloma size or granuloma formation in a subject in need thereof relative to a control, the method comprising administering to the subject a first dose of about 0.5 mg/kg to about 6.0 mg/kg of a composition comprising an anti-TNFα antibody sialylated with one or more N-acetylneuraminic acid molecules about once every two weeks for a first period of time.

5. The method of any one of claims 1-4, wherein the first period of time is about 6 weeks to about 24 weeks.

6. The method of claim 5, wherein the first period of time is about 8 weeks.

7. The method of claim 5, wherein the first period of time is about 12 weeks.

8. The method of claim 5, wherein the first period of time is about 24 weeks.

9. The method of any one of claims 1-8, wherein the first dose is about 2 mg/kg of the composition.

10. The method of any one of claims 1-8, wherein the first dose is about 4 mg/kg of the composition.

11. The method of any one of claims 1-10, wherein the anti-TNFα antibody comprises a complementarity determining region (CDR) 100% identical to SEQ ID NO: 3, 4, 5, 6, 7, 8, 10, 12, 13, 14, or 15, or YA.

12. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time, and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

13. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

14. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every four weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

15. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

16. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

17. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

18. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every four weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

19. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 4 mg/kg of the composition administered once every two weeks for a first period of time; and
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

20. The method of any one of claims 1-19, further comprising administering to the subject a second dose of the composition for a second period of time.

21. The method of claim 20, wherein the second period of time is about 6 weeks to about 16 weeks.

22. The method of claim 21, wherein the second period of time is about 8 weeks.

23. The method of claim 21, wherein the second period of time is about 12 weeks.

24. The method of any one of claims 20-23, wherein the second dose comprises about 1 mg/kg to about 5 mg/kg.

25. The method of claim 24, wherein the second dose is about 2 mg/kg.

26. The method of claim 24, wherein the second dose is about 4 mg/kg.

27. The method of claim 24, wherein the second dose is about two times greater than the first dose.

28. The method of claim 27, wherein the administration frequency of the second dose comprises the same administration frequency of the first dose.

29. The method of claim 27, wherein the administration frequency of the second dose consists of the same administration frequency of the first dose.

30. The method of claim 28 or 29, wherein the first dose and the second dose are each administered about once every four weeks.

31. The method of claim 20, wherein the second dose is about the same dose as the first dose.

32. The method of claim 20, wherein the second dose is the same dose as the first dose.

33. The method of claim 31 or 32, wherein the first dose and the second dose are about 2 mg/kg.

34. The method of claim 31 or 32, wherein the administration frequency of the second dose is about two times greater than the administration frequency of the first dose.

35. The method of claim 33 or 34, wherein the first dose is administered about once every four weeks and the second dose is administered about once every two weeks.

36. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.

37. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.

38. The method of claim 36 or 37, wherein the first period of time is about 6 weeks to about 12 weeks.

39. The method of claim 38, wherein the first period of time is about 8 weeks.

40. The method of claim 38, wherein the first period of time is about 12 weeks.

41. The method of any one of claims 36-40, wherein the second period of time is about 6 weeks to about 12 weeks.

42. The method of claim 41, wherein the second period of time is about 8 weeks.

43. The method of claim 41, wherein the second period of time is about 12 weeks.

44. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

45. A method of treating or reducing sarcoidosis in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time; and
the second dose comprising (i) about 2 mg/kg of the composition administered once every two weeks, or (iii) about 4 mg/kg of the composition administered once every four weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

46. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every four weeks for a first period of time;
the second dose comprising (i) about 2 mg/kg of the composition administered once every two weeks, or (iii) about 4 mg/kg of the composition administered once every four weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

47. A method of reducing granuloma size or granuloma formation in a subject in need thereof relative to a control, the method comprising administering to the subject a first dose and a second dose of a composition comprising an anti-TNFα antibody sialylated with one or more N-acetylneuraminic acid molecules;
the first dose comprising about 2 mg/kg of the composition administered once every two weeks for a first period of time;
the second dose comprising (i) about 2 mg/kg of the composition administered once every two weeks, or (iii) about 4 mg/kg of the composition administered once every four weeks, for a second period of time;
the anti-TNFα antibody comprising:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

48. The method of any one of claims 20-47, further comprising administering (c) a third dose of about 4 mg/kg of the composition about once every two weeks, for a third period of time.

49. The method of claim 48, wherein the third period of time is at least about 24 weeks.

50. The method of any one of claims 1-49, wherein the anti-TNFα antibody comprises a variable heavy (V_{H}) domain having an amino acid sequence at least about 80%, 85%, 90%, 95%, 99%, or 100% identical to SEQ ID NO: 16.

51. The method of any one of claims 1-49, wherein the anti-TNFα antibody comprises a variable light (V_{L}) domain having an amino acid sequence at least about 80%, 85%, 90%, 95%, 99%, or 100% identical to SEQ ID NO: 9.

52. The method of any one of claims 1-51, wherein the anti-TNFα antibody comprises an asparagine-linked glycosylation site within a constant Fc region on at least one of the heavy chains.

53. The method of claim 52, wherein the anti-TNFα antibody comprises an asparagine-linked glycosylation site within the constant Fc region on both of the heavy chains.

54. The method of any one of claims 50-53, wherein the anti-TNFα antibody is sialylated at Asn 297.

55. The method of any one of claims 1-54, wherein the anti-TNFα antibody comprises no N-Glycolylneuraminic acid (Neu5Gc).

56. The method of any one of claims 1-55, wherein the anti-TNFα antibody is an immunoglobulin G (IgG) antibody.

57. The method of claim 43, wherein the IgG antibody is an IgG1 antibody.

58. The method of claim 57, wherein the IgG1 antibody comprises a kappa isotype.

59. The method of any one of claims 1-58, wherein the anti-TNFα antibody is a chimeric antibody.

60. The method of any one of claims 1-59, wherein the anti-TNFα antibody comprises a C-terminal lysine on at least one of the heavy chains.

61. The method of claim 60, wherein the anti-TNFα antibody comprises a C-terminal lysine on both of the heavy chains.

62. The method of claim 60 or 61, wherein the C-terminal lysine is not clipped.

63. The method of any one of claims 1-62, wherein the composition is administered by an infusion.

64. The method of claim 63, wherein the infusion is an intravenous (IV) infusion.

65. The method of claim 63 or 64, wherein the infusion is administered over a period of at least about 2 hours.

66. The method of claim 63 or 64, wherein the infusion is administered over a period of about 2 hours.

67. The method of any one of claims 1-66, wherein the subject receives a cumulative dose of about 6 mg/kg of the composition during the first period of time.

68. The method of any one of claims 1-66, wherein the subject receives a cumulative dose of about 8 mg/kg of the composition during the first period of time.

69. The method of any one of claims 1-66, wherein the subject receives a cumulative dose of about 16 mg/kg of the composition during the first period of time.

70. The method of any one of claims 41-69, wherein the subject receives a cumulative dose of about 10 mg/kg to about 16 mg/kg of the composition during the second period of time.

71. The method of claim 70, wherein the subject receives a cumulative dose of about 10 mg/kg of the composition during the second period of time.

72. The method of claim 70, wherein the subject receives a cumulative dose of about 12 mg/kg of the composition during the second period of time.

73. The method of claim 70, wherein the subject receives a cumulative dose of about 14 mg/kg of the composition during the second period of time.

74. The method of claim 70, wherein the subject receives a cumulative dose of about 16 mg/kg of the composition during the second period of time.

75. The method of any one of claims 48-74, wherein the subject receives a cumulative dose of about 28 mg/kg of the composition.

76. The method of any one of claims 1-75, wherein the subject is administered a cumulative dose of about 30 mg/kg to about 60 mg/kg of the composition.

77. The method of claim 76, wherein the subject is administered a cumulative dose of about 46 mg/kg of the composition.

78. The method of claim 76, wherein the subject is administered a cumulative dose of about 48 mg/kg of the composition.

79. The method of claim 76, wherein the subject is administered a cumulative dose of about 50 mg/kg of the composition.

80. The method of claim 76, wherein the subject is administered a cumulative dose of about 52 mg/kg of the composition.

81. The method of any one of claims 1-80, wherein the subject is administered at least 3 doses of the composition.

82. The method of any one of claims 1-81, wherein the subject is administered at least 4 doses of the composition.

83. The method of any one of claims 1-82, wherein the subject is administered at least 6 doses of the composition.

84. The method of any one of claims 1-83, wherein the subject is administered at least 7 doses of the composition.

85. The method of claim 81, wherein the at least 3 doses are administered over a 4-week period of time.

86. The method of claim 81, wherein the at least 3 doses are administered over an 8-week period of time.

87. The method of claim 81, wherein the at least 3 doses are administered over a 12-week period of time.

88. The method of claim 82, wherein the at least 4 doses are administered over a 9-week period of time.

89. The method of claim 82, wherein the at least 4 doses are administered over a 10-week period of time.

90. The method of claim 82, wherein the at least 4 doses are administered over an 11-week period of time.

91. The method of claim 82, wherein the at least 4 doses are administered over a 12-week period of time.

92. The method of claim 83, wherein the at least 6 doses are administered over a 10-week period of time.

93. The method of claim 83, wherein the at least 6 doses are administered over a 13-week period of time.

94. The method of claim 83, wherein the at least 6 doses are administered over a 16-week period of time.

95. The method of claim 83, wherein the at least 6 doses are administered over a 17-week period of time.

96. The method of claim 83, wherein the at least 6 doses are administered over a 20-week period of time.

97. The method of claim 84, wherein the at least 7 doses are administered over a 12-week period of time.

98. The method of claim 84, wherein the at least 7 doses are administered over a 15-week period of time.

99. The method of claim 84, wherein the at least 7 doses are administered over a 21-week period of time.

100. The method of claim 82, wherein the at least 7 doses are administered over a 28-week period of time.

101. The method of claim 84, wherein the at least 7 doses are administered over a 24-week period of time.

102. The method of any one of claims 1-101, wherein administration of the composition reduces a level of one or more transcripts selected from the group consisting of angiotensin-converting enzyme 2 (ACE2), interleukin 6 (IL6), interleukin 2 receptor (IL2R), tumor necrosis factor alpha (TNFα), C-Reactive Protein (CRP), interleukin 1B (IL1B), interleukin 12 (IL12), interleukin 18 (IL18), interferon gamma (IFNG), interleukin 8 (IL8), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JunB), mitogen-activated protein kinase (MKP1), TNF alpha induced protein 1 (TNFAIP1), TNF alpha induced protein 2 (TNFAIP2), TNF alpha induced protein 3 (TNFAIP3), syndecan 4 (SDC4), superoxide dismutase 2 (SOD2), cytochrome c oxidase subunit II (COX2), interleukin 32 (IL32), intercellular adhesion molecule 1 (ICAM-1), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), and vascular adhesion molecule 1 (VCAM-1), relative to a control.

103. The method of claim 102, wherein the level of the one or more transcripts is reduced by at least about 10%, 20%, 30%, 40%, 50%, 60%, 70%, 80%, 90%, or 100% relative to the control.

104. The method of any one of claims 1-103, wherein administration of the composition reduces a level of one or more proteins selected from the group consisting of angiotensin-converting enzyme 2 (ACE2), interleukin 6 (IL-6), interleukin 2 receptor (IL-2R), tumor necrosis factor alpha (TNF-α), C-Reactive Protein (CRP), interleukin 1B (IL-1B), interferon gamma (IFN-γ), interleukin 8 (IL-8), interleukin 12 (IL-12), interleukin 18 (IL-18), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JUNB), mitogen-activated protein kinase phosphatase 1 (MKP-1), TNF alpha-induced protein 1 (TNFAIP1), TNF alpha-induced protein 2 (TNFAIP2), TNF alpha-induced protein 3 (TNFAIP3), syndecan 4 (SDC4), superoxide dismutase 2 (SOD2), cytochrome c oxidase subunit II (COX2), interleukin 32 (IL-32), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), intercellular adhesion molecule 1 (ICAM-1), and vascular adhesion molecule 1 (VCAM-1) relative to a control.

105. The method of claim 104, wherein the level of the one or more proteins is reduced by at least about 10%, 20%, 30%, 40%, 50%, 60%, 70%, 80%, 90%, or 100% relative to the control.

106. The method of any one of claims 1-105, wherein administration of the composition increases one or more of a Forced Vital Capacity (FVC) measurement, a Forced Expiratory Volume in 1 second (FEV1) measurement, a Leicester Cough Questionnaire (LCQ) score, a Short Form 36 Health Survey (SF-36) score, a King's Sarcoidosis Questionnaire (KSQ) score, a Steroid-toxicity scale (STS) score, or a 6-minute walk test (6MWT) distance relative to a control.

107. The method of claim 106, wherein the FVC measurement is increased by at least about 10%, 20%, 30%, 40%, 50%, 60%, 70%, 80%, 90%, or 100% relative to the control.

108. The method of claim 106, wherein the FEV1 measurement is increased by at least about 10%, 20%, 30%, 40%, 50%, 60%, 70%, 80%, 90%, or 100% relative to the control.

109. The method of claim 106, wherein the LCQ score is increased by at least about 1, 2, 3, 4, 5, 6, 7, 8, 9, 10, 11, 12, 13, 14, 15, 16, 17, or 18 points relative to the control.

110. The method of claim 106, wherein the SF-36 score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.

111. The method of claim 106, wherein the KSQ score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.

112. The method of claim 106 or 111, wherein the KSQ score is a KSQ General Health Status Module or a KSQ lung score.

113. The method of claim 106, wherein the STS score is increased by at least 1, 2, 3, 4, 5, or 6 points relative to the control.

114. The method of claim 106, wherein the 6MWT distance is increased by at least 10 m, 20 m, 30 m, 40 m, 50 m, 60 m, 70 m, 80 m, 90 m, 100 m, 125 m, 150 m, 175 m, 200 m, 250 m, 300 m, 350 m, 400 m, 450 m, or 500 m relative to the control.

115. The method of any one of claims 1-114, wherein administration of the composition reduces one or more of a Patient Global Assessment (PGA) score, a Fatigue Assessment Scale (FAS) score, Modified Medical Research Council (mMRC) Dyspnea Scale score, a Saint George's Respiratory Questionnaire (SGRQ) score, or a Borg's CR10 dyspnea score relative to a control.

116. The method of claim 115, wherein the PGA score is reduced by at least about 1, 2, 3, 4, 5, 6, 7, 8, 9, or 10 points relative to the control.

117. The method of claim 115, wherein the FAS score is reduced by at least about 1, 2, 3, or 4 points relative to a control.

118. The method of claim 115, wherein the mMRC Scale score is reduced by at least about 1, 2, 3, or 4 grades relative to the control.

119. The method of claim 115, wherein the SGRQ score is reduced by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.

120. The method of claim 115, wherein the Borg's CR10 dyspnea score is reduced by at least about 1, 2, 3, 4, 5, 6, 7, 8, 9, or 10 points relative to the control.

121. The method of any one of claims 1-120, wherein administration of the composition reduces a need for an anti-inflammatory medication relative to a control.

122. The method of claim 121, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.

123. The method of claim 122, wherein the corticosteroid is selected from the group consisting of prednisone, prednisolone, dexamethasone, hydrocortisone, methylprednisolone, betamethasone, cortisone, fludrocortisone, and triamcinolone.

124. The method of any one of claims 121-123, wherein the reduced need for anti-inflammatory medication comprises a reduction in frequency of, dosage of, or duration of administration of the anti-inflammatory medication.

125. The method of any one of claims 1-123, wherein administration of the composition reduces a granuloma formation or a granuloma size in the subject by about 1%, 2%, 3%, 4%, 5%, 10%, 15%, 20%, 25%, 30%, 40%, 50%, 60%, 70%, 80%, 90%, or 100%, relative to a control.

126. The method of claim 125, wherein the granuloma is a skin granuloma, a lung granuloma, a lymph node granuloma, an ocular granuloma, a liver granuloma, a spleen granuloma, a cardiac granuloma, or a rheumatoid granuloma.

127. The method of any one of claims 1-126, wherein administration of the composition reduces a calcitriol level in the subject relative to a control.

128. The method of claim 127, wherein the calcitriol level is reduced by at least about 5%, 10%, 15%, 20%, 25%, 30%, 40%, 50%, 60%, 70%, 80%, 90%, or 100% relative to the control.

129. The method of any one of claims 1-128, wherein administration of the composition reduces a neopterin level in the subject relative to a control.

130. The method of claim 129, wherein the neopterin level is reduced by at least about 5%, 10%, 15%, 20%, 25%, 30%, 40%, 50%, 60%, 70%, 80%, 90%, or 100% relative to the control.

131. The method of any one of claims 1-130, wherein administration of the composition reduces a lysozyme level in the subject relative to a control.

132. The method of claim 131, wherein the lysozyme level is reduced by at least about 5%, 10%, 15%, 20%, 25%, 30%, 40%, 50%, 60%, 70%, 80%, 90%, or 100% relative to the control.

133. The method of any one of claims 1-132, wherein administration of the composition reduces a soluble IL-2R (sIL-2R) level in the subject relative to a control.

134. The method of claim 132, wherein the sIL-2R level is reduced by at least about 5%, 10%, 15%, 20%, 25%, 30%, 40%, 50%, 60%, 70%, 80%, 90%, or 100% relative to the control.

135. The method of any one of claims 1-134, wherein the subject has one or more of acute sarcoidosis, chronic sarcoidosis, progressive sarcoidosis, pulmonary sarcoidosis, remitting sarcoidosis, refractory sarcoidosis, advanced sarcoidosis, ocular sarcoidosis, cutaneous sarcoidosis, or neurosarcoidosis.

136. The method of any one of claims 1-135, wherein the subject has received an anti-inflammatory medication.

137. The method of any one of claims 1-136, wherein the subject is receiving an anti-inflammation medication.

138. The method of claim 136 or 137, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.

139. The method of claim 138, wherein the corticosteroid is selected from the group consisting of prednisone, prednisolone, dexamethasone, hydrocortisone, methylprednisolone, betamethasone, cortisone, fludrocortisone, and triamcinolone.

140. A composition comprising an anti-TNFα antibody sialylated with one or more N-acetylneuraminic acid molecules for use in a method of treating or reducing sarcoidosis in a subject in need thereof relative to a control, wherein the composition is administered as a first dose of about 0.5 mg/kg to about 6.0 mg/kg about once every four weeks for a first period of time.

141. A composition comprising an anti-TNFα antibody sialylated with one or more N-acetylneuraminic acid molecules for use in a method of treating or reducing sarcoidosis in a subject in need thereof relative to a control, wherein the composition is administered as a first dose of about 0.5 mg/kg to about 6.0 mg/kg about once every two weeks for a first period of time.

142. A composition comprising an anti-TNFα antibody sialylated with one or more N-acetylneuraminic acid molecules for use in a method of reducing granuloma size or granuloma formation in a subject in need thereof relative to a control, wherein the composition is administered as a first dose of about 0.5 mg/kg to about 6.0 mg/kg about once every four weeks for a first period of time.

143. A composition comprising an anti-TNFα antibody sialylated with one or more N-acetylneuraminic acid molecules for use in a method of reducing granuloma size or granuloma formation in a subject in need thereof relative to a control, wherein the composition is administered as a first dose of about 0.5 mg/kg to about 6.0 mg/kg about once every two weeks for a first period of time.

144. The composition of any one of claims 140-143, wherein the first period of time is about 6 weeks to about 24 weeks.

145. The composition of claim 144, wherein the first period of time is about 8 weeks.

146. The composition of claim 144, wherein the first period of time is about 12 weeks.

147. The composition of claim 144, wherein the first period of time is about 24 weeks.

148. The composition of any one of claims 140-147, wherein the first dose is about 2 mg/kg of the composition.

149. The composition of any one of claims 140-147, wherein the first dose is about 4 mg/kg of the composition.

150. The composition of any one of claims 140-149, wherein the anti-TNFα antibody comprises a complementarity determining region (CDR) 100% identical to SEQ ID NO: 3, 4, 5, 6, 7, 8, 10, 12, 13, 14, or 15, or YA.

151. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 2.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

152. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 2.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

153. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 4.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

154. A composition comprising an anti-TNFα antibody sialylated with one or more N-acetylneuraminic acid molecules for use in a method of treating or reducing sarcoidosis in a subject in need thereof relative to a control,
wherein the composition is administered as a first dose of about 4.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

155. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 2.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

156. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 2.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

157. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 4.0 mg/kg about once every four weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

158. A composition comprising an anti-TNFα antibody sialylated with one or more N-acetylneuraminic acid molecules for use in a method of reducing granuloma size or granuloma formation in a subject in need thereof relative to a control,
wherein the composition is administered as a first dose of about 4.0 mg/kg about once every two weeks for a first period of time, and
the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

159. The composition of any one of claims 140-158, wherein the method further comprises administering to the subject a second dose of the composition for a second period of time

160. The composition of claim 159, wherein the second period of time is about 6 weeks to about 16 weeks.

161. The composition of claim 60, wherein the second period of time is about 8 weeks.

162. The composition of claim 60, wherein the second period of time is about 12 weeks.

163. The composition of any one of claims 159-162, wherein the second dose comprises about 1 mg/kg to about 5 mg/kg.

164. The composition of claim 163, wherein the second dose is about 2 mg/kg.

165. The composition of claim 163, wherein the second dose is about 4 mg/kg.

166. The composition of claim 163, wherein the second dose is about two times greater than the first dose.

167. The composition of claim 166, wherein the administration frequency of the second dose comprises the same administration frequency of the first dose.

168. The composition of claim 166, wherein the administration frequency of the second dose consists of the same administration frequency of the first dose.

169. The composition of claim 167 or 168, wherein the first dose and the second dose are each administered about once every four weeks.

170. The composition of claim 159, wherein the second dose is about the same dose as the first dose.

171. The composition of claim 159, wherein the second dose is the same dose as the first dose.

172. The composition of claim 170 or 171, wherein the first dose and the second dose are about 2 mg/kg.

173. The composition of claim 170 or 171, wherein the administration frequency of the second dose is about two times greater than the administration frequency of the first dose.

174. The composition of claim 172 or 173, wherein the first dose is administered about once every four weeks and the second dose is administered about once every two weeks.

175. A composition comprising an anti-TNFα antibody sialylated with one or more N-acetylneuraminic acid molecules for use in a method of treating or reducing sarcoidosis in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.

176. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time.

177. The composition of claim 175 or 176, wherein the first period of time is about 6 weeks to about 12 weeks.

178. The composition of claim 177, wherein the first period of time is about 8 weeks.

179. The composition of claim 177, wherein the first period of time is about 12 weeks.

180. The composition of any one of claims 175-179, wherein the second period of time is about 6 weeks to about 12 weeks.

181. The composition of claim 180, wherein the second period of time is about 8 weeks.

182. The composition of claim 180, wherein the second period of time is about 12 weeks.

183. A composition comprising an anti-TNFα antibody sialylated with one or more N-acetylneuraminic acid molecules for use in a method of treating or reducing sarcoidosis in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time,
wherein the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

184. A composition comprising an anti-TNFα antibody sialylated with one or more N-acetylneuraminic acid molecules for use in a method of treating or reducing sarcoidosis in a subject in need thereof relative to a control, the method comprising administering to the subject a first dose and a second dose of the composition;
the first dose comprising about 2 mg/kg of the composition administered once every two weeks for a first period of time; and
the second dose comprising (i) about 2 mg/kg of the composition administered once every two weeks, or (iii) about 4 mg/kg of the composition administered once every 4 weeks, for a second period of time,
wherein the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

185. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time,
wherein the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

186. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every four weeks for a first period of time; and
the second dose comprising (i) about 2 mg/kg of the composition administered once every two weeks, or (iii) about 4 mg/kg of the composition administered once every 4 weeks, for a second period of time,
wherein the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

187. A composition comprising an anti-TNFα antibody sialylated with one or more N-acetylneuraminic acid molecules for use in a method of reducing granuloma size or granuloma formation in a subject in need thereof relative to a control, the method comprising administering to the subject a first dose and a second dose of the composition;
the first dose comprising about 2 mg/kg of the composition administered once every two weeks for a first period of time; and
the second dose comprising (i) about 2 mg/kg of the composition administered once every two weeks, or (iii) about 4 mg/kg of the composition administered once every 4 weeks, for a second period of time,
wherein the anti-TNFα antibody comprises:
(i) a first CDR having an amino acid sequence at least about 80%, 85%, 90%, 95%, 99%, or 100% identical to SEQ ID NO: 3;
(ii) a second CDR having an amino acid sequence at least about 80%, 85%, 90%, 95%, 99%, or 100% identical to SEQ ID NO: 4;
(iii) a third CDR having an amino acid sequence at least about 80%, 85%, 90%, 95%, 99%, or 100% identical to SEQ ID NO: 5;
(iv) a fourth CDR having an amino acid sequence at least about 80%, 85%, 90%, 95%, 99%, or 100% identical to SEQ ID NO: 6;
(v) a fifth CDR having an amino acid sequence at least about 80%, 85%, 90%, 95%, 99%, or 100% identical to SEQ ID NO: 7;
(vi) a sixth CDR having an amino acid sequence at least about 80%, 85%, 90%, 95%, 99%, or 100% identical to SEQ ID NO: 8;
(vii) a seventh CDR having an amino acid sequence at least about 80%, 85%, 90%, 95%, 99%, or 100% identical to SEQ ID NO: 10;
(viii) an eighth CDR having an amino acid sequence at least about 80%, 85%, 90%, 95%, 99%, or 100% identical to YA;
(ix) a ninth CDR having an amino acid sequence at least about 80%, 85%, 90%, 95%, 99%, or 100% identical to SEQ ID NO: 12;
(x) a tenth CDR having an amino acid sequence at least about 80%, 85%, 90%, 95%, 99%, or 100% identical to SEQ ID NO: 13;
(xi) an eleventh CDR having an amino acid sequence at least about 80%, 85%, 90%, 95%, 99%, or 100% identical to SEQ ID NO: 14; and
(xii) a twelfth CDR having an amino acid sequence at least about 80%, 85%, 90%, 95%, 99%, or 100% identical to SEQ ID NO: 15.

188. The composition of any one of claims 160-187, further comprising administering (c) a third dose of about 4 mg/kg of the composition about once every two weeks, for a third period of time.

189. The composition of claim 187, wherein the third period of time is at least about 24 weeks.

190. The composition of any one of claims 140-189, wherein the anti-TNFα antibody comprises a variable heavy (VH) domain having an amino acid sequence at least about 80%, 85%, 90%, 95%, 99%, or 100% identical to SEQ ID NO: 16.

191. The composition of any one of claims 140-189, wherein the anti-TNFα antibody comprises a variable light (VL) domain having an amino acid sequence at least about 80%, 85%, 90%, 95%, 99%, or 100% identical to SEQ ID NO: 9.

192. The composition of any one of claims 140-191, wherein the anti-TNFα antibody comprises an asparagine-linked glycosylation site within a constant Fc region on at least one of the heavy chains.

193. The composition of claim 192, wherein the anti-TNFα antibody comprises an asparagine-linked glycosylation site within the constant Fc region on both of the heavy chains.

194. The composition of c wherein the anti-TNFα antibody is sialylated at Asn 297.

195. The composition of any one of claims 140-194, wherein the anti-TNFα antibody comprises no Neu5Gc.

196. The composition of any one of claims 140-195, wherein the anti-TNFα antibody is an IgG antibody.

197. The composition of claim 196, wherein the IgG antibody is an IgG1 antibody.

198. The composition of claim 197, wherein the IgG1 antibody comprises a kappa isotype.

199. The composition of any one of claims 140-198, wherein the anti-TNFα antibody is a chimeric antibody.

200. The composition of any one of claims 140-199, wherein the anti-TNFα antibody comprises a C-terminal lysine on at least one of the heavy chains.

201. The composition of claim 200, wherein the anti-TNFα antibody comprises a C-terminal lysine on both of the heavy chains.

202. The composition of claim 200 or 201, wherein the C-terminal lysine is not clipped.

203. The composition of any one of claims 140-202, wherein the composition is administered by an infusion.

204. The composition of claim 203, wherein the infusion is an intravenous (IV) infusion.

205. The composition of claim 203 or 204, wherein the infusion is administered over a period of at least about 2 hours.

206. The composition of claim 203 or 204, wherein the infusion is administered over a period of at about 2 hours.

207. The composition of any one of claims 140-206, wherein the subject receives a cumulative dose of about 6 mg/kg of the composition during the first period of time.

208. The composition of any one of claims 140-206, wherein the subject receives a cumulative dose of about 8 mg/kg of the composition during the first period of time.

209. The composition of any one of claims 140-206, wherein the subject receives a cumulative dose of about 16 mg/kg of the composition during the first period of time.

210. The composition of any one of claims 181-209, wherein the subject receives a cumulative dose of about 10 mg/kg to about 16 mg/kg of the composition during the second period of time.

211. The composition of claim 210, wherein the subject receives a cumulative dose of about 10 mg/kg of the composition during the second period of time.

212. The composition of claim 210, wherein the subject receives a cumulative dose of about 12 mg/kg of the composition during the second period of time.

213. The composition of claim 210, wherein the subject receives a cumulative dose of about 14 mg/kg of the composition during the second period of time.

214. The composition of claim 210, wherein the subject receives a cumulative dose of about 16 mg/kg of the composition during the second period of time.

215. The composition of any one of claims 188-214, wherein the subject receives a cumulative dose of about 28 mg/kg of the composition.

216. The composition of any one of claims 140-215, wherein the subject is administered a cumulative dose of about 30 mg/kg to about 60 mg/kg of the composition.

217. The composition of claim 216, wherein the subject is administered a cumulative dose of about 46 mg/kg of the composition.

218. The composition of claim 216, wherein the subject is administered a cumulative dose of about 48 mg/kg of the composition.

219. The composition of claim 216, wherein the subject is administered a cumulative dose of about 50 mg/kg of the composition.

220. The composition of claim 216, wherein the subject is administered a cumulative dose of about 52 mg/kg of the composition.

221. The composition of any one of claims 140-220, wherein the subject is administered at least 3 doses of the composition.

222. The composition of any one of claims 140-221, wherein the subject is administered at least 4 doses of the composition.

223. The composition of any one of claims 140-222, wherein the subject is administered at least 6 doses of the composition.

224. The composition of any one of claims 140-223, wherein the subject is administered at least 7 doses of the composition.

225. The composition of claim 221, wherein the at least 3 doses are administered over a 4-week period of time.

226. The composition of claim 221, wherein the at least 3 doses are administered over an 8-week period of time.

227. The composition of claim 221, wherein the at least 3 doses are administered over a 12-week period of time.

228. The composition of claim 222, wherein the at least 4 doses are administered over a 9-week period of time.

229. The composition of claim 222, wherein the at least 4 doses are administered over a 10-week period of time.

230. The composition of claim 222, wherein the at least 4 doses are administered over an 11-week period of time.

231. The composition of claim 222, wherein the at least 4 doses are administered over a 12-week period of time.

232. The composition of claim 223, wherein the at least 6 doses are administered over a 10-week period of time.

233. The composition of claim 223, wherein the at least 6 doses are administered over a 13-week period of time.

234. The composition of claim 223, wherein the at least 6 doses are administered over a 16-week period of time.

235. The composition of claim 223, wherein the at least 6 doses are administered over a 17-week period of time.

236. The composition of claim 223, wherein the at least 6 doses are administered over a 20-week period of time.

237. The composition of claim 234, wherein the at least 7 doses are administered over a 12-week period of time.

238. The composition of claim 234, wherein the at least 7 doses are administered over a 15-week period of time.

239. The composition of claim 234, wherein the at least 7 doses are administered over a 21-week period of time.

240. The composition of claim 234, wherein the at least 7 doses are administered over a 24-week period of time.

241. The composition of claim 234, wherein the at least 7 doses are administered over a 28-week period of time.

242. The composition of any one of claims 140-241, wherein administration of the composition reduces a level of one or more transcripts selected from the group consisting of angiotensin-converting enzyme 2 (ACE2), interleukin 6 (IL6), interleukin 2 receptor (IL2R), tumor necrosis factor alpha (TNFα), C-Reactive Protein (CRP), interleukin 1B (IL1B), interleukin 12 (IL12), interleukin 18 (IL18), interferon gamma (IFNG), interleukin 8 (IL8), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JunB), mitogen-activated protein kinase (MKP1), TNF alpha induced protein 1 (TNFAIP1), TNF alpha induced protein 2 (TNFAIP2), TNF alpha induced protein 3 (TNFAIP3), syndecan 4 (SDC4), superoxide dismutase 2 (SOD2), cytochrome c oxidase subunit II (COX2), interleukin 32 (IL32), intercellular adhesion molecule 1 (ICAM-1), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), and vascular adhesion molecule 1 (VCAM-1), relative to a control.

243. The composition of claim 242, wherein the level of the one or more transcripts is reduced by at least about 10%, 20%, 30%, 40%, 50%, 60%, 70%, 80%, 90%, or 100% relative to the control.

244. The composition of any one of claims 140-243, wherein administration of the composition reduces a level of one or more proteins selected from the group consisting of angiotensin-converting enzyme 2 (ACE2), interleukin 6 (IL-6), interleukin 2 receptor (IL-2R), tumor necrosis factor alpha (TNF-α), C-Reactive Protein (CRP), interleukin 1B (IL-1B), interferon gamma (IFN-γ), interleukin 8 (IL-8), interleukin 12 (IL-12), interleukin 18 (IL-18), monocyte chemoattractant protein-1 (MCP-1), chemokine ligand 1 (CXCL1), chemokine ligand 2 (CXCL2), chemokine ligand 3 (CXCL3), early growth response 1 (EGR1), JunB proto-oncogene (JUNB), mitogen-activated protein kinase phosphatase 1 (MKP-1), TNF alpha-induced protein 1 (TNFAIP1), TNF alpha-induced protein 2 (TNFAIP2), TNF alpha-induced protein 3 (TNFAIP3), syndecan 4 (SDC4), superoxide dismutase 2 (SOD2), cytochrome c oxidase subunit II (COX2), interleukin 32 (IL-32), Krebs von den Lungen-6 (KL-6), C—X—C Motif Chemokine Ligand 10 (CXCL10), C—X—C Motif Chemokine Ligand 9 (CXCL9), Chitotriosidase 1 (CHIT1), secretoglobin family 1A member 1 (SCGB1A1), serum amyloid A1 (SAA1), intercellular adhesion molecule 1 (ICAM-1), and vascular adhesion molecule 1 (VCAM-1) relative to a control.

245. The composition of claim 244, wherein the level of the one or more proteins is reduced by at least about 10%, 20%, 30%, 40%, 50%, 60%, 70%, 80%, 90%, or 100% relative to the control.

246. The composition of any one of claims 140-245, wherein administration of the composition increases one or more of a Forced Vital Capacity (FVC) measurement, a Forced Expiratory Volume in 1 second (FEV1) measurement, a Leicester Cough Questionnaire (LCQ) score, a Short Form 36 Health Survey (SF-36) score, a King's Sarcoidosis Questionnaire (KSQ) score, a Steroid-toxicity scale (STS) score, or a 6-minute walk test (6MWT) distance relative to a control.

247. The composition of claim 246, wherein the FVC measurement is increased by at least about 10%, 20%, 30%, 40%, 50%, 60%, 70%, 80%, 90%, or 100% relative to the control.

248. The composition of claim 246, wherein the FEV1 measurement is increased by at least about 10%, 20%, 30%, 40%, 50%, 60%, 70%, 80%, 90%, or 100% relative to the control.

249. The composition of claim 246, wherein the LCQ score is increased by at least about 1, 2, 3, 4, 5, 6, 7, 8, 9, 10, 11, 12, 13, 14, 15, 16, 17, or 18 points relative to the control.

250. The composition of claim 246, wherein the SF-36 score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.

251. The composition of claim 246, wherein the KSQ score is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.

252. The composition of claim 246 or 251, wherein the KSQ score is a KSQ General Health Status Module or a KSQ lung score.

253. The composition of claim 246, wherein the STS score is increased by at least 1, 2, 3, 4, 5, or 6 points relative to the control.

254. The composition of claim 246, wherein the 6MWT distance is increased by at least 10 m, 20 m, 30 m, 40 m, 50 m, 60 m, 70 m, 80 m, 90 m, 100 m, 125 m, 150 m, 175 m, 200 m, 250 m, 300 m, 350 m, 400 m, 450 m, or 500 m relative to the control.

255. The composition of any one of claims 140-254, wherein administration of the composition reduces one or more of a Patient Global Assessment (PGA) score, a Fatigue Assessment Scale (FAS) score, Modified Medical Research Council (mMRC) Dyspnea Scale score, a Saint George's Respiratory Questionnaire (SGRQ) score, or a Borg's CR10 dyspnea score relative to a control.

256. The composition of claim 255, wherein the PGA score is reduced by at least about 1, 2, 3, 4, 5, 6, 7, 8, 9, or 10 points relative to the control.

257. The composition of claim 255, wherein the FAS score is reduced by at least about 1, 2, 3, or 4 points relative to a control.

258. The composition of claim 255, wherein the mMRC Scale score is reduced by at least about 1, 2, 3, or 4 grades relative to the control

259. The composition of claim 255, wherein the SGRQ score is reduced by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 95, 96, 97, 98, 99, or 100 points relative to the control.

260. The composition of claim 255, wherein the Borg's CR10 dyspnea score is reduced by at least about 1, 2, 3, 4, 5, 6, 7, 8, 9, or 10 points relative to the control.

261. The composition of any one of claims 140-260, wherein administration of the composition reduces a need for an anti-inflammatory medication relative to a control.

262. The composition of claim 261, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.

263. The composition of claim 262, wherein the corticosteroid is selected from the group consisting of prednisone, prednisolone, dexamethasone, hydrocortisone, methylprednisolone, betamethasone, cortisone, fludrocortisone, and triamcinolone.

264. The composition of any one of claims 261-263, wherein the reduced need for anti-inflammatory medication comprises a reduction in frequency of, dosage of, or duration of administration of the anti-inflammatory medication.

265. The composition of any one of claims 140-264, wherein administration of the composition reduces a granuloma formation or a granuloma size in the subject by about 1%, 2%, 3%, 4%, 5%, 10%, 15%, 20%, 25%, 30%, 40%, 50%, 60%, 70%, 80%, 90%, or 100%, relative to a control.

266. The composition of claim 265, wherein the granuloma is a skin granuloma, a lung granuloma, a lymph node granuloma, an ocular granuloma, a liver granuloma, a spleen granuloma, a cardiac granuloma, or a rheumatoid granuloma.

267. The composition of any one of claims 140-266, wherein administration of the composition reduces a calcitriol level in the subject relative to a control.

268. The composition of claim 267, wherein the calcitriol level is reduced by at least about 5%, 10%, 15%, 20%, 25%, 30%, 40%, 50%, 60%, 70%, 80%, 90%, or 100% relative to the control.

269. The composition of any one of claims 140-268, wherein administration of the composition reduces a neopterin level in the subject relative to a control.

270. The composition of claim 260, wherein the neopterin level is reduced by at least about 5%, 10%, 15%, 20%, 25%, 30%, 40%, 50%, 60%, 70%, 80%, 90%, or 100% relative to the control.

271. The composition of any one of claims 140-270, wherein administration of the composition reduces a lysozyme level in the subject relative to a control.

272. The composition of claim 271, wherein the lysozyme level is reduced by at least about 5%, 10%, 15%, 20%, 25%, 30%, 40%, 50%, 60%, 70%, 80%, 90%, or 100% relative to the control.

273. The composition of any one of claims 140-272, wherein administration of the composition reduces a soluble IL-2R (sIL-2R) level in the subject relative to a control.

274. The composition of claim 273, wherein the sIL-2R level is reduced by at least about 5%, 10%, 15%, 20%, 25%, 30%, 40%, 50%, 60%, 70%, 80%, 90%, or 100% relative to the control.

275. The composition of any one of claims 140-274, wherein the subject has one or more of acute sarcoidosis, chronic sarcoidosis, progressive sarcoidosis, pulmonary sarcoidosis, remitting sarcoidosis, refractory sarcoidosis, advanced sarcoidosis, ocular sarcoidosis, cutaneous sarcoidosis, or neurosarcoidosis.

276. The composition of any one of claims 140-275, wherein the subject has received an anti-inflammatory medication.

277. The composition of any one of claims 140-276, wherein the subject is receiving an anti-inflammation medication.

278. The composition of claim 276 or 277, wherein the anti-inflammatory medication is selected from the group consisting of a corticosteroid, methotrexate, azathioprine, pentoxifylline, thalidomide, leflunomide, mycophenolate, cyclophosphamide, chloroquine, repository corticotropin (RCI), hydroxychloroquine, efzofitimod, rituximab, adalimumab, golimumab, namilumab, and infliximab.

279. The composition of claim 278, wherein the corticosteroid is selected from the group consisting of prednisone, prednisolone, dexamethasone, hydrocortisone, methylprednisolone, betamethasone, cortisone, fludrocortisone, and triamcinolone.
